(19) United States
(12) Patent Application Publication
(10) Pub. No.: US 2024/0228506 A1
(43) Pub. Date: Jul. 11, 2024

(54) RIPK1 INHIBITORS AND METHODS OF USE

(71) Applicants: Erin F. DiMauro, Boston, MA (US); Xavier Fradera, Boston, MA (US); Peter H. Fuller, Boston, MA (US); Min Lu, Boston, MA (US); Joey L. Methot, Boston, MA (US); Matthew J. Mitcheltree, Boston, MA (US); Andrew J. Musacchio, Boston, MA (US); Phieng Siliphaivanh, Boston, MA (US); Jing Su, Kenilworth, NJ (US); Merck Sharp & Dohme LLC, Rahway, NJ (US)

(72) Inventors: Erin F. DiMauro, Cambridge, MA (US); Xavier Fradera, Boston, MA (US); Peter H. Fuller, Ashland, MA (US); Min Lu, Brookline, MA (US); Joey L. Methot, Westwood, MA (US); Matthew J. Mitcheltree, Jamaica Plain, MA (US); Andrew J. Musacchio, Westborough, MA (US); Phieng Siliphaivanh, Newton, MA (US); Jing Su, Scotch Plains, NJ (US)

(73) Assignee: Merck Sharp & Dohme LLC, Rahway, NJ (US)

(21) Appl. No.: 18/556,222

(22) PCT Filed: Apr. 21, 2022
(86) PCT No.: PCT/US2022/025667

§ 371 Date: Oct. 19, 2023

Related U.S. Application Data
(60) Provisional application No. 63/180,218, filed on Apr. 27, 2021.

(51) Int. Cl. C07D 491/18; A61K 31/439; A61K 31/4545; A61K 31/497; A61K 31/4985; A61K 31/501; A61K 31/506; A61K 31/517; A61K 31/519; A61K 31/529; C07D 471/08; C07D 519/00

(52) U.S. Cl. CPC C07D 491/18; A61K 31/439; A61K 31/4545; A61K 31/497; A61K 31/4985; A61K 31/501; A61K 31/506; A61K 31/517; A61K 31/519; A61K 31/529; C07D 471/08; C07D 519/00

(57) ABSTRACT

Described herein are compounds of Formula II, or a pharmaceutically acceptable salt thereof. The compounds of Formula I act as RIPK1 inhibitors and can be useful in preventing, treating or acting as a remedial agent for RIPK1-related diseases.

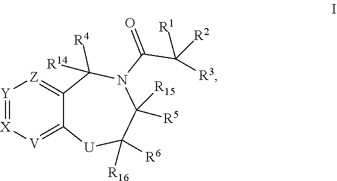

### FIELD OF THE INVENTION

The present invention is directed to RIPK1 inhibitors. Specifically, the RIPK1 inhibitors described herein can be useful in preventing, treating or acting as a remedial agent for RIPK1-related diseases.

### BACKGROUND OF THE INVENTION

Receptor-interacting protein-1 kinase (RIPK1) belongs to the family serine/threonine protein kinase involved in innate immune signaling. RIPK1 has emerged as a promising therapeutic target for the treatment of a wide range of human neurodegenerative, autoimmune, and inflammatory diseases. This is supported by extensive studies which have demonstrated that RIPK1 is a key mediator of apoptotic and necrotic cell death as well as inflammatory pathways.

For example, RIPK1 inhibition has been found to be useful as a treatment of acute kidney injury (AKI), a destructive clinical condition induced by multiple insults including ischemic reperfusion, nephrotoxic drugs and sepsis. It has been found that RIPK1-mediated necroptosis plays an important role in AKI and a RIPK1 inhibitor may serve as a promising clinical candidate for AKI treatment. Wang J N, Liu M M, Wang F, Wei B, Yang Q. Cai Y T, Chen X, Liu X Q, Jiang L, Li C, Hu X W, Yu J T, Ma T T, Jin J, Wu Y G, Li J, Meng X M, RIPK1 Inhibitor Cpd-71 Attenuates Renal Dysfunction in Cisplatin-Treated Mice via Attenuating Necroptosis, Inflammation and Oxidative Stress. Clin Sci (Lond). 2019 Jul. 25; 133(14):1609-1627.

Additionally, human genetic evidence has linked the dysregulation of RIPK1 to the pathogenesis of amyotrophic lateral sclerosis (ALS), Alzheimer's disease and multiple sclerosis as well as other inflammatory and neurodegenerative diseases. Alexei Degterev, Dimitry Ofengeim, and Junying Yuan, Targeting RIPK1 for the treatment of human diseases, PNAS, May 14, 2019, 116 (20), 9714-9722; Ito Y, Ofengeim D, Najafov A, Das S. Saberi S, Li Y, et al., RIPK1 mediates axonal degeneration by promoting inflammation and necroptosis in ALS, Science, 2016, 353.603-8; Caccamo A, Branca C, Piras I S, Ferreira E, Huentelman M J, Liang W S, et al., Necroptosis activation in Alzheimer's disease, Nat Neurosci, 2017, 20:1236-4; Ofengeim D, Ito Y, Najafov A, Zhang Y, Shan B, DeWitt J P, et al., Activation ofnecroptosis in multiple sclerosis, Cell Rep., 2015, 10:1836-49.

It also has been demonstrated that necroptosis is a delayed component of ischemic neuronal injury, thus RIPK1 inhibition may also play a promising role as a treatment for stroke. Degterev A, et al., Chemical inhibitor of nonapoptotic cell death with therapeutic potential for ischemic brain injury, Nat Chem Biol 2005, 1(2):112-119.

Therefore, there is a need for inhibitors of RIPK1 that offer high selectivity which can penetrate the blood-brain barrier, thus offering the possibility to target neuroinflammation and cell death which drive various neurologic conditions including Alzheimer's disease, ALS, and multiple sclerosis as well as acute neurological diseases such as stroke and traumatic brain injuries.

### SUMMARY OF THE INVENTION

Described herein are compounds of Formula I:

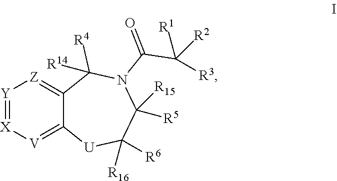

and pharmaceutically acceptable salts thereof, wherein U, V, X, Y, Z, R¹, R², R³, R⁴, R⁵, R⁶, R¹⁴, R¹⁵ and R¹⁶ are described below.

The compounds described herein are RIPK1 inhibitors, which can be useful in the prevention, treatment or amelioration of neurodegenerative, autoimmune, inflammatory diseases and other RIPK1-related diseases.

Also described herein are methods of treating neurodegenerative, autoimmune, and inflammatory diseases comprising administering to a patient in need thereof a compound described herein, or a pharmaceutically acceptable salt thereof

Also described herein are uses of a compound described herein, or a pharmaceutically acceptable salt thereof, to treat neurodegenerative, autoimmune, and inflammatory diseases in a patient in need thereof.

Also described herein are pharmaceutical compositions comprising a compound described herein, or a pharmaceutically acceptable salt thereof and a pharmaceutically acceptable carrier.

Also described herein are pharmaceutical compositions comprising a compound described herein and a pharmaceutically acceptable carrier.

Also described herein are methods of treating neurodegenerative, autoimmune, and inflammatory diseases comprising administering to a patient in need thereof a compound described herein, or a pharmaceutically acceptable salt thereof, and at least one additional therapeutic agent.

Also described herein are uses of a compound described herein, or a pharmaceutically acceptable salt thereof, in combination with at least one additional agent, to treat neurodegenerative, autoimmune, and inflammatory diseases in a patient in need thereof.

Also described herein are pharmaceutical compositions comprising a compound described herein, or a pharmaceutically acceptable salt thereof, at least one additional therapeutic agent and a pharmaceutically acceptable carrier.

Also described herein are pharmaceutical compositions comprising a compound described herein, at least one additional therapeutic agent and a pharmaceutically acceptable carrier.

### DETAILED DESCRIPTION OF THE INVENTION

Described herein are compounds of Formula I:

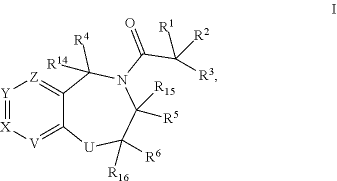

or a pharmaceutically acceptable salt thereof, wherein:
   U is O, S, NR¹¹ or CR¹²R¹³,
V is N or CR⁷;
   X is N or CR⁸;
   Y is N or CR⁹;
   Z is N or CR¹⁰; wherein at least one of V, X, Y or Z is N and wherein V, X, Y and Z are not simultaneously N;
   R¹ is hydrogen. OH, C₁-C₆alkylOH, CN, C₁-C₆alkylCN, C₁-C₆alkyl, C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl), haloalkoxy or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
   R² is hydrogen, OH, C₁-C₆alkylOH, CN, C₁-C₆alkylCN, C₁-C₆alkyl, C₃-C₆cycloalkyl, haloC₁-C₆alkyl, halogen. NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
   R³ is OH. C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen. CN, C₁-C₆alkyl, alkoxy, aryl, C₃-C₆cycloalkyl and haloC₁-C₆alkyl;
   R⁴ is hydrogen, C₁-C₆alkyl, or wherein when taken with R⁵ or R⁶ form a —CH₂—, —CH₂(CH₂)— or —CH₂CH₂— bridge;
   R⁵ is hydrogen, C₁-C₆alkyl, or wherein when taken with R⁴ form a —CH₂— or —CH₂CH₂-bridge;
   R⁶ is hydrogen, C₁-C₆alkyl, or wherein when taken with R⁴ form a —CH₂—, —CH₂(CH₂)— or —CH₂CH₂— bridge;
   R⁷ is hydrogen, OH. C₁-C₆alkylOH, CN, C₁-C₆alkylCN, C₁-C₆alkyl, C₁-C₆ alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, C₃-C₁₀cycloalkyl, NH₂, NHC₁-C₆alkyl, N(C₁-C₆alkyl)₂, haloC₁-C₆alkyl, halogen, alkoxy, haloalkoxy;
   R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
   R⁹ is hydrogen, OH, C₁-C₆alkylOH, CN, C₁-C₆alkylCN. C₁-C₆alkyl, C₁-C₆alkylaryl. C₂-C₆alkynyl, haloC₁-C₆alkyl, halogen, or alkoxy;
   R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
   R¹¹ is hydrogen, C₁-C₆alkyl, or C₃-C₆cycloalkyl;
   R¹² is hydrogen, OH, C₁-C₆alkylOH, CN, C₁-C₆alkylCN, C₁-C₆alkyl, haloC₁-C₆alkyl, halogen, or alkoxy;
   R¹³ is hydrogen, OH, C₁-C₆alkylOH, CN, C₁-C₆alkylCN. C₁-C₆alkyl, haloC₁-C₆alkyl, halogen, or alkoxy;
   R¹⁴ is hydrogen, OH, C₁-C₆alkylOH, CN, C₁-C₆alkylCN, C₁-C₆alkyl, haloC₁-C₆alkyl, halogen, or alkoxy;
   R¹⁵ is hydrogen, OH, C₁-C₆alkylOH, CN, C₁-C₆alkylCN, C₁-C₆alkyl, haloC₁-C₆alkyl, halogen, or alkoxy; and
   R¹⁶ is hydrogen, OH, C₁-C₆alkylOH, CN, C₁-C₆alkylCN, C₁-C₆alkyl, haloC₁-C₆alkyl, halogen, or alkoxy.

In one embodiment, disclosed herein are compounds of Formula I:

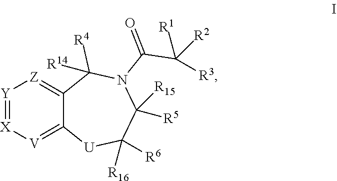

or a pharmaceutically acceptable salt thereof, wherein:
   U is O, S, NR¹¹ or CR¹²R¹³;
V is N or CR⁷;
   X is N or CR⁸;
   Y is N or CR⁹;
   Z is N or CR¹⁰; wherein at least one of V, X, Y or Z is N and wherein V, X, Y and Z are not simultaneously N;
   R¹ is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
   R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl;
   R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
   R⁴ is hydrogen, C₁-C₆alkyl, or wherein when taken with R³ or R⁶ form a —CH₂— or —CH₂CH₂— bridge;
   R⁵ is hydrogen. C₁-C₆alkyl, or wherein when taken with R⁴ form a —CH₂— or —CH₂CH₂-bridge;
   R⁶ is hydrogen, C₁-C₆alkyl, or wherein when taken with R⁴ form a —CH₂— or —CH₂CH₂-bridge;
   R⁷ is hydrogen, OH. C₁-C₆alkylOH, CN, C₁-C₆alkylCN, C₁-C₆alkyl, C₁-C₆ alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
   R⁸ is hydrogen, OH. C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
   R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
   R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
   R¹¹ is hydrogen, C₁-C₆alkyl, or C₃-C₆cycloalkyl;
   R¹² is hydrogen, OH, C₁-C₆alkylOH, CN, C₁-C₆alkylCN, C₁-C₆alkyl, haloC₁-C₆alkyl, halogen, or alkoxy;
   R¹³ is hydrogen, OH, C₁-C₆alkylOH, CN, C₁-C₆alkylCN, C₁-C₆alkyl, haloC₁-C₆alkyl, halogen, or alkoxy;
   R¹⁴ is hydrogen, OH, C₁-C₆alkylOH, CN, C₁-C₆alkylCN, C₁-C₆alkyl, haloC₁-C₆alkyl, halogen, or alkoxy;
   R¹⁵ is hydrogen, OH, C₁-C₆alkylOH, CN, C₁-C₆alkylCN, C₁-C₆alkyl, haloC₁-C₆alkyl, halogen, or alkoxy; and
   R¹⁶ is hydrogen, OH, C₁-C₆alkylOH, CN. C₁-C₆alkylCN, C₁-C₆alkyl, haloC₁-C₆alkyl, halogen, or alkoxy.

In regard to the compounds described herein, U is O, S, NR¹¹ or CR¹²R¹³. In certain embodiments, U is O. In other embodiments, U is S. In still other embodiments, U is NR¹¹. In still other embodiments. U is CR¹²R¹³.

When U is NR¹¹, R¹¹ is hydrogen, C₁-C₆alkyl, or C₃-C₆cycloalkyl. In certain embodiments, R¹¹ is hydrogen. In other embodiments R¹¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹¹ is methyl or ethyl. In other embodiments, R¹¹ is C₃-C₆cycloalkyl. Suitable cycloalkyls include, but are not limited to, cyclopropyl, cyclobutyl, cyclopentyl and cyclohexyl.

When U is CR¹²R¹³, R¹² is hydrogen, OH, C₁-C₆alkylOH, CN, C₁-C₆alkylCN, C₁-C₆alkyl, haloC₁-C₆alkyl, halogen, or alkoxy. In certain embodiments, R¹² is hydrogen. In certain embodiments, R¹² is OH. In certain embodiments, R¹² is C₁-C₆alkylOH. Suitable alcohols include, but are not limited to, methanol, ethanol, propanol and butanol.

In certain embodiments, R¹² is CN. In certain embodiments, R¹² is C₁-C₆alkylCN. In certain embodiments, R¹² is

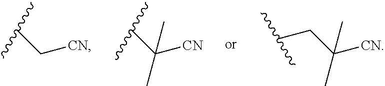

In certain embodiments, R¹² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹² is methyl or ethyl.

In certain embodiments, R¹² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹² is difluoromethyl. In certain embodiments, R¹² is trifluoromethyl. In certain embodiments, R¹² is difluoromethyl or trifluoromethyl. In certain embodiments, R¹² is halogen. Suitable halogens include, but are not limited to, fluorine, chlorine, bromine or iodine. In certain embodiments, R¹² is fluorine or chlorine. In certain embodiments, R¹² is alkoxy. Suitable alkoxys include, but are not limited to, methoxy, ethoxy, n-propoxy, isopropoxy and n-butoxy. In certain embodiments. R¹² is methoxy.

When U is CR¹²R¹³, R¹³ is hydrogen, OH, C₁-C₆alkylOH, CN, C₁-C₆alkylCN, C₁-C₆alkyl, haloC₁-C₆alkyl, halogen, or alkoxy. In certain embodiments, R¹¹ is hydrogen. In certain embodiments, R¹¹ is OH. In certain embodiments, R¹³ is C₁-C₆alkylOH. Suitable alcohols include, but are not limited to, methanol, ethanol, propanol and butanol.

In certain embodiments, R¹³ is CN. In certain embodiments, R¹¹ is C₁-C₆alkylCN. In certain embodiments, R¹³ is

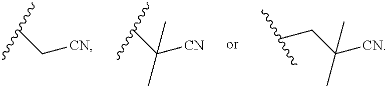

In certain embodiments, R¹³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, I-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹³ is methyl or ethyl.

In certain embodiments, R¹³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments. R¹³ is difluoromethyl. In certain embodiments, R¹³ is trifluoromethyl. In certain embodiments, R¹³ is difluoromethyl or trifluoromethyl. In certain embodiments, R¹¹ is halogen. Suitable halogens include, but are not limited to, fluorine, chlorine, bromine or iodine. In certain embodiments, R¹³ is fluorine or chlorine. In certain embodiments, R¹³ is alkoxy. Suitable alkoxys include, but are not limited to, methoxy, ethoxy, n-propoxy, isopropoxy and n-butoxy. In certain embodiments. R¹¹ is methoxy.

In regard to the compounds described R¹ is hydrogen, OH, C₁-C₆alkylOH, CN, C₁-C₆alkylCN, C₁-C₆alkyl, C₂—C(alkenyl, C₂-C₆alkynyl, C₃-C₆cycloalkyl, haloC₁-C₆alkyl, halogen, NH₂, N(C₁-C₆alkyl)₂, NH(C₁-C₆alkyl), haloalkoxy or alkoxy, or wherein when R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl.

In certain embodiments, R¹ is hydrogen. In certain embodiments, R¹ is OH. In certain embodiments, R¹ is C₁-C₆alkylOH. Suitable alcohols include, but are not limited to, methanol, ethanol, propanol and butanol.

In certain embodiments, R¹ is CN. In certain embodiments, R¹ is C₁-C₆alkylCN. In certain embodiments, R¹ is

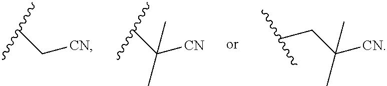

In certain embodiments, R¹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, l-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments. R¹ is methyl, ethyl and isopropyl. In certain embodiments, R¹ is C₃-C₆ cycloalkyl. Suitable cycloalkyls include, but are not limited to, cyclopropyl, cyclobutyl, cyclopentyl and cyclohexyl. In certain embodiments, R¹ is cyclopropyl. In certain embodiments, R¹ is cyclobutyl.

In certain embodiments, R¹ is C₂-C₆alkenyl. Suitable alkenyls include, but are not limited to, ethenyl, propenyl, butenyl, and hexenyl.

In certain embodiments, R¹ is C₂-C₆alkynyl. Suitable alkynyls include, but are not limited to, ethynyl, propynyl, butynyl, and hexynyl.

In certain embodiments, R¹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹ is difluoromethyl. In certain embodiments, R¹ is trifluoromethyl. In certain embodiments, R¹ is difluoromethyl or trifluoromethyl. In certain embodiments, R¹ is difluoroethyl. In certain embodiments, R¹ is fluoromethyl, difluoromethyl, trifluoromethyl, difluoroethyl, trifluoroethyl or difluoropropyl. In certain embodiments, R¹ is halogen. Suitable halogens include, but are not limited to, fluorine, chlorine, bromine or iodine. In certain embodiments, R¹ is fluorine or chlorine. In certain embodiments, R¹ is fluorine.

In certain embodiments, R¹ is NH₂. In certain embodiments. R¹ is N(C₁-C₆alkyl)₂. In certain embodiments, R¹ is N(CH₃)₂. In certain embodiments. R¹ is NH(C₁-C₆alkyl). In certain embodiments, R¹ is alkoxy. Suitable alkoxys include, but are not limited to, methoxy, ethoxy, n-propoxy, isopropoxy and n-butoxy. In certain embodiments. R¹ is methoxy.

In certain embodiments, R¹ is methyl, ethyl, isopropyl, cyclopropyl, cyclobutyl, difluoromethyl, fluoromethyl, fluorine, difluoroethyl, trifluoromethyl, methoxy, trifluoroethyl, difluoropropyl or CH₂CN.

In certain embodiments, R¹ is taken with R² and forms an oxo group.

In certain embodiments, R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy. CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² or R² and R³ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy. CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the C₃-C₁₀cycloalkyl is

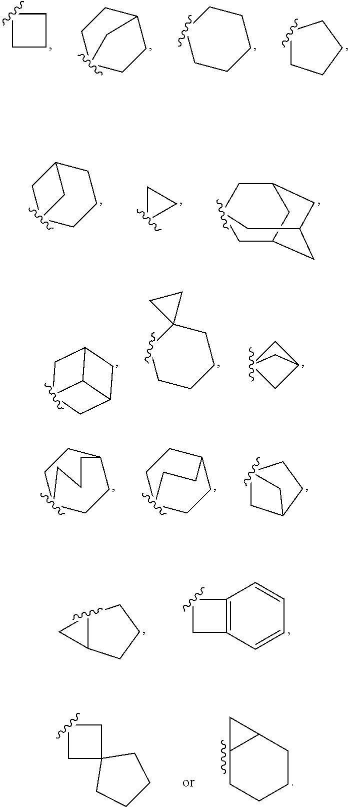

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with methyl. In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with difluoromethyl or trifluoromethyl.

In certain embodiments, the C₃-C₁₀cycloalkyl is substituted with halogen. Suitable halogens include, but are not limited to, fluorine, chlorine, bromine or iodine. In certain embodiments, the C₃-C₁₀cycloalkyl is substituted with fluorine.

In certain embodiments, the C₃-C₁₀cycloalkyl is substituted with COOC₁-C₆alkyl. In certain embodiments, the C₃-C₁₀cycloalkyl is substituted with

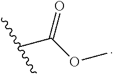 [SMILES: *C(=O)OC]

In certain embodiments, the C₃-C₁₀cycloalkyl is substituted with COaryl. In certain embodiments, the C₃-C₁₀cycloalkyl is substituted with

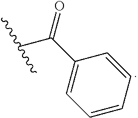 [SMILES: *C(=O)c1ccccc1]

In certain embodiments, the C₃-C₁₀cycloalkyl is substituted with alkoxy. In certain embodiments, the C₃-C₁₀cycloalkyl is substituted with methoxy. In certain embodiments, the C₃-C₁₀cycloalkyl is substituted with aryl. In certain embodiments, the C₃-C₁₀cycloalkyl is substituted with phenyl. In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₃-C₁₀cycloalkyl is substituted with heteroaryl, wherein the heteroaryl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with chlorine or fluorine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy.

In certain embodiments, the heteroaryl is substituted with methoxy. In certain embodiments, the heteroaryl is substituted with aryl. Suitable aryls include, but are not limited to, phenyl and naphthyl. In certain embodiments, the heteroaryl is substituted with phenyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with cyclopropyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² or R² and R³, and forms a C₃-C₁₀cycloalkyl, the C₃-C₁₀cycloalkyl is

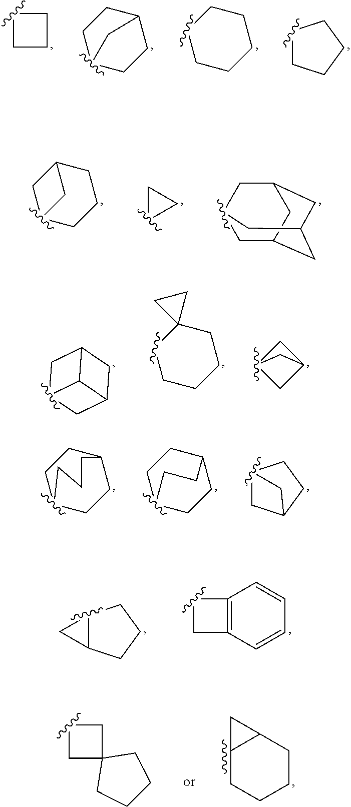

wherein the C₃-C₁₀cycloalkyl is unsubstituted or substituted with one to four substituents selected from the group halogen, C₁-C₆alkyl, haloC₁-C₆alkyl, alkoxy, CN, COOC₁-C₆alkyl.

In certain embodiments, R¹ is taken with R² and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

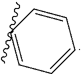

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with halogen. Suitable halogens include, but are not limited to, fluorine, chlorine, bromine or iodine.

In certain embodiments, the aryl is substituted with COOC₁-C₆alkyl. In certain embodiments, the aryl is substituted with

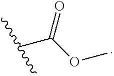 [SMILES: *C(=O)OC]

In certain embodiments, the aryl is substituted with COaryl. In certain embodiments, the aryl is substituted with

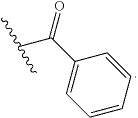 [SMILES: *C(=O)c1ccccc1]

In certain embodiments, the aryl is substituted with alkoxy. In certain embodiments, the aryl is substituted with methoxy. In certain embodiments, the aryl is substituted with aryl. In certain embodiments, the aryl is substituted with phenyl.

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN. C₁-C₆alkyl, alkoxy, aryl, C₃-C₆cycloalkyl and haloC₁-C₆alkyl.

In certain embodiments, the heteroaryl is pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl or isoquinolyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with halogen. Suitable halogens include, but are not limited to, fluorine, chlorine, bromine or iodine.

In certain embodiments, the heteroaryl is substituted with COOC₁-C₆alkyl. In certain embodiments, the heteroaryl is substituted with

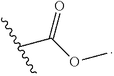 [SMILES: *C(=O)OC]

In certain embodiments, the heteroaryl is substituted with COaryl. In certain embodiments, the heteroaryl is substituted with

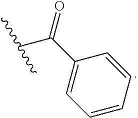 [SMILES: *C(=O)c1ccccc1]

In certain embodiments, the heteroaryl is substituted with alkoxy. In certain embodiments, the heteroaryl is substituted with methoxy. In certain embodiments, the heteroaryl is substituted with aryl. In certain embodiments, the heteroaryl is substituted with phenyl.

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy.

In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R¹ is taken with R² or R² and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R¹ is taken with R² and forms a heterocycloalkyl, wherein the heterocycloalkyl is unsubstituted or substituted with one to four substituents selected from the group consisting of halogen. C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen. CN, C₁-C₆alkyl, alkoxy, aryl, C₃-C₆cycloalkyl and haloC₁-C₆alkyl.

In certain embodiments, R¹ is taken with R² and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

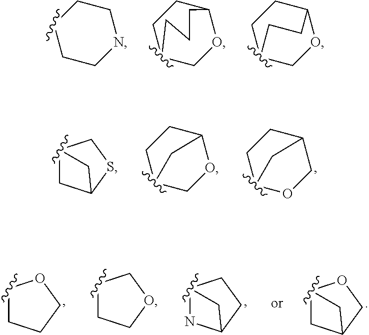

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, I-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with methyl.

In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with difluoromethyl or trifluoromethyl. In certain embodiments, the heterocycloalkyl is substituted with halogen. Suitable halogens include, but are not limited to, fluorine, chlorine, bromine or iodine. In certain embodiments, the heterocycloalkyl is substituted with fluorine.

In certain embodiments, the heterocycloalkyl is substituted with COOC₁-C₆alkyl. In certain embodiments, the heterocycloalkyl is substituted with

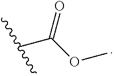 [SMILES: *C(=O)OC]

In certain embodiments, the heterocycloalkyl is substituted with COaryl. In certain embodiments, the heterocycloalkyl is substituted with

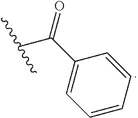 [SMILES: *C(=O)c1ccccc1]

In certain embodiments, the heterocycloalkyl is substituted with alkoxy. In certain embodiments, the heterocycloalkyl is substituted with methoxy. In certain embodiments, the heterocycloalkyl is substituted with aryl. In certain embodiments, the heterocycloalkyl is substituted with phenyl.

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heterocycloalkyl is substituted with heteroaryl, wherein the heteroaryl is pyridine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, I-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R¹ is taken with R² or R² and R³ and forms a heterocycloalkyl, the heterocycloalkyl is

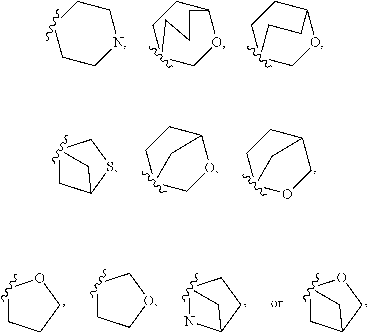

wherein the heterocycloalkyl is unsubstituted or substituted with one to four substituents selected from the group halogen, C₁-C₆alkyl, haloC₁-C₆alkyl, COaryl, and heteroaryl, and wherein the heteroaryl is unsubstituted or substituted with one to four substituents selected from the group halogen, CN, C₁-C₆alkyl and haloC₁-C₆alkyl.

In certain embodiments, when R¹ is taken with R² or R² and R³ and forms a heterocycloalkyl, the heterocycloalkyl is

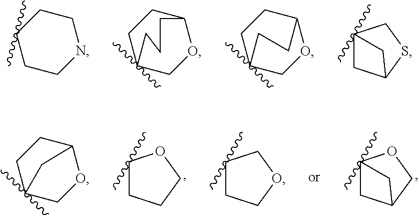

wherein the heterocycloalkyl is unsubstituted or substituted with one to four substituents selected from the group halogen, C₁-C₆alkyl, haloC₁-C₆alkyl, COaryl, and heteroaryl, and wherein the heteroaryl is unsubstituted or substituted with one to four substituents selected from the group halogen, CN, C₁-C₆alkyl and haloC₁-C₆alkyl.

In certain embodiments, wherein when the heterocycloalkyl is

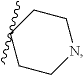

the heterocycloalkyl is substituted with heteroaryl, wherein the heteroaryl is

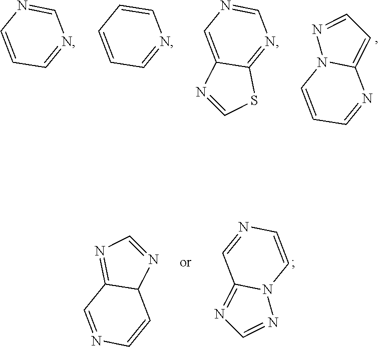

and wherein the heteroaryl is unsubstituted or substituted with CN.

With regard to the compounds described herein, R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein when R² is taken with R¹ and forms an oxo group or wherein when R² is taken with R¹ or R¹ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is hydrogen. In certain embodiments, R² is OH. In certain embodiments, R² is C₁-C₆alkylOH. Suitable alcohols include, but are not limited to, methanol, ethanol, propanol and butanol.

In certain embodiments, R² is CN. In certain embodiments, R² is C₁-C₆alkylCN. In certain embodiments, R² is

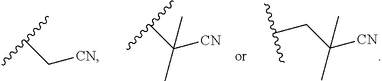

In certain embodiments, R² is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, I-ethyl-2-methylpropyl and 1-ethyl-1-methylpropyl. In certain embodiments, R² is methyl, ethyl or isopropyl. In certain embodiments, R² is C₃-C₆cycloalkyl. Suitable cycloalkyls include, but are not limited to, cyclopropyl, cyclobutyl, cyclopentyl and cyclohexyl. In certain embodiments, R² is cyclopropyl.

In certain embodiments, R² is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R² is difluoromethyl. In certain embodiments, R² is trifluoromethyl. In certain embodiments, R² is difluoromethyl or trifluoromethyl. In certain embodiments. R² is fluoromethyl, difluoromethyl, trifluoromethyl, difluoromethyl or difluoropropyl. In certain embodiments, R² is halogen. Suitable halogens include, but are not limited to, fluorine, chlorine, bromine or iodine. In certain embodiments, R² is fluorine or chlorine. In certain embodiments, R² is fluorine.

In certain embodiments, R² is NH₂. In certain embodiments, R² is N(C₁-C₆alkyl)₂. In certain embodiments, R² is NH(C₁-C₆alkyl). In certain embodiments, R² is alkoxy. Suitable alkoxys include, but are not limited to, methoxy, ethoxy, n-propoxy, isopropoxy and n-butoxy.

In certain embodiments, R¹ is methoxy.

In certain embodiments, R² is methyl, isopropyl, fluoromethyl, difluoromethyl, fluorine, difluoroethyl, trifluoromethyl, ethyl, methoxy, difluoropropyl or CH₂CN.

In certain embodiments, R² is taken with R¹ and forms an oxo group.

In certain embodiments, R² is taken with R′ or R′ and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN. C₁-C₆alkyl, alkoxy, aryl, C₃-C₆cycloalkyl and haloC₁-C₆alkyl.

In certain embodiments, R² is taken with R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen. CN, C₁-C₆alkyl, alkoxy, aryl, C₃-C₆cycloalkyl and haloC₁-C₆alkyl.

In certain embodiments, R² is taken with R′ or R′ and R³ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the C₃-C₁₀cycloalkyl is

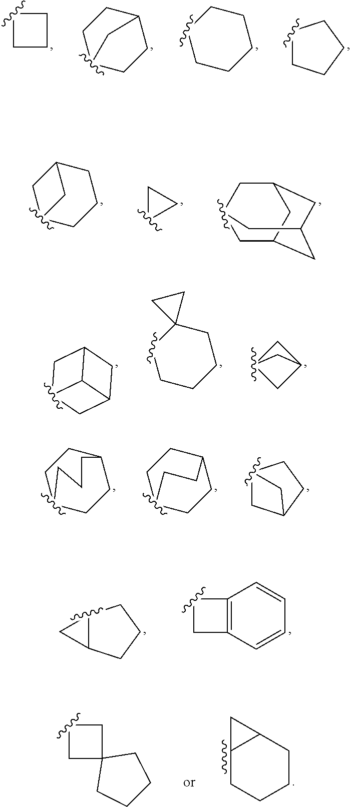

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with methyl. In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with difluoromethyl and trifluoromethyl. In certain embodiments, the C₃-C₁₀cycloalkyl is substituted with halogen. Suitable halogens include, but are not limited to, fluorine, chlorine, bromine or iodine. In certain embodiments, the C₃-C₁₀cycloalkyl is substituted with fluorine.

In certain embodiments, the C₃-C₁₀cycloalkyl is substituted with COOC₁-C₆alkyl. In certain embodiments, the C₃-C₁₀cycloalkyl is substituted with

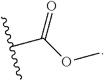 [SMILES: *C(=O)OC]

In certain embodiments, the C₃-C₁₀cycloalkyl is substituted with COaryl. In certain embodiments, the C₃-C₁₀cycloalkyl is substituted with

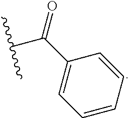 [SMILES: *C(=O)c1ccccc1]

In certain embodiments, the C₃-C₁₀cycloalkyl is substituted with alkoxy. In certain embodiments, the C₃-C₁₀cycloalkyl is substituted with methoxy. In certain embodiments, the C₃-C₁₀cycloalkyl is substituted with aryl. In certain embodiments, the C₃-C₁₀cycloalkyl is substituted with phenyl.

In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₃-C₁₀cycloalkyl is substituted with heteroaryl, wherein the heteroaryl is pyridine sub with C₁, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R² is taken with R′ or R′ and R³, the C₃-C₁₀cycloalkyl is

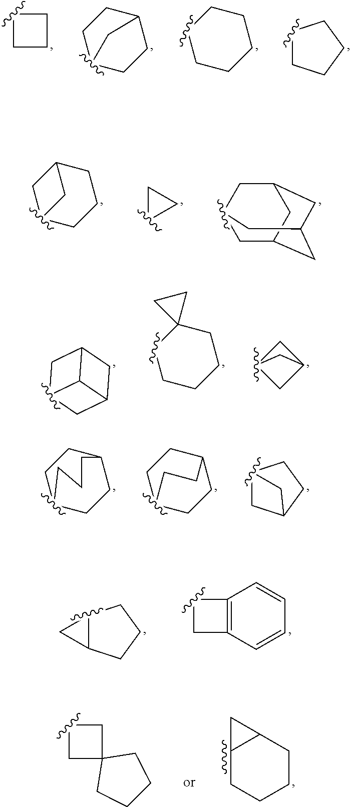

wherein the C₃-C₁₀cycloalkyl is unsubstituted or substituted with one to four substituents selected from the group halogen, C₁-C₆alkyl, haloC₁-C₆alkyl, alkoxy. CN, COOC₁-C₆alkyl.

In certain embodiments, R² is taken with R¹ and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

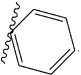

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with halogen. Suitable halogens include, but are not limited to, fluorine, chlorine, bromine or iodine.

In certain embodiments, the aryl is substituted with COOC₁-C₆alkyl. In certain embodiments, the aryl is substituted with

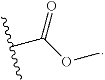 [SMILES: *C(=O)OC]

In certain embodiments, the aryl is substituted with COaryl. In certain embodiments, the aryl is substituted with

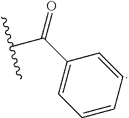 [SMILES: *C(=O)c1ccccc1]

In certain embodiments, the aryl is substituted with alkoxy. In certain embodiments, the aryl is substituted with methoxy. In certain embodiments, the aryl is substituted with aryl. In certain embodiments, the aryl is substituted with phenyl.

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is taken with R¹ and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heteroaryl is pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl or isoquinolyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with halogen. Suitable halogens include, but are not limited to, fluorine, chlorine, bromine or iodine.

In certain embodiments, the heteroaryl is substituted with COOC₁-C₆alkyl. In certain embodiments, the heteroaryl is substituted with

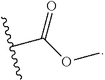 [SMILES: *C(=O)OC]

In certain embodiments, the heteroaryl is substituted with COaryl. In certain embodiments, the heteroaryl is substituted with

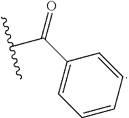 [SMILES: *C(=O)c1ccccc1]

In certain embodiments, the heteroaryl is substituted with alkoxy. In certain embodiments, the heteroaryl is substituted with methoxy. In certain embodiments, the heteroaryl is substituted with aryl. In certain embodiments, the heteroaryl is substituted with phenyl.

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with heteroaryl, wherein the heteroaryl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R² is taken with R′ or R′ and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R² is taken with R¹ and R³ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

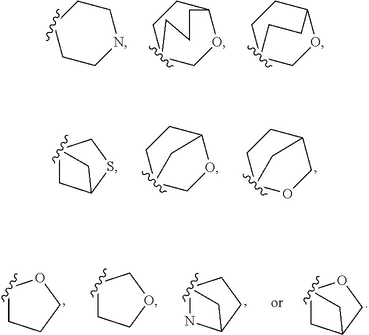

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with methyl. In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with difluoromethyl or trifluoromethyl. In certain embodiments, the heterocycloalkyl is substituted with halogen. Suitable halogens include, but are not limited to, fluorine, chlorine, bromine or iodine. In certain embodiments, the heterocycloalkyl is substituted with fluorine.

In certain embodiments, the heterocycloalkyl is substituted with COOC₁-C₆alkyl. In certain embodiments, the heterocycloalkyl is substituted with

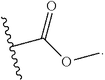 [SMILES: *C(=O)OC]

In certain embodiments, the heterocycloalkyl is substituted with COaryl. In certain embodiments, the heterocycloalkyl is substituted with

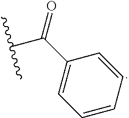 [SMILES: *C(=O)c1ccccc1]

In certain embodiments, the heterocycloalkyl is substituted with alkoxy. In certain embodiments, the heterocycloalkyl is substituted with methoxy. In certain embodiments, the heterocycloalkyl is substituted with aryl. In certain embodiments, the heterocycloalkyl is substituted with phenyl.

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heterocycloalkyl is pyridine, pyrazolopyrimidine, pyrimidinyl, thiazolopyrimidinyl or triazolopyrimidinyl.

In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, when R² is taken with R¹ or R¹ and R³ and forms a heterocycloalkyl, the heterocycloalkyl is

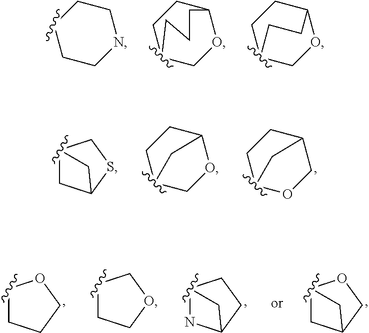

wherein the heterocycloalkyl is unsubstituted or substituted with one to four substituents selected from the group consisting of halogen, C₁-C₆alkyl, haloC₁-C₆alkyl, alkoxy, COaryl, and heteroaryl, wherein the heteroaryl is unsubstituted or substituted with one to four substituents selected from the group consisting of halogen. CN, C₁-C₆alkyl and haloC₁-C₆alkyl.

In certain embodiments, wherein when the heterocycloalkyl is

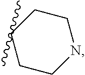

the heterocycloalkyl is substituted with heteroaryl, wherein the heteroaryl is

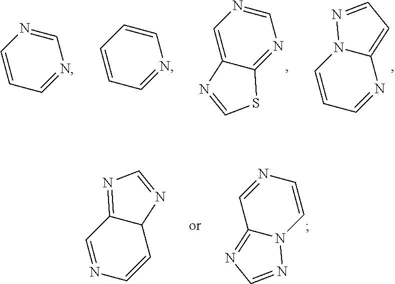

and wherein the heteroaryl is unsubstituted or substituted with CN.

With regard to the compounds described herein, R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkylalkyl, alkoxy, NHC₁-C₆alkylaryl, CONHC₁-C₆alkylaryl, aryl, or C₁-C₆alkylaryl, wherein the, NHC₁-C₆alkylaryl CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl.

In certain embodiments, R³ is OH. In certain embodiments, R³ is C₁-C₆alkylOH. Suitable alcohols include, but are not limited to, methanol, ethanol, propanol and butanol.

In certain embodiments, R³ is CN. In certain embodiments, R³ is C₁-C₆alkylCN. In certain embodiments, R³ is

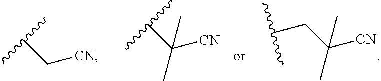

In certain embodiments, R³ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl. I-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments. R³ is C₃-C₆cycloalkyl. Suitable cycloalkyls include, but are not limited to, cyclopropyl, cyclobutyl, cyclopentyl and cyclohexyl.

In certain embodiments, R³ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments. R³ is difluoromethyl. In certain embodiments, R³ is difluoroethyl. In certain embodiments, R³ is trifluoromethyl. In certain embodiments, R¹ is difluoromethyl or trifluoromethyl. In certain embodiments, R³ is halogen. Suitable halogens include, but are not limited to, fluorine, chlorine, bromine or iodine. In certain embodiments, R³ is fluorine or chlorine. In certain embodiments, R³ is fluorine.

In certain embodiments, R³ is C₁-C₆alkylOhaloC₁-C₆alkyl. In certain embodiments, R³ is alkoxy. Suitable alkoxys include, but are not limited to, methoxy, ethoxy, n-propoxy, isopropoxy and n-butoxy. In certain embodiments, R³ is methoxy. In certain embodiments, R³ is CONHC₁-C₆alkylaryl. In certain embodiments, R³ is NHCH₂phenyl. In certain embodiments, R³ is CONHCH₂phenyl. In certain embodiments, R³ is aryl. Suitable aryls include, but are not limited to, phenyl and naphthyl. In certain embodiments, R³ is C₁-C₆alkylaryl. In certain embodiments, R³ is CH₂phenyl.

In certain embodiments, the NHC₁-C₆alkylaryl, CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl. In certain embodiments, the NHC₁-C₆alkylaryl, CONHC₁-C₆alkylaryl, aryl, or C₁-C₆alkylaryl is substituted with one substituent selected from the group consisting of halogen, C₁-C₆alkyl and haloC₁-C₆alkyl. In certain embodiments, the NHC₁-C₆alkylaryl, CONHC₁-C₆alkylaryl, aryl, or C₁-C₆alkylaryl is two substituents selected from the group consisting of halogen, C₁-C₆alkyl and haloC₁-C₆alkyl. In certain embodiments, the NHC₁-C₆alkylaryl, CONHC₁-C₆alkylaryl, aryl, or C₁-C₆alkylaryl is substituted with three substituents selected from the group consisting of halogen, C₁-C₆alkyl and haloC₁-C₆alkyl. In certain embodiments, the NHC₁-C₆alkylaryl, CONHC₁-C₆alkylaryl, aryl, or C₁-C₆alkylaryl is substituted with four substituents selected from the group consisting of halogen, C₁-C₆alkyl and haloC₁-C₆alkyl.

In certain embodiments, the NHC₁-C₆alkylaryl, CONHC₁-C₆alkylaryl, aryl, or C₁-C₆alkylaryl is substituted with halogen. Suitable halogens include, but are not limited to, fluorine, chlorine, bromine or iodine. In certain embodiments, the NHC₁-C₆alkylaryl, CONHC₁-C₆alkylaryl, aryl, or C₁-C₆alkylaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the NHC₁-C₆alkylaryl, CONHC₁-C₆alkylaryl NHC₁-C₆alkylaryl, CONHC₁-C₆alkylaryl, aryl, or C₁-C₆alkylaryl is substituted with haloC₁-C₆alkyl.

In certain embodiments, R³ is methyl, difluoromethyl, fluorine, difluoroethyl, trifluoromethyl or ethyl.

In certain embodiments, R³ is taken with R² or R² and R¹ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen. C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen. CN, C₁-C₆alkyl, alkoxy, aryl, C₃-C₆cycloalkyl and haloC₁-C₆alkyl.

In certain embodiments, R³ is taken with R² and R¹ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² or R² and R¹ and forms a C₃-C₁₀cycloalkyl, wherein the C₃-C₁₀cycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the C₃-C₁₀cycloalkyl is

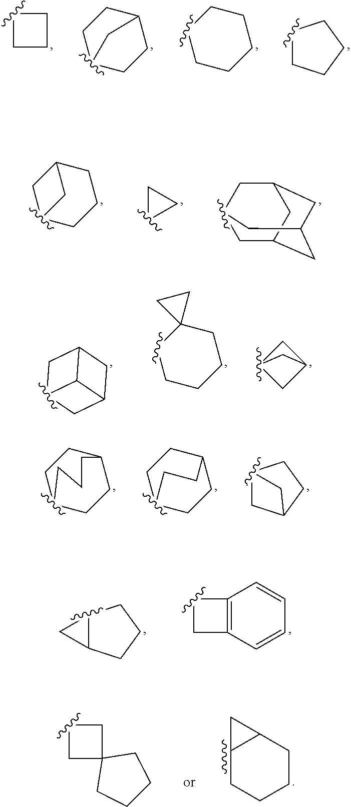

In certain embodiments, the C₃-C₁₀cycloalkyl is unsubstituted. In certain embodiments, the C₃-C₁₀cycloalkyl is substituted with one substituent. In certain embodiments, the C₃-C₁₀cycloalkyl is substituted with two substituents. In certain embodiments, the C₃-C₁₀cycloalkyl is substituted with three substituents. In certain embodiments, the C₃-C₁₀cycloalkyl is substituted with four substituents.

In certain embodiments, the C₃-C₁₀cycloalkyl is substituted with CN. In certain embodiments, the C₃-C₁₀cycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the C₃-C₁₀cycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₃-C₁₀cycloalkyl is substituted with halogen. Suitable halogens include, but are not limited to, fluorine, chlorine, bromine or iodine.

In certain embodiments, the C₃-C₁₀cycloalkyl is substituted with COOC₁-C₆alkyl. In certain embodiments, the C₃-C₁₀cycloalkyl is substituted with

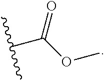 [SMILES: *C(=O)OC]

In certain embodiments, the C₃-C₁₀cycloalkyl is substituted with COaryl. In certain embodiments, the C₃-C₁₀cycloalkyl is substituted with

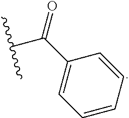 [SMILES: *C(=O)c1ccccc1]

In certain embodiments, the C₃-C₁₀cycloalkyl is substituted with alkoxy. In certain embodiments, the C₃-C₁₀cycloalkyl is substituted with methyl. In certain embodiments, the C₃-C₁₀cycloalkyl is substituted with aryl. In certain embodiments, the C₃-C₁₀cycloalkyl is substituted with phenyl.

In certain embodiments, the C₃-C₁₀cycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R¹ or R¹ and R² and forms a C₃-C₁₀cycloalkyl, the C₃-C₁₀cycloalkyl is

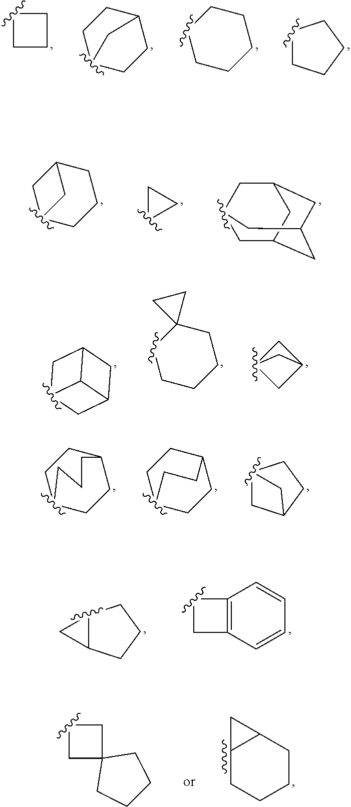

and wherein the C₃-C₁₀cycloalkyl is unsubstituted or substituted with one to four substituents selected from the group halogen, C₁-C₆alkyl, haloC₁-C₆alkyl, alkoxy. CN, COOC₁-C₆alkyl.

In certain embodiments, R³ is taken with R² and forms an aryl, wherein the aryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the aryl is

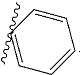

In certain embodiments, the aryl is unsubstituted. In certain embodiments, the aryl is substituted with one substituent. In certain embodiments, the aryl is substituted with two substituents. In certain embodiments, the aryl is substituted with three substituents. In certain embodiments, the aryl is substituted with four substituents.

In certain embodiments, the aryl is substituted with CN. In certain embodiments, the aryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the aryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the aryl is substituted with halogen. Suitable halogens include, but are not limited to, fluorine, chlorine, bromine or iodine.

In certain embodiments, the aryl is substituted with COOC₁-C₆alkyl. In certain embodiments, the aryl is substituted with

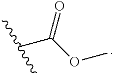 [SMILES: *C(=O)OC]

In certain embodiments, the aryl is substituted with COaryl. In certain embodiments, the aryl is substituted with alkoxy. In certain embodiments, the aryl is substituted with methoxy. In certain embodiments, the aryl is substituted with aryl. In certain embodiments, the aryl is substituted with phenyl.

In certain embodiments, the aryl is substituted with

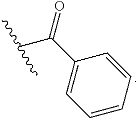 [SMILES: *C(=O)c1ccccc1]

In certain embodiments, the aryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² and forms a heteroaryl, wherein the heteroaryl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl.

In certain embodiments, the heteroaryl is pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl or isoquinolyl.

In certain embodiments, the heteroaryl is unsubstituted. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heteroaryl is substituted with halogen. Suitable halogens include, but are not limited to, fluorine, chlorine, bromine or iodine.

In certain embodiments, the heteroaryl is substituted with COOC₁-C₆alkyl. In certain embodiments, the heteroaryl is substituted with

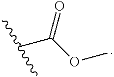 [SMILES: *C(=O)OC]

In certain embodiments, the heteroaryl is substituted with COaryl. In certain embodiments, the heteroaryl is substituted with

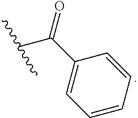 [SMILES: *C(=O)c1ccccc1]

In certain embodiments, the heteroaryl is substituted with alkoxy. In certain embodiments, the heteroaryl is substituted with methoxy. In certain embodiments, the heteroaryl is substituted with aryl. In certain embodiments, the heteroaryl is substituted with phenyl.

In certain embodiments, the heteroaryl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, I-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy. In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² or R² and R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, R³ is taken with R² and R¹ and forms a heterocycloalkyl, wherein the heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl.

In certain embodiments, the heterocycloalkyl is

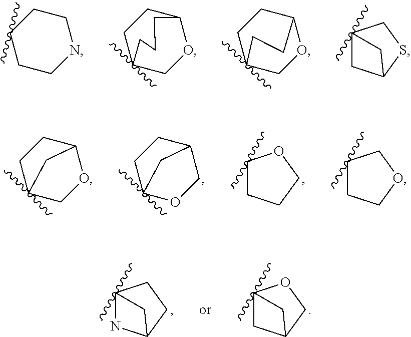

In certain embodiments, the heterocycloalkyl is unsubstituted. In certain embodiments, the heterocycloalkyl is substituted with one substituent. In certain embodiments, the heterocycloalkyl is substituted with two substituents. In certain embodiments, the heterocycloalkyl is substituted with three substituents. In certain embodiments, the heterocycloalkyl is substituted with four substituents.

In certain embodiments, the heterocycloalkyl is substituted with CN. In certain embodiments, the heterocycloalkyl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, the heterocycloalkyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the heterocycloalkyl is substituted with halogen. Suitable halogens include, but are not limited to, fluorine, chlorine, bromine or iodine.

In certain embodiments, the heterocycloalkyl is substituted with COOC₁-C₆alkyl. In certain embodiments, the heterocycloalkyl is substituted with

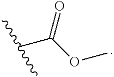 [SMILES: *C(=O)OC]

In certain embodiments, the heterocycloalkyl is substituted with COaryl. In certain embodiments, the heterocycloalkyl is substituted with

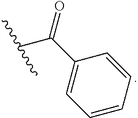 [SMILES: *C(=O)c1ccccc1]

In certain embodiments, the heterocycloalkyl is substituted with alkoxy. In certain embodiments, the heterocycloalkyl is substituted with methoxy. In certain embodiments, the heterocycloalkyl is substituted with aryl. In certain embodiments, the heterocycloalkyl is substituted with phenyl.

In certain embodiments, the heterocycloalkyl is substituted with heteroaryl, wherein the heteroaryl is unsubstituted or substituted. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the heteroaryl is substituted with one substituent. In certain embodiments, the heteroaryl is substituted with two substituents. In certain embodiments, the heteroaryl is substituted with three substituents. In certain embodiments, the heteroaryl is substituted with four substituents.

In certain embodiments, the heteroaryl is substituted with halogen. Suitable halogens include, but are not limited to, fluorine, chlorine, bromine or iodine. In certain embodiments, the heteroaryl is substituted with CN. In certain embodiments, the heteroaryl is substituted with C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, I-ethylbutyl, 1,1,2-trimethylpropyl, 1,2,2-trimethylpropyl, 1-ethyl-2-methylpropyl and 1-ethyl-1-methylpropyl. In certain embodiments, the heteroaryl is substituted with alkoxy. Suitable alkoxys include, but are not limited to, methoxy, ethoxy, n-propoxy, isopropoxy and n-butoxy.

In certain embodiments, the heteroaryl is substituted with aryl. Suitable aryls include, but are not limited to, phenyl and naphthyl. In certain embodiments, the heteroaryl is substituted with C₃-C₆cycloalkyl. Suitable examples of cycloalkyls include, but are not limited to, cyclopropyl, cyclobutyl, cyclopentyl and cyclohexyl. In certain embodiments, the heteroaryl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R³ is taken with R² or R² and R¹ and forms a heterocycloalkyl, wherein the heterocycloalkyl is

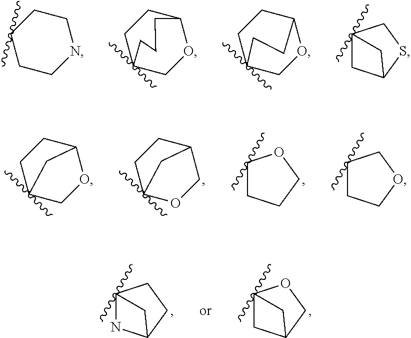

and wherein the heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, CN, C₁-C₆alkyl and haloC₁-C₆alkyl.

In certain embodiments, wherein when the heterocycloalkyl is

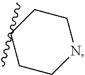

the heterocycloalkyl is substituted with heteroaryl, wherein the heteroaryl is

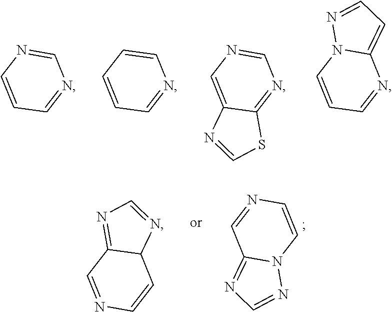

and wherein the heteroaryl is unsubstituted or substituted with CN.

In certain embodiments, R¹ and R² are independently selected from the group consisting of methyl, difluoromethyl, fluorine, difluoroethyl, trifluoromethyl, ethyl, N(CH₃)₂, CN and CH₂CN, and R³ is independently selected from the group consisting of methyl, difluoromethyl, fluorine, difluoroethyl, trifluoromethyl and ethyl.

In certain embodiments, R² is taken with R³ or R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl.

In certain embodiments, R³ is taken with R² or R¹ and R² and forms a C₃-C₁₀cycloalkyl or heterocycloalkyl, wherein the C₃-C₁₀cycloalkyl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group consisting of halogen, CN, C₁-C₆alkyl and haloC₁-C₆alkyl.

In certain embodiments, R³ is methyl, difluoromethyl, CH₂OCF₃, cyclobutyl, difluoroethyl, ethyl, cyclopropyl, CN, CH₂CN, CH₂phenyl, CONHCH₂phenyl, wherein the CH₂phenyl or CONHCH₂phenyl is substituted with one substituent selected from the group consisting of chlorine and methoxy.

In certain embodiments, R¹, R² and R³ are independently selected from the group consisting of methyl, ethyl, propyl, fluoromethyl, difluoromethyl, fluorine, difluoroethyl, trifluoromethyl, trifluoroethyl, difluoropropyl, methoxy, cyclopropyl cyclobutyl. CN and CH₂CN.

In certain embodiments, R² is taken with R³ or R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, alkoxy, CN, C₁-C₆alkyl, aryl, C₃-C₆cycloalkyl, and haloC₁-C₆alkyl.

In certain embodiments, R³ is taken with R² or R¹ and R² and forms a C₃-C₁₀cycloalkyl or heterocycloalkyl, wherein the C₃-C₁₀cycloalkyl or heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, alkoxy, CN. C₁-C₆alkyl, aryl, C₃-C₆cycloalkyl, and haloC₁-C₆alkyl.

In regard to the compounds described herein, R⁴ is hydrogen, C₁-C₆alkyl, or wherein when taken with R⁵ or R⁶ form a —CH₂— or —CH₂CH₂— bridge. In certain embodiments, R⁴ is hydrogen. In certain embodiments, R⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is methyl.

In certain embodiments, R⁴ is taken with R⁵ or R⁶ and forms a —CH₂— or —CH₂CH₂-bridge. In certain embodiments, R⁴ is taken with R⁵ and forms a —CH₂— bridge. In certain embodiments, R⁴ is taken with R⁵ and forms a —CH₂CH₂— bridge. In certain embodiments, R⁴ is taken with R⁶ and forms a —CH₂— bridge. In certain embodiments, R⁴ is taken with R⁶ and forms a —CH₂CH₂— bridge.

In regard to the compounds described herein, R⁵ is hydrogen, C₁-C₆alkyl, or wherein when taken with R⁴ form a —CH₂— or —CH₂CH₂— bridge. In certain embodiments, R⁵ is hydrogen. In certain embodiments. R is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl. In certain embodiments, R⁵ is taken with R⁴ and forms a —CH₂— bridge. In certain embodiments, R⁵ is taken with R⁴ and forms a —CH₂CH₂— bridge.

In regard to the compounds described herein, R⁶ is hydrogen, C₁-C₆alkyl, or wherein when taken with R⁴ form a —CH₂— or —CH₂CH₂— bridge. In certain embodiments, R⁶ is hydrogen. In certain embodiments. R⁶ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments. R⁶ is methyl. In certain embodiments, R⁶ is taken with R⁴ and forms a —CH₂— bridge. In certain embodiments, R⁶ is taken with R⁴ and forms a —CH₂CH₂— bridge.

Such embodiments, are shown below:

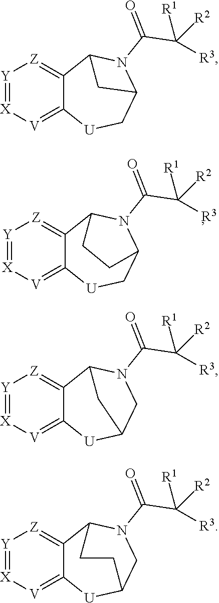

In certain embodiments, when U is O or CR¹²R¹³, R⁴ forms a —CH₂— or —CH₂CH₂— bridge with R⁵ or R⁶. In certain embodiments, when U is O or CR¹²R¹³, R⁴ forms a —CH₂— bridge with R⁵ or R⁶.

In certain embodiments, when U is O or CR¹²R¹³, R⁴ forms a —CH₂— bridge with R⁵.

In certain embodiments, when U is O or CR¹²R¹³, R⁴ forms a —CH₂— bridge with R⁶.

In regard to the compounds described herein, V is N or CR⁷. In certain embodiments, V is N. In certain embodiments, V is CR⁷.

When V is CR⁷, R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl. C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, C₃-C₁₀cycloalkyl, NH₂, NHC₁-C₆alkyl, N(C₁-C₆alkyl)₂, haloC₁-C₆alkyl, halogen, alkoxy, haloalkoxy.

In certain embodiments, R¹ is hydrogen. In certain embodiments, R¹ is OH. In certain embodiments, R⁷ is C₁-C₆alkylOH. Suitable alcohols include, but are not limited to, methanol, ethanol, propanol and butanol.

In certain embodiments, R⁷ is CN. In certain embodiments, R⁷ is C₁-C₆alkylCN. In certain embodiments, R⁷ is

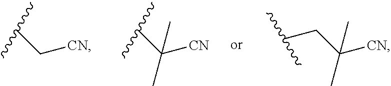

In certain embodiments, R⁷ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, I-ethyl-2-methylpropyl and 1-ethyl-1-methylpropyl. In certain embodiments, R⁷ is methyl or ethyl. In certain embodiments, R⁷ is C₁-C₆alkylaryl. In certain embodiments, R⁷ is

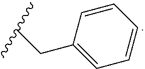 [SMILES: *Cc1ccccc1]

In certain embodiments, R⁷ is C₂-C₆alkynyl. Suitable alkynyls include, but are not limited to, ethynyl, propynyl, butynyl, and hexynyl. In certain embodiments, the C₂-C₆alkynyl is unsubstituted. In other embodiments, the C₂-C₆alkynyl is substituted.

In certain embodiments, the C₂-C₆alkynyl is substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with one substituent selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-Chalkyl, N(C₁-C₆alkyl)₂, haloC₁-C₆alkyl, halogen, alkoxy, haloalkoxy.

In certain embodiments, the C₂-C₆alkynyl is substituted with two substituents selected from the group consisting of OH, C₁-C₁₀alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl. C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl. C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

In certain embodiments, the C₂-C₆alkynyl is substituted with OH. In certain embodiments, the C₂-C₆alkynyl is substituted with C₁-C₆alkylOH. Suitable alcohols include, but are not limited to, methanol, ethanol, propanol and butanol. In certain embodiments, the C₂-C₆alkynyl is substituted with CN. In certain embodiments, the C₂-C₆alkynyl is substituted with C₁-C₆alkylCN. In certain embodiments, C₂-C₆alkynyl is substituted with

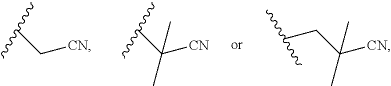

In certain embodiments, the C₂-C₆alkynyl is substituted with NH₂. In certain embodiments, the C₂-C₆alkynyl is substituted with NHC₁-C₆alkyl. In certain embodiments, the C₂-C₆alkynyl is substituted with N(C₁-C₆alkyl)₂. Suitable examples include but are not limited to N(CH₃)₂ and N(CH₂CH₃)₂.

In certain embodiments, the C₂-C₆alkynyl is substituted with haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, the C₂-C₆alkynyl is substituted with halogen. Suitable halogens include, but are not limited to, fluorine, chlorine, bromine or iodine. In certain embodiments, the C₂-C₆alkynyl is substituted with alkoxy. In certain embodiments, the C₂-C₆alkynyl is substituted with haloalkoxy. Suitable alkoxys include, but are not limited to, methoxy, ethoxy, n-propoxy, isopropoxy and n-butoxy.

In certain embodiments, the C₂-C₆alkynyl is substituted with aryl. Suitable alkoxys include, but are not limited to, phenyl and naphthyl. In certain embodiments, the C₂-C₆alkynyl is substituted with C₃-C₁₀cycloalkyl. Suitable examples of cycloalkyls include, but are not limited to, cyclopropyl, cyclobutyl, cyclopentyl and cyclohexyl. In certain embodiments, the C₂-C₆alkynyl is substituted with heteroaryl. Suitable heteroaryls include, but are not limited to, pyridyl (pyridinyl), oxazolyl, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, and isoquinolyl. In certain embodiments, the C₂-C₆alkynyl is substituted with heterocycloalkyl. Suitable examples of heterocycloalkyls include, but are not limited to, piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof.

In certain embodiments, when R⁷ is alkynyl and the alkynyl is substituted with aryl, C₃-C₁₀cycloalkyl, heteroaryl and heterocycloalkyl, the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are unsubstituted. In certain embodiments, the aryl, C₃-C₁₀cycloalkyl, heteroaryl and heterocycloalkyl are substituted with one substituent selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl. C₃-C₁₀cycloalkyl, heteroaryl and heterocycloalkyl are substituted with two substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are substituted with three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy. In certain embodiments, the aryl, C₃-C₁₀cycloalkyl, heteroaryl and heterocycloalkyl are unsubstituted or substituted with one to three substituents selected from the group consisting of OH, fluorine, trifluoromethyl, methyl, CN, NH₂, N(CH₃)₂, methoxy and trifluoromethoxy.

In certain embodiments, R⁷ is

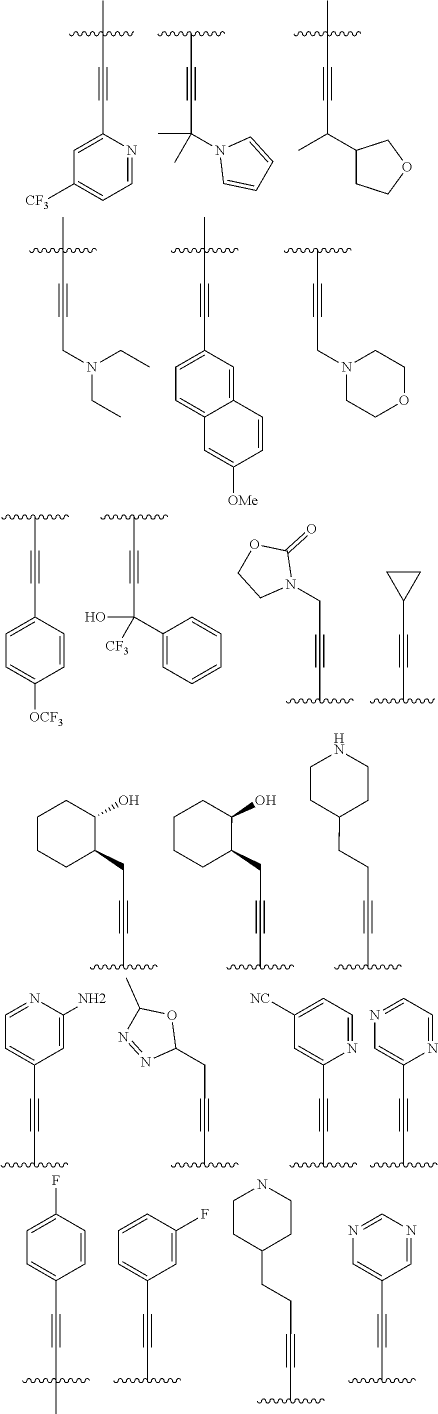

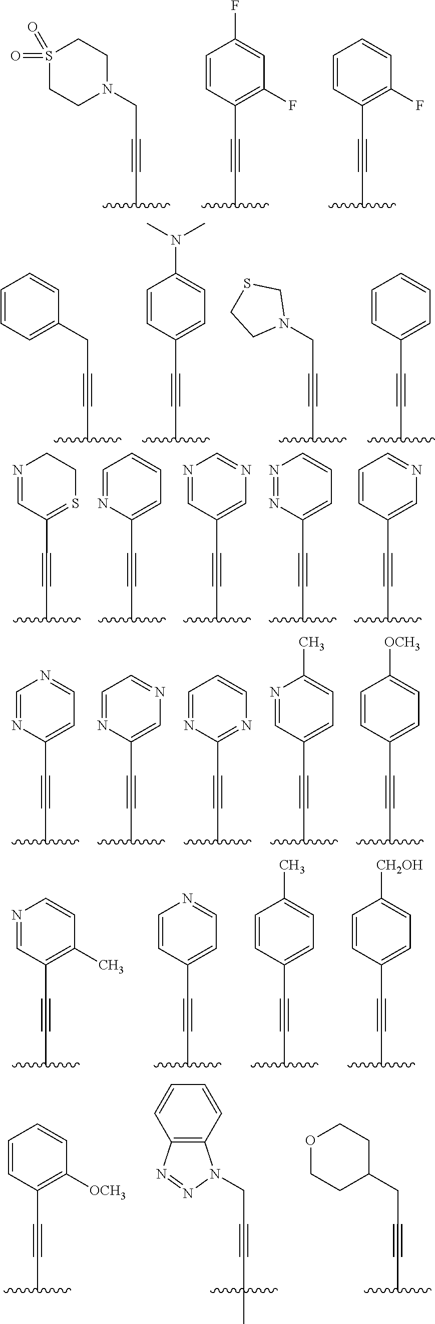

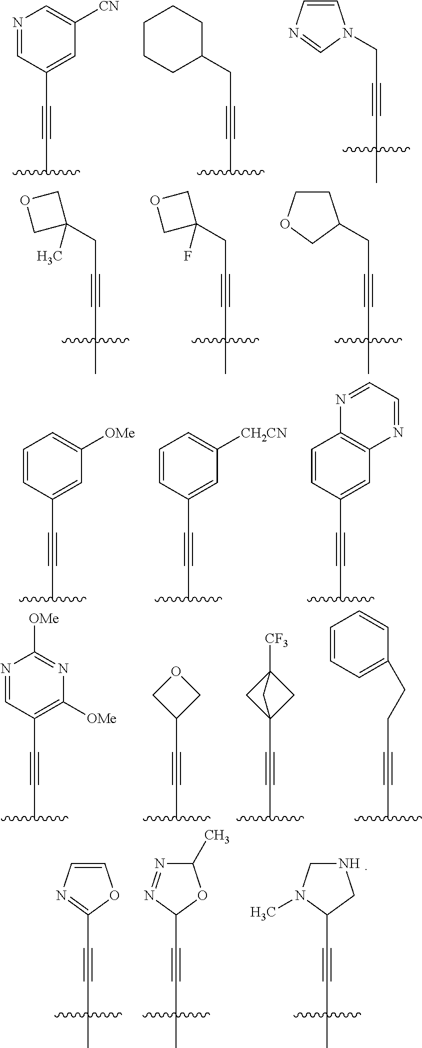

In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁷ is difluoromethyl. In certain embodiments, R⁷ is trifluoromethyl. In certain embodiments, R⁷ is difluoromethyl or trifluoromethyl. In certain embodiments, R⁷ is halogen. Suitable halogens include, but are not limited to, fluorine, chlorine, bromine or iodine. In certain embodiments, R⁷ is fluorine or chlorine. In certain embodiments, R⁷ is alkoxy. Suitable alkoxys include, but are not limited to, methoxy, ethoxy, n-propoxy, isopropoxy and n-butoxy. In certain embodiments, R⁷ is methoxy.

In certain embodiments, R⁷ is hydrogen, chlorine, CN, methyl, bromine, —C═CH₂, —C═C-phenyl, or fluorine.

In regard to the compounds described herein, X is N or CR⁸. In certain embodiments, X is N. In certain embodiments, X is CR⁸.

When X is CR⁸, R⁸ is hydrogen. OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments, R⁸ is hydrogen. In certain embodiments, R⁸ is OH. In certain embodiments, RN is C₁-C₆alkylOH. Suitable alcohols include, but are not limited to, methanol, ethanol, propanol and butanol.

In certain embodiments, R⁸ is CN. In certain embodiments, R⁵ is C₁-C₆alkylCN. In certain embodiments, R⁸ is

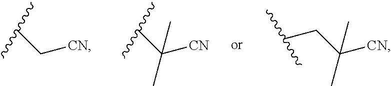

In certain embodiments, R⁸ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁸ is methyl or ethyl. In certain embodiments, R⁸ is C₁-C₆alkylaryl. In certain embodiments, R⁸ is

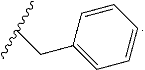 [SMILES: *Cc1ccccc1]

In certain embodiments, R⁸ is C₂-C₆alkynyl. Suitable alkynyls include, but are not limited to, ethynyl, propynyl, butynyl, and hexynyl.

In certain embodiments, R⁸ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁸ is difluoromethyl. In certain embodiments, R⁸ is trifluoromethyl. In certain embodiments, R⁸ is difluoromethyl or trifluoromethyl. In certain embodiments, RN is halogen. Suitable halogens include, but are not limited to, fluorine, chlorine, bromine or iodine. In certain embodiments, R⁸ is fluorine or chlorine. In certain embodiments, R⁸ is alkoxy. Suitable alkoxys include, but are not limited to, methoxy, ethoxy, n-propoxy, isopropoxy and n-butoxy. In certain embodiments. R⁸ is methoxy.

In certain embodiments, R⁸ is hydrogen or chlorine.

In regard to the compounds described herein, Y is N or CR⁹. In certain embodiments, Y is N. In certain embodiments, Y is CR⁹.

When Y is CR⁹, R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments, R⁹ is hydrogen. In certain embodiments, R⁹ is OH. In certain embodiments, R⁹ is C₁-C₆alkylOH. Suitable alcohols include, but are not limited to, methanol, ethanol, propanol and butanol.

In certain embodiments, R⁹ is CN. In certain embodiments, R⁹ is C₁-C₆alkylCN. In certain embodiments, R⁹ is

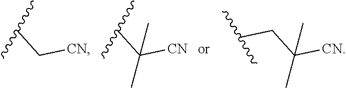

In certain embodiments, R⁹ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁹ is methyl or ethyl. In certain embodiments, R⁹ is C₁-C₆alkylaryl. In certain embodiments, R⁹ is

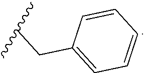 [SMILES: *Cc1ccccc1]

In certain embodiments, R⁹ is C₂-C₆alkynyl. Suitable alkynyls include, but are not limited to, ethynyl, propynyl, butynyl, and hexynyl.

In certain embodiments, R⁹ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁹ is difluoromethyl. In certain embodiments, R⁹ is trifluoromethyl. In certain embodiments, R⁹ is difluoromethyl or trifluoromethyl. In certain embodiments, R⁹ is halogen. Suitable halogens include, but are not limited to, fluorine, chlorine, bromine or iodine. In certain embodiments, R⁹ is fluorine or chlorine. In certain embodiments, R⁹ is alkoxy. Suitable alkoxys include, but are not limited to, methoxy, ethoxy, n-propoxy, isopropoxy and n-butoxy. In certain embodiments, R⁹ is methoxy.

In certain embodiments, R⁹ is hydrogen chlorine, CN, methyl or fluorine.

In regard to the compounds described herein, Z is N or CR¹⁰. In certain embodiments, Z is N. In certain embodiments, Z is CR¹⁰.

When Z is CR¹⁰, R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy. In certain embodiments. R¹⁰ is hydrogen. In certain embodiments, R¹⁰ is OH. In certain embodiments, R¹⁰ is C₁-C₆alkylOH. Suitable alcohols include, but are not limited to, methanol, ethanol, propanol and butanol.

In certain embodiments, R¹⁰ is CN. In certain embodiments, R¹⁰ is C₁-C₆alkylCN. In certain embodiments, R¹⁰ is

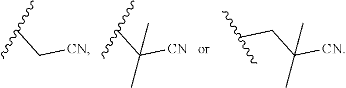

In certain embodiments, R¹⁰ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁰ is methyl or ethyl. In certain embodiments, R¹⁰ is C₁-C₆alkylaryl. In certain embodiments, R¹⁰ is

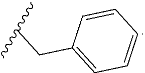 [SMILES: *Cc1ccccc1]

In certain embodiments, R¹⁰ is C₂-C₆alkynyl. Suitable alkynyls include, but are not limited to, ethynyl, propynyl, butynyl, and hexynyl.

In certain embodiments, R¹⁰ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R¹⁰ is difluoromethyl. In certain embodiments, R¹⁰ is trifluoromethyl. In certain embodiments, R¹⁰ is difluoromethyl or trifluoromethyl. In certain embodiments, R¹⁰ is halogen. Suitable halogens include, but are not limited to, fluorine, chlorine, bromine or iodine. In certain embodiments, R¹⁰ is fluorine or chlorine. In certain embodiments, R¹⁰ is alkoxy. Suitable alkoxys include, but are not limited to, methoxy, ethoxy, n-propoxy, isopropoxy and n-butoxy. In certain embodiments. R¹⁰ is methoxy.

With regard to the compounds described herein, at least one of V, X, Y or Z is N and wherein V, X, Y and Z are not simultaneously N. In certain embodiments, Z is N and V is CR⁷, X is CR⁸ and Y is CR⁹. In certain embodiments, Y is N and V is CR⁷, X is CR³ and Z is CR¹⁰. In certain embodiments, X is N and V is CR⁷, Z is CR¹⁰ and Y is CR⁹. In certain embodiments, V is N and Z is CR¹⁰, X is CR⁸ and Y is CR⁹. In certain embodiments. V and Y are N and Z is CR¹⁰ and X is CR⁸. In certain embodiments, Z and X are N and V is CR⁷ and Y is CR⁹. In certain embodiments, V is N, X is CH, Y is CH and Z is CR¹⁰. In certain embodiments, V is CH, X is CR⁸, Y is CH and Z is N. In certain embodiments, V is CR⁷, X is N, Y is CR⁹ and Z is N.

In certain embodiments, V is CR7, X is CR8, Y is N and Z is CH. In certain embodiments, V is N. X is CH, Y is CR9 and Z is CH. In certain embodiments, V is N. X is CR8, Y is N and Z is CH.

In regard to the compounds described herein, R¹⁴ is hydrogen, OH, C₁-C₆alkylOH, CN, C₁-C₆alkylCN, C₁-C₆alkyl, haloC₁-C₆alkyl, halogen, or alkoxy. In certain embodiments, R¹¹ is hydrogen. In certain embodiments, R¹⁴ is OH. In certain embodiments, R¹⁴ is C₁-C₆alkylOH. Suitable alcohols include, but are not limited to, methanol, ethanol, propanol and butanol.

In certain embodiments, R¹⁴ is CN. In certain embodiments, R¹¹ is C₁-C₆alkylCN. In certain embodiments, R¹¹ is

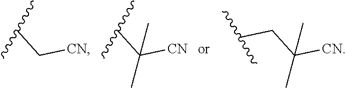

In certain embodiments, R¹⁴ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments. R⁴ is methyl or ethyl.

In certain embodiments, R¹⁴ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments. R¹⁴ is difluoromethyl. In certain embodiments, R¹⁴ is trifluoromethyl. In certain embodiments, R¹⁴ is difluoromethyl or trifluoromethyl. In certain embodiments, R¹⁴ is halogen. Suitable halogens include, but are not limited to, fluorine, chlorine, bromine or iodine. In certain embodiments, R¹⁴ is fluorine or chlorine. In certain embodiments, R⁴ is alkoxy. Suitable alkoxys include, but are not limited to, methoxy, ethoxy, n-propoxy, isopropoxy and n-butoxy. In certain embodiments, R¹⁴ is methoxy.

In regard to the compounds described herein, R¹⁵ is hydrogen, OH, C₁-C₆alkylOH, CN, C₁-C₆alkylCN, C₁-C₆alkyl, haloC₁-C₆alkyl, halogen, or alkoxy. In certain embodiments, R¹⁵ is hydrogen. In certain embodiments, R¹⁵ is OH. In certain embodiments, R¹⁵ is C₁-C₆alkylOH. Suitable alcohols include, but are not limited to, methanol, ethanol, propanol and butanol.

In certain embodiments, R¹⁵ is CN. In certain embodiments, R¹⁵ is C₁-C₆alkylCN. In certain embodiments, R¹⁵ is

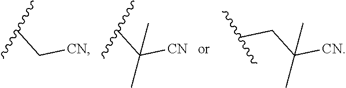

In certain embodiments, R¹⁵ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments. R¹⁵ is methyl or ethyl.

In certain embodiments, R¹⁵ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments. R¹⁵ is difluoromethyl. In certain embodiments, R¹⁵ is trifluoromethyl. In certain embodiments, R¹⁵ is difluoromethyl or trifluoromethyl. In certain embodiments, R¹⁵ is halogen. Suitable halogens include, but are not limited to, fluorine, chlorine, bromine or iodine. In certain embodiments, R¹⁵ is fluorine or chlorine. In certain embodiments, R¹⁵ is alkoxy. Suitable alkoxys include, but are not limited to, methoxy, ethoxy, n-propoxy, isopropoxy and n-butoxy. In certain embodiments, R¹⁵ is methoxy.

In regard to the compounds described herein, R¹⁶ is hydrogen. OH, C₁-C₆alkylOH, CN, C₁-C₆alkylCN, C₁-C₆alkyl, haloC₁-C₆alkyl, halogen, or alkoxy. In certain embodiments, R¹⁶ is hydrogen. In certain embodiments, R¹⁶ is OH. In certain embodiments, R¹⁶ is C₁-C₆alkylOH. Suitable alcohols include, but are not limited to, methanol, ethanol, propanol and butanol.

In certain embodiments, R¹⁶ is CN. In certain embodiments, R¹⁶ is C₁-C₆alkylCN. In certain embodiments, R¹⁶ is

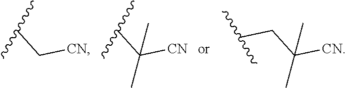

In certain embodiments, R¹⁶ is C₁-C₆alkyl. Suitable alkyls include, but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹⁶ is methyl or ethyl.

In certain embodiments, R¹⁶ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments. R¹⁶ is difluoromethyl. In certain embodiments, R¹⁶ is trifluoromethyl. In certain embodiments, R¹⁶ is difluoromethyl or trifluoromethyl. In certain embodiments, R¹⁶ is halogen. Suitable halogens include, but are not limited to, fluorine, chlorine, bromine or iodine. In certain embodiments, R¹⁶ is fluorine or chlorine. In certain embodiments, R¹⁶ is alkoxy. Suitable alkoxys include, but are not limited to, methoxy, ethoxy, n-propoxy, isopropoxy and n-butoxy. In certain embodiments, R¹⁶ is methoxy.

Also described herein are compounds of Formula II:

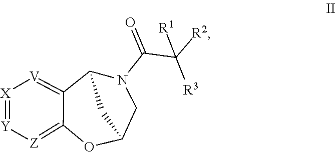

or a pharmaceutically acceptable salt thereof, wherein:
V is N or CR⁷;
X is N or CR⁸;
Y is N or CR⁹;
Z is N or CR¹⁰; wherein at least one of V, X, Y or Z is N and wherein V, X, Y and Z are not simultaneously N;
R¹ is hydrogen, OH, C₁-C₆alkylOH, CN, C₁-C₆alkylCN, C₁-C₆alkyl, C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl), haloalkoxy or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl,
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂. N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH. C₁-C₆alkylOH, CN, C₁-C₆alkylCN, C₁-C₆alkyl, C₃-C₁₀cycloalkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, alkynyl, alkynylaryl, haloC₁-C₆alkyl, halogen, or alkoxy;
R¹⁰ is hydrogen, OH, C₁-C₆alkylOH, CN, C₁-C₆alkylCN. C₁-C₆alkyl, C₁-C₆alkylaryl, alkynyl, alkynylaryl, haloC₁-C₆alkyl, halogen, or alkoxy.

Also described herein are compounds of Formula III:

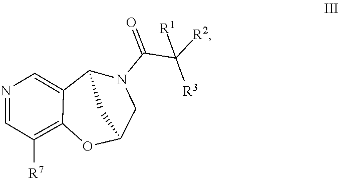

or a pharmaceutically acceptable salt thereof, wherein:
R¹ is hydrogen, OH, C₁-C₆alkylOH, CN, C₁-C₆alkylCN, C₁-C₆alkyl. C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl), haloalkoxy or alkoxy, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl. C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂. N(C₁-C₆alkyl)₂, NH(C₁-C₆alkyl) or alkoxy, or wherein R² is taken with R¹ and forms an oxo group, or wherein when R² is taken with R′ or R′ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₁₀alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN. C₁-C₆alkyl, C₃-C₆cycloalkyl, haloC₁-C₆alkyl, halogen. C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl; and
R⁷ is hydrogen. OH, C₁-C₆alkylOH, CN, C₁-C₆alkylCN. C₁-C₆alkyl, C₁-C₆alkylaryl. C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl. C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, C₃-C₁₀cycloalkyl, NH₂, NHC₁-C₆alkyl, N(C₁-C₆alkyl)₂, haloC₁-C₆alkyl, halogen, alkoxy, haloalkoxy.

Also, described herein are the following compounds:

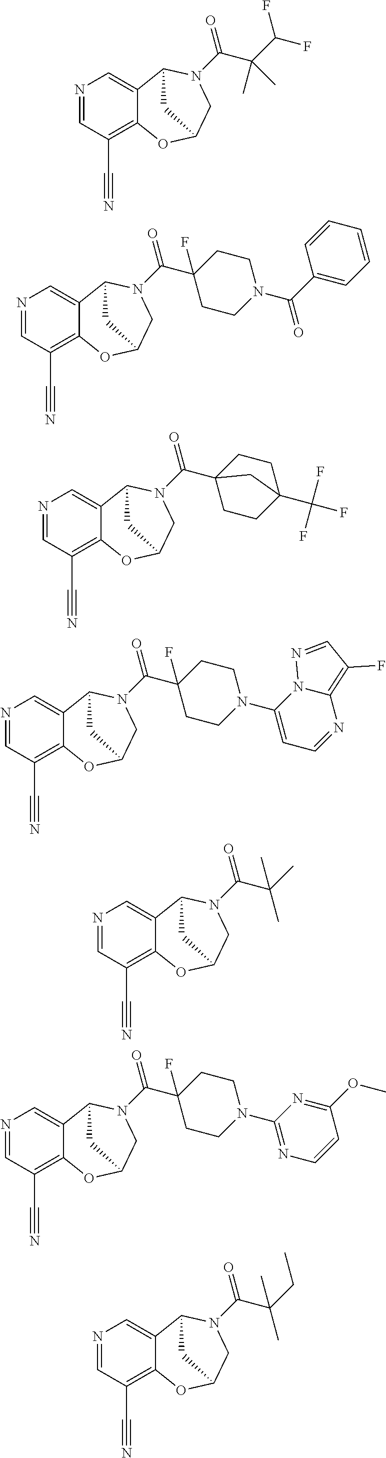

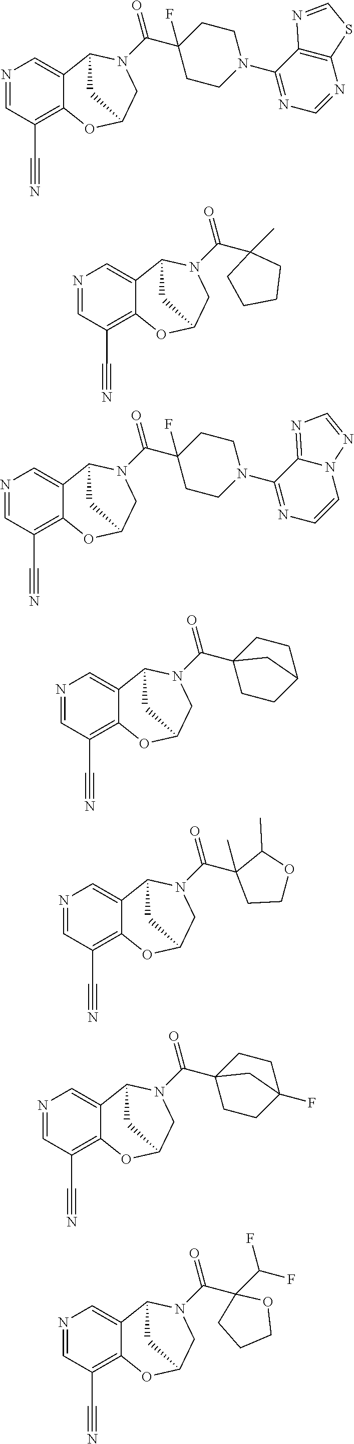

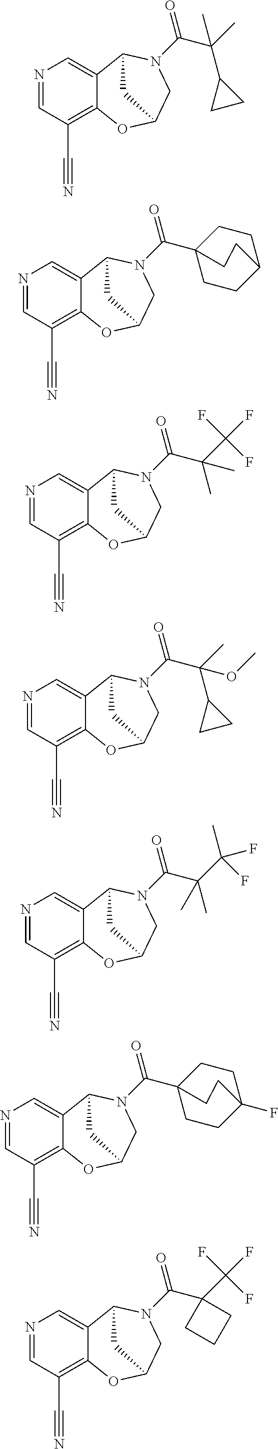

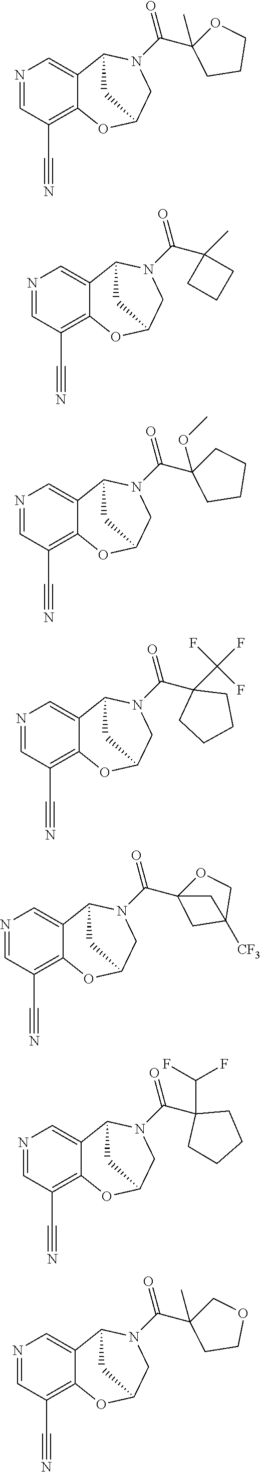

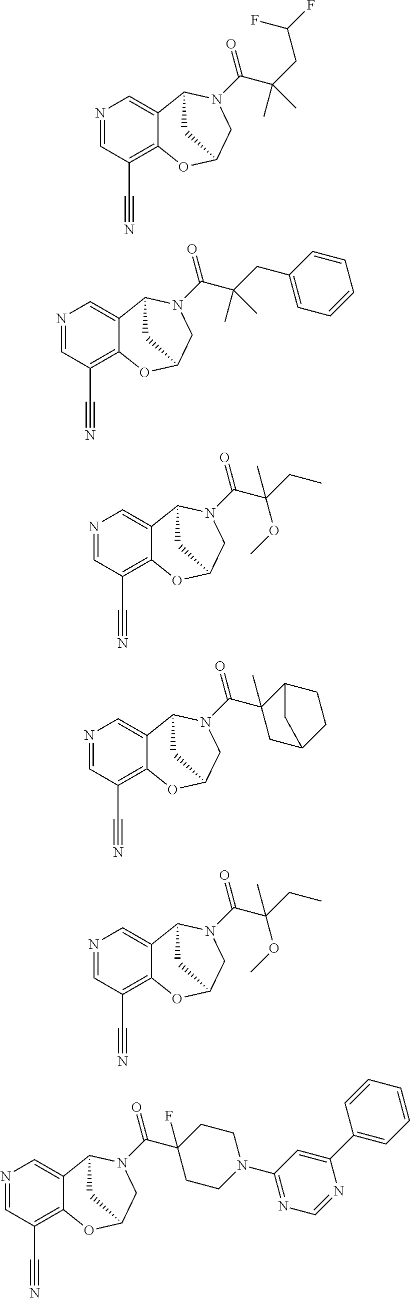 [SMILES: CC(C)(CC(F)F)C(=O)N1C[C@@H]2C[C@H]1c1cncc(C#N)c1O2 ; CC(C)(Cc1ccccc1)C(=O)N1C[C@@H]2C[C@H]1c1cncc(C#N)c1O2 ; CCC(C)(OC)C(=O)N1C[C@@H]2C[C@H]1c1cncc(C#N)c1O2 ; CC1(C(=O)N2C[C@@H]3C[C@H]2c2cncc(C#N)c2O3)CC2CCC1C2 ; CCC(C)(OC)C(=O)N1C[C@@H]2C[C@H]1c1cncc(C#N)c1O2 ; N#Cc1cncc2c1O[C@H]1C[C@@H]2N(C(=O)C2(F)CCN(c3cc(-c4ccccc4)ncn3)CC2)C1]

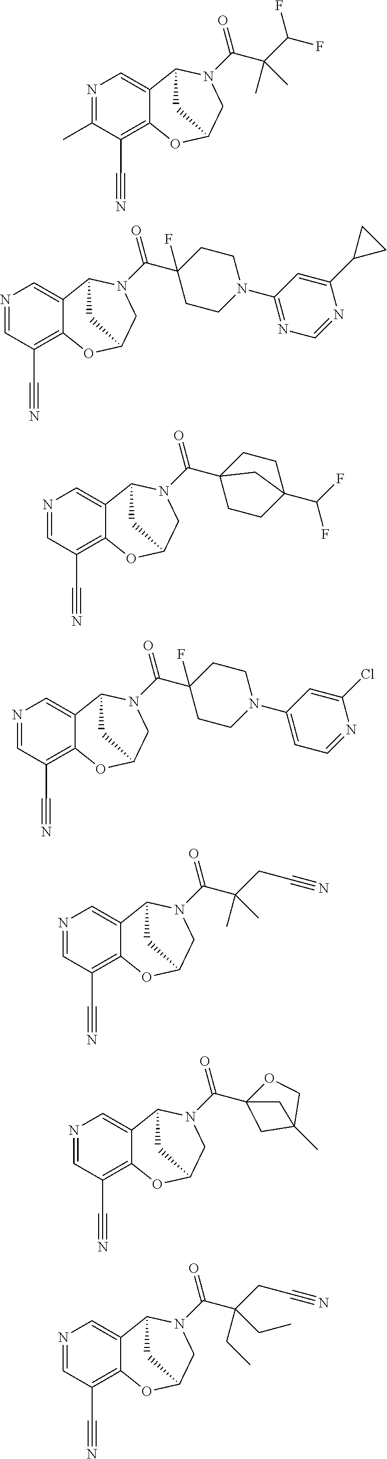

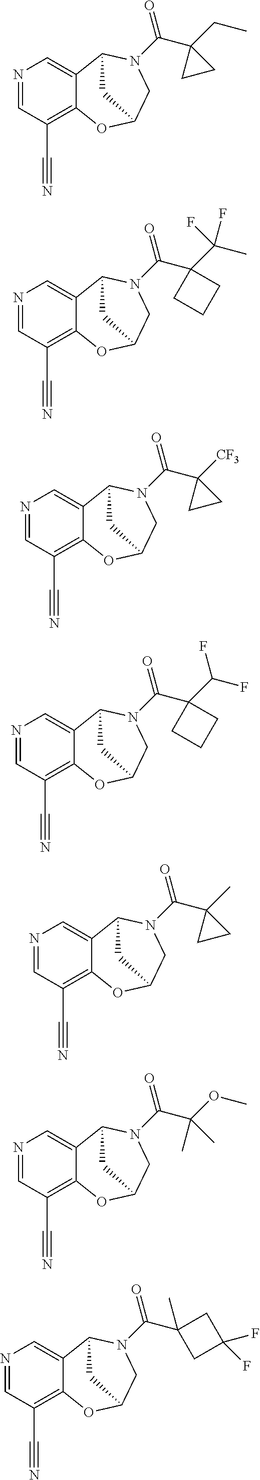

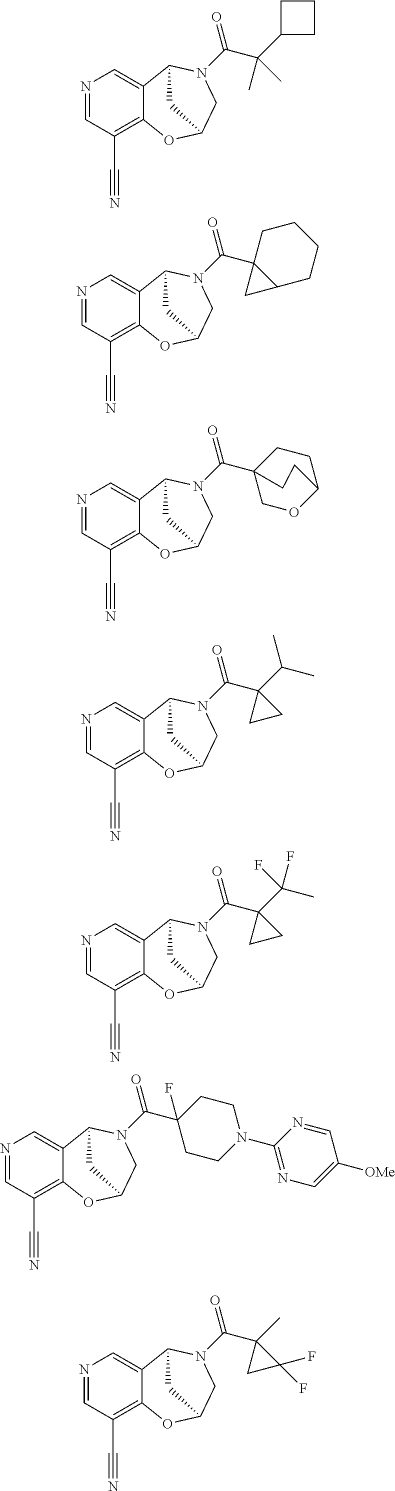

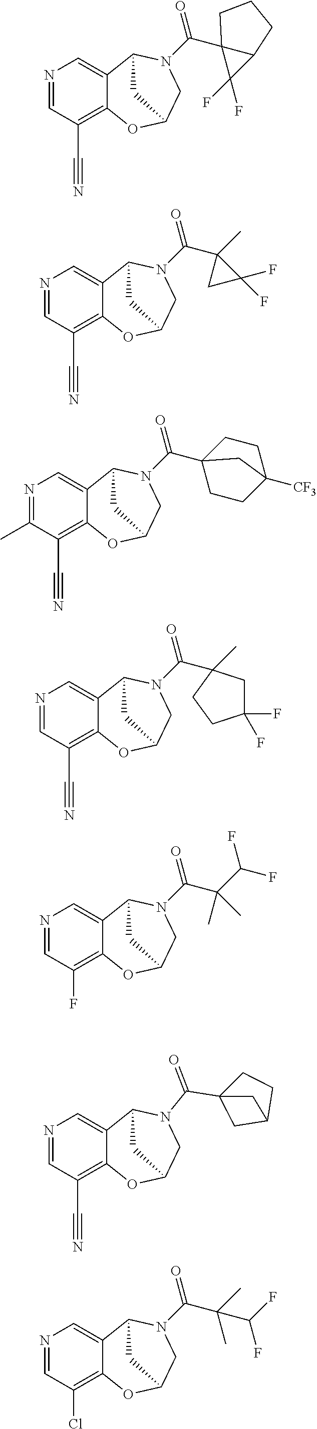

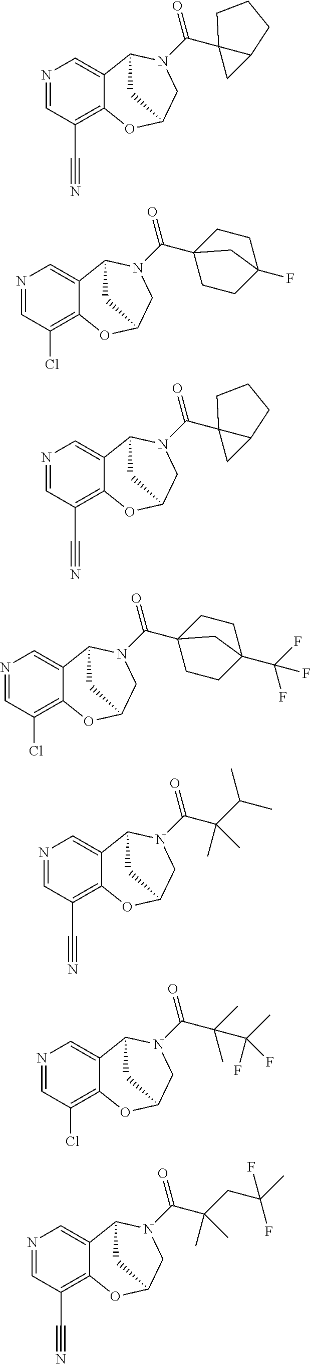

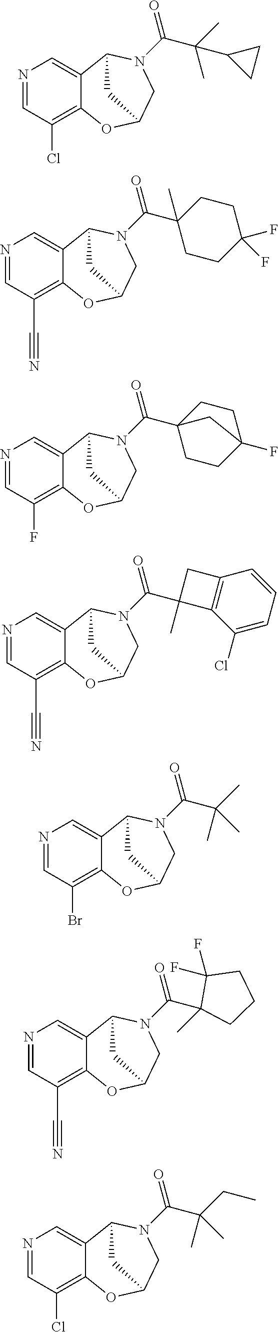

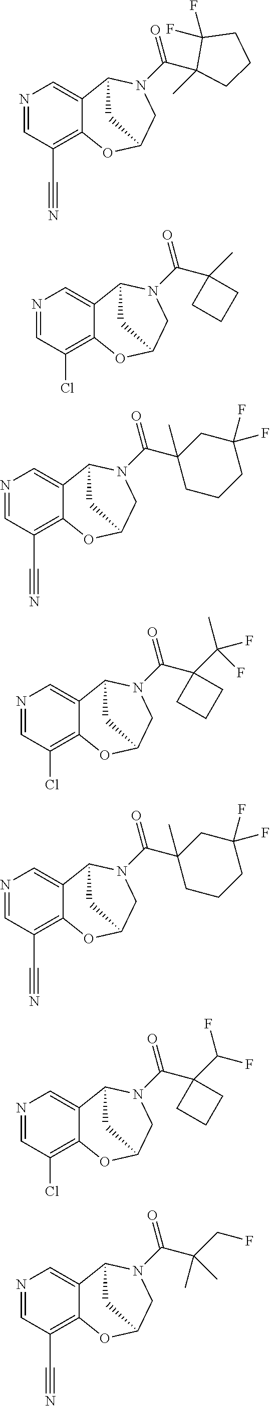

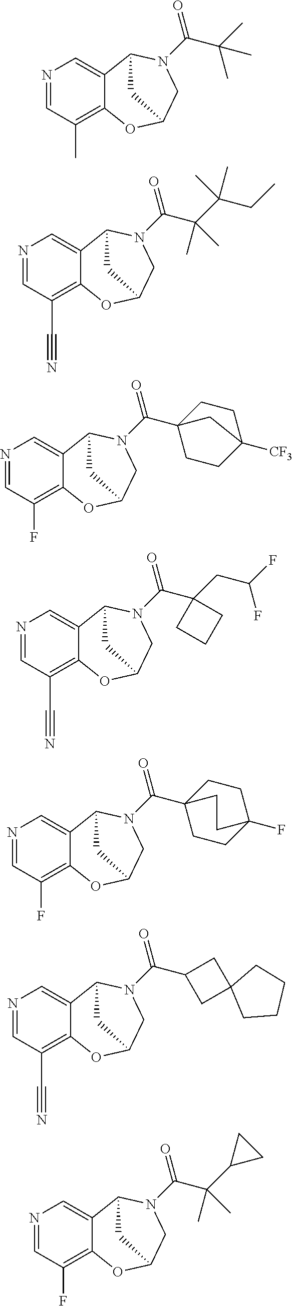

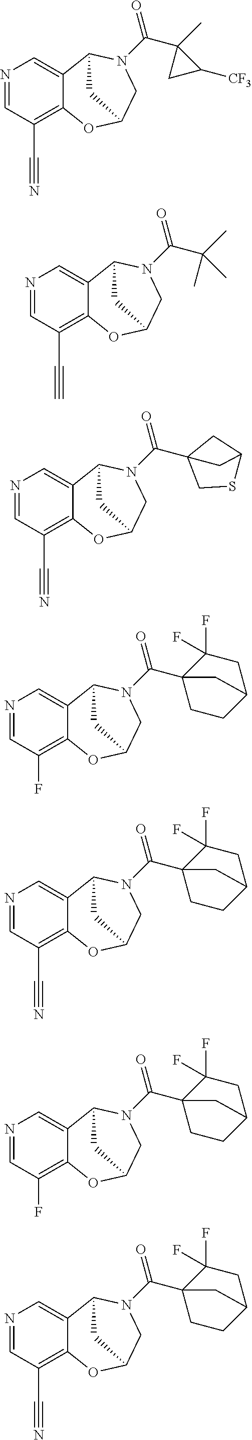

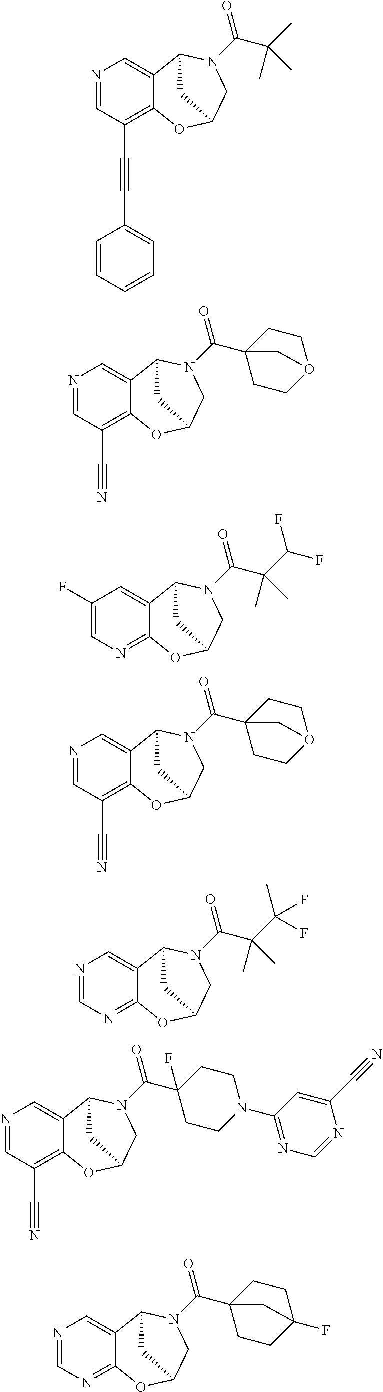

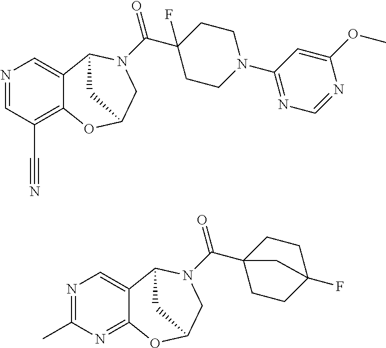

### Definitions

“Alkoxy” means an alkyl-O— group in which the alkyl group encompasses straight alkyl having a carbon number of 1 to 10 and branched alkyl having a carbon number of 3 to 10. Non-limiting examples of suitable alkoxy groups include methoxy, ethoxy, n-propoxy, isopropoxy and n-butoxy. The bond to the parent moiety is through the ether oxygen.

The term “halogen” includes fluorine, chlorine, bromine or iodine.

The term “C₁-C₆alkyl” encompasses straight alkyl having a carbon number of 1 to 6 and branched alkyl having a carbon number of 3 to 6. Specific examples thereof include methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl, 1-ethyl-1-methylpropyl, and the like.

The term “C₃-C₆cycloalkyl” encompasses bridged, saturated or unsaturated cycloalkyl groups having 3 to 6 carbons. Examples of cycloalkyl include cyclopropyl, cyclobutyl, cyclopentyl and cyclohexyl.

The term “C₃-C₁₀cycloalkyl” encompasses bridged, saturated or unsaturated cycloalkyl groups having 3 to 10 carbons. “Cycloalkyl” also includes non-aromatic rings as well as monocyclic, non-aromatic rings fused to a saturated cycloalkyl group. Examples of cycloalkyl include cyclopropyl, cyclobutyl, cyclopentyl, cyclohexyl, cycloheptyl, tetrahydronaphthyl, decahydronaphthyl, indanyl and the like. Examples described by structure include,

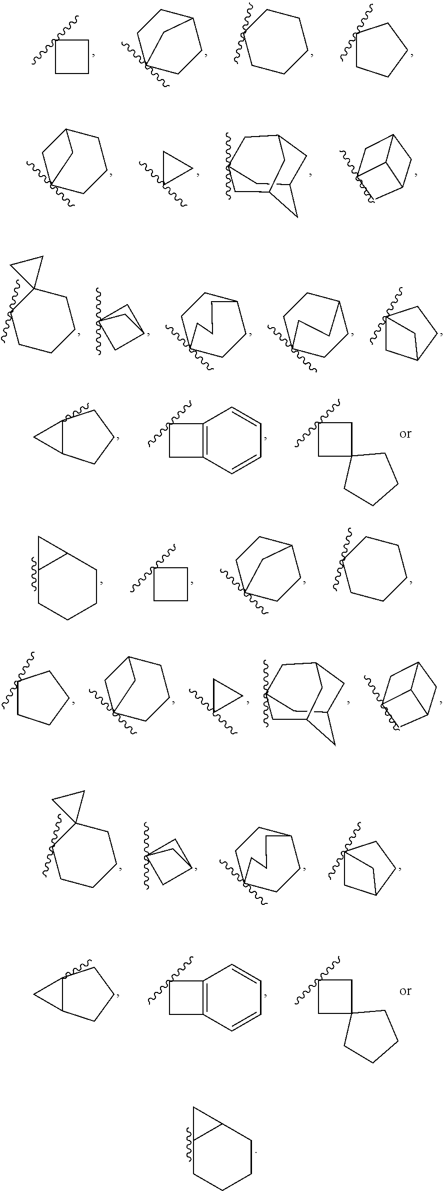

The term “heteroaryl” means a monocyclic or multicyclic, including bicyclic, aromatic heterocycloalkyl that contains at least one ring heteroatom selected from O, S and N. Examples of heteroaryl groups include pyridyl (pyridinyl), oxazolyl, azabenzothiazole, benzothiazole, imidazolyl, triazolyl, furyl, triazinyl, thienyl, pyrimidyl, pyridazinyl, indolizinyl, cinnolinyl, phthalazinyl, quinazolinyl, naphthyridinyl, quinoxalinyl, purinyl, benzimidazolyl, quinolyl, isoquinolyl, and the like.

The term “heterocycloalkyl” means mono- or bicyclic or bridged partially unsaturated and saturated rings containing at least one heteroatom selected from N, S and O, each of said rings having from 3 to 10 atoms in which the point of attachment may be carbon or nitrogen. Examples include azetidine, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or n-substituted-(1H, 3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. Examples described by structure include,

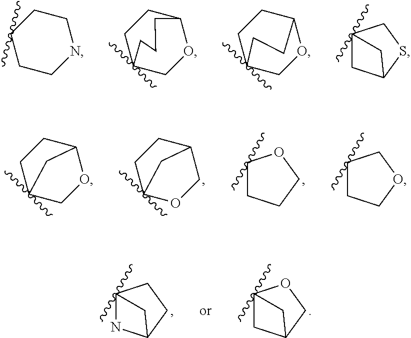

The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, n-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline. N,N-dibenzylethylenediamine, diethylamine, 2-diethylaminoethanol, 2-dimethylaminoethanol, ethanolamine, ethylenediamine, n-ethylmorpholine, n-ethylpiperidinyl, glucamine, glucosamine, histidine, hydrabamine, isopropylamine, lysine, methylglucamine, morpholine, piperazine, piperidinyl, polyamine resins, procaine, purines, theobromine, triethylamine, trimethylamine, tripropylamine, tromethamine, and the like.

The term “patient” refers to a mammalian patient, preferably a human patient, receiving or about to receive medical treatment.

The compounds of the present invention may contain one or more asymmetric centers and can thus occur as racemates, racemic mixtures, single enantiomers, diastereomeric mixtures, and individual diastereomers. The present invention is meant to comprehend all such isomeric forms of these compounds.

Some of the compounds described herein contain olefinic double bonds, and unless specified otherwise, are meant to include both E and Z geometric isomers.

Some of the compounds described herein contain substituted cycloalkanes having cis- and trans-isomers, and unless specified otherwise, are meant to include both cis- and trans-geometric isomers.

The independent syntheses of these diastereomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

It will be understood that the present invention is meant to include the pharmaceutically acceptable salts, and also salts that are not pharmaceutically acceptable, of the compounds described herein, when they are used as precursors to the free compounds or their pharmaceutically acceptable salts or in other synthetic manipulations.

Solvates, and in particular, the hydrates of the compounds of the structural formulas described herein are included in the present invention as well.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

In the compounds described herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the formulas described herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. A ³H, ¹¹C, ¹⁸F labeled compound may be used for PET or SPECT or other imaging studies. Isotopically-enriched compounds can be prepared without undue experimentation by conventional techniques well known to those skilled in the art or by processes analogous to those described in the Schemes and Examples herein using appropriate isotopically-enriched reagents or Intermediates.

It should be noted that chemically unstable compounds are excluded from the embodiments contained herein.

### Methods of Treatment

The compounds described herein may be particularly useful for the prevention, treatment or amelioration of RIPK1-mediated diseases or disorders. Such RIPK1-mediated diseases or disorders are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFL D), kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CV A, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, bums, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the Formulae described herein, or a pharmaceutically acceptable salt thereof, may be particularly useful for the treatment of the following RIPK1-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH) autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)), Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease. Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, bums, multiple sclerosis, type I diabetes, type II diabetes, obesity, Wegener's granulomatosis, pulmonary sarcoidosis, Behcet's disease, interleukin-I converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP 2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly. Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the Formulae described herein, or a pharmaceutically acceptable salt thereof, may be useful for the treatment of glaucoma.

The compounds of the Formulae described herein, or a pharmaceutically acceptable salt thereof, may be particularly useful for treatment of pancreatic ductal adenocarcinoma, hepatocellular carcinoma, mesothelioma, or melanoma.

The compounds of the Formulae described herein, or a pharmaceutically acceptable salt thereof, may be particularly useful for the treatment of the following RIPK1-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen. The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g. cisplatin.

The compounds of this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of the Formulae described herein, or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of the Formulae described herein, or a pharmaceutically acceptable salt thereof, during transplant surgery.

Amelioration of such tissue damage may also be achieved by short-term treatment of a patient with a compound of the Formulae described herein, or a pharmaceutically acceptable salt thereof, after transplant surgery.

In one embodiment, the compounds of the Formulae described herein, or a pharmaceutically acceptable salt thereof, may be useful for the treatment of retinal detachment, macular degeneration, and retinitis pigmentosa.

In another embodiment, the compounds of the Formulae described herein, or a pharmaceutically acceptable salt thereof, may be useful for the treatment of multiple sclerosis.

In one embodiment, the compounds of the Formulae described herein, or a pharmaceutically acceptable salt thereof, may be useful for the treatment of traumatic brain injury.

In another embodiment, the compounds of the Formulae described herein, or a pharmaceutically acceptable salt thereof, may be useful for the treatment of Huntington's Disease or Niemann-Pick disease.

In another embodiment, the compounds of the Formulae described herein, or a pharmaceutically acceptable salt thereof, may be useful for the treatment of amyotrophic lateral sclerosis (ALS), progressive supranuclear palsy (PSP), and Alzheimers disease.

In another embodiment, the compounds of the Formulae described herein, or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease. Alzheimer's Disease, amyotrophic lateral sclerosis, and Niemann-Pick disease may concern, more specifically, the amelioration of organ injury or damage sustained as a result of these diseases/disorders. For example, the compounds described herein may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury. Huntington's Disease, Alzheimer's Disease, amyotrophic lateral sclerosis, and Niemann-Pick disease.

In another embodiment, the compounds of the Formulae described herein, or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of the Formulae described herein, or a pharmaceutically acceptable salt thereof, may be useful for the treatment of psoriasis, rheumatoid arthritis, and ulcerative and colitis.

In another embodiment, the compounds of the Formulae described herein, or a pharmaceutically acceptable salt thereof, may be useful for the treatment of lupus, inflammatory bowel disease (IBD), Crohn's disease, and ulcerative colitis.

In another embodiment, the compounds of the Formulae described herein, or a pharmaceutically acceptable salt thereof, may be useful for the treatment of cerebrovascular accident (CVA, stroke), Huntington's disease. Alzheimer's disease, amyotrophic lateral sclerosis (ALS), traumatic brain injury, multiple sclerosis, Gaucher disease, Niemann-Pick disease, and spinal cord injury.

In another embodiment, the compounds of the Formulae described herein, or a pharmaceutically acceptable salt thereof, may be useful for the treatment of amyotrophic lateral sclerosis (ALS).

In another embodiment, the compounds of the Formulae described herein, or a pharmaceutically acceptable salt thereof, may be useful for the treatment of multiple sclerosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the Formulae described herein, or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the Formulae described herein, or a pharmaceutically acceptable salt thereof, may be useful for the treatment of pancreatic ductal adenocarcinoma (PDAC).

In another embodiment, the compounds of the Formulae described herein, or a pharmaceutically acceptable salt thereof, may be useful for the treatment of intracerebral hemorrhage and subarachnoid hemorrhage.

In another embodiment, the compounds of the Formulae described herein, or a pharmaceutically acceptable salt thereof, may be useful for the treatment of type II diabetes and obesity.

In another embodiment, the compounds of the Formulae described herein, or a pharmaceutically acceptable salt thereof, may be useful for the treatment of atherosclerosis.

In another embodiment, the compounds of the Formulae described herein, or a pharmaceutically acceptable salt thereof, may be useful for the treatment of vasculitis.

In another embodiment, the compounds of the Formulae described herein, or a pharmaceutically acceptable salt thereof, may be useful for the treatment of dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis.

In another embodiment, the compounds of the Formulae described herein, or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the Formulae described herein, or pharmaceutically acceptable salt thereof, may be useful for the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of the Formulae described herein, or a pharmaceutically acceptable salt thereof, may be particularly useful for the treatment of the RIPK1-mediated, cancer-related diseases or disorders. Gong et al., The role of necroptosis in cancer biology and therapy, Molecular Cancer (2019) 18:100. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC). EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma. Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma. Hodgkin's lymphoma, Non-Hodgkin's lymphoma, lymphoblastic T cell lymphoma. Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma. Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

### Pharmaceutical Compositions

Compounds described herein may be administered orally or parenterally. As formulated into a dosage form suitable for administration, the compounds described herein can be used as a pharmaceutical composition for the prevention, treatment, or remedy of the above diseases.

In clinical use of the compounds described herein, usually, the compound is formulated into various preparations together with pharmaceutically acceptable additives according to the dosage form and may then be administered. By “pharmaceutically acceptable” it is meant the additive, carrier, diluent or excipient must be compatible with the other ingredients of the formulation and not deleterious to the recipient thereof. As such, various additives ordinarily used in the field of pharmaceutical preparations are usable. Specific examples thereof include gelatin, lactose, sucrose, titanium oxide, starch, crystalline cellulose, hydroxypropyl methylcellulose, carboxymethylcellulose, corn starch, microcrystalline wax, white petrolatum, magnesium metasilicate aluminate, anhydrous calcium phosphate, citric acid, trisodium citrate, hydroxypropylcellulose, sorbitol, sorbitan fatty acid ester, polysorbate, sucrose fatty acid ester, polyoxyethylene, hardened castor oil, polyvinylpyrrolidone, magnesium stearate, light silicic acid anhydride, talc, vegetable oil, benzyl alcohol, gum arabic, propylene glycol, polyalkylene glycol, cyclodextrin, hydroxypropyl cyclodextrin, and the like.

Preparations to be formed with those additives include, for example, solid preparations such as tablets, capsules, granules, powders and suppositories; and liquid preparations such as syrups, elixirs and injections. These may be formulated according to conventional methods known in the field of pharmaceutical preparations. The liquid preparations may also be in such a form that may be dissolved or suspended in water or in any other suitable medium in their use. Especially for injections, if desired, the preparations may be dissolved or suspended in physiological saline or glucose liquid, and a buffer or a preservative may be optionally added thereto.

The pharmaceutical compositions may contain the compound of the invention in an amount of from 1 to 99.9 by weight, preferably from 1 to 60% by weight of the composition. The compositions may further contain any other therapeutically-effective compounds.

In case where the compounds of the invention are used for prevention or treatment for the above-mentioned diseases, the dose and the dosing frequency may be varied, depending on the sex, the age, the body weight and the disease condition of the patient and on the type and the range of the intended remedial effect. In general, when orally administered, the dose may be from 0.001 to 50 mg/kg of body weight/day, and it may be administered at a time or in several times. In specific embodiments, the dose is from about 0.01 to about 25 mg/kg/day, in particular embodiments, from about 0.05 to about 10 mg/kg/day. For oral administration, the compositions are preferably provided in the form of tablets or capsules containing from 0.01 mg to 1,000 mg. In specific embodiments, the dose is 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 or 1,000 milligrams of a compound described herein. This dosage regimen may be adjusted to provide the optimal therapeutic response.

### Combination Therapy

The compounds of the present invention are further useful in methods for the prevention or treatment of the aforementioned diseases, disorders and conditions in combination with other therapeutic agents.

The compounds of the present invention may be used in combination with one or more other drugs in the treatment, prevention, suppression or amelioration of diseases or conditions for which compounds described herein or the other drugs may have utility, where the combination of the drugs together are safer or more effective than either drug alone. Such other drug(s) may be administered in an amount commonly used therefore, contemporaneously or sequentially with a compound described herein or a pharmaceutically acceptable salt thereof. When a compound described herein is used contemporaneously with one or more other drugs, the pharmaceutical composition may in specific embodiments contain such other drugs and the compound described herein or its pharmaceutically acceptable salt in unit dosage form. However, the combination therapy may also include therapies in which the compound described herein or its pharmaceutically acceptable salt and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound described herein or a pharmaceutically acceptable salt thereof.

### EXAMPLES

### Abbreviations

The abbreviations used herein have the following tabulated meanings. Abbreviations not tabulated below have their meanings as commonly used unless specifically stated otherwise.

| | |
|---|---|
| ACN | acetonitrile |
| AcOH | acetic acid |
| BPin | 4,4,5,5-tetramethyl-1,3,2-dioxaborolan-2-yl |
| B[Pin]₂ | 4,4,4′,4′,5,5,5′,5′-octamethyl-2,2′-bi(1,3,2-dioxaborolane) |
| Boc | tert-butoxycarbamate |
| Boc₂O | di-tert-butyl dicarbonate |
| BrettPhos Pd G3 | [(2-di-cyclohexylphosphino-3,6-dimethoxy-2′,4′,6′- triisopropyl-1,1′- |
| | biphenyl)-2-(2′-amino-1,1′ -biphenyl)]palladium(II) |
| | methanesulfonate methanesulfonate |
| CMBP | cyanomethylenetributylphosphorane |
| DBAD | di-tert-butyl (E)-diazene-1,2-dicarboxylate |
| DCM | dichloromethane |
| DEAD | diethyl azodicarboxylate |
| DIAD | diisopropyl azodicarboxylate |
| DIEA | diisopropylethylamine |
| DMA | dimethylacetamide |
| DMAP | 4-dimethylaminopyridine |
| DMF | dimethylformamide |
| DMSO | dimethylsulfoxide |
| Dppf | 1,1′-Bis(diphenylphosphino)ferrocene, |
| Dtbpy or DTBPY | 4,4′-di-tert-butyl-2,2′-dipyridyl |
| EI | electron ionization |
| EtOAc | ethyl acetate |
| EtOH | ethanol |
| h | hour(s) |
| HATU | (1-[bis(dimethylamino)methylene]-1H-1,2,3-triazolo[4,5- |
| | b]pyridinium 3-oxide hexafluorophosphate |
| ¹H NMR | proton nuclear magnetic resonance |
| HPLC | high performance liquid chromatography |
| i-Pr₂NEt | diisopropylethylamine |
| Ir[dF(CF₃)ppy]₂(dtbpy) | [4,4′-bis(1,1-dimethylethyl)-2,2′-bipyridine-N1,N1′]bis[3,5-difluoro- |
| | 2-[5-(trifluoromethyl)-2-pyridinyl-N]phenyl-C]Iridium(III) |
| Ir[dF(CF₃)ppy]₂(dtbpy)PF₆ | [4,4′-Bis(1,1-dimethylethyl)-2,2′-bipyridine-N1,N1′]bis[3,5-difluoro- |
| | 2-[5-(trifluoromethyl)-2-pyridinyl-N]phenyl-C]Iridium(III) |
| | hexafluorophosphate |
| K₂CO₃ | Potassium Carbonate |
| LC/MS | liquid chromatography coupled to mass spectrometer |
| LDA | lithium diisopropylamide |
| MeCN | acetonitrile |
| MeOH | methanol |
| MHz | megahertz |
| min | minute(s) |
| mL | milliliter(s) |
| MS | mass spectrum |
| MsCl | methanesulfonyl chloride |
| MTBE | methyl tert-butyl ether |
| NaBH₄ | Sodium borohydride |
| NaOH | Sodium hydroxide |
| Na₂SO₄ | Sodium sulfate |
| NaCl | Sodium chloride |
| NBS | N-bromosuccinamide |
| NCS | N-chlorosuccinamide |
| NH₄OH | Ammonium hydroxide |
| NH₄HCO₃ | Ammonium bicarbonate |
| NiCl₂ | Nickel chloride |
| NMO | N-methylmorpholine N-oxide |
| NMR | nuclear magnetic resonance |
| Pd₂(dba)₃ | Tris(dibenzylideneacetone)dipalladium(0) |
| Ph₃P | triphenylphosphine |
| P(o-tol)₃ Pd G2 | chloro[tri(o-tolyl)phosphine][2-(2′-amino-1,1′- |
| | biphenyl)]palladium(II) |
| RT | room temperature |
| sat. | saturated |
| Selectfluor | 1-chloromethyl-4-fluoro-1,4-diazoniabicyclo[2.2.2]octane |
| | bis(tetrafluoroborate) |
| T3P | 1-propanephosphonic anhydride |
| TBAF | tetra-n-butylammonium fluoride |
| TBS | tert-butyldimethylsilyl |
| TBSCl | tert-butyldimethylsilyl chloride |
| tBu-terpy | 4,4′,4″-tri-tert-butyl-2,2′:6′,2″-terpyridine |
| TEA | triethylamine |
| TFA | trifluoroacetic acid |
| TfOH | Triflic acid |
| THF | tetrahydrofuran |
| TIPS | Triisopropylsilyl |
| TLC | thin layer chromatography |
| TsCl | Tosyl chloride |
| TsOH | toluenesulfonic acid |
| XPhos-Pd-G3 | (2-dicyclohexylphosphino-2′,4′,6′-triisopropyl-1,1′-biphenyl)[2-(2′- |
| | amino-1,1′-biphenyl)]palladium(II) methanesulfonate |

### General Synthetic Schemes

One approach to (2S,5S)-2,3,4,5-tetrahydro-2,5-methanopyrido[3,4-f][1,4]oxazepine amides is described in General Scheme I.

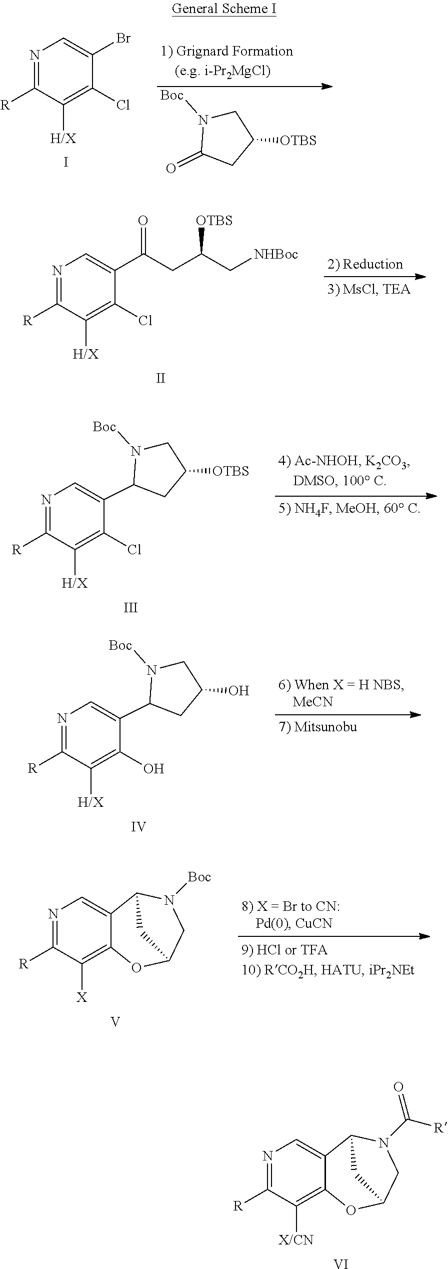

A Grignard reagent is prepared from a bromopyridine and reacted with tert-butyl (R)-4-((tert-butyldimethylsilyl)oxy)-2-oxopyrrolidine-1-carboxylate giving II. Reduction of the resulting ketone, conversion to a methanesulfonyl and cyclization provides pyrrolidine III. The chloropyridine is converted to an hydroxypyridine, with deprotection of the TBS protective group giving IV. The phenol provides an opportunity to further functionalize with halogenation agents (e.g. NBS), prior to a Mitsunobu cyclization providing the S,S core V. The halogenated core V can optionally be converted to cyano core with Pd(O) catalysis and either Zn(CN)₂ or CuCN. The final inhibitors VI can be prepared by acid-mediated deprotection and amide bond formation using standard coupling conditions (e.g. HATU). Another approach relies upon an Ir-catalyzed photoredox coupling between a proline derivative and aryl bromide 1, giving VII directly as a mixture of diastereomers. Pyridyl chloride hydrolysis, halogenation and Mitsunobu reactions gave V. Finally, an optional cyanation, deprotection and amide bond formation provided inhibitors VI in a fashion analogous to the approach described in Scheme I.

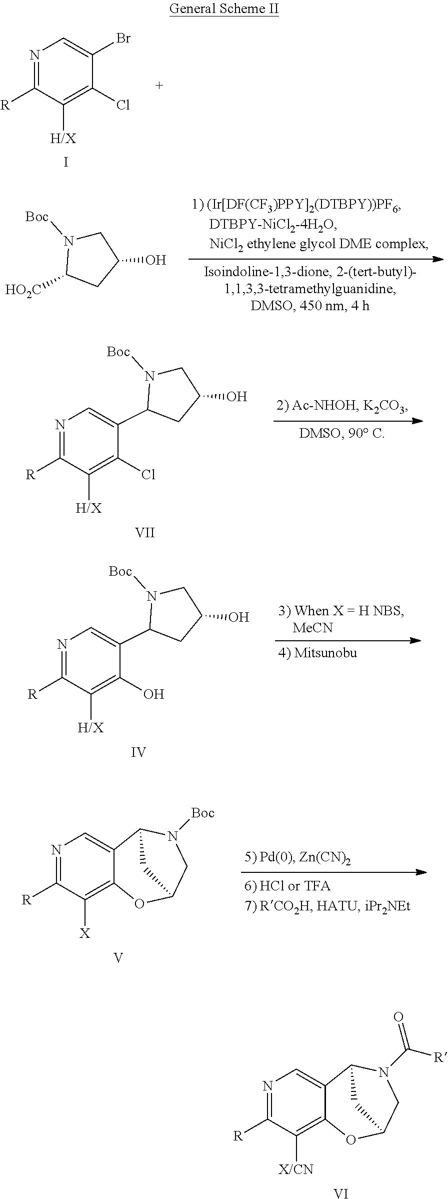

The halogen at C(9) of the core can be converted to other groups besides a nitrile. For example, alkynes can be obtained by a Sonogashira coupling with an acetylene and Pd catalysis with VIII, giving IX (General Scheme III).

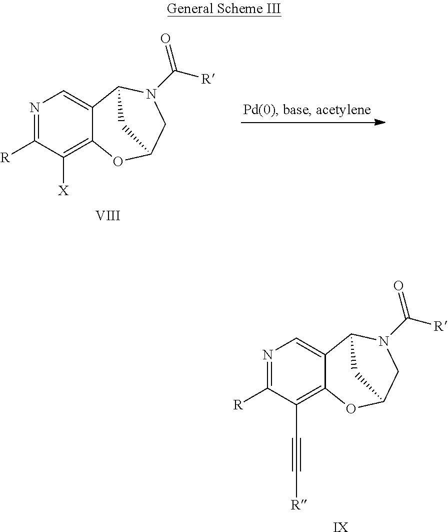

This overall approach can be used for a variety of heterocycles, pyridine isomers and pyrimidines. For example, methoxy pyridine X (General Scheme IV) can be arylated with a photoredox coupling with pyrrolidine acids, giving XI. The methoxy group is converted to a pyridone XII which then undergoes Mitsunobu cyclization to bridged bicycles XIII. Finally, the Boc protective group is cleaved and the amine coupled by a carboxylic acid to obtain final inhibitors XIV.

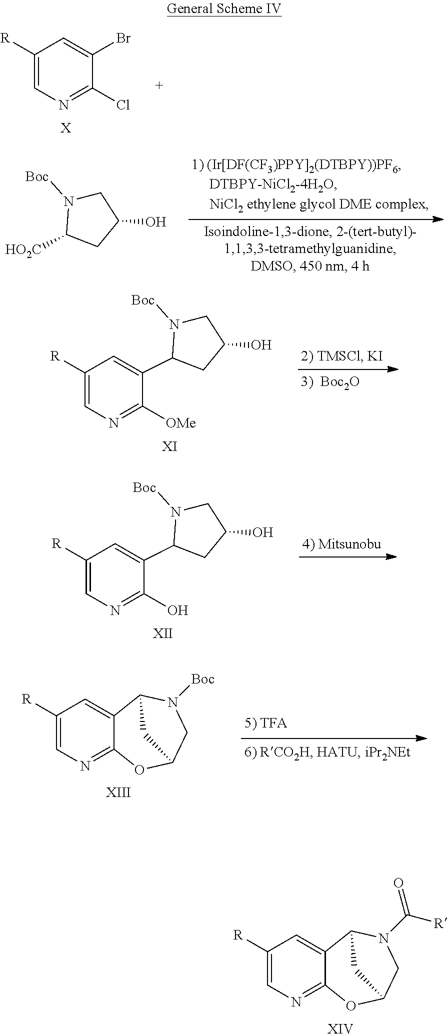

### Preparation of Synthetic Intermediates

### Preparation of tert-Butyl (R)-4-((tert-Butyldimethylsilyl)oxy)-2-oxopyrrolidine-1-carboxylate

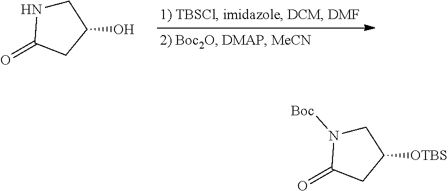 [SMILES: O=C1C[C@@H](O)CN1 ; CC(C)(C)OC(=O)N1C[C@H](O[Si](C)(C)C(C)(C)C)CC1=O]

Step 1. This reaction was conducted in two vessels in parallel. For each, a solution of (R)-4-hydroxypyrrolidin-2-one (238 g, 2.35 mol) in DMF (475 mL) and DCM (2375 mL) was treated at 0° C. with imidazole (240 g, 3.52 mol) and t-butyldimethylchlorosilane (425 g, 2.82 mol), and then stirred for 12 h at 20′CC. The mixtures were combined and quenched by addition to water (5 L) and extracted with DCM (2 L). The organic layer was washed with 2 L of 0.5 N HCL, 1 L of brine, dried over anhydrous sodium sulfate and concentrated under vacuum, to provide a combined 1.01 kg of (R)-4-[(tert-butyldimethylsilyl)oxy]pyrrolidin-2-one. ¹H NMR (400 MHz, CDCl₃) δ 6.78 (s, 1H), 4.53 (m, 1H), 3.58 (m, 1H), 3.23 (m, 1H), 2.53 (dd, J=6.8 Hz, 1H), 2.26 (dd, =4.4 Hz, 1H), 0.87 (s, 9H), 0.06 (s, 6H).

Step 2. This reaction was conducted in three round bottom flasks. For each, a solution of (R)-4-[(tert-butyldimethylsilyl)oxy]pyrrolidin-2-one (337 g, 1.56 mol) in acetonitrile (3.4 L) was charged with 4-dimethylaminopyridine (19.1 g, 156 mmol) and di-tert-butyl dicarbonate (409 g, 1.88 mol). The reactions were stirred for 20 h at 20° C., then combined by a quench with 15 L of water. The mixture was then extracted with DCM (3×3 L), and the organic layer washed with 5 L of 0.5 N HCl and 5 L of brine. The organic layer was dried over anhydrous sodium sulfate and concentrated under vacuum. The residue was purified by trituration with hexane (3.6 L) for 1 h and filtered to collect a combined 1.20 kg of tert-butyl (R)-4-[(tert-butyldimethylsilyl)oxy]-2-oxopyrrolidine-1-carboxylate. ¹H NMR (400 MHz, DMSO-d₆) δ 4.42 (t, J=5.2 Hz, 1H), 3.84 (dd, J=4.8 Hz, 1H), 3.47 (d. J=11.2 Hz, 1H), 2.81 (dd, J=5.6 Hz, 1H), 2.20 (d. J=17.2 Hz, 1H), 1.44 (s, 9H), 0.85 (s, 9H), 0.007 (s, 6H).

### Preparation of Intermediate I. (2S,5S)-2,3,4,5-Tetrahydro-2,5-methanopyrido[3,4-f][1,4]oxazepine-9-carbonitrile

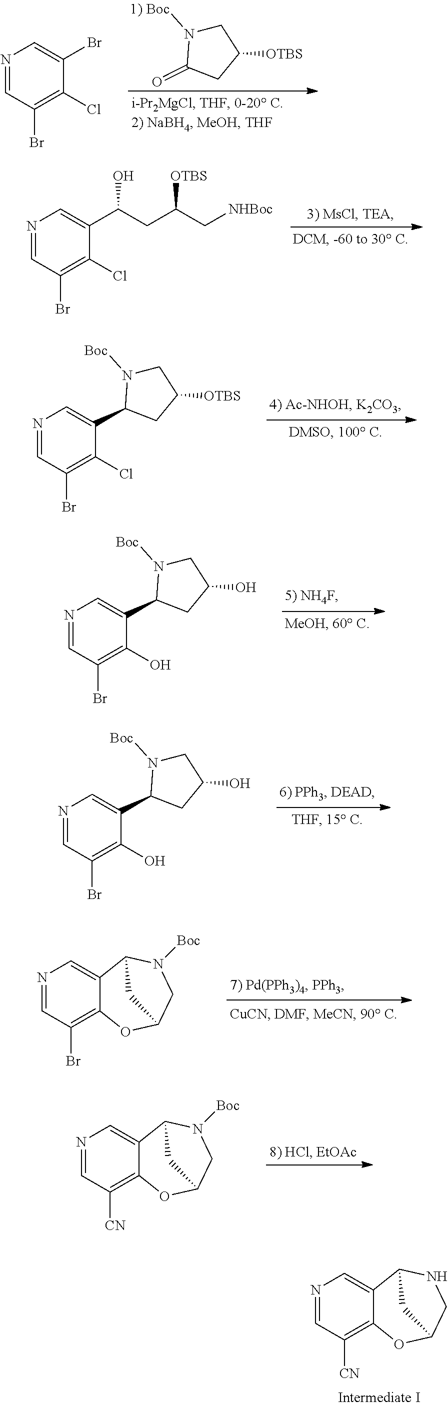

Step 1. This reaction was conducted in seven round bottom flasks. For each, a solution of 3,5-dibromo-4-chloropyridine (132 g, 486 mmol) in 3.0 L of THF was treated dropwise at 0° C. with a 2 M solution of i-PrMgCl in THF (219 mL, 438 mmol). The mixtures were stirred for 1 h. Next, a solution of tert-butyl (R)-4-[(tert-butyldimethylsilyl)oxy]-2-oxopyrrolidine-1-carboxylate (167 g, 528 mmol) in 680 mL of THF was added dropwise at 0° C. to each reaction. The mixtures were stirred for 5 h at 20° C., combined and quenched by the addition to 10 L of saturated aqueous ammonium chloride. The mixture was extracted with EtOAc (2 L), and the organic layer washed with brine (5 L), dried over anhydrous sodium sulfate, filtered and concentrated. The crude product tert-butyl (R)-(4-(5-bromo-4-chloropyridin-3-yl)-2-((tert-butyldimethylsilyl)oxy)-4-oxobutyl)carbamate (1.64 kg) was carried forward without further purification.

Step 2. This step was completed in four vessels in parallel. For each, a solution of tert-butyl (R)-(4-(5-bromo-4-chloropyridin-3-yl)-2-((tert-butyldimethylsilyl)oxy)-4-oxobutyl)carbamate (410 mg, 807 mmol) in 1 L of THF and 1.8 mL of MeOH was treated with NaBH₄ (45.8 g, 1.21 mol) and stirred for 1.5 h. The mixtures were combined, quenched with 10 L of saturated ammonium chloride and extracted with EtOAc (2×1 L). The combined organic layers were washed with brine (5 L), dried over anhydrous magnesium sulfate, filtered and concentrated. The residue was purified by chromatography on silica gel (gradient of 100:1 to 0:1 petroleum ether/EtOAc) to obtain tert-butyl ((2R,4R)-4-(5-bromo-4-chloropyridin-3-yl)-2-((tert-butyldimethylsilyl)oxy)-4-hydroxybutyl)carbamate. ¹H NMR (400 MHz, CDCl₃) δ 8.72 (s, 1H), 8.66 (s, 1H), 5.31 (d. J=9.6 Hz, 1H), 4.83 (s, 1H), 4.34 (d. J=2.8 Hz, 1H), 4.18 (m, 1H), 3.46 (m, 1H), 3.33 (m, 1H), 1.93 (m, 1H), 1.80 (m, 1H), 1.78 (s, 9H), 0.94 (s, 9H), 0.15 (s, 6H).

Step 3. This transformation was conducted in two vessels. For each, a solution of tert-butyl ((2R,4R)-4-(5-bromo-4-chloropyridin-3-yl)-2-((tert-butyldimethylsilyl)oxy)-4-hydroxybutyl)carbamate (135 g, 265 mmol) in 675 mL of DCM was cooled to −60° C. and treated dropwise with triethylamine (80.4 g, 794 mmol) and methanesulfonyl chloride (47.1 g, 411 mmol) over a 1 h period. The reaction mixtures were stirred for 2 h at −60° C. warmed to 30° C. and stirred for 45 min. The reaction mixtures were combined, poured into 50) mL of water and extracted with 200 mL of DCM. The organic layers were washed with brine (200 mL), dried over sodium sulfate and concentrated. The residue was purified by chromatography on silica gel (100:1 to 0:1 petroleum ether/EtOAc) to obtain tert-butyl (2S,4R)-2-(5-bromo-4-chloropyridin-3-yl)-4-((tert-butyldimethylsilyl)oxy)pyrrolidine-1-carboxylate. ¹H NMR (400 MHz, CDCl₃) δ 8.63 (m, 1H), 8.34 (m, 1H), 5.28 (m, 1H), 4.41 (m, 1H), 3.60-3.70 (m, 2H), 2.05-2.44 (m, 1H), 1.81-1.90 (m, 1H), 1.45 (s, 9H), 0.90 (s, 9H), 0.09 (s, 6H); MS (EI) calculated for C₂₀H₃₃BrClN₂O₃Si [M+H]+, 491, 493; found, 491, 493.

Step 4. A solution of tert-butyl (2S,4R)-2-(5-bromo-4-chloropyridin-3-yl)-4-((tert-butyldimethylsilyl)oxy)pyrrolidine-1-carboxylate (249 g, 506 mmol) in DMSO (1.7 L) was treated with K₂CO₃ (420 g, 3.04 mmol) and acetylhydroxamic acid (152 g, 2.02 mmol). The reaction mixture was stirred at 100° C. for 12 h, then poured into 5 L of water. The resulting precipitate that formed was filtered and collected. The filter cake was washed with water (5 L) and acetone (3 L), then dried under reduced pressure, providing tert-butyl (2S,4R)-2-(5-bromo-4-hydroxypyridin-3-yl)-4-((tert-butyldimethylsilyl)oxy)pyrrolidine-1-carboxylate. ¹H NMR (400 MHz, DMSO-d₆) δ 8.05 (s, 1H), 7.45 (m, 1H), 4.75 (m, 1H), 4.36 (m, 1H), 3.38-3.56 (m, 2H), 2.05-2.10 (m, 2H), 1.32, 1.36 (2s, 9H), 1.13 (s, 9H), 0.05 (s, 6H).

Step 5. A solution of tert-butyl (2S,4R)-2-(5-bromo-4-hydroxypyridin-3-yl)-4-((tert-butyldimethylsilyl)oxy)pyrrolidine-1-carboxylate (213 g, 450 mmol) in MeOH (1280 mL) was treated with ammonium fluoride (50 g, 1.35 mol) and stirred at 60° C. for 48 h. The mixture was concentrated, and the residue purified by chromatography on silica gel (5:1 to 0:1 DCM/MeOH) to provide tert-butyl (2S,4R)-2-(5-bromo-4-hydroxypyridin-3-yl)-4-hydroxypyrrolidine-1-carboxylate. ¹H NMR (400 MHz, CD₃OD) δ 8.14 (m, 1H), 7.59 (m, 1H), 4.96 (m, 1H), 4.37 (s, 1H), 3.67-3.71 (m, 1H), 3.59 (m, 1H), 2.34 (m, 1H), 2.08 (m, 1H), 1.27, 1.45 (s, 9H).

Step 6. A solution of tert-butyl (2S,4R)-2-(5-bromo-4-hydroxypyridin-3-yl)-4-hydroxypyrrolidine-1-carboxylate (120 g, 334 mmol) in THF (840 mL) was treated at 15° C. with triphenylphosphine (105 g, 401 mmol) and DEAD (69.8 g, 401 mmol). The mixture was stirred at 15° C. for 15 h, then poured into water (1 L) and extracted with EtOAc (1 L). The organic layer was washed with brine, dried over sodium sulfate and concentrated. The residue was purified by chromatography on silica gel (1:3 to 1:4 petroleum ether/EtOAc) to provide tert-butyl (2S,5S)-9-bromo-2,3-dihydro-2,5-methanopyrido[3,4-f][1,4]oxazepine-4(5H)-carboxvlate. ¹H NMR (400 MHz, CDCl₃) δ 8.45 (s, 1H), 8.19 (s, 1H), 5.19 (s, 1H), 4.84 (m, 1H), 3.64-3.83 (m, 2H), 2.19-2.36 (m, 2H), 1.43 (s, 9H); MS (EI) calculated for C₁₄H₁₈BrN₂O [M+H]⁺, 341, 343; found, 341, 343.

Step 7. This step was performed in three vessels. For each, a solution of tert-butyl (2S,5S)-9-bromo-2,3-dihydro-2,5-methanopyrido[3,4-f][1,4]oxazepine-4(5H)-carboxylate (28.0 g, 82.1 mmol) in DMF (140 mL) and MeCN (420 mL) was treated with CuCN (7.35 g, 82.1 mmol), Pd(PPh₃)₄ (19.0 g, 16.4 mmol) and PPh₃ (45.2 g, 172 mmol). The mixtures were stirred at 90° C. for 1.5 h, cooled and combined. The combined mixture was poured into water (3 L), resulting in a suspension which was filtered, and the precipitate was washed with EtOAc (3×2 L). The combined organic layer was washed with brine (1 L), dried over sodium sulfate and concentrated. The crude product was purified by chromatography on SiO₂ (100:1 to 0:1 petroleum ether/EtOAc) to provide tert-butyl (2S,5S)-9-cyano-2,3-dihydro-2,5-methanopyrido[3,4-f][1,4]oxazepine-4(5H)-carboxylate. ¹H NMR (400 MHz, CDCl₃) δ 8.57 (s, 1H), 8.42 (s, 1H), 5.25 (s, 1H), 4.95 (m, 1H), 3.67-3.87 (m, 2H), 2.40 (m, 1H), 2.21 (m, 1H), 1.43 (s, 9H).

Step 8. A solution of tert-butyl (2S,5S)-9-cyano-2,3-dihydro-2,5-methanopyrido[3,4-f][1,4]oxazepine-4(5H)-carboxylate (43.0 g, 150 mmol) in EtOAc (215 mL) was treated at 0° C. with a 4 M solution of HCl in EtOAc (374 mL). The mixture was stirred for 1 h at 15° C., filtered and the precipitate triturated with MeCN (100 mL) for 2 h. The product was collected and dried in the vacuum to provide (2S,5S)-2,3,4,5-tetrahydro-2,5-methanopyrido[3,4-f][1,4]oxazepine-9-carbonitrile, HCl (Intermediate I). ¹H NMR (400 MHz, D₂O) δ 8.90 (s, 1H), 8.68 (s, 1H), 5.62 (s, 1H), 5.21 (s, 1H), 3.80-3.93 (m, 2H), 2.54 (s, 2H); MS (EI) calculated for C₁₀H₁₀N₃O [M+H]⁺, 188; found, 188.

### Preparation of Intermediate II. (2S,5S)-9-Chloro-2,3,4,5-tetrahydro-2,5-methanopyrido[3,4-f][1,4]oxazepine

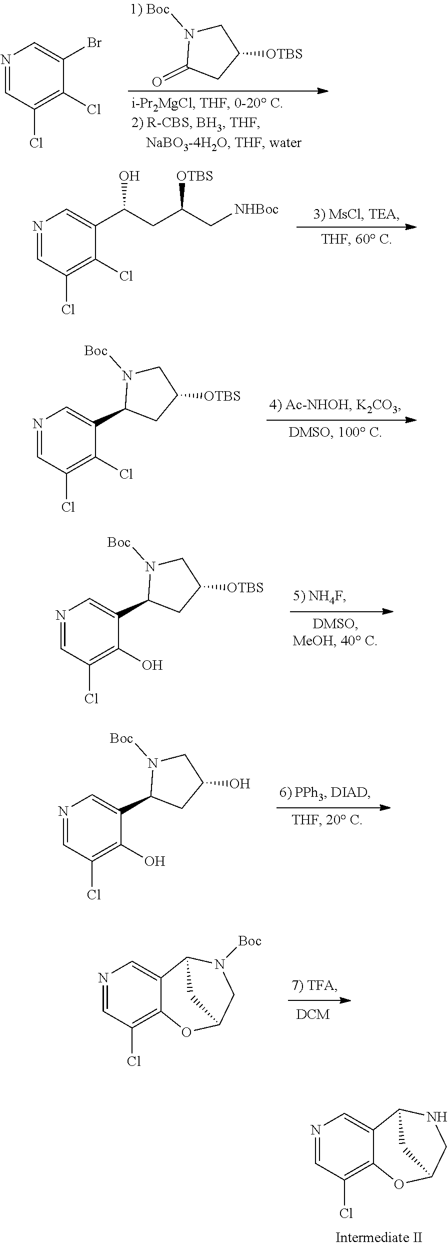

Step 1. A mixture of 3-bromo-4,5-dichloropyridine (10.0 g, 44.1 mmol) in THF (100 mL) was treated dropwise at 0° C. with a 2.0 M solution of isopropyl magnesium chloride in THF (24.2 mL, 48.5 mmol). The suspension was stirred at 0° C. for 60 min, then treated with a solution of tert-butyl (R)-4-((tert-butyldimethylsilyl)oxy)-2-oxopyrrolidine-1-carboxylate (16.7 g, 52.9 mmol) in THF (50 mL). The mixture was stirred at 20° C. for 4 h, quenched with saturated aqueous ammonium chloride, and extracted with EtOAc. The combined organic layers were washed with 1 N NaOH, brine, dried over anhydrous Na₂SO₄, filtered, concentrated, and purified by chromatography on silica gel (120 g, eluting with gradient of 0-10% EtOAc/petroleum ether) to give tert-butyl (R)-(2-((tert-butyldimethylsilyl)oxy)-4-(4,5-dichloropyridin-3-yl)-4-oxobutyl)carbamate. ¹H NMR (400 MHz, CDCl₃) δ 8.70 (s, 1H), 8.57 (s, 1H), 4.77 (br s, 1H), 4.46 (m, 1H), 3.33-3.42 (m, 1H), 3.12-3.18 (m, 2H), 1.44 (s, 9H), 0.85 (s, 9H), 0.12 (s, 3H), 0.05 (s, 3H); MS (EI) calculated for C₂₀H₃₃C₁₂N₂O₄Si [M+H]+, 463, 465; found, 463, 465.

Step 2. (R)-1-Methyl-3,3-diphenyltetrahydro-1H,3H-pyrrolo[1,2-c][1,3,2]oxazaborole (2.76 mL, 2.76 mmol) was added to a mixture of borane dimethyl sulfide complex (2.76 mL, 27.6 mmol) in THF (50 mL) and stirred for 15 min. Next, tert-butyl (R)-(2-((tert-butyldimethylsilyl)oxy)-4-(4,5-dichloropyridin-3-yl)-4-oxobutyl)carbamate (6.40 g, 13.8 mmol) in THF (100 mL) was added, and the mixture was stirred at 20° C. for 1 h. The reaction was quenched with water (200 mL) and extracted with EtOAc. The combined organic layers were washed with brine, dried over anhydrous Na₂SO₄, filtered and concentrated. The residual oil was dissolved in 100 mL of 1:2 THF/water containing sodium perborate tetrahydrate (8.50 g, 55.2 mmol) and stirred for 16 h. The mixture was extracted with EtOAc, and the combined organic layers were washed with brine, dried over anhydrous Na₂SO₄, filtered, concentrated and purified by chromatography on silica gel (80 g, eluting with 0-20% EtOAc/petroleum ether) to give tert-butyl ((2R,4S)-2-((tert-butyldimethylsilyl)oxy)-4-(4,5-dichloropyridin-3-yl)-4-hydroxybutyl)carbamate and tert-butyl ((2R,4R)-2-((tert-butyldimethylsilyl)oxy)-4-(4,5-dichloropyridin-3-yl)-4-hydroxybutyl)carbamate. Minor (undesired R,S isomer): ¹H NMR (400 MHz, CDCl₃) δ 8.76 (s, 1H), 8.58 (s, 1H), 5.24-5.34 (m, 1H), 4.81 (br s, 1H), 4.13-4.19 (m, 1H), 3.18-3.39 (m, 2H), 1.95-2.03 (m, 1H), 1.73-1.84 (m, 1H), 1.45 (s, 9H), 0.93 (s, 9H), 0.15 (2s, 6H); MS (EI) calculated for C₂₀H₃₅Cl₂N₂O₄Si [M+H]⁺, 465, 467; found, 465, 467. Major (desired R,R isomer): ¹H NMR (400 MHz, CDCl₃) δ 8.75 (s, 1H), 8.58 (s, 1H), 5.34 (br d, J=10 Hz, 1H), 4.82 (br s, 1H), 4.15-4.23 (m, 1H), 3.46 (br s, 1H), 3.35 (br s, 1H), 1.95 (br d, J=12 Hz, 1H), 1.74-1.85 (m, 1H), 1.46 (s, 9H), 0.95 (s, 9H), 0.16 (2s, 6H); MS (EI) calculated for C₂₀H₃₅C₁₂N₂O₄Si [M+H]⁺, 465, 467; found, 465, 467.

Step 3. A mixture of tert-butyl ((2R,4R)-2-((tert-butyldimethylsilyl)oxy)-4-(4,5-dichloropyridin-3-yl)-4-hydroxybutyl)carbamate (2.70 g, 5.80 mmol) in THF (150 mL) was treated with TEA (16 mL, 116 mmol) and methanesulfonyl chloride (7.0 mL, 90 mmol), and stirred at 60° C. for 80 h. The mixture was quenched with water (300 mL) and extracted EtOAc. The combined organic layers were washed with brine, dried over anhydrous Na₂SO₄, filtered and concentrated. The residue was purified by chromatography on silica gel (40 g, eluting with a gradient of 0-20% EtOAc/petroleum ether) to give tert-butyl (2S,4R)-4-((tert-butyldimethylsilyl)oxy)-2-(4,5-dichloropyridin-3-yl)pyrrolidine-1-carboxylate. ¹H NMR (400 MHz, CDCl₃) δ 8.53 (br s, 1H), 8.33 (br s, 1H), 5.21-5.41 (m, 1H), 4.42 (br d, J=15 Hz, 1H), 3.54-3.77 (m, 2H), 2.43 (br d, J=10 Hz, 1H), 1.75-1.95 (m, 1H), 1.20, 1.46 (2 s, 9H), 0.90 (s, 9H), 0.09 (s, 6H); MS (EI) calculated for C₂₀H₃₃C₁₂N₂O₃Si [M+H]⁺, 447; found, 447.

Steps 4 and 5. A mixture of tert-butyl (2S,4R)-4-((tert-butyldimethylsilyl)oxy)-2-(4,5-dichloropyridin-3-yl)pyrrolidine-1-carboxylate (1.25 g, 2.79 mmol) in DMSO (10 mL) was treated with K₂CO₃ (2.32 g, 16.8 mmol) and N-hydroxyacetamide (0.629 g, 8.38 mmol). The mixture was stirred at 100° C. for 16 h and filtered. The filtrate was diluted with MeOH (10 mL) and treated with ammonium fluoride (1.03 g, 27.9 mmol). The mixture was stirred at 40° C. for 16 h, filtered, and purified by reverse phase chromatography (gradient of 5 to 35% MeCN/water with 0.05% NH₄OH and 10 mM NH₄CO₃) to give tert-butyl (2S,4R)-2-(5-chloro-4-hydroxypyridin-3-yl)-4-hydroxypyrrolidine-1-carboxylate. MS (EI) calculated for C₁₄H₂₀ClN₂O₄[M+H]⁺, 315; found, 315.

Step 6. A solution of tert-butyl (2S,4R)-2-(5-chloro-4-hydroxypyridin-3-yl)-4-hydroxypyrrolidine-1-carboxylate (560 mg, 1.78 mmol) in THF (30 mL) was treated with Ph₃P (280 mg, 10.7 mmol), DIAD (2.08 mL, 10.7 mmol). The mixture was stirred at 20° C. for 16 h and concentrated. The residue was purified by chromatography on silica gel (25 g, gradient of 0-40% EtOAc/petroleum ether) to give tert-butyl (2S,5S)-9-chloro-2,3-dihydro-2,5-methanopyrido[3,4-f][1,4]oxazepine-4(5H)-carboxylate. MS (EI) calculated for C₁₄H₁₈ClN₂O₃ [M+H]⁺, 297; found, 297.

Step 7. A solution of tert-butyl (2S,5S)-9-chloro-2,3-dihydro-2,5-methanopyrido[3,4-f][1,4]oxazepine-4(5H)-carboxylate (620 mg, 1.67 mmol) in DCM (3 mL) was treated with TFA (1 mL) and stirred for 4 h. The mixture was concentrated, and the residue purified by reverse phase chromatography (gradient of 0-20% MeCN/water with 0.1% TFA) to give (2S,5S)-9-chloro-2,3,4,5-tetrahydro-2,5-methanopyrido[3,4-f][1,4]oxazepine, TFA salt. ¹H NMR (400 MHz, CD₃OD) δ 8.55 (s, 1H), 8.38 (s, 1H), 5.51 (br s, 1H), 5.12 (d, J=4.40 Hz, 1H), 3.73-3.88 (m, 2H), 2.43-2.62 (m, 2H); MS (EI) calculated for C₉H₁₀ClN₂O [M+H]⁺, 197; found, 197.

### Preparation of Intermediate III. (2S,5S)-9-Fluoro-2,3,4,5-tetrahydro-2,5-methanopyrido[3,4-f][1,4]oxazepine

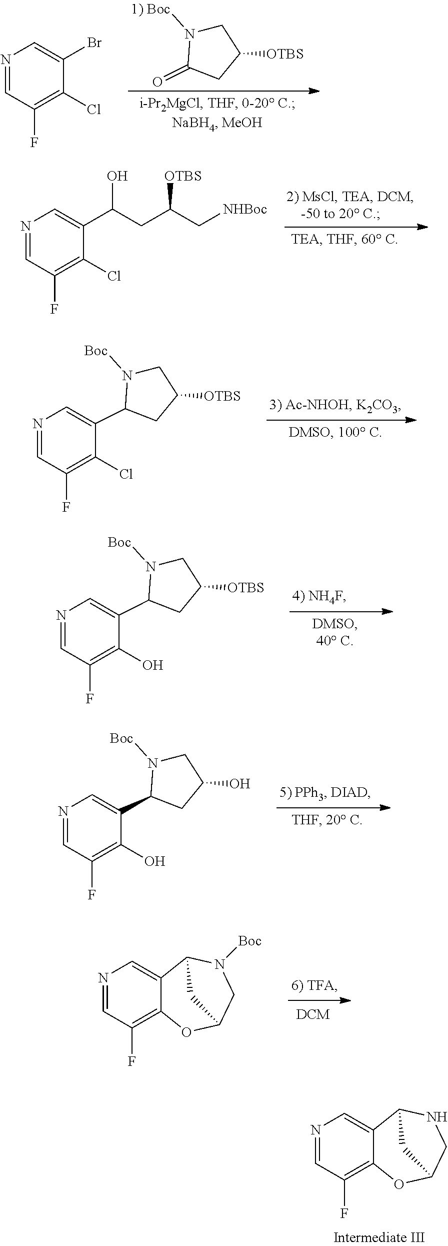

Step 1. A mixture containing 3-bromo-4-chloro-5-fluoropyridine (2.50 g, 11.9 mmol) in THF (50 mL) was treated at 0° C. with a 2 M solution of isopropylmagnesium chloride in THF (6.50 mL, 13.0 mmol). The mixture was stirred for 30 mm, then treated with a solution of tert-butyl (R)-4-((tert-butyldimethylsilyl)oxy)-2-oxopyrrolidine-1-carboxylate (3.00 g, 9.51 mmol) in 20 mL of THF. Stirred at RT for 4 hours, poured into saturated ammonium chloride (1 L) and extracted with EtOAc. Organic layer was dried (Na₂SO₄) and concentrated to dryness. The oil was redissolved in THF (50 mL), treated with methanol (1.0 mL, 25 mmol) followed by NaBH₄ (0.75 g, 20 mmol). The reaction was stirred for 2 h, poured into saturated ammonium chloride (500 mL) and extracted with EtOAc. The organic layer was dried (Na₂SO₄) and concentrated. Chromatography on SiO₂ (gradient of 0 to 100% EtOAc/DCM, 120 g silica gel) gave the desired intermediate alcohol as a mixture of diastereomers. MS (EI) calculated for C₁₆H₂₇ClFN₂O₄Si [M-tert-Bu+H]⁺, 393; found, 393.

Step 2. A mixture containing tert-butyl ((2R)-2-((tert-butyldimethylsilyl)oxy)-4-(4-chloro-5-fluoropyridin-3-yl)-4-hydroxybutyl)carbamate (1.35 g, 3.01 mmol) in DCM (30 mL) was treated with TEA (2.1 ml, 15.07 mmol) and cooled to −50° C. in a cooling bath. Methanesulfonyl chloride (0.50 mL, 6.42 mmol) was added and the resulting mixture was stirred for 15 min, then removed from the cooling bath, allowed to warm to ambient temperature, and stirred for 2 hours, LC/MS analysis indicated good conversion to the mesylate intermediate. The reaction mixture was concentrated, dissolved in THF (30 mL) and TEA (10 mL) and stirred overnight at 60° C. The mixture was concentrated, taken-up in EtOAc and washed with saturated ammonium chloride. The organic layer was dried (Na₂SO₄), concentrated, and the residue was purified by chromatography on silica gel (40 g, gradient of 0-50% EtOAc/DCM) giving tert-butyl (4R)-4-((tert-butyldimethylsilyl)oxy)-2-(4-chloro-5-fluoropyridin-3-yl)pyrrolidine-1-carboxylate, as a mixture of diastereomers. MS (EI) calculated for C₂₀H₃₃ClFN₂O₃Si [M+H]⁺, 432; found, 432.

Steps 3 and 4. A mixture containing tert-butyl (4R)-4-((tert-butyldimethylsilyl)oxy)-2-(4-chloro-5-fluoropyridin-3-yl)pyrrolidine-1-carboxylate as a mixture of diastereomers (394 mg, 0.914 mmol) in DMSO (4 mL) was treated with K₂CO₃ (650 mg, 4.70 mmol) and N-hydroxyacetamide (350 mg, 4.66 mmol). The reaction mixture was warmed to 100° C. and stirred overnight. The resulting suspension was filtered, and the filtrate was treated with ammonium fluoride (340 mg, 9.18 mmol) and stirred for 2 h at 40° C. The mixture was again filtered and purified by reverse phase chromatography (2-55% MeCN/water with 0.1% TFA) to provide two products in a 1.25:1 ratio. Isomer 1, the more polar was tert-butyl (2S,4R)-2-(5-fluoro-4-hydroxypyridin-3-yl)-4-hydroxypyrrolidine-1-carboxylate and was carried forward to the next reaction. MS (EI) calculated for C₁₄H₂₀FN₂O₄[M+H]⁺, 299; found, 299. Isomer 2, the less polar on reverse phase column, was tert-butyl (2R,4R)-2-(5-fluoro-4-hydroxypyridin-3-yl)-4-hydroxypyrrolidine-1-carboxylate and discarded.

Steps 5 and 6. A mixture containing tert-butyl (2S,4R)-2-(5-fluoro-4-hydroxypyridin-3-yl)-4-hydroxypyrrolidine-1-carboxylate (62 mg, 0.15 mmol) in THF (1 mL) was treated with DIAD (0.10 mL, 0.51 mmol) and triphenylphosphine (130 mg, 0.496 mmol). The mixture was stirred for 3 h, then diluted with MeOH (3 mL), filtered and purified by reverse phase chromatography (gradient of 2-55% MeCN/water with 0.1% TFA) to obtain tert-butyl (2S,5S)-9-fluoro-2,3-dihydro-2,5-methanopyrido[3,4-f][1,4]oxazepine-4(5H)-carboxylate. MS (EI) calculated for C₁₄H₁₈FN₂O₃ [M+H]⁺, 281; found, 281. A solution of tert-butyl (2S,5S)-9-fluoro-2,3-dihydro-2,5-methanopyrido[3,4-f][1,4]oxazepine-4(5H)-carboxylate (55 mg, 0.14 mmol) in 1 mL of DCM was treated with 1 mL of TFA. The solution was aged for 1 h and concentrated to obtain (2S,5S)-9-fluoro-2,3,4,5-tetrahydro-2,5-methanopyrido[3,4-f][1,4]oxazepine, TFA (45 mg, 100%). MS (EI) calculated for C₉H₁₀FN₂O [M+H]⁺181, found, 181.

### Preparation of Intermediate IV. (2S,5S)-8-Methyl-2,3,4,5-tetrahydro-2,5-methanopyrido[3,4-f][1,4]oxazepine-9-carbonitrile

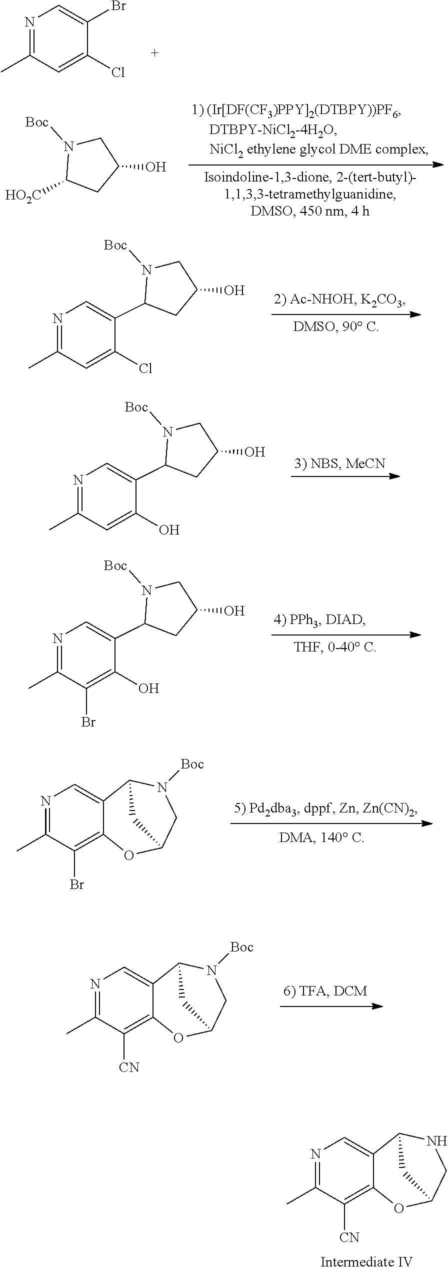

Step 1. A solution of 5-bromo-4-chloro-2-methylpyridine (300 mg, 1.45 mmol), (2R,4R)-1-(tert-butoxycarbonyl)-4-hydroxypyrrolidine-2-carboxylic acid (504 mg, 2.18 mmol), isoindoline-1,3-dione (214 mg, 1.45 mmol), DTBPY-NiCl₂-4H₂O (87 mg, 0.22 mmol), NiCl₂ ethylene glycol DME complex (32 mg, 0.15 mmol), 2-(tert-butyl)-1,1,3,3-tetramethylguanidine (373 mg, 2.18 mmol) in DMSO (16 mL) was treated with (Ir[DF(CF₃)PPY]₂(DTBPY))PF₆ (33 mg, 0.029 mmol). The reaction mixture was stirred in a photoreactor for 4 h with 450 nm irradiation. The mixture was diluted with water (25 mL) and extracted with EtOAc (3×25 mL). The combined organic layers were washed with brine, dried over anhydrous Na₂SO₄, filtered and concentrated. The residue was purified by chromatography on silica gel (1:3 petroleum ether/EtOAc) gave tert-butyl (4R)-2-(4-chloro-6-methylpyridin-3-yl)-4-hydroxypyrrolidine-1-carboxylate. MS (EI) calculated for C₁₈H₂₂ClN₂O₃ [M+H]⁺, 313; found, 313.

Step 2. A mixture of tert-butyl (4R)-2-(4-chloro-6-methylpyridin-3-yl)-4-hydroxypyrrolidine-1-carboxylate (220 mg, 0.703 mmol) in DMSO (1 mL) was treated with K₂CO₃ (583 mg, 4.22 mmol) and acetohydroxamic acid (158 mg, 2.11 mmol). The mixture was stirred at 90° C. for 12 h, filtered and purified by reverse phase chromatography (gradient of 5 to 35% MeCN/water with 0.05% NH₄OH and 10 mM NH₄HCO₃) to give tert-butyl (4R)-4-hydroxy-2-(4-hydroxy-6-methylpyridin-3-yl)pyrrolidine-1-carboxylate. ¹H NMR (5(0) MHz, CDCl₃) δ 7.43 (s, 1H), 6.12 (s, 1H), 4.51 (m, 1H), 4.35 (m, 1H), 3.74 (m, 1H), 3.61 (m, 1H), 2.70 (m, 1H), 2.33 (m, 1H), 2.09 (m, 1H), 2.02 (m, 4H), 1.43 (s, 9H); MS (EI) calculated for C₁₅H₂₃N₂O₄ [M+H]⁺, 295; found, 295.

Step 3. A solution of tert-butyl (4R)-4-hydroxy-2-(4-hydroxy-6-methylpyridin-3-yl)pyrrolidine-1-carboxylate (120 mg, 0.408 mmol) in MeCN (4 ml) was treated with NBS (73 mg, 0.41 mmol). The reaction mixture was stirred at 20° C. for 0.5 h, concentrated, and purified by reverse phase chromatography (gradient of 0 to 30% MeCN/water with 0.1% TFA) to provide tert-butyl (4R)-2-(5-bromo-4-hydroxy-6-methylpyridin-3-yl)-4-hydroxypyrrolidine-1-carboxylate. ¹H NMR (400 MHz, CD₃OD) δ 7.51 (m, 1H), 4.97 (m, 1H), 4.37 (m, 1H), 3.68 (m, 1H), 3.59 (m, 1H), 2.53 (s, 3H), 2.32 (m, 1H), 2.10 (m, 1H), 1.45, 1.28 (2s, 9H); MS (EI) calculated for C₁₅H₂₂BrN₂O₄ [M+H]⁺, 373, 375; found, 373, 375.

Step 4. A solution of tert-butyl (4R)-2-(5-bromo-4-hydroxy-6-methylpyridin-3-yl)-4-hydroxypyrrolidine-1-carboxylate (120 mg, 0.322 mmol) in THF (3 mL) was treated with Ph₃P (506 mg, 1.93 mmol) and DIAD (0.375 mL, 1.93 mmol) at 0° C. The mixture was stirred at 40° C. for 12 h, concentrated and purified by chromatography on SiO₂ (1:1 EtOAc/petroleum ether) to give tert-butyl (2S,5S)-9-bromo-8-methyl-2,3-dihydro-2,5-methanopyrido[3,4-f][1,4]oxazepine-4(5H)-carboxylate. MS (EI) calculated for C₁₅H₂₀BrN₂O₃[M+H]⁺, 355, 357; found, 355, 357.

Step 5. A mixture of tert-butyl (2S,5S)-9-bromo-8-methyl-2,3-dihydro-2,5-methanopyrido[3,4-f][1,4]oxazepine-4(5H)-carboxylate (40 mg, 0.11 mmol), zinc (4.4 mg, 0.068 mmol), dicyanozinc (66 mg, 0.56 mmol) in DMA (1 mL) was treated with Pd₂(dba)₃ (52 mg, 0.056 mmol) and dppf (62 mg, 0.11 mmol). The mixture was stirred at 150° C. for 2 h, quenched with water (5 mL) and extracted with EtOAc (3×5 mL). The combined organic layers were washed with brine, dried over anhydrous Na₂SO₄, filtered and concentrated. The residue was purified by chromatography on silica gel (1:1 EtOAc/petroleum ether) to give tert-butyl (2S,5S)-9-cyano-8-methyl-2,3-dihydro-2,5-methanopyrido[3,4-f][1,4]oxazepine-4(5H)-carboxylate. MS (EI) calculated for C₁₆H₂₀N₃O₃ [M+H]⁺, 302; found, 302.

Step 6. A solution of tert-butyl (2S,5S)-9-cyano-8-methyl-2,3-dihydro-2,5-methanopyrido[3,4-f][1,4]oxazepine-4(5H)-carboxylate (25 mg, 0.083 mmol) in DCM (2 mL) and TFA (0.5 mL) was stirred at 25° C. for 1 h. The mixture was concentrated to give (2S,5S)-8-methyl-2,3,4,5-tetrahydro-2,5-methanopyrido[3,4-f][1,4]oxazepine-9-carbonitrile. MS (EI) calculated for C₁₁H₁₂N₃O [M+H]⁺, 202; found, 202.

### Preparation of Intermediate V. (2S,5S)-9-Fluoro-8-methyl-2,3,4,5-tetrahydro-2,5-methanopyrido[3,4-f][1,4]oxazepine

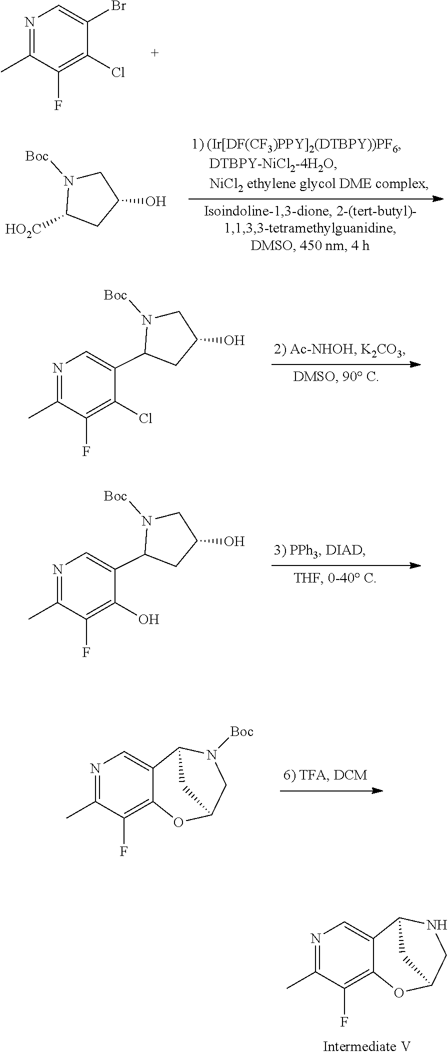

Step 1. A solution of 5-bromo-4-chloro-3-fluoro-2-methylpyridine (0.80 g, 3.6 mmol), 2-(tert-butyl)-1,1,3,3-tetramethylguanidine (0.916 g, 5.35 mmol), isoindoline-1,3-dione (0.524 g, 3.56 mmol), DTBPY-NiCl₂-4H₂O (0.355 g, 0.891 mmol), NiCl₂ ethylene glycol dimethyl ether complex (0.078 g, 0.36 mmol) and (2R,4R)-1-(tert-butoxycarbonyl)-4-hydroxypyrrolidine-2-carboxylic acid (0.989 g, 4.28 mmol) in DMSO (2 mL) was treated with (Ir[DF(CF₃)PPY]₂(DTBPY))PF₆ (0.080 g, 0.071 mmol) under N₂. The reaction mixture was stirred with irradiation with Photoreactor at 450 nm for 4 h. The mixture was purified by reverse phase chromatography (gradient of 40-70% MeCN/water with 0.05% NH₄OH), to provide tert-butyl (2S,4R)-2-(4-chloro-5-fluoro-6-methylpyridin-3-yl)-4-hydroxypyrrolidine-1-carboxylate. ¹H NMR (400 MHz, CDCl₃) δ 7.88-8.17 (m, 1H), 5.11-5.28 (m, 1H), 4.29-4.42 (m, 1H), 3.52-3.73 (m, 2H), 2.40-2.56 (m, 3H), 1.78-1.98 (m, 1H), 1.42 (br d, J=6.65 Hz, 1H), 0.97-1.39 (m, 9H); MS (EI) calculated for C₁₅H₂₁ClFN₂O₃ [M+H]⁺, 331; found, 331.

Step 2. To a mixture of tert-butyl (2S,4R)-2-(4-chloro-5-fluoro-6-methylpyridin-3-yl)-4-hydroxypyrrolidine-1-carboxylate (100 mg, 0.302 mmol) in DMSO (2 mL) was added K₂CO₃ (251 mg, 1.81 mmol) and N-hydroxyacetamide (68 mg, 0.91 mmol) at 20° C. Then the mixture was stirred at 90° C. for 20 h. The mixture was purified by reverse phase chromatography (gradient of 0-30% MeCN/water with 0.05% NH₄OH) to provide tert-butyl (2S,4R)-2-(5-fluoro-4-hydroxy-6-methylpyridin-3-yl)-4-hydroxypyrrolidine-1-carboxylate as a solid. ¹H NMR (400 MHz, CDCl₃) δ 7.27 (s, 1H), 4.84-5.16 (m, 1H), 4.39 (br s, 1H), 3.52-3.76 (m, 2H), 2.26-2.55 (m, 4H), 1.92-2.19 (m, 1H), 1.14-1.59 (m, 9H); MS (EI) calculated for C₁₅H₂₂FN₂O₄[M+H]⁺, 313; found, 313.

Step 3. A solution of tert-butyl (2S,4R)-2-(5-fluoro-4-hydroxy-6-methylpyridin-3-yl)-4-hydroxypyrrolidine-1-carboxylate (70 mg, 0.22 mmol) in THF (3 mL) was treated with Ph₃P (353 mg, 1.35 mmol) and DIAD (0.26 mL, 1.3 mmol). Then it was stirred at 20° C. for 16 h. The mixture was purified by prep-TLC (SiO₂, Pet. Ether/EtOAc=2/1) to give tert-butyl (2S,5S)-9-fluoro-8-methyl-2,3-dihydro-2,5-methanopyrido[3,4-f][1,4]oxazepine-4(5H)-carboxylate as a solid. ¹H NMR (400 MHz, CDCl₃) δ 7.88-8.07 (m, 1H), 5.11 (br s, 1H), 4.81-5.02 (m, 1H), 3.56-3.84 (m, 2H), 2.43 (d, J=2.7 Hz, 3H), 2.11-2.34 (m, 2H), 1.35-1.51 (m, 9H); MS (EI) calculated for C₁₅H₂₀FN₂O₃ [M+H]⁺, 295; found, 295.

Step 4. A solution of tert-butyl (2S,5S)-9-fluoro-8-methyl-2,3-dihydro-2,5-methanopyrido[3,4-f][1,4]oxazepine-4(5H)-carboxylate (70 mg, 0.17 mmol) in DCM (2 mL) was treated with TFA (0.7 mL). Then it was stirred at 20° C. for 1 h. The mixture was concentrated to give (2S,5S)-9-fluoro-8-methyl-2,3,4,5-tetrahydro-2,5-methanopyrido[3,4-f][1,4]oxazepine (Intermediate V) as an oil which was used directly. MS (EI) calculated for C₁₀H₁₂FN₂O [M+H]⁺, 195; found, 195.

### Preparation of Intermediate VI. (2S,5S)-2,3,4,5-Tetrahydro-2,5-methanopyrido[3,4-f][1,4]thiazepine-9-carbonitrile

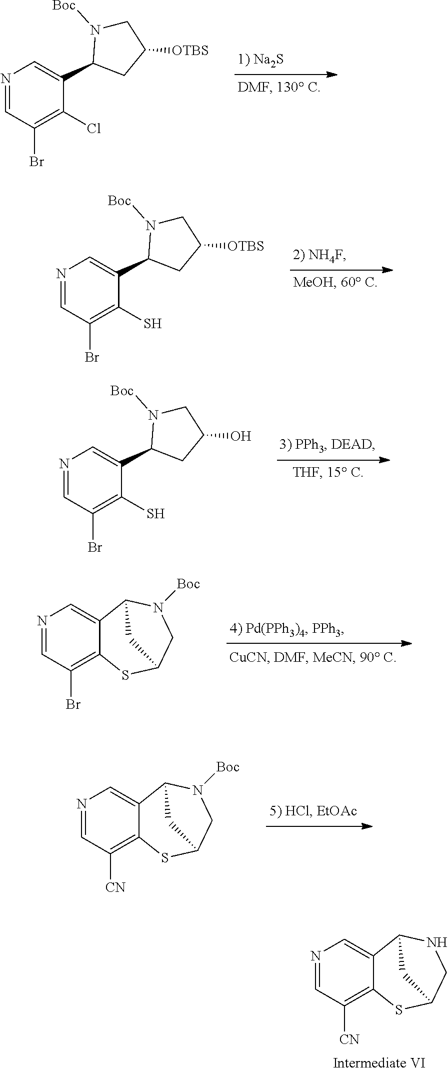 [SMILES: CC(C)(C)OC(=O)N1C[C@H](O[Si](C)(C)C(C)(C)C)C[C@H]1c1cncc(Br)c1Cl ; CC(C)(C)OC(=O)N1C[C@H](O[Si](C)(C)C(C)(C)C)C[C@H]1c1cncc(Br)c1S ; CC(C)(C)OC(=O)N1C[C@H](O)C[C@H]1c1cncc(Br)c1S ; CC(C)(C)OC(=O)N1C[C@@H]2C[C@H]1c1cncc(Br)c1S2 ; CC(C)(C)OC(=O)N1C[C@@H]2C[C@H]1c1cncc(C#N)c1S2 ; N#Cc1cncc2c1S[C@@H]1CN[C@H]2C1]

Step 1. A solution of tert-butyl (2S,4R)-2-(5-bromo-4-chloropyridin-3-yl)-4-((tert-butyldimethylsilyl)oxy)pyrrolidine-1-carboxylate (149 g, 303 mmol) in DMF (1700 mL) was treated with Na₂S (47.3 g, 25.4 mL, 600 mmol). The mixture was stirred overnight at 130° C., then poured into water (5 L). The solid precipitate was collected, washed with water (5 L) and acetone (3 L), and the solid dried under vacuum giving crude tert-butyl (2S,4R)-2-(5-bromo-4-mercaptopyridin-3-yl)-4-((tert-butyldimethylsilyl)oxy)pyrrolidine-1-carboxylate.

Step 2. A mixture containing tert-butyl (2S,4R)-2-(5-bromo-4-mercaptopyridin-3-yl)-4-((tert-butyldimethylsilyl)oxy)pyrrolidine-1-carboxylate (150 g, 307 mmol) in MeOH (1000 mL) was treated with NH₄F (34.0 g, 900 mmol) and stirred for 12 h at 60° C. The mixture was concentrated giving crude tert-butyl (2S,4R)-2-(5-bromo-4-mercaptopyridin-3-yl)-4-hydroxypyrrolidine-1-carboxylate.

Step 3. A mixture containing tert-butyl (2S,4R)-2-(5-bromo-4-mercaptopyridin-3-yl)-4-hydroxypyrrolidine-1-carboxylate (110 g, 290 mmol) in THF (700 mL) was treated with PPh₃ (92 g, 350 mmol) and DEAD (61 g, 350 mmol) at 0° C. The mixture was stirred for 12 h at RT, poured into water and extracted with EtOAc. The organic layer was washed with brine, dried (Na₂SO₄) and concentrated. Chromatography on silica gel (1:3 to 1:4 petroleum ether/EtOAc) gave tert-butyl (2S,5S)-9-bromo-2,3-dihydro-2,5-methanopyrido[3,4-f][1,4]thiazepine-4(5H)-carboxylate.

Step 4. A mixture of tert-butyl (2S,5S)-9-bromo-2,3-dihydro-2,5-methanopyrido[3,4-f][1,4]thiazepine-4(5H)-carboxylate (75 g, 200 mmol) in DMF (450 mL) and MeCN (1100 mL) was treated with CuCN (18.8 g, 200 mmol), Pd(PPh₃)₄ (49 g, 40 mmol) and PPh₃ (116 g, 440 mmol). The mixture was stirred at 90° C. for 1.5 h, then poured into water. The suspension was filtered and the precipitate collected by filtration through a pad of CELITE (diatomaceous earth). The pad was washed with excess EtOAc and the organic layers washed with brine, dried (Na₂SO₄) and concentrated. Chromatography on silica gel (100:1 to 0:1 petroleum ether/EtOAc) gave tert-butyl (2S,5S)-9-cyano-2,3-dihydro-2,5-methanopyrido[3,4-f][1,4]thiazepine-4(5H)-carboxylate.

Step 5. A mixture of tert-butyl (2S,5S)-9-cyano-2,3-dihydro-2,5-methanopyrido[3,4-f][1,4]thiazepine-4(5H)-carboxylate (41 g, 130 mmol) in EtOAc (200 mL) was treated with 4 M HCl/EtOAc (337 mL, 1350 mmol) and stirred for 12 h. The precipitate was collected by filtration to give (2S,5S)-2,3,4,5-tetrahydro-2,5-methanopyrido[3,4-f][1,4]thiazepine-9-carbonitrile. ¹H NMR (400 MHz, D₂) δ 8.74 (s, 1H), 8.48 (s, 1H), 5.18 (d, J=8 Hz, 1H), 4.20-4.23 (m, 1H), 3.89-4.01 (m, 1H), 3.84-3.87 (m, 1H), 2.75-2.79 (m, 1H), 2.48 (d, J=12 Hz, 1H); MS (EI) calculated for C₁₀H₁₀N₃S [M+H]⁺, 204; found, 204.

### Preparation of Intermediate VII. (3R,6S)-3,4,5,6-Tetrahydro-2H-3,6-epiminooxocino[3,2-c](pyridine-10-carbonitrile

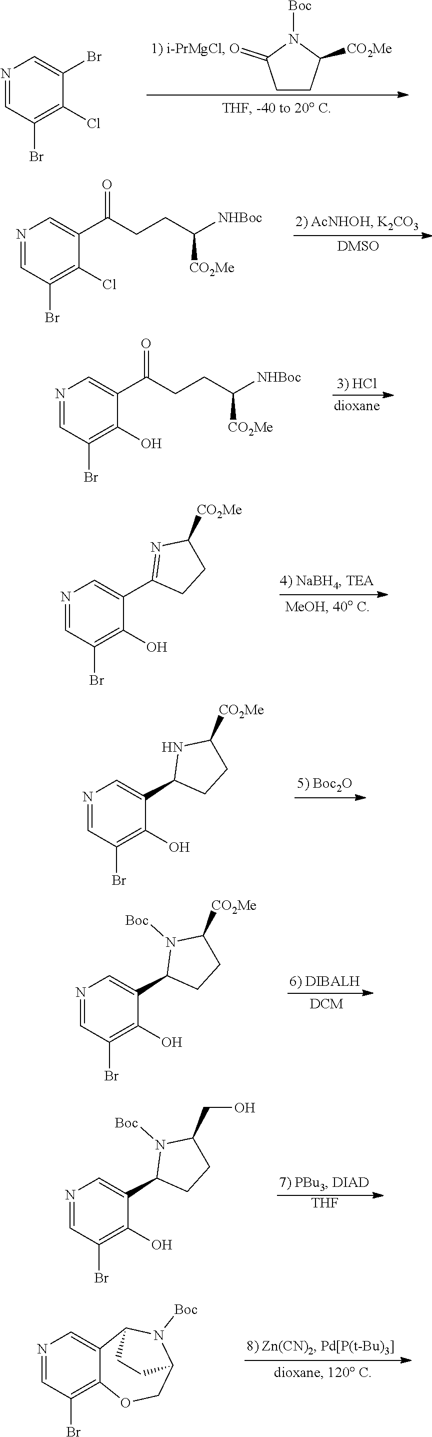

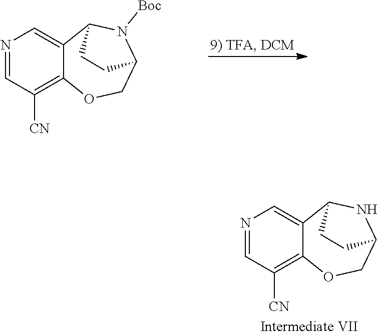 [SMILES: CC(C)(C)OC(=O)N1[C@@H]2CC[C@H]1c1cncc(C#N)c1OC2 ; N#Cc1cncc2c1OC[C@H]1CC[C@@H]2N1]

Step 1. A mixture of 3,5-dibromo-4-chloropyridine (8.37 g, 30.8 mmol) in THF (75 mL) was treated dropwise with a solution of isopropyl magnesium chloride in THF (14.4 mL, 28.8 mmol) at −40 TC. The yellow suspension mixture was stirred at 0° C. for 60 min. Then the mixture was cooled to −40 TC. Next, I-(tert-butyl) 2-methyl (R)-5-oxopyrrolidine-1,2-dicarboxylate (5.0 g, 20.6 mmol) in THF (25 mL) was added dropwise at −40 TC. The mixture was stirred at 20° C. for 1 h, then quenched with sat. NH₄Cl. The mixture was extracted with EtOAc, washed with brine, dried over anhydrous Na₂SO₄, filtered and concentrated to give an oil which was purified by silica gel chromatography (0 to 15% EtOAc/Pet, ether) to give methyl (R)-5-(5-bromo-4-chloropyridin-3-yl)-2-((tert-butoxycarbonyl)amino)-5-oxopentanoate as an oil. ¹H NMR (400) MH-z, CDCl₃) S 8.82 (s, 1H), 8.58 (s, 1H), 5.12 (br s, 1H), 4.40 (br s, 1H), 3.78 (s, 3H), 3.01-3.15 (m, 1H), 2.30-2.38 (m, 1H), 2.05-2.11 (m, 1H), 1.43 (s, 9H); MS (EI) calculated for C₁₆H₂₁BrClN₂O₅ [M+H]⁺, 437; found, 437.

Step 2. A mixture of methyl (R)-5-(5-bromo-4-chloropyridin-3-yl)-2-((tert-butoxycarbonyl)amino)-5-oxopentanoate (1.50 g, 3.44 mmol) in DMSO (40 mL) was treated with K₂CO₃ (9.52 g, 68.9 mmol) and acetohydroxamic acid (2.58 g, 34.4 mmol). The mixture was stirred at 20° C. for 1 h, and the crude product was purified by reverse phase chromatography to give methyl (R)-5-(5-bromo-4-hydroxypyridin-3-yl)-2-((tert-butoxycarbonyl)amino)-5-oxopentanoate as an oil. ¹H NMR (400 MHz, CD₃OD) δ 8.30 (s, 1H), 8.21 (d, J=1.5 Hz, 1H), 4.18 (br dd, J=5.0 Hz, 9.2 Hz, 1H), 3.72 (s, 3H), 3.22 (br t, J=7.0 Hz, 2H), 2.16-2.24 (m, 1H), 1.88-2.01 (m, 1H), 1.41 (s, 9H); MS (EI) calculated for C₁₆H₂₀BrN₂O₅ [M-water+H]⁺, 401; found, 401.

Step 3. A flask with methyl (R)-5-(5-bromo-4-hydroxypyridin-3-yl)-2-((tert-butoxycarbonyl)amino)-5-oxopentanoate (1.20 g, 2.88 mmol) was treated with 4M solution of HCl in dioxane (30 mL). Then the mixture was stirred at 20° C. for 1 h, then concentrated to dryness giving methyl (R)-5-(5-bromo-4-hydroxypyridin-3-yl)-3,4-dihydro-2H-pyrrole-2-carboxylate. MS (EI) calculated for C₁₁H₁₂BrN₂O₃[M+H]⁺, 301; found, 301.

Step 4. A mixture of methyl (R)-5-(5-bromo-4-hydroxypyridin-3-yl)-3,4-dihydro-2H-pyrrole-2-carboxylate (900 mg, 3.01 mmol) in MeOH (15 mL) was treated with triethylamine (1520 mg, 15.0 mmol) until pH>7, then treated with NaBH₄ (1370 mg, 36.1 mmol). The mixture was stirred at 40° C. for 12 h, then concentrated in vacuum to give product methyl (2R,5S)-5-(5-bromo-4-hydroxypyridin-3-yl)pyrrolidine-2-carboxylate as a solid. MS (EI) calculated for C₁₁H₁₄BrN₂O₃ [M+H]⁺, 303; found, 303.

Step 5. A mixture of methyl (2R,5S)-5-(5-bromo-4-hydroxypyridin-3-yl)pyrrolidine-2-carboxylate (700 mg, 2.33 mmol) in MeOH (20 mL) was treated with di-tert-butyl dicarbonate (507 mg, 2.33 mmol). The mixture was stirred at 25° C. for 1 h and concentrated. The residue was purified by reverse phase chromatography (gradient of 8-38% MeCN/water with 0.05% NH₄OH) to give 1-(tert-butyl) 2-methyl (2R,5S)-5-(5-bromo-4-hydroxypyridin-3-yl)pyrrolidine-1,2-dicarboxylate as a solid. ¹H NMR (400 MHz, CDCl₃) δ 8.17-8.57 (m, 1H), 7.75-8.07 (m, 1H), 5.20 (br s, 1H), 4.33 (br s, 1H), 3.43-3.98 (m, 3H), 2.26 (br s, 2H), 1.75-2.11 (m, 2H), 1.17-1.46 (m, 9H); MS (EI) calculated for C₁₆H₂₂BrN₂O₅ [M+H]⁺, 403; found, 403.

Step 6. A stirred solution of 1-(tert-butyl) 2-methyl (2R,5S)-5-(5-bromo-4-hydroxypyridin-3-yl)pyrrolidine-1,2-dicarboxylate (200 mg, 0.498 mmol) in DCM (5 mL) was treated with DIBAL-H (1.50 mL, 1.50 mmol) at 0° C. The reaction was stirred at 20° C. for 1 h, and concentrated. The residue was purified by reverse phase chromatography (gradient of 15-45% MeCN/water with 0.1% TFA) to give tert-butyl (2S,5R)-2-(5-bromo-4-hydroxypyridin-3-yl)-5-(hydroxymethyl)pyrrolidine-1-carboxylate as a solid. ¹H NMR (400 MHz, DMSO-d₆) δ 11.72 (br s, 1H), 8.16 (br s, 1H), 7.66 (br s, 1H), 4.74 (t, J=6.8 Hz, 1H), 3.66-3.79 (m, 2H), 3.54 (br d, J=10.1 Hz, 1H), 2.07-2.18 (m, 1H), 1.75-1.89 (m, 2H), 1.70 (br s, 1H), 1.12-1.45 (m, 9H); MS (EI) calculated for C₁₅H₂₂BrN₂O₄[M+H]⁺, 375; found, 375.

Step 7. A stirred solution of tert-butyl (2S,5R)-2-(5-bromo-4-hydroxypyridin-3-yl)-5-(hydroxymethyl)pyrrolidine-1-carboxylate (50 mg, 0.13 mmol) in THF (1 mL) was treated with PBu₃ (0.167 ml, 0.670 mmol) and DIAD (0.13 mL, 0.67 mmol). After stirring at 25° C. for 12 h, a mixture was concentrated in vacuo. The residue was purified by prep. TLC (3:1 pet, ether/EtOAc) to give tert-butyl (3R,6S)-10-bromo-3,4,5,6-tetrahydro-2H-3,6-epiminooxocino[3,2-c]pyridine-11-carboxylate as an oil. MS (EI) calculated for C₁₅H₂₀BrN₂O₃ [M+H]⁺, 357; found, 357.

Step 8. A solution of tert-butyl (3R,6S)-10-bromo-3,4,5,6-tetrahydro-2H-3,6-epiminooxocino[3,2-c]pyridine-11-carboxylate (40 mg, 0.11 mmol) in dioxane (1 mL) was treated with dicyanozinc (26 mg, 0.23 mmol) and bis(tri-tert-butylphosphine)palladium(0) (17 mg, 0.034 mmol). The mixture heated at 120° C. for 12 h, then concentrated in vacuum. The residue was purified by prep. TLC (2:1 Pet, ether/EtOAc) to give tert-butyl (3R,6S)-10-cyano-3,4,5,6-tetrahydro-2H-3,6-epiminooxocino[3,2-c]pyridine-11-carboxylate as an oil. MS (EI) calculated for C₁₆H₂₀N₃O₃ [M+H]⁺, 302; found, 302.

Step 9. A solution of tert-butyl (3R,6S)-10-cyano-3,4,5,6-tetrahydro-2H-3,6-epiminooxocino[3,2-c]pyridine-11-carboxylate (80 mg, 0.27 mmol) in 2:1 DCM/TFA (3 mL) was stirred at 20° C. for 1 h. The reaction mixture was concentrated under reduced pressure to give the product, Intermediate VII, (3R,6S)-3,4,5,6-tetrahydro-2H-3,6-epiminooxocino[3,2-c]pyridine-10-carbonitrile as an oil. MS (EI) calculated for C₁₁H₁₂N₃O [M+H]⁺, 202; found, 202.

### Preparation of Intermediate VIII. 1-((2S,5S)-9-Bromo-2,3-dihydro-2,5-methanopyrido[3,4-f][1,4]oxazepin-4(5H)-yl)-3,3-difluoro-2,2-dimethylpropan-1-one

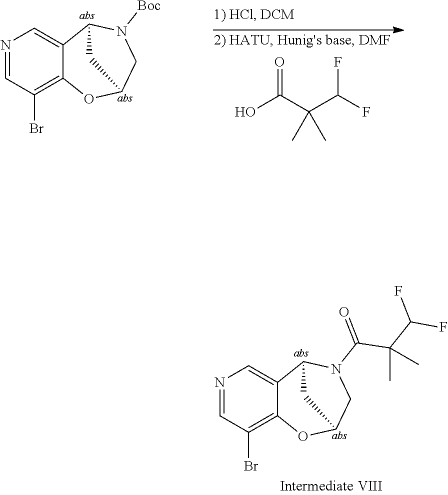

Step 1. A solution of tert-butyl (2S,5S)-9-bromo-2,3-dihydro-2,5-methanopyrido[3,4-f][1,4]oxazepine-4(5H)-carboxylate (1.0 g, 2.93 mmol) in DCM (14.7 mL) was treated with a 4 M solution of HCl in dioxane (2.90 mL, 11.7 mmol) and stirred overnight. The reaction was concentrated, giving (2S,5S)-9-bromo-2,3,4,5-tetrahydro-2,5-methanopyrido[3,4-f][1,4]oxazepine, HCl. MS (EI) Calculated for C₉H₁₁BrN₂O [M+H]⁺, 241, 243; found, 241, 243.

Step 2. A solution of 3,3-difluoro-2,2-dimethylpropanoic acid (0.349 g, 2.52 mmol) in DMF (34 mL) was treated with Hunig's Base (1.47 ml, 8.41 mmol) then HATU (1.28 g, 3.37 mmol) and stirred at RT for 10 min. (2S,5S)-9-Bromo-2,3,4,5-tetrahydro-2,5-methanopyrido[3,4-f][1,4]oxazepine, HCl (0.467 g, 1.68 mmol) was added. The mixture was stirred at RT for 2 h. The reaction was quenched with water and aqueous sodium bicarbonate, then extracted 3 times with DCM. The combined organic extracts were washed twice with aqueous LiCl, then dried over sodium sulfate, filtered, and concentrated. The residue was purified by chromatography on silica gel (24 g silica gel, 0-20% MeOH/DCM) giving Intermediate VIII, 1-((2S,5S)-9-bromo-2,3-dihydro-2,5-methanopyrido[3,4-f][1,4]oxazepin-4(5H)-yl)-3,3-difluoro-2,2-dimethylpropan-1-one. ¹H NMR (500 MHz, Chloroform-d) δ 8.52 (s, 1H), 8.40 (s, 1H), 6.01 (t, J=56.7 Hz, 1H), 5.39 (d, J=4.8 Hz, 1H), 5.36 (s, 1H), 4.08 (d, J=12.0 Hz, 1H), 4.00 (dd, J=12.2, 3.4 Hz, 1H), 2.39-2.28 (m, 1H), 2.24 (d, J=12.9 Hz, 1H), 1.31 (s, 3H), 1.20 (s, 3H). MS (EI) Calculated for C₁₄H₁₆BrF₂N₂O₂ [M+H]⁺, 361, 363; found, 361, 363.

### Preparation of Intermediate IX. ((2S,5S)-9-Bromo-2,3-dihydro-2,5-methanopyrido[3,4-f][1,4]oxazepin-4(5H)-yl)(4-(trifluoromethyl)bicyclo[2.2.1]heptan-1-yl)methanone

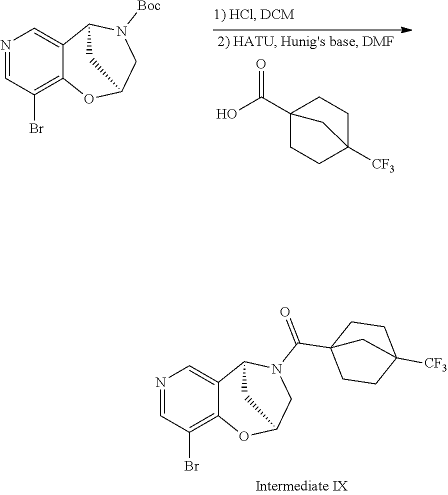 [SMILES: CC(C)(C)OC(=O)N1C[C@@H]2C[C@H]1c1cncc(Br)c1O2 ; O=C(O)C12CCC(C(F)(F)F)(CC1)C2 ; O=C(N1C[C@@H]2C[C@H]1c1cncc(Br)c1O2)C12CCC(C(F)(F)F)(CC1)C2]

Intermediate IX was prepared in a fashion analogous to the synthesis of intermediate VIII, substituting 3,3-difluoro-2,2-dimethylpropanoic acid for the appropriate carboxylic acid. ¹H NMR (5(0) MHz, Chloroform-d) δ 8.54 (s, 1H), 8.42 (s, 1H), 5.40 (s, 2H), 4.03 (d, J=11.6 Hz, 1H), 3.92 (d, J=10.7 Hz, 1H), 2.33 (d, J=13.0 Hz, 1H), 2.25 (d, J=12.9 Hz, 1H), 1.87 (m, 8H), 1.63 (s, 2H). MS (EI) Calculated for C₁₈H₁₉BrF₃N₂O₂[M+H]⁺, 431, 433; found, 431, 433.

### Preparation of Intermediate X. ((2S,5S)-9-Ethynyl-2,3-dihydro-2,5-methanopyrido[3,4-f][1,4]oxazepin-4(5H)-yl)(4-fluorobicyclo[2.2.1]heptan-1-yl)methanone

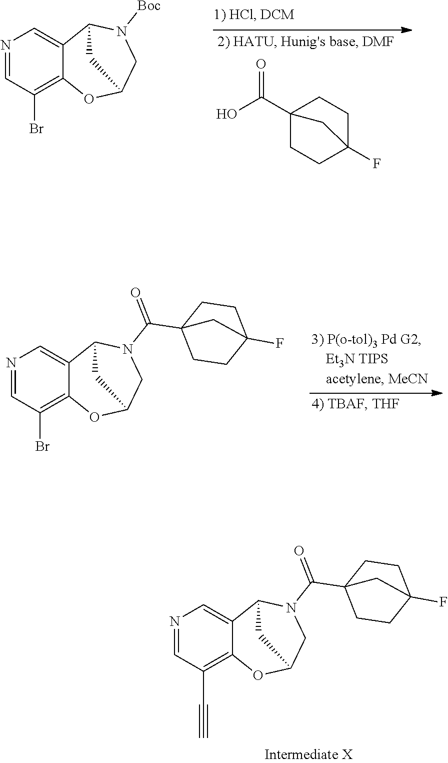 [SMILES: CC(C)(C)OC(=O)N1C[C@@H]2C[C@H]1c1cncc(Br)c1O2 ; O=C(O)C12CCC(F)(CC1)C2 ; O=C(N1C[C@@H]2C[C@H]1c1cncc(Br)c1O2)C12CCC(F)(CC1)C2 ; C#Cc1cncc2c1O[C@H]1C[C@@H]2N(C(=O)C23CCC(F)(CC2)C3)C1]

Step 1. A solution of tert-butyl (2S,5S)-9-bromo-2,3-dihydro-2,5-methanopyrido[3,4-f][1,4]oxazepine-4(5H)-carboxylate (2.0 g, 5.86 mmol) in DCM (29.3 mL) was treated with a 4 M solution of HCl in dioxane (8.8 mL, 35 mmol) and stirred overnight. The reaction was concentrated, giving (2S,5S)-9-bromo-2,3,4,5-tetrahydro-2,5-methanopyrido[3,4-f][1,4]oxazepine, HCl. MS (EI) Calculated for C₉H₁₁BrN₂O [M+H]⁺, 241, 243; found, 241, 243.

Step 2. A solution of 4-fluorobicyclo[2.2.1]heptane-1-carboxylic acid (1.39 g, 8.79 mmol) and (2S,5S)-9-bromo-2,3,4,5-tetrahydro-2,5-methanopyrido[3,4-f][1,4]oxazepine, HCl (1.67 g, 5.86 mmol) in DMF (117 mL) was treated with Hunig's Base (5.12 mL, 29.3 mmol) then HATU (4.46 g, 11.7 mmol) and stirred at RT overnight. The reaction was quenched with water, then extracted 3 times with EtOAc. The combined organic extracts were washed twice with aqueous LiCl, then dried over sodium sulfate, filtered, and concentrated. The residue was purified by chromatography on silica gel (24 g silica gel, 0-20% MeOH/DCM) giving ((2S,5S)-9-bromo-2,3-dihydro-2,5-methanopyrido[3,4-f][1,4]oxazepin-4(5H)-yl)(4-fluorobicyclo[2.2.1]heptan-1-yl)methanone. MS (EI) Calculated for C₇H₁₉BrFN₂O₂[M+H]⁺, 381, 383; found, 381, 383.

Step 3. ((2S,5S)-9-Bromo-2,3-dihydro-2,5-methanopyrido[3,4-f][1,4]oxazepin-4(5H)-yl)(4-fluorobicyclo[2.2.1]heptan-1-yl)methanone (1.80 g, 4.71 mmol) and chloro[tri(o-tolyl)phosphine][2-(2′-amino-1,1′-biphenyl)]palladium(II) (0.289 g, 0.471 mmol) were added to a vial. The vial was degassed and backfilled with nitrogen. MeCN (23.5 mL) was added followed by (triisopropylsilyl)acetylene (1.58 mL, 1.29 mmol) and Et₃N (1.97 mL, 14.1 mmol), then the reaction was heated to 90° C. overnight. The reaction was cooled to RT and concentrated. The residue was purified by chromatography on silica gel (40 g silica gel, 0-100% EtOAc/Hexanes) giving (4-fluorobicyclo[2.2.1]heptan-1-yl)((2S,5S)-9-((triisopropylsilyl)ethynyl)-2,3-dihydro-2,5-methanopyrido[3,4-f][1,4]oxazepin-4(5H)-yl)methanone. MS (EI) Calculated for C₂₈H₄₀FN₂O₂Si [M+H]⁺, 483; found, 483.

Step 4. A solution of (4-fluorobicyclo[2.2.1]heptan-1-yl)((2S,5S)-9-((triisopropylsilyl)ethynyl)-2,3-dihydro-2,5-methanopyrido[3,4-f][1,4]oxazepin-4(5H)-yl)methanone (1.97 g, 4.09 mmol) in THF (41 mL) was treated with a 1 M solution TBAF in THF (4.5 mL, 4.5 mmol). The reaction was allowed to stir at RT for 1 h. The solution was concentrated and the residue purified by chromatography on silica gel (24 g silica gel, 0-100% EtOAc/Hexanes) giving Intermediate X, ((2S,5S)-9-ethynyl-2,3-dihydro-2,5-methanopyrido[3,4-f][1,4]oxazepin-4(5H)-yl)(4-fluorobicyclo[2.2.1]heptan-1-yl)methanone. ¹H NMR (500 MHz, Chloroform-d) δ 8.47 (s, 1H), 8.38 (s, 1H), 5.35 (s, 1H), 5.26 (s, 1H), 3.96 (d, J=11.5 Hz, 1H), 3.85 (d, J=9.4 Hz, 1H), 3.40 (s, 1H), 2.19 (s, 2H), 2.03 (m, 2H), 1.95 (m, 4H), 1.79 (m, 4H). MS (EI) Calculated for C₁₉H₂₀FN₂O₂ [M+H]⁺, 327; found, 327.

### Preparation of Intermediate XI. (2S,5S)-9-((4-fluorophenyl)ethynyl)-2,3,4,5-tetrahydro-2,5-methanopyrido[3,4-f][1,4]oxazepine

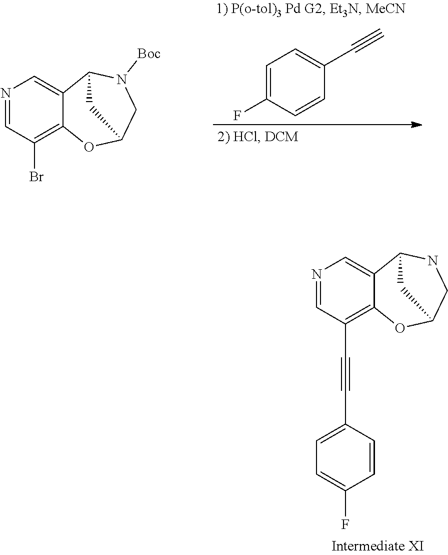

Step 1. Tert-Butyl (2S,5S)-9-bromo-2,3-dihydro-2,5-methanopyrido[3,4-f][1,4]oxazepine-4(5H)-carboxylate (136 mg, 0.4 mmol) and chloro[tri(o-tolyl)phosphine][2-(2′-amino-1,1′-biphenyl)]palladium(II) (25 mg, 0.04 mmol) were added to a vial. The vial was degassed and backfilled with nitrogen. MeCN (2.0 mL) was added followed by 1-ethynyl-4-fluorobenzene (72 mL, 0.6 mmol) and Et₃N (137 μL, 1.2 mmol), then the reaction was heated to 90° C. overnight. The reaction was cooled to RT and concentrated. The residue was purified by chromatography on silica gel (4 g silica gel, 0-70% EtOAc/Hexanes) giving tert-butyl (2S,5S)-9-((4-fluorophenyl)ethynyl)-2,3-dihydro-2,5-methanopyrido[3,4-f][1,4]oxazepine-4(5H)-carboxylate. MS (EI) Calculated for C₂₂H₂₂FN₂O₃ [M+H]⁺, 381; found, 381.

Step 2. A solution of tert-butyl (2S,5S)-9-((4-fluorophenyl)ethynyl)-2,3-dihydro-2,5-methanopyrido[3,4-f][1,4]oxazepine-4(5H)-carboxylate (37 mg, 0.097 mmol) in DCM (486 μL) was treated with a 4 M solution of HCl in dioxane (195 μL, 0.778 mmol) and stirred overnight. The reaction was concentrated, giving Intermediate XI, (2S,5S)-9-((4-fluorophenyl)ethynyl)-2,3,4,5-tetrahydro-2,5-methanopyrido[3,4-f][1,4]oxazepine, HCL. MS (EI) Calculated for C₁₇H₁₄FN₂O [M+H]⁺, 281; found, 281.

### Preparation of Intermediate XII. (2R,5R,10S)-10-Methyl-2,3,4,5-tetrahydro-2,5-methanopyrido[3,4-f][1,4]oxazepine-9-carbonitrile

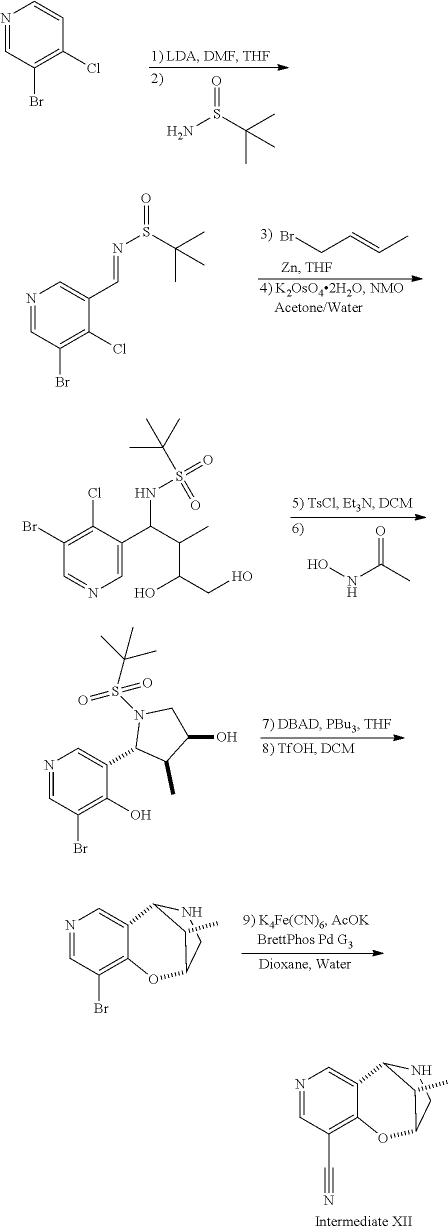

Step 1. To a mixture of diisopropylamine (15.3 mL, 109 mmol) in THF (100 mL) was added butyllithium (40.5 mL, 101 mmol) at −78° C. under N₂. The mixture was stirred at 0° C. for 30 min. Then the mixture was cooled to −78° C. 3-Bromo-4-chloropyridine (15 g, 78 mmol) in THF (90 mL) was added dropwise at −78° C. under N₂. After the mixture was stirred at −78° C. for 2 h, N,N-dimethylformamide (7.41 g, 101 mmol) in THF (10 mL) was added at −78° C. The temperature was allowed to warm to 20° C. and the mixture was stirred at 20° C. for 16 h. The reaction was quenched with sat. NH₄Cl. The organic layer was separated and the aqueous was re-extracted twice with EtOAc. The combined organic layers were washed with brine, dried over anhydrous Na₂SO₄, filtered and concentrated to give an oil which was purified by flash silica gel chromatography (80 g silica gel, Eluent of 10% EtOAc/Pet, ether gradient @20 mL/min) to give 5-bromo-4-chloronicotinaldehyde. ¹H NMR (400 MHz, Chloroform-d) δ 10.38-10.54 (m, 1H), 8.94 (d, J=2.8 Hz, 2H).

Step 2. To a stirred mixture of 5-bromo-4-chloronicotinaldehyde (4 g, 18.1 mmol) in THF (100 mL) was added 2-methylpropane-2-sulfinamide (2.86 g, 23.6 mmol) and tetraisopropoxytitanium (8.06 mL, 27.2 mmol) at 20° C. The mixture was stirred at 50° C. for 16 h under N?, then was added to sat. NaCl. After stirring for 10 min the resulting mixture was filtered and washed three times with EtOAc. The filtrate was extracted three times with EtOAc. The combined organic layers were dried over Na₂SO₄, filtered and concentrated. The residue was purified by flash silica gel chromatography (40 g silica gel, eluent of 20% ethyl acetate/pet, ether gradient @40 mL/min) to give (E)-N-((5-bromo-4-chloropyridin-3-yl)methylene)-2-methylpropane-2-sulfinamide. MS (EST) Calculated for C₁₀H₁₃BrClN₂OS [M+H]⁺, 323, 325; found, 323, 325.

Step 3. To a stirred solution of (E)-N-((5-bromo-4-chloropyridin-3-yl)methylene)-2-methylpropane-2-sulfinamide (5 g, 15.5 mmol) in THF (100 mL) was added zinc (3.03 g, 46.3 mmol) followed by (E)-1-bromobut-2-ene (3.18 mL, 30.9 mmol) at 25° C. The mixture was stirred at 25° C. for 2 h under N₂, then filtered. The filtrate was concentrated. H₂O and EtOAc were added. The aqueous layer was extracted twice with EtOAc. The combined organic layers were dried over Na₂SO₄, filtered and concentrated to give a residue which was purified by flash silica gel chromatography (40 g silica gel, eluent of 60% ethyl acetate/pet, ether gradient @ii& 40 mL/min) to give (E)-N-(1-(5-bromo-4-chloropyridin-3-yl)-2-methylbut-3-en-1-ylidene)-2-methylpropane-2-sulfinamide. ¹H NMR (400 MHz, Chloroform-d) δ 8.68 (s, 1H), 8.47-8.53 (m, 1H), 5.69-5.86 (m, 1H), 5.09-5.35 (m, 2H), 4.71-5.05 (m, 1H), 3.66-3.97 (m, 1H), 2.47-2.90 (m, 1H), 1.15-1.23 (m, 9H), 0.97-1.07 (m, 3H).

Step 4. To a solution of N-(1-(5-bromo-4-chloropyridin-3-yl)-2-methylbut-3-en-1-yl)-2-methylpropane-2-sulfinamide (5.8 g, 15.3 mmol) in acetone (60 mL) and water (20 mL) was added NMO (4.47 g, 38.2 mmol) and potassium osmate(vi) dihydrate (1.17 g, 3.05 mmol). The resulting mixture was stirred at 25° C. for 16 h, then quenched with aq. Na₂S₂O₃ and extracted three times with ethyl acetate. The combined organic layers were washed with brine, dried over Na₂SO₄, filtered and concentrated. The residue was purified by flash silica gel chromatography (80 g silica gel, eluent of 100% ethyl acetate/pet, ether gradient @, 40 mL/min) to give N-(1-(5-bromo-4-chloropyridin-3-yl)-3,4-dihydroxy-2-methylbutyl)-2-methylpropane-2-sulfonamide. MS (ESI) Calculated for C₁₄H₂₃BrClN₂O₄S [M+H]⁺, 429, 431; found, 429, 431.

Step 5. To a solution of N-(1-(5-bromo-4-chloropyridin-3-yl)-3,4-dihydroxy-2-methylbutyl)-2-methylpropane-2-sulfinamide (4 g, 9.67 mmol), Et₃N (4.0 mL, 29 mmol), DMAP (0.236 g, 1.93 mmol) in DCM (40 mL) was added a solution of Ts-Cl (2.03 g, 10.6 mmol) in DCM (20 mL) at 0° C. The mixture was stirred at 25° C. for 16 h, then poured into H₂O and extracted three times with DCM. The combined organic layers were dried over Na₂SO₄, filtered and concentrated. The residue was purified by flash silica gel chromatography (80 g silica gel, eluent of 50% ethyl acetate/pet, ether gradient @40 mL/min) to give 5-(5-bromo-4-chloropyridin-3-yl)-1-(tert-butylsulfonyl)-4-methylpyrrolidin-3-ol. MS (ESI) Calculated for C₁₄H₂₁BrClN₂O₃S [M+H]⁺, 411, 413; found, 411, 413.

Step 6. To a mixture of 5-(5-bromo-4-chloropyridin-3-yl)-1-(tert-butylsulfonyl)-4-methylpyrrolidin-3-ol (2.2 g, 5.34 mmol) in DMSO (30 mL) was added K₂CO₃ (4.43 g, 32.1 mmol) and N-hydroxyacetamide (1.20 g, 16.0 mmol) at 20° C. The resulting mixture was stirred at 80° C. for 16 h, then filtered and purified by prep-HPLC (Instrument EJ; Method Column Boston Green ODS 150 mm*30 mm*5 um; Condition water (TFA)-ACN Begin B 15 End B 35 Gradient Time (min) 10 100% B Hold Time (min) 2 FlowRate (mL/min) 25; Injections 40) to give 3-bromo-5-((2R,3S,4S)-1-(tert-butylsulfonyl)-4-hydroxy-3-methylpyrrolidin-2-yl)pyridin-4-ol. ¹H NMR (400 MHz, Methanol-d₄) δ 8.16 (d, J=1.2 Hz, 1H), 7.68 (d, J=1.2 Hz, 1H), 4.48 (br d, J=9.2 Hz, 1H), 4.15 (br s, 1H), 3.69-3.82 (m, 2H), 2.91 (br d, J=4.8 Hz, 1H), 1.13-1.24 (m, 9H), 0.99 (d, J=6.8 Hz, 3H). MS (ESI) Calculated for C₁₄H₂₂BrN₂O₄S [M+H], 393, 395; found, 393, 395.

Step 7. To a mixture of 3-bromo-5-((2R,3S,4S)-1-(tert-butylsulfonyl)-4-hydroxy-3-methylpyrrolidin-2-yl)pyridin-4-ol (560 mg, 1.424 mmol) and tributylphosphane (576 mg, 2.85 mmol) in THF (25 mL) was added di-tert-butyl (E)-diazene-1,2-dicarboxylate (656 mg, 2.85 mmol) at 20° C. The mixture was stirred at 80° C. for 16 h under N₂, then concentrated to give an oil which was purified by flash silica gel chromatography (12 g silica gel, eluent of 15% ethyl acetate/pet, ether gradient @ 30 mL/min) to give (2R,5R,10S)-9-bromo-4-(tert-butylsulfonyl)-10-methyl-2,3,4,5-tetrahydro-2,5-methanopyrido[3,4-f][1,4]oxazepine. MS (ESI) Calculated for C₁₄H₂₀BrN₂O₃S [M+H]⁺, 375; found, 375.

Step 8. (2R,5R,10S)-9-Bromo-4-(tert-butylsulfonyl)-10-methyl-2,3,4,5-tetrahydro-2,5-methanopyrido[3,4-f][1,4]oxazepine (300 mg, 0.799 mmol) in TfOH/DCM(V/V, 1/10) (10 mL) was stirred at 25° C. for 0.5 h under N₂. Sat. NaHCO₃ and DCM were added. The organic layer was separated, and the aqueous was re-extracted three times with DCM. The combined organic layers were washed with brine, dried over anhydrous Na₂SO₄, filtered and concentrated to give (2R,5R,10S)-9-bromo-10-methyl-2,3,4,5-tetrahydro-2,5-methanopyrido[3,4-f][1,4]oxazepine which was used directly. MS (ESI) Calculated for C₁₀H₁₂BrN₂O [M+H]⁺, 255, 257; found, 255, 257.

Step 9. To a stirred solution of (2R,5R,10S)-9-bromo-10-methyl-2,3,4,5-tetrahydro-2,5-methanopyrido[3,4-f][1,4]oxazepine (200 mg, 0.674 mmol) in Dioxane (3 mL) and water (3 mL) was added potassium ferrocyanide trihydrate (285 mg, 0.674 mmol), potassium acetate (199 mg, 2.023 mmol) and Brettphos Pd G₃ (122 mg, 0.135 mmol) at 25° C. After the addition was finished, the reaction was stirred at 100° C. under N₂ atmosphere for 30 h. The mixture was concentrated in vacuo. The residue was purified by HPLC (Instrument EJ; Method Column Boston Green ODS 150 mm*30 mm*5 um; Condition water (TFA)-ACN Begin B 0 End B 50 Gradient Time (min) 10 100% B Hold Time (min) 2; FlowRate (mL/min) 25; Injections 6) to give Intermediate XII, (2R,5R,10S)-10-methyl-2,3,4,5-tetrahydro-2,5-methanopyrido[3,4-f][1,4]oxazepine-9-carbonitrile. ¹H NMR (400 MHz, Methanol-d₄) δ 8.73-8.83 (m, 1H), 8.56 (s, 1H), 5.25 (d, J=3.6 Hz, 1H), 4.86 (br s, 1H), 3.83-4.00 (m, 2H), 2.90-3.00 (m, 1H), 1.25 (d, J=7.2 Hz, 3H). MS (EST) Calculated for C H₁₂N₃O [M+H]⁺, 202; found, 202.

### Preparation of Intermediate XIII. 6,7,8,9-Tetrahydro-5H-6,9-methanopyrido[3,4-c]azepine-4-carbonitrile

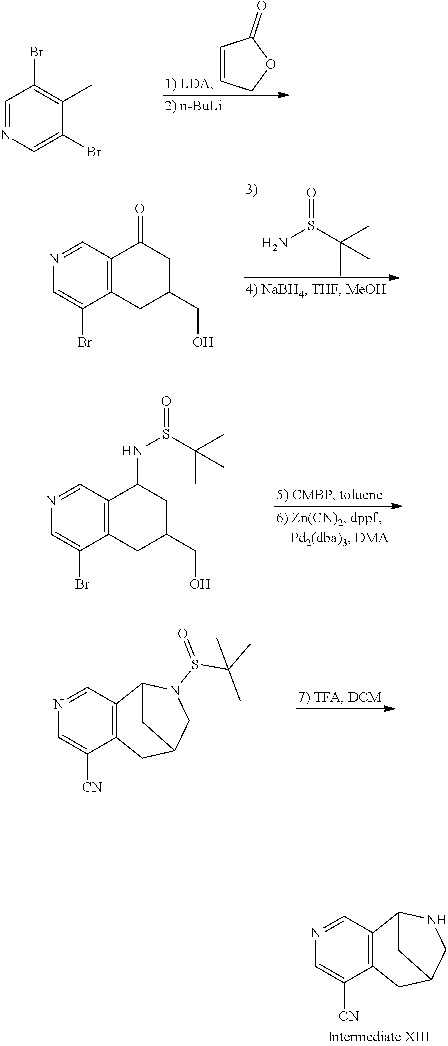

Step 1. To a solution of 3,5-dibromo-4-methylpyridine (3.7 g, 14.8 mmol) in THF (100 mL) was added LDA (11.1 mL, 22.1 mmol) at −78° C. The mixture was stirred at −78° C. for 30 min. Then a solution of furan-2(5H)-one (1.86 g, 22.1 mmol) in THF (5 mL) was added dropwise. The mixture was stirred at −78° C. for 2 h, then was slowly poured into 1N HCl, and extracted three times with EtOAc. The combined organic layers were washed with brine, dried over Na₂SO₄, filtered and concentrated in vacuum. The residue was purified by flash silica gel chromatography (40 g silica gel, Eluent of 0-30% EtOAc/Pet.ether gradient @, 25 mL/min) to give 4-((3,5-dibromopyridin-4-yl)methyl)dihydrofuran-2(3H)-one. ¹H NMR (400 MHz, Chloroform-d) δ 8.64 (s, 2H), 4.39 (dd, J=7.2, 8.8 Hz, 1H), 4.11-4.20 (m, 1H), 3.15-3.24 (m, 2H), 3.17-3.19 (m, 1H), 2.57-2.67 (m, 1H), 2.41-2.51 (m, 1H).

Step 2. To a solution of 4-((3,5-dibromopyridin-4-yl)methyl)dihydrofuran-2(3H)-one (1.0 g, 2.99 mmol) in THF (100 mL) was added BuLi (1.79 mL, 4.48 mmol) dropwise at −78° C. After the addition, the mixture was stirred at −78° C. for 2 h, then was slowly poured into 1N HCl, and extracted three times with EtOAc. The combined organic layers were washed with brine, dried over Na₂SO₄, filtered and concentrated in vacuum. The residue was purified by flash silica gel chromatography (12 g silica gel, Eluent of 10-60% EtOAc/Pet.ether gradient @25 mL/min) to give 4-bromo-6-(hydroxymethyl)-6,7-dihydroisoquinolin-8(5H)-one. MS (ESI) Calculated for C₁₀H₁₁BrNO₂ [M+H]⁺, 256; found, 256.

Step 3. To a solution of 4-bromo-6-(hydroxymethyl)-6,7-dihydroisoquinolin-8(5H)-one (270 mg, 1.05 mmol) in toluene (3 mL) was added 2-methylpropane-2-sulfinamide (256 mg, 2.11 mmol) and Titanium(IV) isopropoxide (0.624 ml, 2.11 mmol). The mixture was stirred at 100° C. for 16 h, then cooled to RT, and quenched with sat. aq. NaHCO₃ and EtOAc. The suspension was filtered through a pad of CELITE (diatomaceous earth), and washed with EtOAc. The organic layer was separated and washed with brine, dried over Na₂SO₄, filtered and concentrated in vacuum to give crude (E)-N-(4-bromo-6-(hydroxymethyl)-6,7-dihydroisoquinolin-8(5H)-ylidene)-2-methylpropane-2-sulfinamide, which was used for next step directly. MS (EST) Calculated for C₁₄H₂₀BrN₂O₂S [M+H]⁺, 359; found, 359.

Step 4. To a solution of (E)-N-(4-bromo-6-(hydroxymethyl)-6,7-dihydroisoquinolin-8(5H)-ylidene)-2-methylpropane-2-sulfinamide (300 mg, 0.825 mmol) in THF (3 mL) and MeOH (3 mL) was added NaBH₄ (63 mg, 1.67 mmol). The mixture was stirred at 20° C. for 30 min, then concentrated in vacuum. The residue was purified by flash silica gel chromatography (4 g silica gel, Eluent of 10˜100% EtOAc/Pet.ether gradient @ 25 mL/min) to give N-(4-bromo-6-(hydroxymethyl)-5,6,7,8-tetrahydroisoquinolin-8-yl)-2-methylpropane-2-sulfinamide. MS (ESI) Calculated for C₁₄H₂₂BrN₂O₂S [M+H]⁺, 361; found, 361.

Step 5. A mixture of N-(4-bromo-6-(hydroxymethyl)-5,6,7,8-tetrahydroisoquinolin-8-yl)-2-methylpropane-2-sulfamide (80 mg, 0.22 mmol) and cyanomethylenetributylphosphorane (160 mg, 0.664 mmol) in toluene (5 mL) was bubbled with N₂ for 1 min. The mixture was then stirred at 100° C. for 24 h. The residue was concentrated in vacuum and was purified by prep-TLC (SiO₂, Pet.ether:EtOAc=1:3) to give 4-bromo-8-(tert-butylsulfinyl)-6,7,8,9-tetrahydro-5H-6,9-methanopnyrido[3,4-c]azepine. MS (ESI) Calculated for C₁₄H₂₀BrN₂OS [M+H]⁺, 343; found, 343.

Step 6. To a solution of 4-bromo-8-(tert-butylsulfinyl)-6,7,8,9-tetrahydro-5H-6,9-methanopyrido[3,4-c]azepine (40 mg, 0.117 mmol) and zinc cyanide (21 mg, 0.175 mmol) in DMA (1 mL) was added DPPF (13 mg, 0.023 mmol) and Pd₂dba₃ (11 mg, 0.012 mmol). The mixture was stirred at 120° C. under N₂ for 12 h, then diluted with EtOAc, washed with water, and brine. The organic layer was dried over Na₂SO₄, filtered and concentrated in vacuum. The residue was purified by prep-HPLC (TFA) to give 8-(tert-butylsulfinyl)-6,7,8,9-tetrahydro-5H-6,9-methanopyrido[3,4-c]azepine-4-carbonitrile. MS (ESI) Calculated for C₁₅H₂₀N₃OS [M+H]⁺, 290; found, 290.

Step 7. A mixture of 8-(tert-butylsulfinyl)-6,7,8,9-tetrahydro-5H-6,9-methanopyrido[3,4-c]azepine-4-carbonitrile (70 mg, 0.24 mmol) in DCM (4 mL) was stirred at 20° C. for 2 h. The mixture was concentrated in vacuum to give crude Intermediate XIII, 6,7,8,9-tetrahydro-5H-6,9-methanopyrido[3,4-c]azepine-4-carbonitrile, which was used for next directly. MS (ESI) Calculated for C₁₁H₁₂N₃ [M+H]⁺, 186; found, 186.

### Table 1 Examples

### Example 1A. Preparation of (2S,5S)-4-(3,3-Difluoro-2,2-dimethylpropanoyl)-2,3,4,5-tetrahydro-2,5-methanopyrido[3,4-f][1,4]oxazepine-9-carbonitrile (1-1)

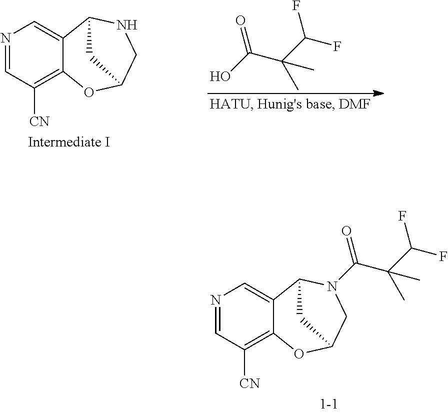

A solution of (2S,5S)-2,3,4,5-tetrahydro-2,5-methanopyrido[3,4-f][1,4]oxazepine-9-carbonitrile, Intermediate I (220 mg, 1.18 mmol), HATU (894 mg, 2.35 mmol) and Hunig's base (1.00 mL, 5.88 mmol) in DMF (5 mL) was treated with 3,3-difluoro-2,2-dimethylpropanoic acid (220 mg, 1.59 mmol) and the mixture stirred for 12 h. The mixture was concentrated, and the residue was purified by reverse phase chromatography (gradient of 1545% MeCN/water with 0.1% TFA, and then a gradient of 15-70% MeCN/water with 0.1% NH₄OH) to provide the desired product. The oil was dissolved in MeCN (3 mL) and water (3 mL) and dried by lyophilization to provide (2S,5S)-4-(3,3-difluoro-2,2-dimethylpropanoyl)-2,3,4,5-tetrahydro-2,5-methanopyrido[3,4-f][1,4]oxazepine-9-carbonitrile. ¹H NMR (500 MHz, DMSO-d₆) δ 8.69 (s, 1H), 8.46 (s, 1H), 6.20 (t, J=57 Hz, 1H), 5.44 (s, 1H), 5.26 (s, 1H), 4.11 (s, 1H), 4.01 (m, 1H), 2.34 (m, 1H), 2.01-2.24 (m, 1H), 1.15 (2s, 6H); MS (EI) calculated for C₁₅H₁₆F₂N₃O₂ [M+H]⁺, 308; found, 308.

### Example 1B. Preparation of (2S,5S)-4-(4-(Trifluoromethyl)bicyclo[2.2.1]heptane-1-carbonyl)-2,3,4,5-tetrahydro-2,5-methanopyrido[3,4-f][1.4]oxazepine-9-carbonitrile (1-2)

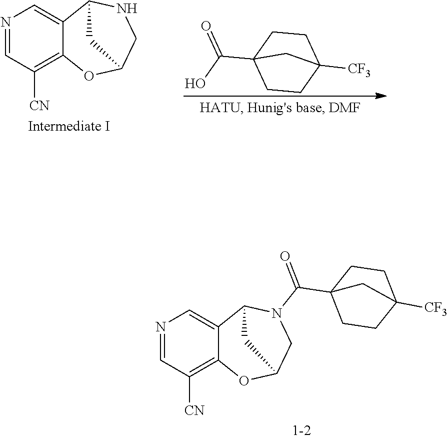 [SMILES: N#Cc1cncc2c1O[C@@H]1CN[C@H]2C1 ; O=C(O)C12CCC(C(F)(F)F)(CC1)C2 ; N#Cc1cncc2c1O[C@H]1C[C@@H]2N(C(=O)C23CCC(C(F)(F)F)(CC2)C3)C1]

A mixture containing 4-(trifluoromethyl)bicyclo[2.2.1]heptane-1-carboxylic acid (140 mg, 0.672 mmol) in DMF (3 mL) was treated with Hunig's base (0.20 mL, 1.15 mmol) and HATU (250 mg, 0.657 mmol). The mixture was stirred for 15 min, then treated with Intermediate I (150 mg, 0.361 mmol) and stirred overnight. The mixture was then purified by reverse phase chromatography (gradient of 15-70% MeCN/water with 0.1% NH₄OH). ¹H NMR (500 MHz, DMSO-d) S 8.67 (s, 1H), 8.42 (s, 1H), 5.44 (s, 1H), 5.22 (d, J=4.8 Hz, 1H), 3.82-4.13 (m, 2 H), 2.32 (ddd, J=12.7, 5.0, 2.5 Hz, 1H), 2.17 (d, J=12.8 Hz, 1H), 1.75-1.90 (m, 6H), 1.50-1.68 (m, 4H); MS (EI) cal'd for C₁₉H₁₉F₃N₃O₂ [M+H]⁺, 378; found, 378.

### Example 1C. Preparation of (2S,5S)-4-(2,2-Dimethylbutanoyl)-2,3,4,5-tetrahydro-2,5-methanopyrido[3,4-f][1,4]oxazepine-9-carbonitrile (1-4)

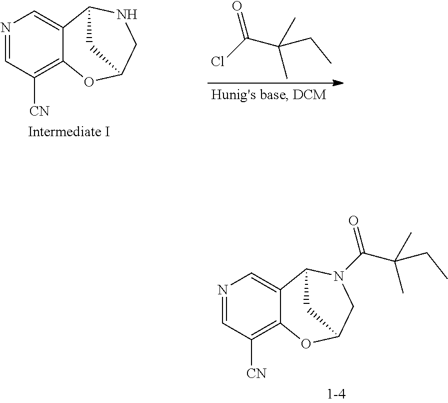

A solution of (2S,5S)-2,3,4,5-tetrahydro-2,5-methanopyrido[3,4-f][1,4]oxazepine-9-carbonitrile (15 mg, 0.080 mmol) in DCM (0.5 mL) was added treated with DIEA (0.042 mL, 0.24 mmol) and 2,2-dimethylbutyryl chloride (16 mg, 0.12 mmol). The mixture was stirred at 20° C. for 1 h, concentrated and purified by reverse phase chromatography (gradient of 24-54% MeCN/water with 10 mM NH₄HCO₃) to give (2S,5S)-4-(2,2-dimethylbutanoyl)-2,3,4,5-tetrahydro-2,5-methanopyrido[3,4-f][1,4]oxazepine-9-carbonitrile, 1H NMR (400 MHz, CDCl₃) δ 8.61 (m, 2H), 5.39 (m, 2H), 4.10 (d, J=12 Hz, 1H), 3.96 (d, J=12 Hz, 1H), 2.31 (m, 1H), 2.22 (m, 1H), 1.58 (m, 1H), 1.47 (m, 1H), 1.17 (s, 3H), 1.15 (s, 3H), 0.66 (t, J=7.6 Hz, 3H); MS (EI) calculated for C₁₆H₂₀N₃O₂ [M+H]⁺, 286; found, 286.

### Example 1D. Preparation of (2S,5S)-4-(3,3,3-Trifluoro-2,2-dimethylpropanoyl)-2,3,4,5-tetrahydro-2,5-methanopyrido[3,4-f][1,4]oxazepine-9-carbonitrile (1-9)

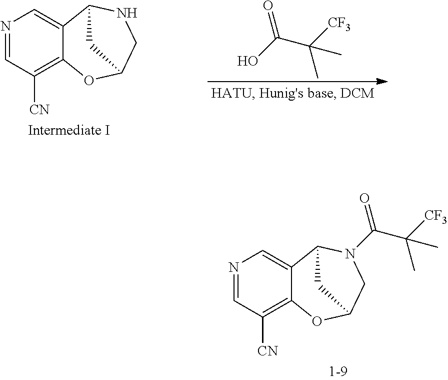

A solution of (2S,5S)-2,3,4,5-tetrahydro-2,5-methanopyrido[3,4-f][1,4]oxazepine-9-carbonitrile (20 mg, 0.11 mmol), 2-(3H-[1,2,3]triazolo[4,5-b]pyridin-3-yl)-1,1,3,3-tetramethylisouronium hexafluorophosphate(V) (81 mg, 0.21 mmol) and DIEA (0.10 mL, 0.53 mmol) in DMF (1 mL) was treated with 3,3,3-trifluoro-2,2-dimethylpropionic acid (25 mg, 0.16 mmol), and the reaction stirred at 20° C. for 12 h. The mixture was concentrated to give a residue which was purified by reverse phase chromatography (gradient of 22 to 52% MeCN/water with 0.1% TFA) to give (2S,5S)-4-(3,3,3-trifluoro-2,2-dimethylpropanoyl)-2,3,4,5-tetrahydro-2,5-methanopyrido[3,4-f][1,4]oxazepine-9-carbonitrile. ¹H NMR (500 MHz, CDCl₃) δ 8.75 (s, 1H), 8.70 (s, 1H), 5.46 (m, 2H), 4.25 (m, 1H), 4.06 (m, 1H), 2.45 (m, 1H), 2.30 (m, 1H); 1.47 (s, 3H), 1.46 (s, 3H); MS (ET) calculated for C₁₅H₁₅F₃N₃O₂[M+H]⁺ 326; found, 326.

### Example 1E. Preparation of (2S,5S)-4-(3,3-Difluoro-2,2-dimethylpropanoyl)-8-methyl-2,3,4,5-tetrahydro-2,5-methanopyrido[3,4-f][1.4]oxazepine-9-carbonitrile (1-18)

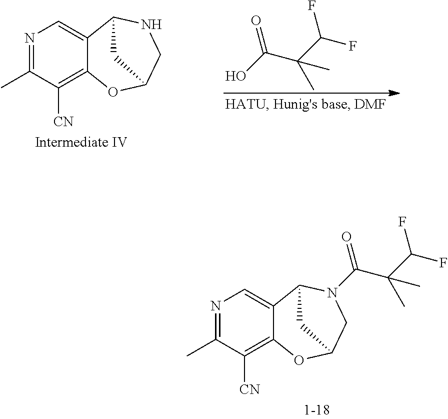

A stirred mixture of (2S,5S)-8-methyl-2,3,4,5-tetrahydro-2,5-methanopyrido[3,4-f][1,4]oxazepine-9-carbonitrile (17 mg, 0.084 mmol), 2-(3H-[1,2,3]triazolo[4,5-b]pyridin-3-yl)-1,1,3,3-tetramethylisouronium hexafluorophosphate(V) (64 mg, 0.17 mmol) and DIEA (0.074 mL, 0.42 mmol) in DMF (1 mL) was treated with 3,3-difluoro-2,2-dimethylpropanoic acid (18 mg, 0.13 mmol), and the mixture was stirred at 20° C. for 12 h. The mixture was concentrated to give a residue which was purified by reverse phase chromatography (gradient of 20-40% MeCN/water with 0.1% TFA) to give (2S,5S)-4-(3,3-difluoro-2,2-dimethylpropanoyl)-8-methyl-2,3,4,5-tetrahydro-2,5-methanopyrido[3,4-f][1,4]oxazepine-9-carbonitrile. ¹H NMR (400 MHz, CDCl₃) δ 8.61 (s, 1H), 5.98 (t, J=56 Hz, 1H), 5.45 (m, 1H), 5.40 (m, 1H), 4.14 (m, 1H), 4.02 (m, 1H), 2.82 (s, 3H), 2.39 (m, 1H), 2.23 (m, 1H), 1.33 (s, 3H), 1.23 (s, 3H); MS (EI) calculated for C₁₆H₁₈F₂N₃O₂ [M+H]⁺ 322; found, 322.

### Example 1F. Preparation of (2S,5S)-4-(4-Fluoro-1-(3-fluoropyrazolo[1,5-a]pyrimidin-7-yl)piperidine-4-carbonyl)-2,3,4,5-tetrahydro-2,5-methanopyrido[3,4-f][1,4]oxazepine-9-carbonitrile (1-55)

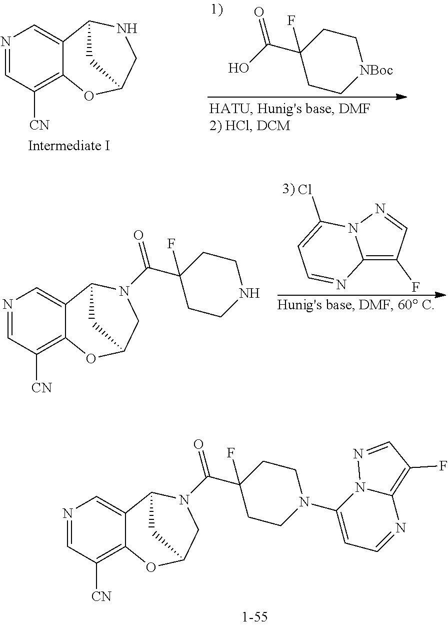 [SMILES: N#Cc1cncc2c1O[C@@H]1CN[C@H]2C1 ; CC(C)(C)OC(=O)N1CCC(F)(C(=O)O)CC1 ; N#Cc1cncc2c1O[C@H]1C[C@@H]2N(C(=O)C2(F)CCNCC2)C1 ; Fc1cnn2c(Cl)ccnc12 ; N#Cc1cncc2c1O[C@H]1C[C@@H]2N(C(=O)C2(F)CCN(c3ccnc4c(F)cnn34)CC2)C1]

Steps 1 and 2. A mixture containing 1-(tert-butoxycarbonyl)-4-fluoropiperidine-4-carboxylic acid (250 mg, 1.01 mmol) in DMF (3 mL) was treated with Hunig's Base (0.30 mL, 1.72 mmol) and HATU (400 mg, 1.05 mmol). The mixture was stirred for 15 min. Next, (2S,5S)-2,3,4,5-tetrahydro-2,5-methanopyrido[3,4-f][1,4]oxazepine-9-carbonitrile, HCl (200 mg, 0.894 mmol) was added, and the mixture stirred overnight and concentrated. The residue was taken up in DCM, washed with 1 N NaOH, dried (Na₂SO₄) and concentrated. The residue was purified by chromatography on SiO₂ (24 g silica, gradient of 0-60% EtOAc/DCM) to give tert-butyl 4-((2S,5S)-9-cyano-2,3,4,5-tetrahydro-2,5-methanopyrido[3,4-f][1,4]oxazepine-4-carbonyl)-4-fluoropiperidine-1-carboxylate. MS (EI) calculated for C₁₇H₁₈FN₄O₄[M-tBu+H]⁺, 361; found, 361. A solution of tert-butyl 4-((2S,5S)-9-cyano-2,3,4,5-tetrahydro-2,5-methanopyrido[3,4-f][1,4]oxazepine-4-carbonyl)-4-fluoropiperidine-1-carboxylate (360 mg, 0.864 mmol) in 3 mL of DCM was treated with a 4 M solution of HCl in dioxane (1.00 mL, 4.00 mmol), and the mixture stirred for 2 hours. The mixture was concentrated to dryness giving (2S,5S)-4-(4-fluoropiperidine-4-carbonyl)-2,3,4,5-tetrahydro-2,5-methanopyrido[3,4-f][1,4]oxazepine-9-carbonitrile, 2HCl. MS (EI) calculated for C₁₆H₁₈FN₄O₂ [M+H]⁺, 317; found, 317.

Step 3. A mixture of (2S,5S)-4-(4-fluoropiperidine-4-carbonyl)-2,3,4,5-tetrahydro-2,5-methanopyrido[3,4-f][1,4]oxazepine-9-carbonitrile, 2HCl (20 mg, 0.051 mmol) in DMF (1 mL) was treated with Hunig's Base (0.050 mL, 0.29 mmol) followed by 7-chloro-3-fluoropyrazolo[1,5-a]pyrimidine (15 mg, 0.087 mmol). The reaction was heated to 60° C. and stirred overnight. The mixture was purified by reverse phase chromatography (gradient of 15-70% MeCN/water with 0.1% TFA) to provide (2S,5S)-4-(4-fluoro-1-(3-fluoropyrazolo[1,5-a]pyrimidin-7-yl)piperidine-4-carbonyl)-2,3,4,5-tetrahydro-2,5-methanopyrido[3,4-f][1,4]oxazepine-9-carbonitrile. ¹H NMR (500 MHz, DMSO-d₆) δ 8.74 (m, 1H), 8.43-8.53 (m, 1H), 8.29 (m, 2H), 6.49 (m, 1H), 5.29-5.71 (m, 2H), 4.26-4.46 (m, 2H), 4.05-4.22 (m, 2H), 3.68-3.90 (m, 1H), 3.26-3.54 (m, 1H), 2.38 (m, 2H), 2.24 (m, 2H), 2.04 (m, 2H); MS (EI) calculated for C₂₂H₂₀F₂N₇O₂[M+H]⁺, 452; found, 452.

### Example 1G. Preparation of (2S,5S)-4-(1-Benzoyl-4-fluoropiperidine-4-carbonyl)-2,3,4,5-tetrahydro-2,5-methanopyrido[3,4-f][1,4]oxazepine-9-carbonitrile (1-54)

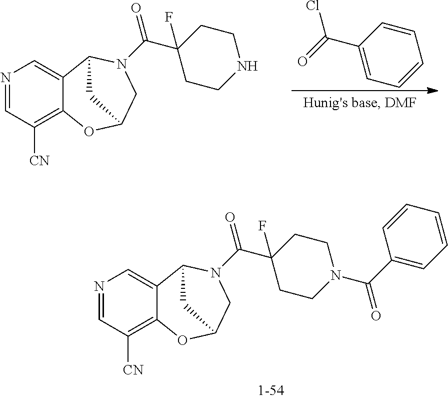 [SMILES: N#Cc1cncc2c1O[C@H]1C[C@@H]2N(C(=O)C2(F)CCNCC2)C1 ; O=C(Cl)c1ccccc1 ; N#Cc1cncc2c1O[C@H]1C[C@@H]2N(C(=O)C2(F)CCN(C(=O)c3ccccc3)CC2)C1]

A mixture of (2S,5S)-4-(4-fluoropiperidine-4-carbonyl)-2,3,4,5-tetrahydro-2,5-methanopyrido[3,4-f][1,4]oxazepine-9-carbonitrile, 2HCl (20 mg, 0.051 mmol) in DMF (1 mL) was treated with Hunig's Base (0.050 mL, 0.29 mmol) followed by benzoyl chloride (12 mg, 0.085 mmol). The reaction mixture was stirred overnight, and the mixture was purified by reverse phase chromatography (gradient of 15-70% MeCN/water with 0.1% TFA) to provide (2S,5S)-4-(1-benzoyl-4-fluoropiperidine-4-carbonyl)-2,3,4,5-tetrahydro-2,5-methanopyrido[3,4-f][1,4]oxazepine-9-carbonitrile. ¹H NMR (500 MHz, DMSO-d₆) δ 8.73 (m, 1H), 8.45 (m, 1H), 7.36-7.47 (m, 5H), 5.26-5.64 (m, 2H), 4.27-4.47 (m, 1H), 4.00-4.15 (m, 1H), 3.76 (m, 1H), 3.40-3.60 (m, 1H), 3.25 (m, 1H), 3.01 (m, 1H), 2.37 (m, 1H), 2.21 (m, 2H), 1.70-2.10 (m, 3H); MS (EI) calculated for C₂₃H₂₂FN₄O₃[M+H]⁺, 421; found, 421.

The following compounds in Table 1 were prepared using the procedure outlined in Example 1B for the preparation of compound 1-2, substituting 4-(trifluoromethyl)bicyclo[2.2.1]heptane-1-carboxylic acid for the appropriate carboxylic acid: compounds 1-5 to 1-15; compounds 1-19 to 1-37; compounds 1-40 to 1-47; compounds 1-59 to 1-69; compounds 1-73 to 1-79, compound 1-81.

Compound 1-3 was prepared using the procedure outlined in Example 1C for the preparation of compound 1-4, substituting 2,2-dimethylbutyl chloride for trimethylacetyl chloride.

Compounds 1-16 and 1-17 were prepared together as a racemate following the procedure outlined for compound 1-2, and then resolved using chiral SCF chromatography. Conditions: racemate dissolved in MeOH; CCO F4 21×250 mm, 5 um column; 215 nm detection; 70 mL/min of 15% MeOH/CO₂ with 0.1% NH₄OH. Peak 1 (3.20 min) isolated as 1-16, peak 2 (3.75 min) isolated as 1-17.

Compounds 1-38 and 1-39 were prepared together as a racemate following the procedure outlined for compound 1-2, and then resolved using chiral SCF chromatography. Conditions: racemate dissolved in 1:1 MeCN/MeOH; AS-H 21×250 mm, 5 um column; 215 nm detection; 70 mL/min of 10% MeOH/CO₂ with 0.1% NH₄OH. Peak 1 (2.58 min) isolated as 1-38, peak 2 (3.05 min) isolated as 1-17.

Compounds 1-48 and 1-49 were prepared together as a racemate following the procedure outlined for compound 1-2, and then resolved using chiral SCF chromatography. Conditions: racemate dissolved in 1:1 MeCN/MeOH; OJ-H 21×250 mm, 5 um column; 215 nm detection; 70 mL/min of 10% MeOH/CO₂ with 0.1% NH₄OH. Peak 1 (4.45 min) isolated as 1-48, peak 2 (5.60 min) isolated as 1-49.

Compounds 1-50 and 1-51 were prepared together as a racemate following the procedure outlined for compound 1-2, and then resolved using chiral SCF chromatography. Conditions: racemate dissolved in 1:1 MeCN/MeOH; (R,R)-Whelk-O 21×250 mm, 5 um column; 215 nm detection; 70 mL/min of 20% MeOH/CO₂ with 0.1% NH₄OH. Peak 1 (7.40 min) isolated as 1-50, peak 2 (8.60 min) isolated as 1-51.

Compounds 1-52, 1-53, 1-56, 1-57, 1-58, 1-70, 1-71, 1-72, and 1-80 were prepared in a fashion analogous to the synthesis of 1-55, substituting 7-chloro-3-fluoropyrazolo[1,5-a]pyrimidine for the appropriate aryl halide.

Compound 1-82 was prepared in a fashion analogous to the synthesis of 1-18, substituting 3,3-difluoro-2,2-dimethylpropanoic acid for 4-(trifluoromethyl)bicyclo[2.2.1]heptane-1-carboxylic acid.

TABLE 1

| Compound | | | Exact Mass |
|---|---|---|---|
| Number | Structure | Name | [M + H]⁺ |
| 1-1 | 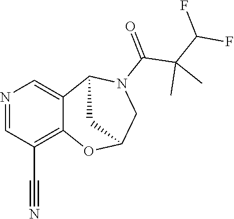 [SMILES: CC(C)(C(=O)N1C[C@@H]2C[C@H]1c1cncc(C#N)c1O2)C(F)F] | (2S,5S)-4-(3,3-difluoro-2,2- dimethylpropanoyl)-2,3,4,5- tetrahydro-2,5- methanopyrido[3,4- f][1,4]oxazepine-9- carbonitrile | Calculated 308, found 308 |
| 1-2 | 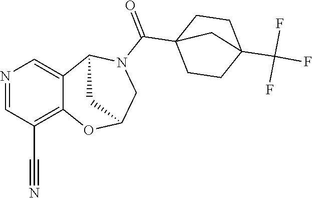 [SMILES: N#Cc1cncc2c1O[C@H]1C[C@@H]2N(C(=O)C23CCC(C(F)(F)F)(CC2)C3)C1] | (2S,5S)-4-[4- (trifluoromethyl)bicyclo[2.2.1] heptane-1-carbonyl]- 2,3,4,5-tetrahydro-2,5- methanopyrido[3,4- f][1,4]oxazepine-9- carbonitrile | Calculated 378, found 378 |
| 1-3 | 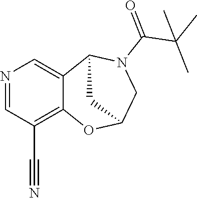 [SMILES: CC(C)(C)C(=O)N1C[C@@H]2C[C@H]1c1cncc(C#N)c1O2] | (2S,5S)-4-(2,2- dimethylpropanoyl)-2,3,4,5- tetrahydro-2,5- methanopyrido[3,4- f][1,4]oxazepine-9- carbonitrile | Calculated 272, found 272 |
| 1-4 | 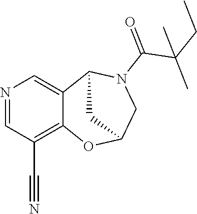 [SMILES: CCC(C)(C)C(=O)N1C[C@@H]2C[C@H]1c1cncc(C#N)c1O2] | (2S,5S)-4-(2,2- dimethylbutanoyl)-2,3,4,5- tetrahydro-2,5- methanopyrido[3,4- f][1,4]oxazepine-9- carbonitrile | Calculated 286, found 286 |
| 1-5 | 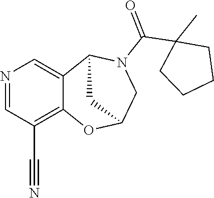 [SMILES: CC1(C(=O)N2C[C@@H]3C[C@H]2c2cncc(C#N)c2O3)CCCC1] | (2S,5S)-4-(1- methylcyclopentane-1- carbonyl)-2,3,4,5-tetrahydro- 2,5-methanopyrido[3,4- f][1,4]oxazepine-9- carbonitrile | Calculated 298, found 298 |
| 1-6 | 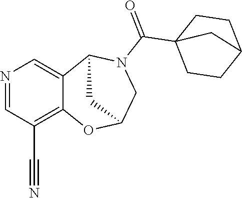 [SMILES: N#Cc1cncc2c1O[C@H]1C[C@@H]2N(C(=O)C23CCC(CC2)C3)C1] | (2S,5S)-4- (bicyclo[2.2.1]heptane-1- carbonyl)-2,3,4,5-tetrahydro- 2,5-methanopyrido[3,4- f][1,4]oxazepine-9- carbonitrile | Calculated 310, found 310 |
| 1-7 | 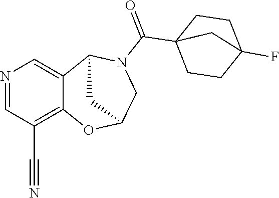 [SMILES: N#Cc1cncc2c1O[C@H]1C[C@@H]2N(C(=O)C23CCC(F)(CC2)C3)C1] | (2S,5S)-4-(4- fluorobicyclo[2.2.1]heptane- 1-carbonyl)-2,3,4,5- tetrahydro-2,5- methanopyrido[3,4- f][1,4]oxazepine-9- carbonitrile | Calculated 328, found 328 |
| 1-8 | 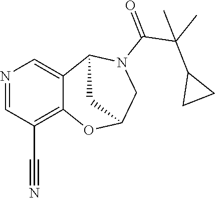 [SMILES: CC(C)(C(=O)N1C[C@@H]2C[C@H]1c1cncc(C#N)c1O2)C1CC1] | (2S,5S)-4-(2-cyclopropyl-2- methylpropanoyl)-2,3,4,5- tetrahydro-2,5- methanopyrido[3,4- f][1,4]oxazepine-9- carbonitrile | Calculated 298, found 298 |
| 1-9 | 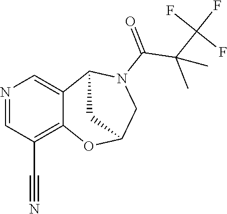 [SMILES: CC(C)(C(=O)N1C[C@@H]2C[C@H]1c1cncc(C#N)c1O2)C(F)(F)F] | (2S,5S)-4-(3,3,3-trifluoro- 2,2-dimethylpropanoyl)- 2,3,4,5-tetrahydro-2,5- methanopyrido[3,4- f][1,4]oxazepine-9- carbonitrile | Calculated 326, found 326 |
| 1-10 | 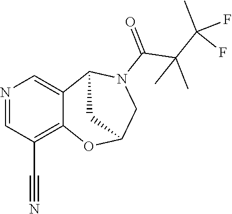 [SMILES: CC(F)(F)C(C)(C)C(=O)N1C[C@@H]2C[C@H]1c1cncc(C#N)c1O2] | (2S,5S)-4-(3,3-difluoro-2,2- dimethylbutanoyl)-2,3,4,5- tetrahydro-2,5- methanopyrido[3,4- f][1,4]oxazepine-9- carbonitrile | Calculated 322, found 322 |
| 1-11 | 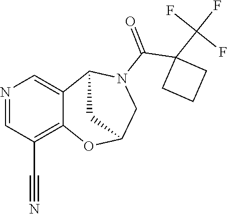 [SMILES: N#Cc1cncc2c1O[C@H]1C[C@@H]2N(C(=O)C2(C(F)(F)F)CCC2)C1] | (2S,5S)-4-[1- (trifluoromethyl)cyclobutane- 1-carbonyl]-2,3,4,5- tetrahydro-2,5- methanopyrido[3,4- f][1,4]oxazepine-9- carbonitrile | Calculated 338, found 338 |
| 1-12 | 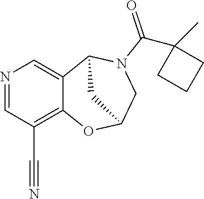 [SMILES: CC1(C(=O)N2C[C@@H]3C[C@H]2c2cncc(C#N)c2O3)CCC1] | (2S,5S)-4-(1- methylcyclobutane-1- carbonyl)-2,3,4,5-tetrahydro- 2,5-methanopyrido[3,4- f][1,4]oxazepine-9- carbonitrile | Calculated 284, found 284 |
| 1-13 | 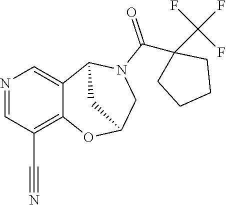 [SMILES: N#Cc1cncc2c1O[C@H]1C[C@@H]2N(C(=O)C2(C(F)(F)F)CCCC2)C1] | (2S,5S)-4-[1- (trifluoromethyl)cyclopentane- 1-carbonyl]-2,3,4,5- tetrahydro-2,5- methanopyrido[3,4- f][1,4]oxazepine-9- carbonitrile | Calculated 352, found 352 |
| 1-14 | 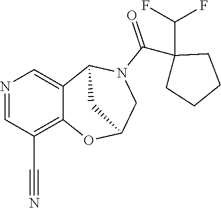 [SMILES: N#Cc1cncc2c1O[C@H]1C[C@@H]2N(C(=O)C2(C(F)F)CCCC2)C1] | (2S,5S)-4-[1- (difluoromethyl)cyclopentane- 1-carbonyl]-2,3,4,5- tetrahydro-2,5- methanopyrido[3,4- f][1,4]oxazepine-9- carbonitrile | Calculated 334, found 334 |
| 1-15 | 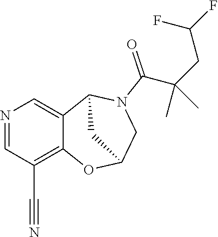 [SMILES: CC(C)(CC(F)F)C(=O)N1C[C@@H]2C[C@H]1c1cncc(C#N)c1O2] | (2S,5S)-4-(4,4-difluoro-2,2- dimethylbutanoyl)-2,3,4,5- tetrahydro-2,5- methanopyrido[3,4- f][1,4]oxazepine-9- carbonitrile | Calculated 322, found 322 |
| 1-16 | 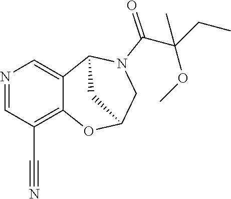 [SMILES: CCC(C)(OC)C(=O)N1C[C@@H]2C[C@H]1c1cncc(C#N)c1O2] | (2S,5S)-4-[2(S or R)- methoxy-2-methylbutanoyl]- 2,3,4,5-tetrahydro-2,5- methanopyrido[3,4- f][1,4]oxazepine-9- carbonitrile | Calculated 302, found 302 |
| 1-17 | 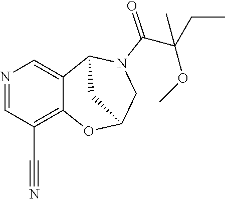 [SMILES: CCC(C)(OC)C(=O)N1C[C@@H]2C[C@H]1c1cncc(C#N)c1O2] | (2S,5S)-4-[2(S or R)- methoxy-2-methylbutanoyl]- 2,3,4,5-tetrahydro-2,5- methanopyrido[3,4- f][1,4]oxazepine-9- carbonitrile | Calculated 302, found 302 |
| 1-18 | 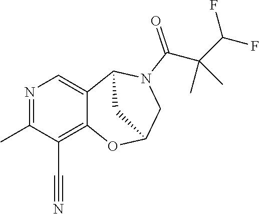 [SMILES: Cc1ncc2c(c1C#N)O[C@H]1C[C@@H]2N(C(=O)C(C)(C)C(F)F)C1] | (2S,5S)-4-(3,3-difluoro-2,2- dimethylpropanoyl)-8- methyl-2,3,4,5-tetrahydro- 2,5-methanopyrido[3,4- f][1,4]oxazepine-9- carbonitrile | Calculated 322, found 322 |
| 1-19 | 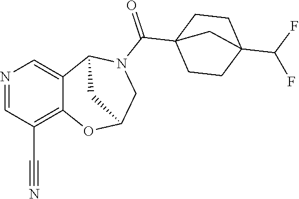 [SMILES: N#Cc1cncc2c1O[C@H]1C[C@@H]2N(C(=O)C23CCC(C(F)F)(CC2)C3)C1] | (2S,5S)-4-[4- (difluoromethyl)bicyclo[2.2.1] heptane-1-carbonyl]-2,3,4,5- tetrahydro-2,5- metanopyrido[3,4- f][1,4]oxazepine-9- carbonitrile | Calculated 360, found 360 |
| 1-20 | 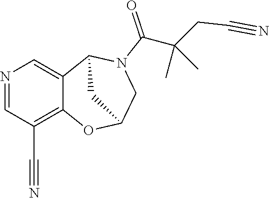 [SMILES: CC(C)(CC#N)C(=O)N1C[C@@H]2C[C@H]1c1cncc(C#N)c1O2] | (2S,5S)-4-(3-cyano-2,2- dimethylpropanoyl)-2,3,4,5- tetrahydro-2,5- methanopyrido[3,4- f][1,4]oxazepine-9- carbonitrile | Calculated 297, found 297 |
| 1-21 | 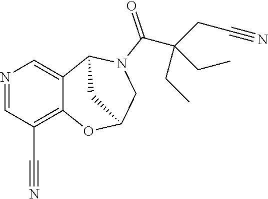 [SMILES: CCC(CC)(CC#N)C(=O)N1C[C@@H]2C[C@H]1c1cncc(C#N)c1O2] | (2S,5S)-4-(2-cyano-2- ethylbutanoyl)-2,3,4,5- tetrahydro-2,5- methanopyrido[3,4- f][1,4]oxazepine-9- carbonitrile | Calculated 311, found 311 |
| 1-22 | 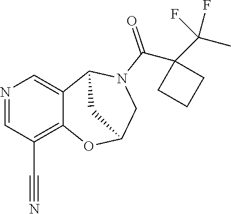 [SMILES: CC(F)(F)C1(C(=O)N2C[C@@H]3C[C@H]2c2cncc(C#N)c2O3)CCC1] | (2S,5S)-4-[1-(1,1- difluoroethyl)cyclobutane-1- carbonyl]-2,3,4,5-tetrahydro- 2,5-methanopyrido[3,4- f][1,4]oxazepine-9- carbonitrile | Calculated 334, found 334 |
| 1-23 | 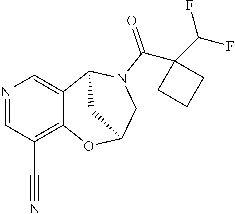 [SMILES: N#Cc1cncc2c1O[C@H]1C[C@@H]2N(C(=O)C2(C(F)F)CCC2)C1] | (2S,5S)-4-[1- (difluoromethyl)cyclobutane- 1-carbonyl]-2,3,4,5- tetrahydro-2,5- methanopyrido[3,4- f][1,4]oxazepine-9- carbonitrile | Calculated 320, found 320 |
| 1-24 | 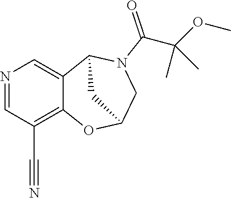 [SMILES: COC(C)(C)C(=O)N1C[C@@H]2C[C@H]1c1cncc(C#N)c1O2] | (2S,5S)-4-(2-methoxy-2- methylpropanoyl)-2,3,4,5- tetrahydro-2,5- methanopyrido[3,4- f][1,4]oxazepine-9- carbonitrile | Calculated 288, found 288 |
| 1-25 | 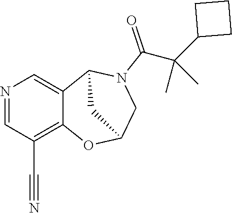 [SMILES: CC(C)(C(=O)N1C[C@@H]2C[C@H]1c1cncc(C#N)c1O2)C1CCC1] | (2S,5S)-4-(2-cyclobutyl-2- methylpropanoyl)-2,3,4,5- tetrahydro-2,5- methanopyrido[3,4- f][1,4]oxazepine-9- carbonitrile | Calculated 312, found 312 |
| 1-26 | 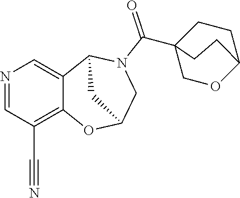 [SMILES: N#Cc1cncc2c1O[C@H]1C[C@@H]2N(C(=O)C23CCC(CC2)OC3)C1] | (2S,5S)-4-(2- oxabicyclo[2.2.2]octane-4- carbonyl)-2,3,4,5-tetrahydro- 2,5-methanopyrido[3,4- f][1,4]oxazepine-9- carbonitrile | Calculated 326, found 326 |
| 1-27 | 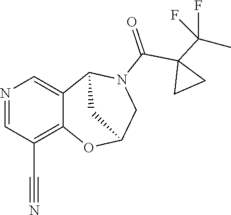 [SMILES: CC(F)(F)C1(C(=O)N2C[C@@H]3C[C@H]2c2cncc(C#N)c2O3)CC1] | (2S,5S)-4-[1-(1,1- difluoroethyl)cyclopropane- 1-carbonyl]-2,3,4,5- tetrahydro-2,5- methanopyrido[3,4- f][1,4]oxazepine-9- carbonitrile | Calculated 320, found 320 |
| 1-28 | 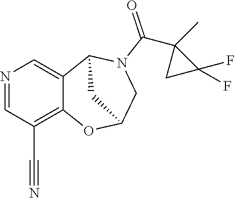 [SMILES: CC1(C(=O)N2C[C@@H]3C[C@H]2c2cncc(C#N)c2O3)CC1(F)F] | (2S,5S)-4-[(S or R)-2,2- difluoro-1- methylcyclopropane-1- carbonyl]-2,3,4,5-tetrahydro- 2,5-methanopyrido[3,4- f][1,4]oxazepine-9- carbonitrile | Calculated 306, found 306 |
| 1-29 | 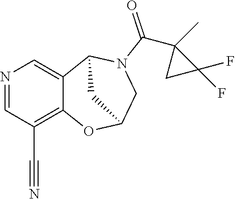 [SMILES: CC1(C(=O)N2C[C@@H]3C[C@H]2c2cncc(C#N)c2O3)CC1(F)F] | (2S,5S)-4-[(S or R)-2,2- difluoro-1- methylcyclopropane-1- carbonyl]-2,3,4,5-tetrahydro- 2,5-methanopyrido[3,4- f][1,4]oxazepine-9- carbonitrile | Calculated 306, found 306 |
| 1-30 | 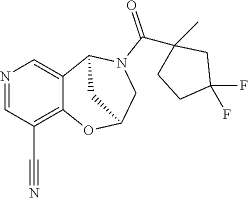 [SMILES: CC1(C(=O)N2C[C@@H]3C[C@H]2c2cncc(C#N)c2O3)CCC(F)(F)C1] | (2S,5S)-4-(3,3-difluoro-1- methylcyclopentane-1- carbonyl)-2,3,4,5-tetrahydro- 2,5-methanopyrido[3,4- f][1,4]oxazepine-9- carbonitrile | Calculated 334, found 334 |
| 1-31 | 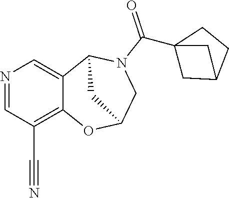 [SMILES: N#Cc1cncc2c1O[C@H]1C[C@@H]2N(C(=O)C23CCC(C2)C3)C1] | (2S,5S)-4- (bicyclo[2.1.1]hexane-1- carbonyl)-2,3,4,5-tetrahydro- 2,5-methanopyrido[3,4- f][1,4]oxazepine-9- carbonitrile | Calculated 296, found 296 |
| 1-32 | 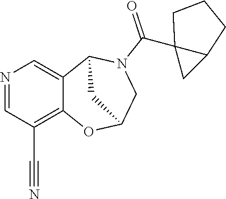 [SMILES: N#Cc1cncc2c1O[C@H]1C[C@@H]2N(C(=O)C23CCCC2C3)C1] | (2S,5S)-4-[(1R,5R or 1S,5S)- bicyclo[3.1.0]hexane-1- carbonyl]-2,3,4,5-tetrahydro- 2,5-methanopyrido[3,4- f][1,4]oxazepine-9- carbonitrile | Calculated 296, found 296 |
| 1-33 | 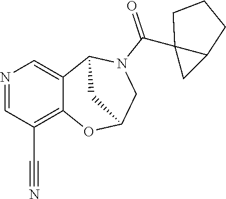 [SMILES: N#Cc1cncc2c1O[C@H]1C[C@@H]2N(C(=O)C23CCCC2C3)C1] | (2S,5S)-4-[(1R,5R or 1S,5S)- bicyclo[3.1.0]hexane-1- carbonyl]-2,3,4,5-tetrahydro- 2,5-methanopyrido[3,4- f][1,4]oxazepine-9- carbonitrile | Calculated 296, found 296 |
| 1-34 | 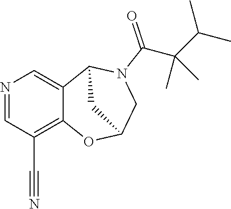 [SMILES: CC(C)C(C)(C)C(=O)N1C[C@@H]2C[C@H]1c1cncc(C#N)c1O2] | (2S,5S)-4-(2,2,3- trimethylbutanoyl)-2,3,4,5- tetrahydro-2,5- methanopyrido[3,4- f][1,4]oxazepine-9- carbonitrile | Calculated 300, found 300 |
| 1-35 | 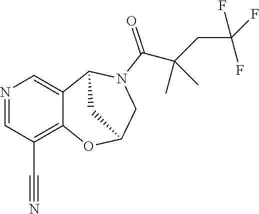 [SMILES: CC(C)(CC(F)(F)F)C(=O)N1C[C@@H]2C[C@H]1c1cncc(C#N)c1O2] | (2S,5S)-4-(4,4,4-trifluoro- 2,2-dimethylbutanoyl)- 2,3,4,5-tetrahydro-2,5- methanopyrido[3,4- f][1,4]oxazepine-9- carbonitrile | Calculated 340, found 340 |
| 1-36 | 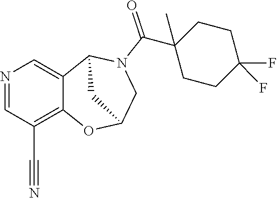 [SMILES: CC1(C(=O)N2C[C@@H]3C[C@H]2c2cncc(C#N)c2O3)CCC(F)(F)CC1] | (2S,5S)-4-(4,4-difluoro-1- methylcyclohexane-1- carbonyl)-2,3,4,5-tetrahydro- 2,5-methanopyrido[3,4- f][1,4]oxazepine-9- carbonitrile | Calculated 348, found 348 |
| 1-37 | 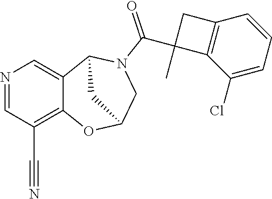 [SMILES: CC1(C(=O)N2C[C@@H]3C[C@H]2c2cncc(C#N)c2O3)Cc2cccc(Cl)c21] | (2S,5S)-4-(5-chloro-7(R or S)-methylbicyclo[4.2.0]octa- 1,3,5-triene-7-carbonyl)- 2,3,4,5-tetrahydro-2,5- methanopyrido[3,4- f][1,4]oxazepine-9- carbonitrile | Calculated 366, found 366 |
| 1-38 | 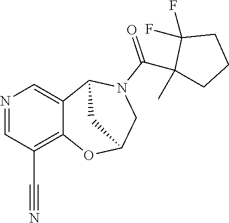 [SMILES: CC1(C(=O)N2C[C@@H]3C[C@H]2c2cncc(C#N)c2O3)CCCC1(F)F] | (2S,5S)-4-(2,2-difluoro-1- methylcyclopentane-1- carbonyl)-2,3,4,5-tetrahydro- 2,5-methanopyrido[3,4- f][1,4]oxazepine-9- carbonitrile | Calculated 334, found 334 |
| 1-39 | 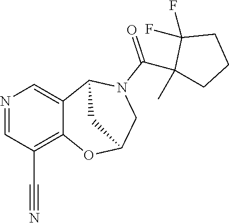 [SMILES: CC1(C(=O)N2C[C@@H]3C[C@H]2c2cncc(C#N)c2O3)CCCC1(F)F] | (2S,5S)-4-(2,2-difluoro-1- methylcyclopentane-1- carbonyl)-2,3,4,5-tetrahydro- 2,5-methanopyrido[3,4- f][1,4]oxazepine-9- carbonitrile | Calculated 334, found 334 |
| 1-40 | 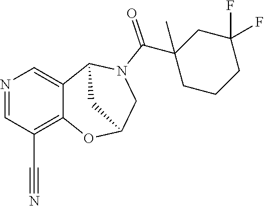 [SMILES: CC1(C(=O)N2C[C@@H]3C[C@H]2c2cncc(C#N)c2O3)CCCC(F)(F)C1] | (2S,5S)-4-[3,3-difluoro-1(R or S)-methylcyclohexane-1- carbonyl]-2,3,4,5-tetrahydro- 2,5-methanopyrido[3,4- f][1,4]oxazepine-9- carbonitrile | Calculated 348, found 348 |
| 1-41 | 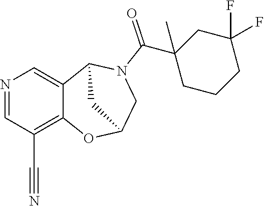 [SMILES: CC1(C(=O)N2C[C@@H]3C[C@H]2c2cncc(C#N)c2O3)CCCC(F)(F)C1] | (2S,5S)-4-[3,3-difluoro-1-(R or S)-methylcyclohexane-1- carbonyl]-2,3,4,5-tetrahydro- 2,5-methanopyrido[3,4- f][1,4]oxazepine-9- carbonitrile | Calculated 348, found 348 |
| 1-42 | 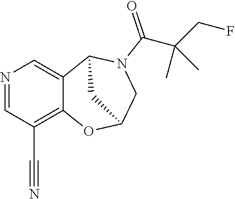 [SMILES: CC(C)(CF)C(=O)N1C[C@@H]2C[C@H]1c1cncc(C#N)c1O2] | (2S,5S)-4-(3-fluoro-2,2- dimethylpropanoyl)-2,3,4,5- tetrahydro-2,5- methanopyrido[3,4- f][1,4]oxazepine-9- carbonitrile | Calculated 290, found 290 |
| 1-43 | 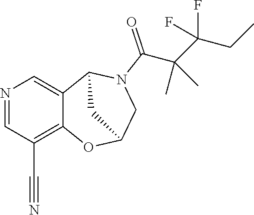 [SMILES: CCC(F)(F)C(C)(C)C(=O)N1C[C@@H]2C[C@H]1c1cncc(C#N)c1O2] | (2S,5S)-4-(3,3-difluoro-2,2- dimethylpentanoyl)-2,3,4,5- tetrahydro-2,5- methanopyrido[3,4- f][1,4]oxazepine-9- carbonitrile | Calculated 336, found 336 |
| 1-44 | 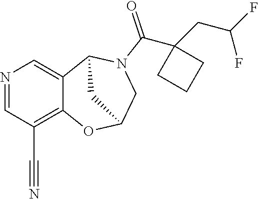 [SMILES: N#Cc1cncc2c1O[C@H]1C[C@@H]2N(C(=O)C2(CC(F)F)CCC2)C1] | (2S,5S)-4-[1-(2,2- difluoroethyl)cyclobutane-1- carbonyl]-2,3,4,5-tetrahydro- 2,5-methanopyrido[3,4- f][1,4]oxazepine-9- carbonitrile | Calculated 334, found 334 |
| 1-45 | 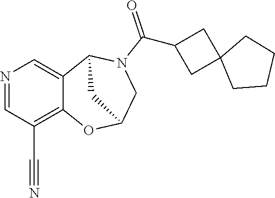 [SMILES: N#Cc1cncc2c1O[C@H]1C[C@@H]2N(C(=O)C2CC3(CCCC3)C2)C1] | (2S,5S)-4-(spiro[3.4]octane- 2-carbonyl)-2,3,4,5- tetrahydro-2,5- methanopyrido[3,4- f][1,4]oxazepine-9- carbonitrile | Calculated 324, found 324 |
| 1-46 | 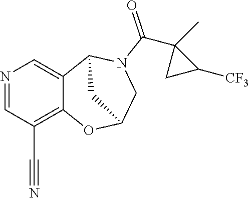 [SMILES: CC1(C(=O)N2C[C@@H]3C[C@H]2c2cncc(C#N)c2O3)CC1C(F)(F)F] | (2S,5S)-4-[1-methyl-2- (trifluoromethyl)cyclopropane- 1-carbonyl]-2,3,4,5- tetrahydro-2,5- methanopyrido[3,4- f][1,4]oxazepine-9- carbonitrile | Calculated 338, found 338 |
| 1-47 | 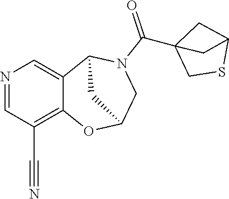 [SMILES: N#Cc1cncc2c1O[C@H]1C[C@@H]2N(C(=O)C23CSC(C2)C3)C1] | (2S,5S)-4-(2- thiabicyclo[2.1.1]hexane-4- carbonyl)-2,3,4,5-tetrahydro- 2,5-methanopyrido[3,4- f][1,4]oxazepine-9- carbonitrile | Calculated 314, found 314 |
| 1-48 | 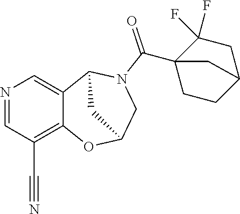 [SMILES: N#Cc1cncc2c1O[C@H]1C[C@@H]2N(C(=O)C23CCC(CC2(F)F)C3)C1] | (2S,5S)-4-[(S or R)-2,2- difluorobicyclo[2.2.1]heptane- 1-carbonyl]-2,3,4,5- tetrahydro-2,5- methanopyrido[3,4- f][1,4]oxazepine-9- carbonitrile | Calculated 346, found 346 |
| 1-49 | 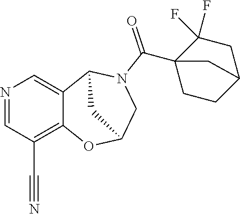 [SMILES: N#Cc1cncc2c1O[C@H]1C[C@@H]2N(C(=O)C23CCC(CC2(F)F)C3)C1] | (2S,5S)-4-[(S or R)-2,2- difluorobicyclo[2.2.1]heptane- 1-carbonyl]-2,3,4,5- tetrahydro-2,5- methanopyrido[3,4- f][1,4]oxazepine-9- carbonitrile | Calculated 346, found 346 |
| 1-50 | 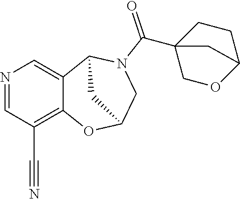 [SMILES: N#Cc1cncc2c1O[C@H]1C[C@@H]2N(C(=O)C23CCC(C2)OC3)C1] | (2S,5S)-4-[(S or R)-2- oxabicyclo[2.2.1]heptane-4- carbonyl]-2,3,4,5-tetrahydro- 2,5-methanopyrido[3,4- f][1,4]oxazepine-9- carbonitrile | Calculated 312, found 312 |
| 1-51 | 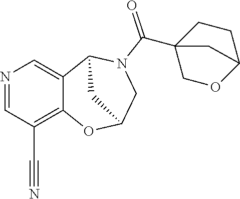 [SMILES: N#Cc1cncc2c1O[C@H]1C[C@@H]2N(C(=O)C23CCC(C2)OC3)C1] | (2S,5S)-4-[(S or R)-2- oxabicyclo[2.2.1]heptane-4- carbonyl]-2,3,4,5-tetrahydro- 2,5-methanopyrido[3,4- f][1,4]oxazepine-9- carbonitrile | Calculated 312, found 312 |
| 1-52 | 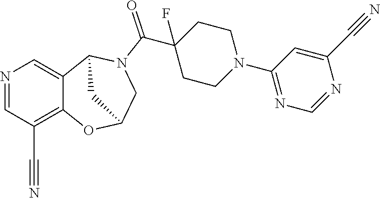 [SMILES: N#Cc1cc(N2CCC(F)(C(=O)N3C[C@@H]4C[C@H]3c3cncc(C#N)c3O4)CC2)ncn1] | (2S,5S)-4-(1-(6- cyanopyrimidin-4-yl)-4- fluoropiperidine-4-carbonyl)- 2,3,4,5-tetrahydro-2,5- methanopyrido[3,4- f][1,4]oxazepine-9- carbonitrile | Calculated 420, found 420 |
| 1-53 | 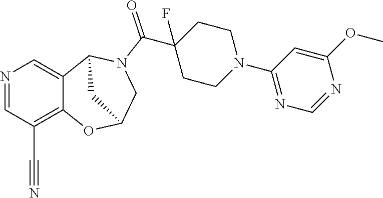 [SMILES: COc1cc(N2CCC(F)(C(=O)N3C[C@@H]4C[C@H]3c3cncc(C#N)c3O4)CC2)ncn1] | (2S,5S)-4-(4-fluoro-1-(6- methoxypyrimidin-4- yl)piperidine-4-carbonyl)- 2,3,4,5-tetrahydro-2,5- methanopyrido[3,4- f][1,4]oxazepine-9- carbonitrile | Calculated 425, found 425 |
| 1-54 | 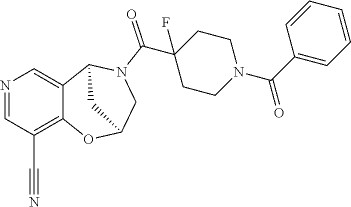 [SMILES: N#Cc1cncc2c1O[C@H]1C[C@@H]2N(C(=O)C2(F)CCN(C(=O)c3ccccc3)CC2)C1] | (2S,5S)-4-(1-benzoyl-4- fluoropiperidine-4-carbonyl)- 2,3,4,5-tetrahydro-2,5- methanopyrido[3,4- f][1,4]oxazepine-9- carbonitrile | Calculated 421, found 421 |
| 1-55 | 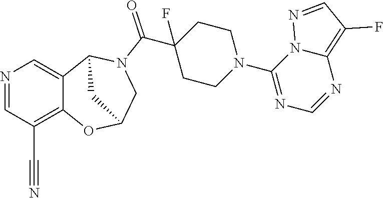 [SMILES: N#Cc1cncc2c1O[C@H]1C[C@@H]2N(C(=O)C2(F)CCN(c3ncnc4c(F)cnn34)CC2)C1] | (2S,5S)-4-(4-fluoro-1-(3- fluoropyrazolo[1,5- a]pyrimidin-7-yl)piperidine- 4-carbonyl)-2,3,4,5- tetrahydro-2,5- methanopyrido[3,4- f][1,4]oxazepine-9- carbonitrile | Calculated 452, found 452 |
| 1-56 | 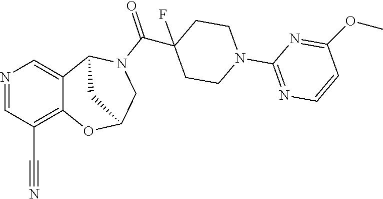 [SMILES: COc1ccnc(N2CCC(F)(C(=O)N3C[C@@H]4C[C@H]3c3cncc(C#N)c3O4)CC2)n1] | (2S,5S)-4-(4-fluoro-1-(4- methoxypyrimidin-2- yl)piperidine-4-carbonyl)- 2,3,4,5-tetrahydro-2,5- methanopyrido[3,4- f][1,4]oxazepine-9- carbonitrile | Calculated 425, found 425 |
| 1-57 | 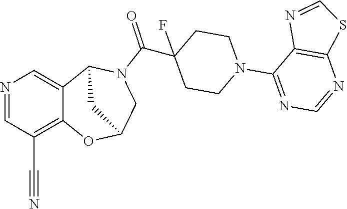 [SMILES: N#Cc1cncc2c1O[C@H]1C[C@@H]2N(C(=O)C2(F)CCN(c3ncnc4scnc34)CC2)C1] | (2S,5S)-4-(4-fluoro-1- (thiazolo[5,4-d]pyrimidin-7- yl)piperidine-4-carbonyl)- 2,3,4,5-tetrahydro-2,5- methanopyrido[3,4- f][1,4]oxazepine-9- carbonitrile | Calculated 452, found 452 |
| 1-58 | 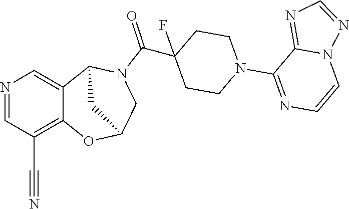 [SMILES: N#Cc1cncc2c1O[C@H]1C[C@@H]2N(C(=O)C2(F)CCN(c3nccn4ncnc34)CC2)C1] | (2S,5S)-4-(1- ([1,2,4]triazolo[1,5- a]pyrazin-8-yl)-4- fluoropiperidine-4-carbonyl)- 2,3,4,5-tetrahydro-2,5- methanopyrido[3,4- f][1,4]oxazepine-9- carbonitrile | Calculated 435, found 435 |
| 1-59 | 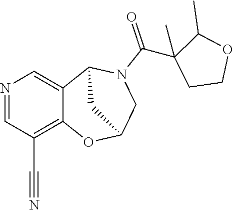 [SMILES: CC1OCCC1(C)C(=O)N1C[C@@H]2C[C@H]1c1cncc(C#N)c1O2] | (2S,5S)-4-(2,3- dimethyltetrahydrofuran-3- carbonyl)-2,3,4,5-tetrahydro- 2,5-methanopyrido[3,4- f][1,4]oxazepine-9- carbonitrile | Calculated 314, found 314 |
| 1-60 | 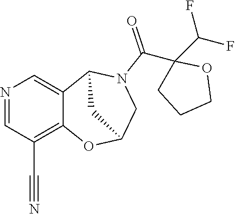 [SMILES: N#Cc1cncc2c1O[C@H]1C[C@@H]2N(C(=O)C2(C(F)F)CCCO2)C1] | (2S,5S)-4-(2- (difluoromethyl)tetrahydro- furan-2-carbonyl)-2,3,4,5- tetrahydro-2,5- methanopyrido[3,4- f][1,4]oxazepine-9- carbonitrile | Calculated 336, found 336 |
| 1-61 | 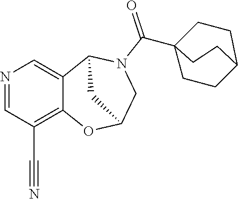 [SMILES: N#Cc1cncc2c1O[C@H]1C[C@@H]2N(C(=O)C23CCC(CC2)CC3)C1] | (2S,5S)-4- (bicyclo[2.2.2]octane-1- carbonyl)-2,3,4,5-tetrahydro- 2,5-methanopyrido[3,4- f][1,4]oxazepine-9- carbonitrile | Calculated 324, found 324 |
| 1-62 | 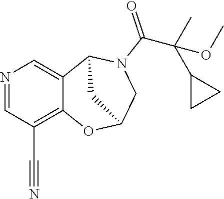 [SMILES: COC(C)(C(=O)N1C[C@@H]2C[C@H]1c1cncc(C#N)c1O2)C1CC1] | (2S,5S)-4-(2-(S and R)- cyclopropyl-2- methoxypropanoyl)-2,3,4,5- tetrahydro-2,5- methanopyrido[3,4- f][1,4]oxazepine-9- carbonitrile | Calculated 314, found 314 |
| 1-63 | 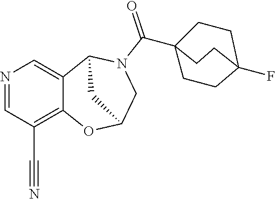 [SMILES: N#Cc1cncc2c1O[C@H]1C[C@@H]2N(C(=O)C23CCC(F)(CC2)CC3)C1] | (2S,5S)-4-(4- fluorobicyclo[2.2.2]octane-1- carbonyl)-2,3,4,5-tetrahydro- 2,5-methanopyrido[3,4- f][1,4]oxazepine-9- carbonitrile | Calculated 342, found 342 |
| 1-64 | 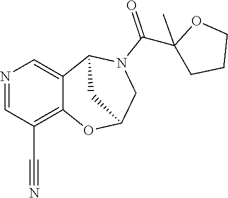 [SMILES: CC1(C(=O)N2C[C@@H]3C[C@H]2c2cncc(C#N)c2O3)CCCO1] | (2S,5S)-4-(2-(S and R)- methyltetrahydrofuran-2- carbonyl)-2,3,4,5-tetrahydro- 2,5-methanopyrido[3,4- f][1,4]oxazepine-9- carbonitrile | Calculated 300, found 300 |
| 1-65 | 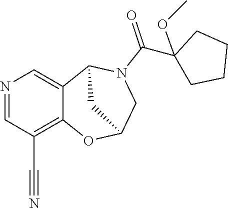 [SMILES: COC1(C(=O)N2C[C@@H]3C[C@H]2c2cncc(C#N)c2O3)CCCC1] | (2S,5S)-4-(1- methoxycyclopentane-1- carbonyl)-2,3,4,5-tetrahydro- 2,5-methanopyrido[3,4- f][1,4]oxazepine-9- carbonitrile | Calculated 314, found 314 |
| 1-66 | 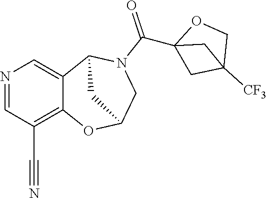 [SMILES: N#Cc1cncc2c1O[C@H]1C[C@@H]2N(C(=O)C23CC(C(F)(F)F)(CO2)C3)C1] | (2S,5S)-4-(4- (trifluoromethyl)-2- oxabicyclo[2.1.1]hexane-1- carbonyl)-2,3,4,5-tetrahydro- 2,5-methanopyrido[3,4- f][1,4]oxazepine-9- carbonitrile | Calculated 366, found 366 |
| 1-67 | 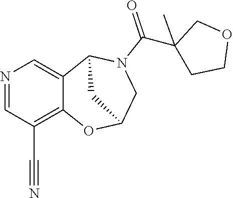 [SMILES: CC1(C(=O)N2C[C@@H]3C[C@H]2c2cncc(C#N)c2O3)CCOC1] | (2S,5S)-4-(3-(S and R)- methyltetrahydrofuran-3- carbonyl)-2,3,4,5-tetrahydro- 2,5-methanopyrido[3,4- f][1,4]oxazepine-9- carbonitrile | Calculated 300, found 300 |
| 1-68 | 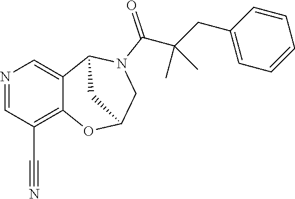 [SMILES: CC(C)(Cc1ccccc1)C(=O)N1C[C@@H]2C[C@H]1c1cncc(C#N)c1O2] | (2S,5S)-4-(2,2-dimethyl-3- phenylpropanoyl)-2,3,4,5- tetrahydro-2,5- methanopyrido[3,4- f][1,4]oxazepine-9- carbonitrile | Calculated 348, found 348 |
| 1-69 | 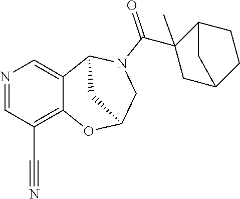 [SMILES: CC1(C(=O)N2C[C@@H]3C[C@H]2c2cncc(C#N)c2O3)CC2CCC1C2] | (2S,5S)-4-(2- methylbicyclo[2.2.1]heptane- 2-carbonyl)-2,3,4,5- tetrahydro-2,5- methanopyrido[3,4- f][1,4]oxazepine-9- carbonitrile | Calculated 324, found 324 |
| 1-70 | 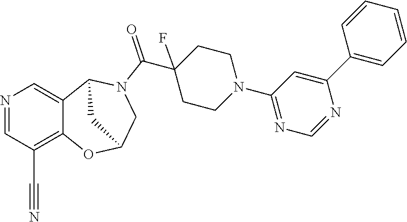 [SMILES: N#Cc1cncc2c1O[C@H]1C[C@@H]2N(C(=O)C2(F)CCN(c3cc(-c4ccccc4)ncn3)CC2)C1] | (2S,5S)-4-(4-fluoro-1-(6- phenylpyrimidin-4- yl)piperidine-4-carbonyl)- 2,3,4,5-tetrahydro-2,5- methanopyrido[3,4- f][1,4]oxazepine-9- carbonitrile | Calculated 471, found 471 |
| 1-71 | 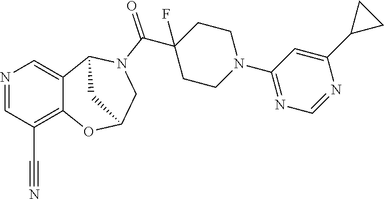 [SMILES: N#Cc1cncc2c1O[C@H]1C[C@@H]2N(C(=O)C2(F)CCN(c3cc(C4CC4)ncn3)CC2)C1] | (2S,5S)-4-(1-(6- cyclopropylpyrimidin-4-yl)- 4-fluoropiperidine-4- carbonyl)-2,3,4,5-tetrahydro- 2,5-methanopyrido[3,4- f][1,4]oxazepine-9- carbonitrile | Calculated 435, found 435 |
| 1-72 | 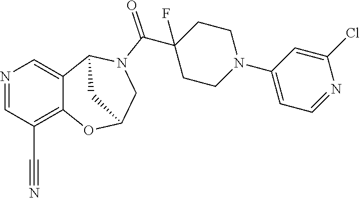 [SMILES: N#Cc1cncc2c1O[C@H]1C[C@@H]2N(C(=O)C2(F)CCN(c3ccnc(Cl)c3)CC2)C1] | (2S,5S)-4-(1-(2- chloropyridin-4-yl)-4- fluoropiperidine-4-carbonyl)- 2,3,4,5-tetrahydro-2,5- methanopyrido[3,4- f][1,4]oxazepine-9- carbonitrile | Calculated 428, found 428 |
| 1-73 | 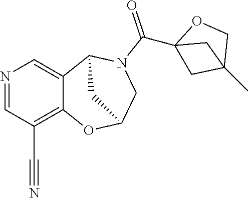 [SMILES: CC12COC(C(=O)N3C[C@@H]4C[C@H]3c3cncc(C#N)c3O4)(C1)C2] | (2S,5S)-4-(4-methyl-2- oxabicyclo[2.1.1]hexane-1- carbonyl)-2,3,4,5-tetrahydro- 2,5-methanopyrido[3,4- f][1,4]oxazepine-9- carbonitrile | Calculated 312, found 312 |
| 1-74 | 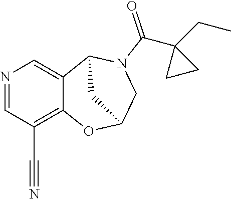 [SMILES: CCC1(C(=O)N2C[C@@H]3C[C@H]2c2cncc(C#N)c2O3)CC1] | (2S,5S)-4-(1- ethylcyclopropane-1- carbonyl)-2,3,4,5-tetrahydro- 2,5-methanopyrido[3,4- f][1,4]oxazepine-9- carbonitrile | Calculated 284, found 284 |
| 1-75 | 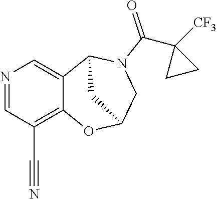 [SMILES: N#Cc1cncc2c1O[C@H]1C[C@@H]2N(C(=O)C2(C(F)(F)F)CC2)C1] | (2S,5S)-4-(1- (trifluoromethyl)cyclopropane- 1-carbonyl)-2,3,4,5- tetrahydro-2,5- methanopyrido[3,4- f][1,4]oxazepine-9- carbonitrile | Calculated 324, found 324 |
| 1-76 | 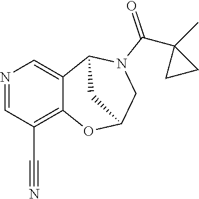 [SMILES: CC1(C(=O)N2C[C@@H]3C[C@H]2c2cncc(C#N)c2O3)CC1] | (2S,5S)-4-(1- methylcyclopropane-1- carbonyl)-2,3,4,5-tetrahydro- 2,5-methanopyrido[3,4- f][1,4]oxazepine-9- carbonitrile | Calculated 270, found 270 |
| 1-77 | 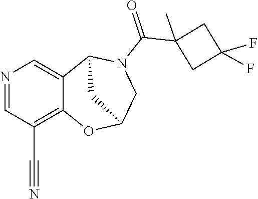 [SMILES: CC1(C(=O)N2C[C@@H]3C[C@H]2c2cncc(C#N)c2O3)CC(F)(F)C1] | (2S,5S)-4-(3,3-difluoro-1- methylcyclobutane-1- carbonyl)-2,3,4,5-tetrahydro- 2,5-methanopyrido[3,4- f][1,4]oxazepine-9- carbonitrile | Calculated 320, found 320 |
| 1-78 | 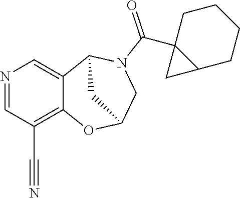 [SMILES: N#Cc1cncc2c1O[C@H]1C[C@@H]2N(C(=O)C23CCCCC2C3)C1] | (2S,5S)-4- (bicyclo[4.1.0]heptane-1- carbonyl)-2,3,4,5-tetrahydro- 2,5-methanopyrido[3,4- f][1,4]oxazepine-9- carbonitrile | Calculated 310, found 310 |
| 1-79 | 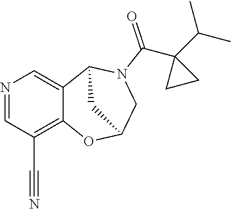 [SMILES: CC(C)C1(C(=O)N2C[C@@H]3C[C@H]2c2cncc(C#N)c2O3)CC1] | (2S,5S)-4-(1- isopropylcyclopropane-1- carbonyl)-2,3,4,5-tetrahydro- 2,5-methanopyrido[3,4- f][1,4]oxazepine-9- carbonitrile | Calculated 298, found 298 |
| 1-80 | 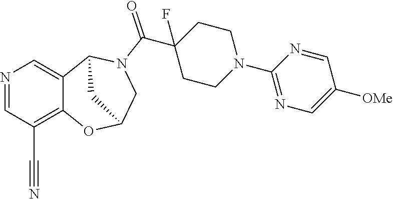 [SMILES: COc1cnc(N2CCC(F)(C(=O)N3C[C@@H]4C[C@H]3c3cncc(C#N)c3O4)CC2)nc1] | (2S,5S)-4-(4-fluoro-1-(5- methoxypyrimidin-2- yl)piperidine-4-carbonyl)- 2,3,4,5-tetrahydro-2,5- methanopyrido[3,4- f][1,4]oxazepine-9- carbonitrile | Calculated 425, found 425 |
| 1-81 | 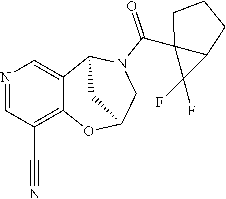 [SMILES: N#Cc1cncc2c1O[C@H]1C[C@@H]2N(C(=O)C23CCCC2C3(F)F)C1] | (2S,5S)-4-(6,6- difluorobicyclo[3.1.0]hexane- 1-carbonyl)-2,3,4,5- tetrahydro-2,5- methanopyrido[3,4- f][1,4]oxazepine-9- carbonitrile | Calculated 332, found 332 |
| 1-82 | 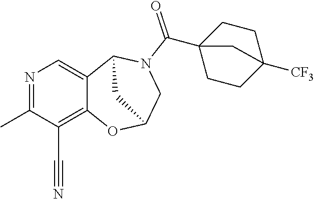 [SMILES: Cc1ncc2c(c1C#N)O[C@H]1C[C@@H]2N(C(=O)C23CCC(C(F)(F)F)(CC2)C3)C1] | (2S,5S)-8-methyl-4-(4- (trifluoromethyl)bicyclo[2.2.1] heptane-1-carbonyl)- 2,3,4,5-tetrahydro-2,5- methanopyrido[3,4- f](1,4]oxazepine-9- carbonitrile | Calculated 392, found 392 |

Compounds 1-83 to 1-113 shown in the following table were prepared as described below.

Compound 1-83 was prepared in a fashion analogous to the synthesis of 1-55, substituting 7-chloro-3-fluoropyrazolo[1,5-a]pyrimidine for the appropriate aryl halide.

Compounds 1-84 and 1-85 were prepared in a fashion analogous to the synthesis of 1-1. The mixture was then resolved using chiral column SFC chromatography. Conditions: Chiralpak IG 250 mm×30 mm, 10 uM column, 80 mL/min of 50% MeOH/CO₂ with 0.1% NH-40H; Peak 1 retention time 3.914 min (1-84 as white solid) and Peak 2 retention time 4.854 min (1-84 as white solid).

Compounds 1-86 and 1-87 were prepared in a fashion analogous to the synthesis of 1-1. The mixture was then resolved using chiral column SFC chromatography. Conditions: racemate dissolved in 1:1 MeCN/MeOH; Chiralpak AD-H 250 mm×21 mm, 5 uM column: 70 mL/min of 25% MeOH/CO₂ with 0.1% NH₄OH; 215 nm UV detection; Peak 1 (faster eluting enantiomer 1-86) and Peak 2 (slower eluting 1-87).

Compounds 1-88 and 1-89 were prepared in a fashion analogous to the synthesis of 1-1. The mixture was then resolved using reverse phase chromatography. Conditions: Boston Green ODS 150 mm×30 mm, 5 uM column: 27 to 47% MeCN/water with 0.1% TFA; 215 nm UV detection; Peak 1 (faster eluting enantiomer 1-88) and Peak 2 (slower eluting 1-89).

Compounds 1-90 to 1-105 were prepared in a fashion analogous to the synthesis of 1-1, substituting 3,3-difluoro-2,2-dimethylpropanoic acid for the corresponding carboxylic acid.

Compounds 1-106 and 1-107 were prepared in a fashion analogous to the synthesis of 1-1. The mixture was then resolved using reverse phase chromatography. Conditions: Boston Green ODS 150 mm×30 mm, 5 uM column: 45 to 65% MeCN/water with 0.1% TFA; 215 nm UV detection; Peak 1 (faster eluting enantiomer 1-106) and Peak 2 (slower eluting 1-107).

Compounds 1-108 to 1-111 were prepared in a fashion analogous to the synthesis of 1-1, substituting 3,3-difluoro-2,2-dimethylpropanoic acid for the corresponding carboxylic acid.

Compounds 1-112 and 1-113 were prepared in a fashion analogous to the synthesis of 1-1. The mixture was then resolved using chiral column SFC chromatography. Conditions: racemate dissolved in 1:1 MeCN/MeOH; OD-H 250 mm×21 mm, 5 uM column; 70 mL/min of 15% MeOH/CO₂ with 0.1% NH₄OH; 215 nm UV detection; Peak I (faster eluting enantiomer at 3.65 min 1-112) and Peak 2 (slower eluting at 4.15 min 1-113).

| | | | |
|---|---|---|---|
| 1-83 | 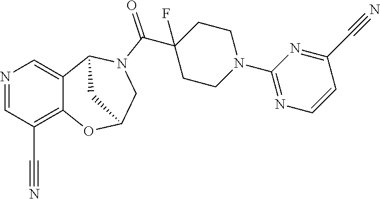 [SMILES: N#Cc1ccnc(N2CCC(F)(C(=O)N3C[C@@H]4C[C@H]3c3cncc(C#N)c3O4)CC2)n1] | (2S,5S)-4-(1-(4- cyanopyrimidin-2-yl)-4- fluoropiperidine-4-carbonyl)- 2,3,4,5-tetrahydro-2,5- methanopyrido[3,4- f][1,4]oxazepine-9-carbonitrile | Calc'd 420, found 420 |
| 1-84 | 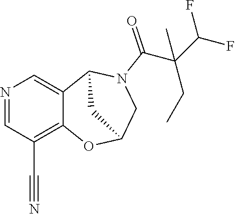 [SMILES: CCC(C)(C(=O)N1C[C@@H]2C[C@H]1c1cncc(C#N)c1O2)C(F)F] | (2S,5S)-4-(2-(R or S)- (difluoromethyl)-2- methylbutanoyl)-2,3,4,5- tetrahydro-2,5- methanopyrido[3,4- f][1,4]oxazepine-9-carbonitrile | Calc'd 322, found 322 |
| 1-85 | 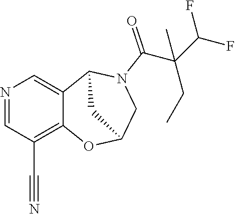 [SMILES: CCC(C)(C(=O)N1C[C@@H]2C[C@H]1c1cncc(C#N)c1O2)C(F)F] | (2S,5S)-4-(2-(R or S)- (difluoromethyl)-2- methylbutanoyl)-2,3,4,5- tetrahydro-2,5- methanopyrido[3,4- f][1,4]oxazepine-9-carbonitrile | Calc'd 322, found 322 |
| 1-86 | 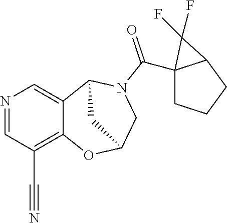 [SMILES: N#Cc1cncc2c1O[C@H]1C[C@@H]2N(C(=O)C23CCCC2C3(F)F)C1] | (2S,5S)-4-((1R,5S or 1S,5R)- 6,6- difluorobicyclo[3.1.0]hexane- 1-carbonyl)-2,3,4,5-tetrahydro- 2,5-methanopyrido[3,4- f][1,4]oxazepine-9-carbonitrile | Calc'd 332, found 332 |
| 1-87 | 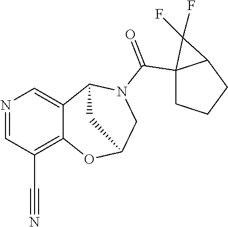 [SMILES: N#Cc1cncc2c1O[C@H]1C[C@@H]2N(C(=O)C23CCCC2C3(F)F)C1] | (2S,5S)-4-((1R,5S or 1S,5R)- 6,6- difluorobicyclo[3.1.0]hexane- 1-carbonyl)-2,3,4,5-tetrahydro- 2,5-methanopyrido[3,4- f][1,4]oxazepine-9-carbonitrile | Calc'd 332, found 332 |
| 1-88 | 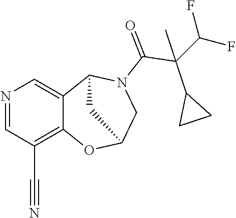 [SMILES: CC(C(=O)N1C[C@@H]2C[C@H]1c1cncc(C#N)c1O2)(C(F)F)C1CC1] | (2S,5S)-4-(2-(R or S)- cyclopropyl-3,3-difluoro-2- methylpropanoyl)-2,3,4,5- tetrahydro-2,5- methanopyrido[3,4- f][1,4]oxazepine-9-carbonitrile | Calc'd 334, found 334 |
| 1-89 | 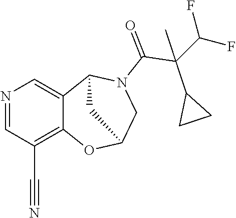 [SMILES: CC(C(=O)N1C[C@@H]2C[C@H]1c1cncc(C#N)c1O2)(C(F)F)C1CC1] | (2S,5S)-4-(2-(R or S)- cyclopropyl-3,3-difluoro-2- methylpropanoyl)-2,3,4,5- tetrahydro-2,5- methanopyrido[3,4- f][1,4]oxazepine-9-carbonitrile | Calc'd 334, found 334 |
| 1-90 | 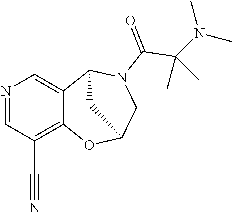 [SMILES: CN(C)C(C)(C)C(=O)N1C[C@@H]2C[C@H]1c1cncc(C#N)c1O2] | (2S,5S)-4-(2-(dimethylamino)- 2-methylpropanoyl)-2,3,4,5- tetrahydro-2,5- methanopyrido[3,4- f][1,4]oxazepine-9-carbonitrile | Calc'd 301, found 301 |
| 1-91 | 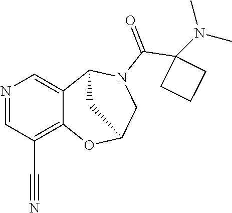 [SMILES: CN(C)C1(C(=O)N2C[C@@H]3C[C@H]2c2cncc(C#N)c2O3)CCC1] | (2S,5S)-4-(1- (dimethylamino)cyclobutane-1- carbonyl)-2,3,4,5-tetrahydro- 2,5-methanopyrido[3,4- f][1,4]oxazepine-9-carbonitrile | Calc'd 313, found 313 |
| 1-92 | 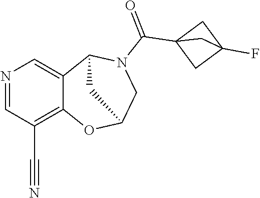 [SMILES: N#Cc1cncc2c1O[C@H]1C[C@@H]2N(C(=O)C23CC(F)(C2)C3)C1] | (2S,5S)-4-(3- fluorobicyclo[1.1.1]pentane-1- carbonyl)-2,3,4,5-tetrahydro- 2,5-methanopyrido[3,4- f][1,4]oxazepine-9-carbonitrile | Calc'd 300, found 300 |
| 1-93 | 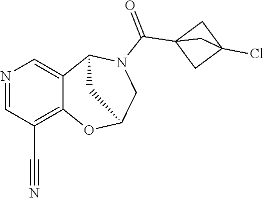 [SMILES: N#Cc1cncc2c1O[C@H]1C[C@@H]2N(C(=O)C23CC(Cl)(C2)C3)C1] | (2S,5S)-4-(3- chlorobicyclo[1.1.1]pentane-1- carbonyl)-2,3,4,5-tetrahydro- 2,5-methanopyrido[3,4- f][1,4]oxazepine-9-carbonitrile | Calc'd 316, found 316 |
| 1-94 | 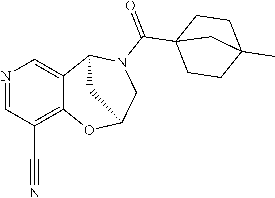 [SMILES: CC12CCC(C(=O)N3C[C@@H]4C[C@H]3c3cncc(C#N)c3O4)(CC1)C2] | (2S,5S)-4-(4- methylbicyclo[2.2.1]heptane-1- carbonyl)-2,3,4,5-tetrahydro- 2,5-methanopyrido[3,4- f][1,4]oxazepine-9-carbonitrile | Calc'd 324, found 324 |
| 1-95 | 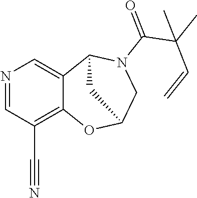 [SMILES: C=CC(C)(C)C(=O)N1C[C@@H]2C[C@H]1c1cncc(C#N)c1O2] | (2S,5S)-4-(2,2-dimethylbut-3- enoyl)-2,3,4,5-tetrahydro-2,5- methanopyrido[3,4- f][1,4]oxazepine-9-carbonitrile | Calc'd 284, found 284 |
| 1-96 | 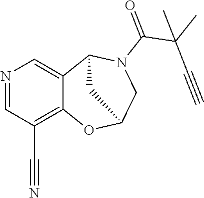 [SMILES: C#CC(C)(C)C(=O)N1C[C@@H]2C[C@H]1c1cncc(C#N)c1O2] | (2S,5S)-4-(2,2-dimethylbut-3- ynoyl)-2,3,4,5-tetrahydro-2,5- methanopyrido[3,4- f][1,4]oxazepine-9-carbonitrile | Calc'd 282, found 282 |
| 1-97 | 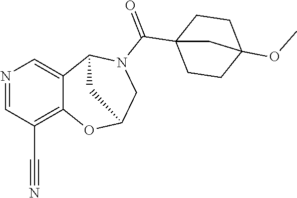 [SMILES: COC12CCC(C(=O)N3C[C@@H]4C[C@H]3c3cncc(C#N)c3O4)(CC1)C2] | (2S,5S)-4-(4- methoxybicyclo[2.2.1]heptane- 1-carbonyl)-2,3,4,5-tetrahydro- 2,5-methanopyrido[3,4- f][1,4]oxazepine-9-carbonitrile | Calc'd 340, found 340 |
| 1-98 | 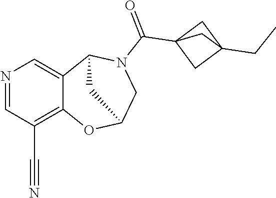 [SMILES: CCC12CC(C(=O)N3C[C@@H]4C[C@H]3c3cncc(C#N)c3O4)(C1)C2] | (2S,5S)-4-(3- ethylbicyclo[1.1.1]pentane-1- carbonyl)-2,3,4,5-tetrahydro- 2,5-methanopyrido[3,4- f][1,4]oxazepine-9-carbonitrile | Calc'd 310, found 310 |
| 1-99 | 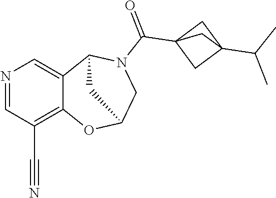 [SMILES: CC(C)C12CC(C(=O)N3C[C@@H]4C[C@H]3c3cncc(C#N)c3O4)(C1)C2] | (2S,5S)-4-(3- isopropylbicyclo[1.1.1]pentane- 1-carbonyl)-2,3,4,5- tetrahydro-2,5- methanopyrido[3,4- f][1,4]oxazepine-9-carbonitrile | Calc'd 324, found 324 |
| 1-100 | 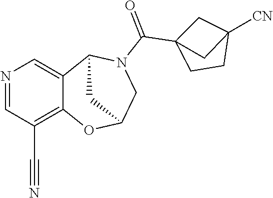 [SMILES: N#Cc1cncc2c1O[C@H]1C[C@@H]2N(C(=O)C23CCC(C#N)(C2)C3)C1] | (2S,5S)-4-(4- cyanobicyclo[2.1.1]hexane-1- carbonyl)-2,3,4,5-tetrahydro- 2,5-methanopyrido[3,4- f][1,4]oxazepine-9-carbonitrile | Calc'd 321, found 321 |
| 1-101 | 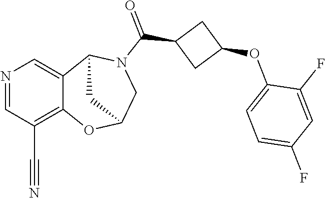 [SMILES: N#Cc1cncc2c1O[C@H]1C[C@@H]2N(C(=O)[C@H]2C[C@@H](Oc3ccc(F)cc3F)C2)C1] | (2S,5S)-4-(cis)-3-(2,4- difluorophenoxy)cyclobutane- 1-carbonyl)-2,3,4,5-tetrahydro- 2,5-methanopyrido[3,4- 1][1,4]oxazepine-9-carbonitrile | Calc'd 398, found 398 |
| 1-102 | 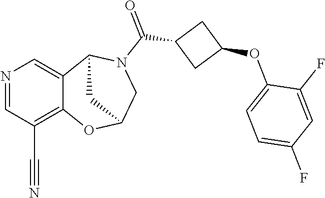 [SMILES: N#Cc1cncc2c1O[C@H]1C[C@@H]2N(C(=O)[C@H]2C[C@H](Oc3ccc(F)cc3F)C2)C1] | (2S,5S)-4-(trans)-3-(2,4- difluorophenoxy)cyclobutane- 1-carbonyl)-2,3,4,5-tetrahydro- 2,5-methanopyrido[3,4- f][1,4]oxazepine-9-carbonitrile | Calc'd 398, found 398 |
| 1-103 | 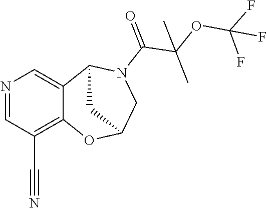 [SMILES: CC(C)(OC(F)(F)F)C(=O)N1C[C@@H]2C[C@H]1c1cncc(C#N)c1O2] | (2S,5S)-4-(2-methyl-2- (trifluoromethoxy)propanoyl)- 2,3,4,5-tetrahydro-2,5- methanopyrido[3,4- f][1,4]oxazepine-9-carbonitrile | Calc'd 342, found 342 |
| 1-104 | 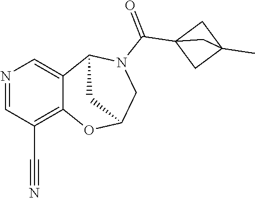 [SMILES: CC12CC(C(=O)N3C[C@@H]4C[C@H]3c3cncc(C#N)c3O4)(C1)C2] | (2S,5S)-4-(3- methylbicyclo[1.1.1]pentane-1- carbonyl)-2,3,4,5-tetrahydro- 2,5-methanopyrido[3,4- f][1,4]oxazepine-9-carbonitrile | Calc'd 296, found 296 |
| 1-105 | 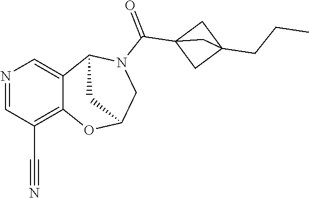 [SMILES: CCCC12CC(C(=O)N3C[C@@H]4C[C@H]3c3cncc(C#N)c3O4)(C1)C2] | (2S,5S)-4-(3- propylbicyclo[1.1.1]pentane-1- carbonyl)-2,3,4,5-tetrahydro- 2,5-methanopyrido[3,4- f][1,4]oxazepine-9-carbonitrile | Calc'd 324, found 324 |
| 1-106 | 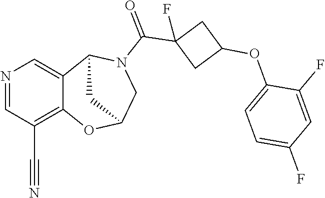 [SMILES: N#Cc1cncc2c1O[C@H]1C[C@@H]2N(C(=O)C2(F)CC(Oc3ccc(F)cc3F)C2)C1] | (2S,5S)-4-(3-(2,4- difluorophenoxy)-1-(R or S)- fluorocyclobutane-1-carbonyl)- 2,3,4,5-tetrahydro-2,5- methanopyrido[3,4- f][1,4]oxazepine-9-carbonitrile | Calc'd 416, found 416 |
| 1-107 | 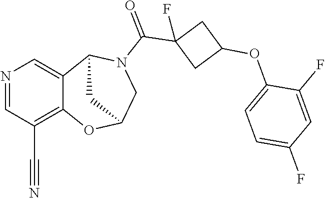 [SMILES: N#Cc1cncc2c1O[C@H]1C[C@@H]2N(C(=O)C2(F)CC(Oc3ccc(F)cc3F)C2)C1] | (2S,5S)-4-(3-(2,4- difluorophenoxy)-1-(R or S)- fluorocyclobutane-1-carbonyl)- 2,3,4,5-tetrahydro-2,5- methanopyrido[3,4- f][1,4]oxazepine-9-carbonitrile | Calc'd 416, found 416 |
| 1-108 | 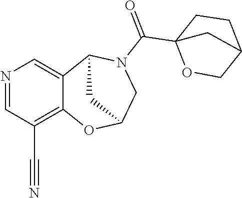 [SMILES: N#Cc1cncc2c1O[C@H]1C[C@@H]2N(C(=O)C23CCC(CO2)C3)C1] | (2S,5S)-4-(2- oxabicyclo[2.2.1]heptane-1- carbonyl)-2,3,4,5-tetrahydro- 2,5-methanopyrido[3,4- f][1,4]oxazepine-9-carbonitrile | Calc'd 312, found 312 |
| 1-109 | 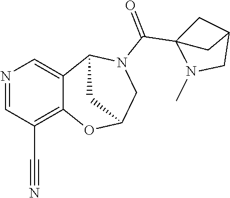 [SMILES: CN1CC2CC1(C(=O)N1C[C@@H]3C[C@H]1c1cncc(C#N)c1O3)C2] | (2S,5S)-4-(2-methyl-2- azabicyclo[2.1.1]hexane-1- carbonyl)-2,3,4,5-tetrahydro- 2,5-methanopyrido[3,4- f][1,4]oxazepine-9-carbonitrile | Calc'd 311, found 311 |
| 1-110 | 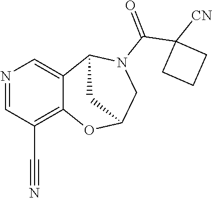 [SMILES: N#Cc1cncc2c1O[C@H]1C[C@@H]2N(C(=O)C2(C#N)CCC2)C1] | (2S,5S)-4-(1- cyanocyclobutane-1-carbonyl)- 2,3,4,5-tetrahydro-2,5- methanopyrido[3,4- f][1,4]oxazepine-9-carbonitrile | Calc'd 295, found 295 |
| 1-111 | 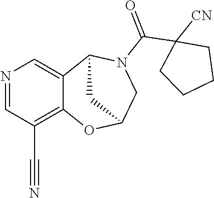 [SMILES: N#Cc1cncc2c1O[C@H]1C[C@@H]2N(C(=O)C2(C#N)CCCC2)C1] | (2S,5S)-4-(1- cyanocyclopentane-1- carbonyl)-2,3,4,5-tetrahydro- 2,5-methanopyrido[3,4- f][1,4]oxazepine-9-carbonitrile | Calc'd 309, found 309 |
| 1-112 | 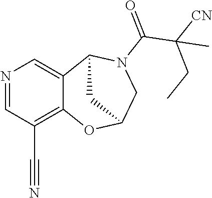 [SMILES: CCC(C)(C#N)C(=O)N1C[C@@H]2C[C@H]1c1cncc(C#N)c1O2] | (2S,5S)-4-(2-(R or S)-cyano-2- methylbutanoyl)-2,3,4,5- tetrahydro-2,5- methanopyrido[3,4- f][1,4]oxazepine-9-carbonitrile | Calc'd 297, found 297 |
| 1-113 | 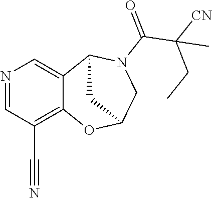 [SMILES: CCC(C)(C#N)C(=O)N1C[C@@H]2C[C@H]1c1cncc(C#N)c1O2] | (2S,5S)-4-(2-(R or S)-cyano-2- methylbutanoyl)-2,3,4,5- tetrahydro-2,5- methanopyrido[3,4- f][1,4]oxazepine-9-carbonitrile | Calc'd 297, found 297 |

### Table 2 Examples

### Example 2A. Preparation of 3,3-Difluoro-1-((2S,5S)-9-fluoro-2,3-dihydro-2,5-methanopyrido[3,4-f][1,4]oxazepin-4(5H)-yl)-2,2-dimethylpropan-1-one (2-1)

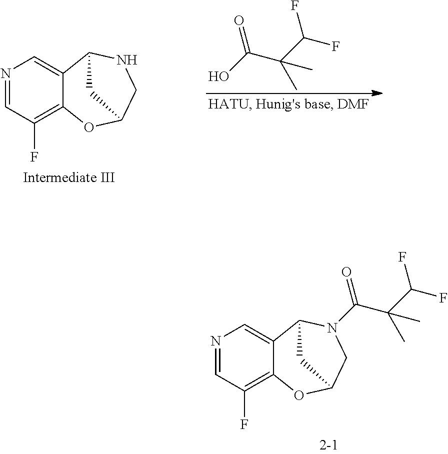

A mixture of 3,3-difluoro-2,2-dimethylpropanoic acid (8.0 mg, 0.058 mmol), HATU (16 mg, 0.041 mmol) and DIEA (10 μL, 0.055 mmol) in DMF (1 mL) was treated with (2S,5S)-9-fluoro-2,3,4,5-tetrahydro-2,5-methanopyrido[3,4-f][1,4]oxazepine (8.3 mg, 0.028 mmol). The mixture was stirred for 16 h, then concentrated to give a residue which was purified reverse phase chromatography (gradient of 22 to 52% MeCN/water with 0.05% NH₄OH and 10 mM NH₄HCO₃) to give 3,3-difluoro-1-((2S,5S)-9-fluoro-2,3-dihydro-2,5-methanopyrido[3,4-f][1,4]oxazepin-4(5H)-yl)-2,2-dimethylpropan-1-one. ¹H NMR (400 MHz, CD₃OD) δ 8.21 (d, J=3 Hz, 1H), 8.16 (s, 1H), 5.93-6.24 (m, 1H), 5.38 (br s, 1H), 5.32 (br s, 1H), 4.10 (br s, 2H), 2.33-2.39 (m, 1H), 2.20-2.26 (m, 1H), 1.30 (s, 3H), 1.18 (br s, 3H); MS (EI) calculated for C₁₄H₁₆F₃N₂O₂[M+H]⁺ 301; found, 301.

### Example 2B. Preparation of 1-((2S,5S)-9-Chloro-2,3-dihydro-2,5-methanopyrido[3,4-f][1,4]oxazepin-4(5H)-yl)-3,3-difluoro-2,2-dimethylpropan-1-one (2-2)

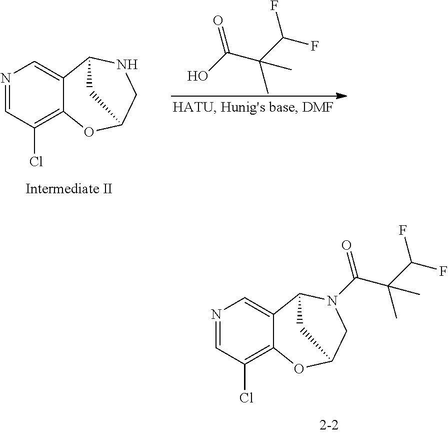

A mixture of DIEA (7.0 μL, 0.041 mmol), 3,3-difluoro-2,2-dimethylpropanoic acid (2.5 mg, 0.016 mmol) and HATU (7.8 mg, 0.021 mmol) in DMF (0.5 mL) was stirred for 10 min. Then (2S,5S)-9-chloro-2,3,4,5-tetrahydro-2,5-methanopyrido[3,4-f][1,4]oxazepine (3.0 mg, 0.014 mmol) was added. The mixture was stirred for 16 h, concentrated and purified by reverse phase chromatography (gradient of 20 to 40% MeCN/water with 0.1% TFA) to give 1-((2S,5S)-9-chloro-2,3-dihydro-2,5-methanopyrido[3,4-f][1,4]oxazepin-4(5H)-yl)-3,3-difluoro-2,2-dimethylpropan-1-one. ¹H NMR (400 MHz, CD₃OD) δ 8.56 (br s, 1H), 8.40 (br s, 1H), 5.89-6.24 (m, 1H), 5.51 (br s, 1H), 5.39 (br d, J=3.91 Hz, 1H), 4.06-4.27 (m, 2H), 2.46 (ddd, J=2.5, 5.2, 13 Hz, 1H), 2.28 (d, J=13 Hz, 1H), 1.30 (s, 3H), 1.19 (s, 3H); MS (EI) calculated for C₁₄H₁₆ClF₂N₂O₂ [M+H]⁺317; found, 317

### Example 2C. Preparation of ((2S,5S)-9-Chloro-2,3-dihydro-2,5-methanopyrido[3,4-f][1,4]oxazepin-4(5H)-yl)(4-fluorobicyclo[2.2.1]heptan-1-yl)methanone (2-3)

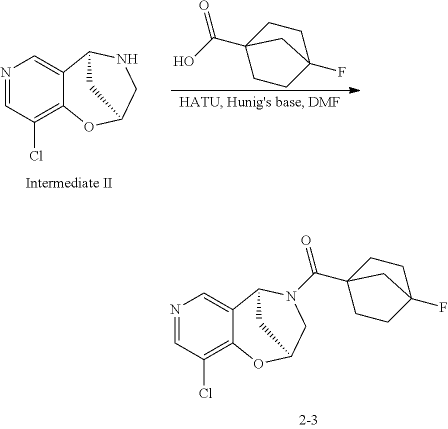 [SMILES: Clc1cncc2c1O[C@@H]1CN[C@H]2C1 ; O=C(O)C12CCC(F)(CC1)C2 ; O=C(N1C[C@@H]2C[C@H]1c1cncc(Cl)c1O2)C12CCC(F)(CC1)C2]

A solution of 4-fluorobicyclo[2.2.1]heptane-1-carboxylic acid (20 mg, 0.13 mmol) and HATU (50 mg, 0.13 mmol) in DMF (1 mL) was treated with Hunig's base (0.050 mL, 0.29 mmol), stirred for 5 min, and then treated with (2S,5S)-9-chloro-2,3,4,5-tetrahydro-2,5-methanopyrido[3,4-f][1,4]oxazepine, TFA (20 mg, 0.064 mmol). The mixture was stirred for 18 h, diluted with MeOH (1 mL), filtered and purified by reverse phase chromatography (gradient of 2-55% MeCN/water with 0.1% TFA) providing ((2S,5S)-9-chloro-2,3-dihydro-2,5-methanopyrido[3,4-f][1,4]oxazepin-4(5H)-yl)(4-fluorobicyclo[2.2.1]heptan-1-yl)methanone. ¹H NMR (500 MHz, DMSO-d₆) 8.48 (s, 1H), 8.25 (s, 1H), 5.43 (m, 1H), 5.23 (m, 1H), 3.94 (m, 2H), 2.30 (m, 1H), 2.13 (m, 1H), 1.92 (m, 2H), 1.65-1.90 (m, 8H); MS (EI) calculated for C₁₇H₁₉ClFN₂O₂[M+H]⁺ 337; found, 337.

### Example 2D. Preparation of (4-fluorobicyclo[2.2.1]heptan-1-yl)[(2S,5S)-9-fluoro-2,3-dihydro-2,5-methanopyrido[3,4-f][1,4]oxazepin-4(5H)-yl]methanone (2-7)

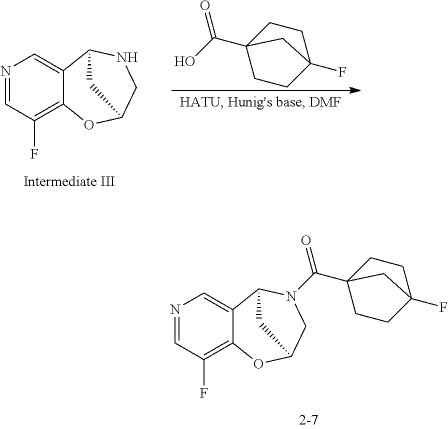 [SMILES: Fc1cncc2c1O[C@@H]1CN[C@H]2C1 ; O=C(O)C12CCC(F)(CC1)C2 ; O=C(N1C[C@@H]2C[C@H]1c1cncc(F)c1O2)C12CCC(F)(CC1)C2]

A solution of 4-fluorobicyclo[2.2.1]heptane-1-carboxylic acid (10 mg, 0.063 mmol) and HATU (20 mg, 0.053 mmol) in DMF (1 mL) was treated with Hunig's base (0.030 mL, 0.17 mmol), stirred for 5 min, and then treated with (2S,5S)-9-fluoro-2,3,4,5-tetrahydro-2,5-methanopyrido[3,4-f][1,4]oxazepine, TFA (9 mg, 0.022 mmol). The mixture was stirred for 18 h, diluted with MeOH (1 mL), filtered and purified by reverse phase chromatography (gradient of 2-55% MeCN/water with 0.1% TFA) providing ((2S,5S)-9-fluoro-2,3-dihydro-2,5-methanopyrido[3,4-f][1,4]oxazepin-4(5H)-yl)(4-fluorobicyclo[2.2.1]heptan-1-yl)methanone. ¹H NMR (500 MHz, DMSO-d) 8.55 (s, 1H), 8.24 (s, 1H), 5.43 (m, 1H), 5.28 (m, 1H), 3.92-4.04 (m, 2H), 2.32 (m, 1H), 2.17 (m, 1H), 1.63-2.02 (m, 10H); MS (EI) calculated for C₁₇H₁₉F₂N₂O₂ [M+H]⁺ 321; found, 321.

### Example 2E. Preparation of 1-((2S,5S)-9-Bromo-2,3-dihydro-2,5-methanopyrido[3,4-][1,4]oxazepin-4(5H)-yl)-2,2-dimethylpropan-1-one (2-8)

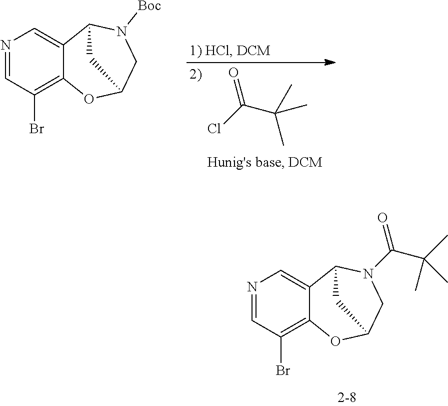

Step 1. A solution of tert-butyl (2S,5S)-9-bromo-2,3-dihydro-2,5-methanopyrido[3,4-f][1,4]oxazepine-4(5H)-carboxylate (500 mg, 1.47 mmol) in DCM (7.3 mL) was treated with a 4 M solution of HCl in dioxane (1.5 mL, 5.9 mmol) and stirred overnight. The reaction was concentrated, giving (2S,5S)-9-bromo-2,3,4,5-tetrahydro-2,5-methanopyrido[3,4-f][1,4]oxazepine, HCl. MS (EI) Calculated for C₉H₁₀BrN₂O [M+H]⁺, 241, 243; found, 241, 243.

Step 2. A mixture consisting of (2S,5S)-9-bromo-2,3,4,5-tetrahydro-2,5-methanopyrido[3,4-f][1,4]oxazepine (353 mg, 1.47 mmol) and Hunig's base (0.770 mL, 4.40 mmol) in DCM (7.3 mL) was treated with pivaloyl chloride (265 mg, 2.20 mmol), and the mixture was stirred for 1 h. The mixture was concentrated, and the residue purified by chromatography on silica gel (24 g silica gel, 0-100/o EtOAc/hexanes) giving 1-((2S,5S)-9-bromo-2,3-dihydro-2,5-methanopyrido[3,4-f][1,4]oxazepin-4(5H)-yl)-2,2-dimethylpropan-1-one. ¹H NMR (500 MHz, DMSO-d₆) δ 8.43 (s, 1H), 8.21 (s, 1H), 5.29 (m, 2H), 3.99 (m, 2H), 2.26 (dd, J=8.0, 2.8 Hz, 1H), 2.08 (d, J=12.7 Hz, 1H), 1.10 (s, 9H); MS (EI) calculated for C₁₄H₁₈BrN₂O₂[M+H]⁺, 325, 327; found, 325, 327.

### Example 2F. Preparation of 2,2-Dimethyl-1-((2S,5S)-9-methyl-2,3-dihydro-2,5-methanopyrido[3,4-f][1,4]oxazepin-4(5H)-1)propan-1-one (2-13)

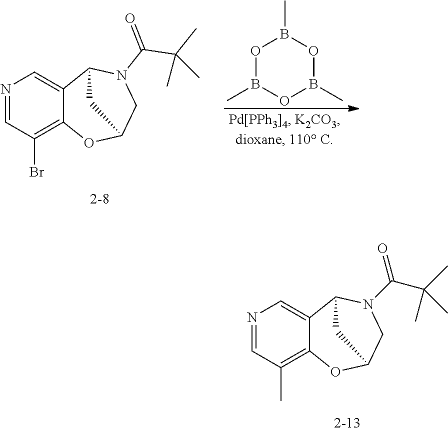

A mixture of 1-((2S,5S)-9-bromo-2,3-dihydro-2,5-methanopyrido[3,4-f][1,4]oxazepin-4(5H)-yl)-2,2-dimethylpropan-1-one (2-8) (16 mg, 0.050 mmol), potassium carbonate (10 mg, 0.075 mmol), and tetrakis(triphenylphosphine)palladium(0) (5.8 mg, 0.005 mmol) in a 2 dram vial was degassed and backfilled with nitrogen. The mixture was dissolved in dioxane (0.4 mL), treated with trimethylboroxine (7.0 μl, 0.050 mmol), and heated to 110° C. overnight. The reaction was then diluted with water, extracted with EtOAc, and the combined organic extracts were dried over sodium sulfate, filtered, and concentrated. The residue was purified by reverse phase chromatography (MeCN/water with 0.1% TFA) to afford 2,2-dimethyl-1-((2S,5S)-9-methyl-2,3-dihydro-2,5-methanopyrido[3,4-f][1,4]oxazepin-4(5H)-1)propan-1-one (2-13). ¹H NMR (500 MHz, DMSO-d₆) δ 8.09 (s, 2H), 5.21 (s, 2H), 3.95 (d, J=12 Hz, 1H), 3.85 (br s, 1H), 2.14-2.26 (m, 1H), 2.06 (s, 3H), 2.01 (d, J=12 Hz, 1H), 1.10 (s, 9H); MS (EI) calculated for C₁₅H₂₁N₂O₂, [M+H]⁺, 261; found, 261.

### Example 2G. Preparation of 1-((2S,5S)-9-Ethynyl-2,3-dihydro-2,5-methanopyrido[3,4-f][1,4]oxazepin-4(5H)-yl)-2,2-dimethylpropan-1-one (2-17)

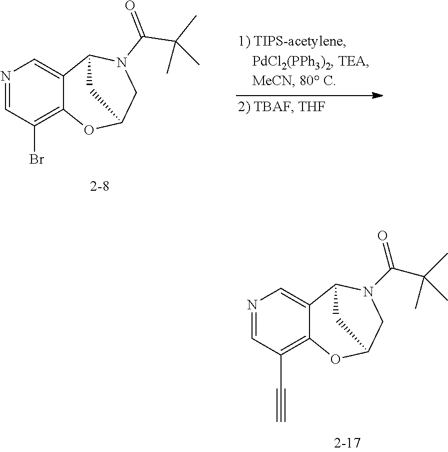 [SMILES: CC(C)(C)C(=O)N1C[C@@H]2C[C@H]1c1cncc(Br)c1O2 ; C#Cc1cncc2c1O[C@H]1C[C@@H]2N(C(=O)C(C)(C)C)C1]

Step 1. A mixture of 1-((2S,5S)-9-bromo-2,3-dihydro-2,5-methanopyrido[3,4-f][1,4]oxazepin-4(5H)-yl)-2,2-dimethylpropan-1-one (16 mg, 0.050 mmol) and bis(triphenylphosphine)palladium(II) dichloride (1.8 mg, 2.5 μmol) in a vial was degassed and backfilled with nitrogen. The mixture was dissolved in MeCN (250 μL), treated with (triisopropyl)acetylene (13 μL, 0.060 mmol), TEA (14 μL, 0.10 mmol) and heated to 80° C. for 10 hours. The mixture was purified by chromatography on silica gel (4 g silica gel, 0-100% EtOAc/Hexanes) giving 2,2-dimethyl-1-((2S,5S)-9-((triisopropylsilyl)ethynyl)-2,3-dihydro-2,5-methanopyrido[3,4-f][[1,4]oxazepin-4(5H)-yl)propan-1-one. MS (EI) calculated for C₂₅H₃₉N₂O₂Si [M+H]⁺427; found 427.

Step 2. A mixture of 2,2-dimethyl-1-((2S,5S)-9-((triisopropylsilyl)ethynyl)-2,3-dihydro-2,5-methanopyrido[3,4-f]11.4]oxazepin-4(5H)-yl)propan-1-one (15 mg, 0.035 mmol) in THE (0.5 mL) was treated with 1 M solution of tetrabutylammonium fluoride (0.039 ml, 0.039 mmol). The mixture was stirred for 1 h, and then purified by chromatography on silica gel (4 g silica gel, 0-100% EtOAc/Hexanes) followed by reverse phase chromatography (MeCN/water with 0.1% TFA) to afford 1-((2S,5S)-9-ethynyl-2,3-dihydro-2,5-methanopyrido[3,4-f][1,4]oxazepin-4(5H)-yl)-2,2-dimethylpropan-1-one. ¹H NMR (500 MHz, DMSO-d₆) δ 8.60 (s, 1H), 8.41 (s, 1H), 5.45 (s, 1H), 5.24 (s, 1H), 4.67 (s, 1H), 3.99 (s, 2H), 2.33 (ddd, J=13 Hz, 5.0, 2.4 Hz, 1H), 2.14 (d, J=13 Hz, 1H), 1.09 (s, 9H); MS (EI) calculated for C₁₆H₁₉N₂O₂ [M+H]⁺, 270; found, 270.

### Example 2H. Preparation of 2,2-Dimethyl-1-((2S,5S)-9-(phenylethynyl)-2,3-dihydro-2,5-methanopyrido[3,4-f][1,4]oxazepin-4(5H)-yl)propan-1-one (2-20)

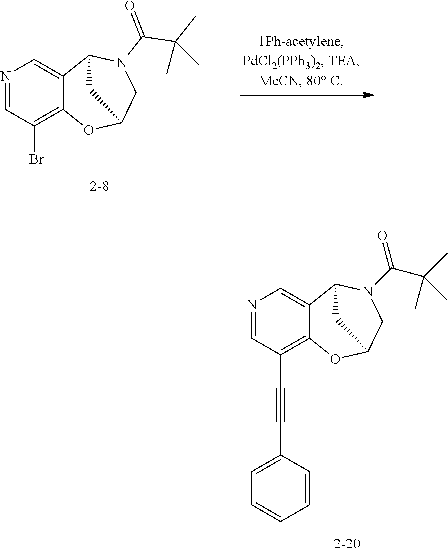 [SMILES: CC(C)(C)C(=O)N1C[C@@H]2C[C@H]1c1cncc(Br)c1O2 ; CC(C)(C)C(=O)N1C[C@@H]2C[C@H]1c1cncc(C#Cc3ccccc3)c1O2]

A mixture of 1-((2S,5S)-9-bromo-2,3-dihydro-2,5-methanopyrido[3,4-f][1,4]oxazepin-4(5H)-yl)-2,2-dimethylpropan-1-one (16 mg, 0.050 mmol) and bis(triphenylphosphine)palladium(II) dichloride (1.8 mg, 2.5 μmol) in a vial was degassed and backfilled with nitrogen. Dissolved in MeCN (0.5 mL), added ethynylbenzene (7 μl, 0.06 mmol), TEA (14 μL, 0.10 mmol) and heated to 80° C. with stirring overnight. Purified by reverse phase chromatography (MeCN/water with 0.1% TFA) to afford 2,2-dimethyl-1-((2S,5S)-9-(phenylethynyl)-2,3-dihydro-2,5-methanopyrido[3,4-f][1.4]oxazepin-4(5H)-yl)propan-1-one. ¹H NMR (500 MHz, DMSO-d₆) δ 8.70 (s, 1H), 8.43 (s, 1H), 7.57 (m, 2H), 7.48 (m, 3H), 5.50 (s, 1H), 5.27 (s, 1H), 4.02 (s, 2H), 2.35 (d, J=12 Hz, 1H), 2.18 (d, J=13 Hz, 1H), 1.10 (s, 9H); MS (EI) calculated for C₂₂H₂₃N₂O₂ [M+H]⁺, 347; found, 347.

### Example 2I. Preparation of ((2S,5S)-9-Fluoro-8-methyl-2,3-dihydro-2,5-methanopyrido[3,4-f][1,4]oxazepin-4(5H)-yl)(4-(trifluoromethyl)bicyclo[2.2.1]heptan-1-yl)methanone (2-27)

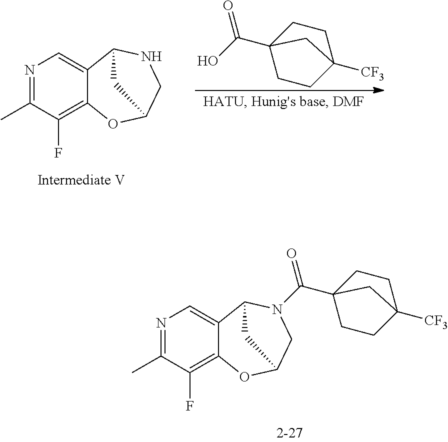 [SMILES: Cc1ncc2c(c1F)O[C@@H]1CN[C@H]2C1 ; O=C(O)C12CCC(C(F)(F)F)(CC1)C2 ; Cc1ncc2c(c1F)O[C@H]1C[C@@H]2N(C(=O)C23CCC(C(F)(F)F)(CC2)C3)C1]

A mixture containing 4-(trifluoromethyl)bicyclo[2.2.1]heptane-1-carboxylic acid (22 mg, 0.11 mmol) in DMF (1 mL) was treated with Hunig's base (0.040 mL, 0.24 mmol) and HATU (46 mg, 0.12 mmol). The mixture was stirred for 15 min, then treated with Intermediate V (20 mg, 0.081 mmol) and stirred overnight. The mixture was then purified by reverse phase chromatography (gradient of 20-50% MeCN/water with 0.1% TFA) to give ((2S,5S)-9-fluoro-8-methyl-2,3-dihydro-2,5-methanopyrido[3,4-f][1,4]oxazepin-4(5H)-yl)(4-(trifluoromethyl)bicyclo[2.2.1]heptan-1-yl)methanone as an oil. ¹H NMR (400 MHz, CD₃OD) δ 8.31 (s, 1H), 5.57 (br s, 1H), 5.40 (br d, J=4.7 Hz, 1H), 4.13-4.21 (m, 1H), 4.01-4.10 (m, 1H), 2.60 (d, J=2.7 Hz, 3H), 2.51 (ddd, J=2.2, 5.3, 13 Hz, 1H), 2.32 (br d, J=13 Hz, 1H), 1.84-2.01 (m, 7H), 1.57-1.81 (m, 3H); MS (EI) cal'd for C₁₉H₂₁F₄N₂O₂[M+H]⁺, 385; found, 385.

### Example 2J. Preparation of Bicyclo[2.2.1]heptan-1-yl((2S,5S)-9-chloro-2,3-dihydro-2,5-methanopyrido[3,4-f][1,4]oxazepin-4(5H)-yl)methanone (2-31)

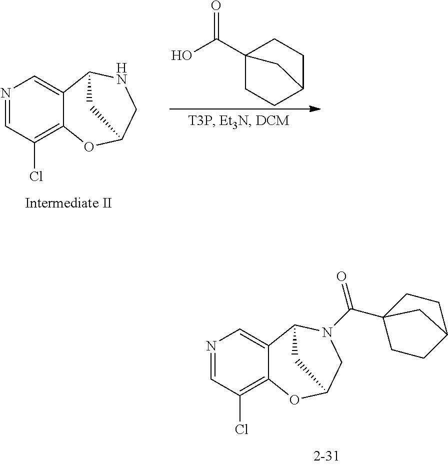 [SMILES: Clc1cncc2c1O[C@@H]1CN[C@H]2C1 ; O=C(O)C12CCC(CC1)C2 ; O=C(N1C[C@@H]2C[C@H]1c1cncc(Cl)c1O2)C12CCC(CC1)C2]

A solution of Intermediate II (47 mg, 0.15 mmol) and bicyclo[2.2.1]heptane-1-carboxylic acid (25 mg, 0.18 mmol) in DCM (750 μl) was treated with Et₃N (84 μl, 0.60 mmol) then 1-propanephosphonic anhydride (50 wt % in EtOAc) (179 μl, 0.30 mmol). The reaction was allowed to stir at RT for 1 h, then loaded directly onto silica and purified by chromatography on silica gel (4 g silica gel, 0-100% EtOAc/Hexanes then 20% MeOH/DCM) giving bicyclo[2.2.1]heptan-1-yl((2S,5S)-9-chloro-2,3-dihydro-2,5-methanopyrido[3,4-f][1,4]oxazepin-4(5H)-yl)methanone. ¹H NMR (500 MHz, Chloroform-d) δ 8.59 (s, 1H), 8.45 (s, 1H), 5.42 (s, 2H), 4.07 (d, J=11.1 Hz, 1H), 3.94 (d, J=10.6 Hz, 1H), 2.37 (d, J=11.3 Hz, 1H), 2.24 (m, 2H), 1.63 (m, 8H), 1.37 (m, 2H). MS (EI) Calculated for C₁₇H₂₀ClN₂O₂ [M+H]⁺, 319; found, 319.

### Example 2K. Preparation of 1-((2S,5S)-9-Ethynyl-2,3-dihydro-2,5-methanopyrido[3,4-f][1,4]oxazepin-4(5H)-yl)-3,3-difluoro-2,2-dimethylpropan-1-one (2-32)

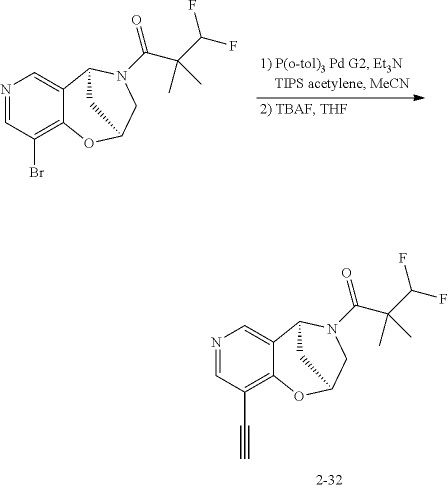 [SMILES: CC(C)(C(=O)N1C[C@@H]2C[C@H]1c1cncc(Br)c1O2)C(F)F ; C#Cc1cncc2c1O[C@H]1C[C@@H]2N(C(=O)C(C)(C)C(F)F)C1]

Step 1. 1-((2S,5S)-9-Bromo-2,3-dihydro-2,5-methanopyrido[3,4-f][1,4]oxazepin-4(5H)-yl)-3,3-difluoro-2,2-dimethylpropan-1-one (0.756 g, 2.09 mmol) and chloro[tri(o-tolyl)phosphine][2-(2′-amino-1,1′-biphenyl)]palladium(II) (0.129 g, 0.21 mmol) were added to a vial. The vial was degassed and backfilled with nitrogen. MeCN (11 mL) was added followed by (triisopropylsilyl)acetylene (0.7 mL, 3.14 mmol) and Et₃N (0.86 mL, 6.28 mmol), then the reaction was heated to 90° C. overnight. The reaction was cooled to RT and concentrated. The residue was purified by chromatography on silica gel (24 g silica gel, 0-100% EtOAc/Hexanes) giving 3,3-difluoro-2,2-dimethyl-1-((2S,5S)-9-((triisopropylsilyl)ethynyl)-2,3-dihydro-2,5-methanopyrido[3,4-f][1,4]oxazepin-4(5H)-yl)propan-1-one. MS (EI) Calculated for C₂₅H₃₇F₂N₂O₂Si [M+H]⁺, 463; found, 463.

Step 2. A solution of 3,3-difluoro-2,2-dimethyl-1-((2S,5S)-9-((triisopropylsilyl)ethynyl)-2,3-dihydro-2,5-methanopyrido[3,4-f][1,4]oxazepin-4(5H)-yl)propan-1-one (787 mg, 1.70 mmol) in THF (1.7 mL) was treated with a 1M solution TBAF in THF (1.87 mL, 1.87 mmol). The reaction was allowed to stir at RT for 1 h. The solution was concentrated and the residue purified by chromatography on silica gel (12 g silica gel, 0-100% EtOAc/Hexanes) giving 1-((2S,5S)-9-ethynyl-2,3-dihydro-2,5-methanopyrido[3,4-f][1,4]oxazepin-4(5H)-yl)-3,3-difluoro-2,2-dimethylpropan-1-one. ¹H NMR (500 MHz, Chloroform-d) δ 8.47 (s, 1H), 8.38 (s, 1H), 6.06 (t, J=56.7 Hz, 1H), 5.37 (s, 1H), 5.28 (d. J=17.1 Hz, 2H), 4.03 (d, J=12.0 Hz, 1H), 3.96 (dd, J=12.1, 3.4 Hz, 1H), 3.40 (s, 1H), 2.21 (s, 2H), 1.30 (s, 3H), 1.19 (s, 3H). MS (EI) Calculated for C₁₆H₁₇F₂N₂O₂ [M+H]⁺, 307; found, 307.

### Example 2L. Preparation of 3,3-Difluoro-2,2-dimethyl-1-((2S,5S)-9-(quinoxalin-6-ylethynyl)-2,3-dihydro-2,5-methanopyrido[3,4-f][1,4]oxazepin-4(5H)-yl)propan-1-one (2-34)

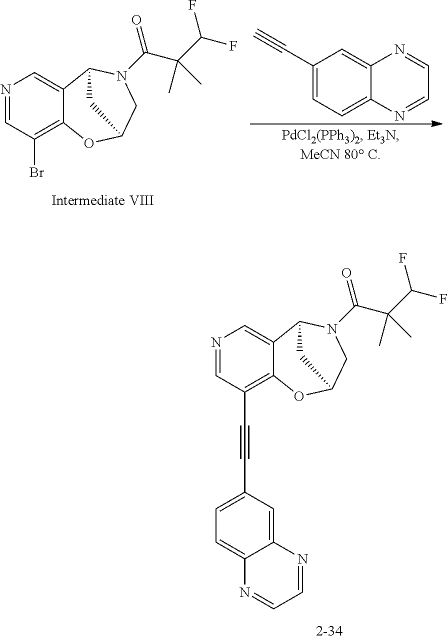

A mixture of Intermediate VIII (10.8 mg, 0.03 mmol) and bis(triphenylphosphine)palladium(II) chloride (1.1 mg, 0.002 mmol) in a vial was degassed and backfilled with nitrogen. The solids were dissolved in MeCN (0.3 mL), then 6-ethynylquinoxaline (5.6 mg, 0.036 mmol) and Et₃N (8.4 μL, 0.06 mmol) were added, and the reaction was heated to 80° C. with stirring overnight.

The product was purified by reverse phase chromatography (MeCN/water with 0.1% TFA) to afford 3,3-difluoro-2,2-dimethyl-1-((2S,5S)-9-(quinoxalin-6-ylethynyl)-2,3-dihydro-2,5-methanopyrido[3,4-f][1,4]oxazepin-4(5H)-yl)propan-1-one. ¹H NMR (500 MHz, DMSO-d₆) δ 9.01 (d, J=1.7 Hz, 1H), 8.98 (d, J=1.7 Hz, 1H), 8.65 (s, 1H), 8.36 (s, 1H), 8.28 (d, J=1.7 Hz, 1H), 8.16 (d, J=8.6 Hz, 1H), 7.96 (dd, J=8.6, 1.8 Hz, 1H), 6.20 (t, J=56.6 Hz, 1H), 5.47 (s, 1H), 5.27 (s, 1H), 2.54 (s, 2H), 2.35 (ddd, J=12.5, 4.8, 2.4 Hz, 1H), 2.18 (d, J=12.7 Hz, 1H), 1.21 (s, 3H), 1.09 (s, 3H). MS (EI) Calculated for C₂₄H₂₁F₂N₄O₂ [M+H]⁺, 435; found, 435.

### Example 2M. Preparation of 3,3-Difluoro-2,2-dimethyl-1-((2S,5S)-9-(phenylethynyl)-2,3-dihydro-2,5-methanopyrido[3,4-f][1,4]oxazepin-4(5H)-yl)propan-1-one (2-47)

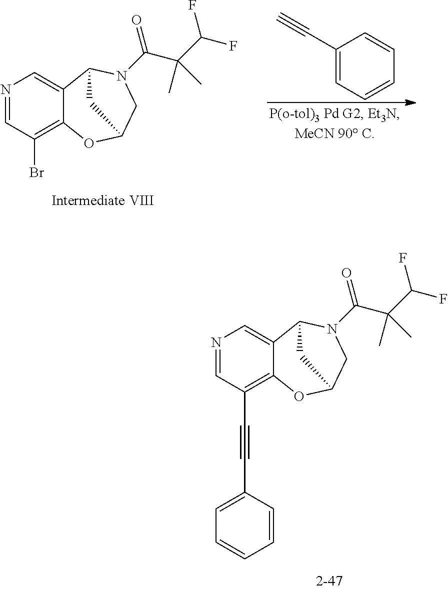

A mixture of Intermediate VIII (72 mg, 0.2 mmol) and chloro[tri(o-tolyl)phosphine][2-(2′-amino-1,1′-biphenyl)]palladium(II) (12 mg, 0.02 mmol) in a vial was degassed and backfilled with nitrogen. The solids were dissolved in MeCN (1.0 mL) then ethynylbenzene (31 mg, 0.3 mmol) and Et₃N (84 μL, 0.6 mmol) were added, and the reaction was heated to 90° C. with stirring overnight. The product was purified by reverse phase chromatography (MeCN/water with 0.1% TFA) to afford 3,3-difluoro-2,2-dimethyl-1-((2S,5S)-9-(phenylethynyl)-2,3-dihydro-2,5-methanopyrido[3,4-f][1,4]oxazepin-4(5H)-yl)propan-1-one. ¹H NMR (500 MHz, DMSO-d₆) δ 8.45 (s, 1H), 8.24 (s, 1H), 7.54 (s, 2H), 7.45 (d, J=2.9 Hz, 3H), 6.22 (t, J=56.4 Hz, 1H), 5.36 (s, 1H), 5.24 (s, 1H), 4.02 (m, 2H), 2.29 (d, J=10.9 Hz, 1H), 2.13 (d, J=12.7 Hz, 1H), 1.22 (s, 3H), 1.09 (s, 3H). MS (EI) calculated for C₂₂H₂₄N₂O₂ [M+H]⁺, 383; found, 383.

### Example 2N. Preparation of ((2S,5S)-9-(Pyrimidin-5-ylethynyl)-2,3-dihydro-2,5-methanopyrido[3,4-f][1,4]oxazepin-4(5H)-yl)(4-(trifluoromethyl)bicyclo[2.2.1]heptan-1-yl)methanone (2-50)

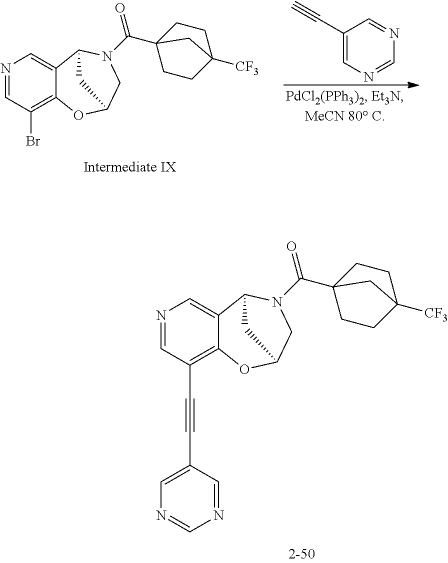

A mixture of Intermediate IX (12.9 mg, 0.03 mmol) and bis(triphenylphosphine)palladium(II) chloride (1.1 mg, 0.002 mmol) in a vial was degassed and backfilled with nitrogen. The solids were dissolved in MeCN (0.3 mL), then 5-ethynylpyrimidine (3.75 mg, 0.036 mmol) and Et₃N (8.4 μL, 0.06 mmol) were added, and the reaction was heated to 80° C. with stirring overnight. The product was purified by reverse phase chromatography (MeCN/water with 0.1% TFA) to afford ((2S,5S)-9-(pyrimidin-5-ylethynyl)-2,3-dihydro-2,5-methanopyrido[3,4-f][1,4]oxazepin-4(5H)-yl)(4-(trifluoromethyl)bicyclo[2.2.1]heptan-1-yl)methanone. ¹H NMR (500 MHz, DMSO-d₆) δ 9.20 (s, 1H), 8.99 (s, 2H), 8.48 (s, 1H), 8.25 (s, 1H), 5.36 (s, 1H), 5.21 (d, J=4.8 Hz, 1H), 3.93 (s, 2H), 2.28 (ddd, J=12.5, 4.9, 2.5 Hz, 1H), 2.13 (s, 1H), 1.95-1.74 (m, 8H), 1.69-1.50 (m, 4H). MS (EI) Calculated for C₂₄H₂₂F₃N₄O₂[M+H]⁺, 455, found, 455.

### Example 2O. Preparation of ((2S,5S)-9-(Pyrazin-2-ylethynyl)-2,3-dihydro-2,5-methanopyrido[3,4-f][1.4]oxazepin-4(5H)-yl)(4-(trifluoromethyl)bicyclo[2.2.1]heptan-1-yl)methanone (2-61)

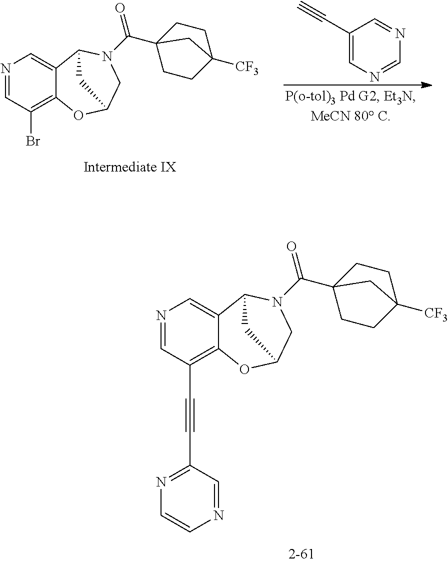

A mixture of Intermediate IX (22 mg, 0.05 mmol) and chloro[tri(o-tolyl)phosphine][2-(2′-amino-1,1′-biphenyl)]palladium(II) (3.1 mg, 0.005 mmol) in a vial was degassed and backfilled with nitrogen. The solids were dissolved in MeCN (0.25 mL), then 2-ethynylpyrazine (7.8 mg, 0.075 mmol) and Et₃N (21 μL, 0.15 mmol) were added and the reaction was heated to 80° C. with stirring overnight. The product was purified by reverse phase chromatography (MeCN/water with 0.1% TFA) to afford ((2S,5S)-9-(pyrazin-2-ylethynyl)-2,3-dihydro-2,5-methanopyrido[3,4-f][1,4]oxazepin-4(5H)-yl)(4-(trifluoromethyl)bicyclo[2.2.1]heptan-1-yl)methanone. ¹H NMR (500 MHz, DMSO-d₆) δ 8.88 (d. J=17.7 Hz, 1H), 8.77-8.63 (m, 2H), 8.54 (d, J=15.0 Hz, 1H), 8.29 (d, J=13.8 Hz, 1H), 5.40 (d, J=12.9 Hz, 1H), 5.23 (d, J=12.5 Hz, 1H), 3.97 (m, 2H), 2.31 (m, 1H), 2.17 (m, 1H), 1.82 (m, 8H), 1.57 (m, 4H). MS (EI) Calculated for C₂₄H₂₂F₃N₄O₂[M+H]⁺, 455; found, 455.

### Example 2P. Preparation of 3,3-Difluoro-2,2-dimethyl-1-((2S,5S)-9-((5-methyl-1,3,4-oxadiazol-2-yl)ethynyl)-2,3-dihydro-2,5-methanopyrido[3,4-f][1,4]oxazepin-4(5H)-yl)propan-1-one (2-71)

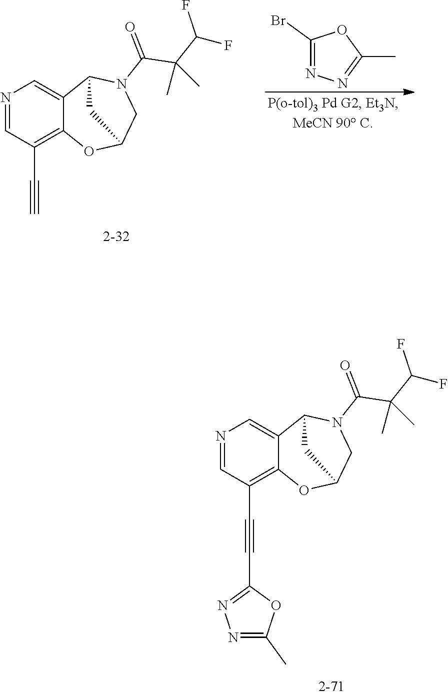

A mixture of 1-((2S,5S)-9-ethynyl-2,3-dihydro-2,5-methanopyrido[3,4-f][1,4]oxazepin-4(5H)-yl)-3,3-difluoro-2,2-dimethylpropan-1-one (15 mg, 0.05 mmol) and chloro[tri(o-tolyl)phosphine][2-(2′-amino-1,1′-biphenyl)]palladium(II) (3.1 mg, 0.005 mmol) in a vial was degassed and backfilled with nitrogen. The solids were dissolved in MeCN (0.5 mL), then 2-bromo-5-methyl-1,3,4-oxadiazole (12 mg, 0.075 mmol) and Et₃N (21 μL, 0.15 mmol) were added, and the reaction was heated to 90° C. with stirring overnight. The product was purified by reverse phase chromatography (MeCN/water with 0.1% TFA) to afford 3,3-difluoro-2,2-dimethyl-1-((2S,5S)-9-((5-methyl-1,3,4-oxadiazol-2-yl)ethynyl)-2,3-dihydro-2,5-methanopyrido[3,4-f][1,4]oxazepin-4(5H)-yl)propan-1-one. ¹H NMR (499 MHz, DMSO-dc) S 8.59 (s, 1H), 8.35 (s, 1H), 6.20 (t, J=56.8 Hz, 1H), 5.41 (s, 1H), 5.24 (s, 1H), 4.09 (s, 1H), 4.01 (d, J=10.4 Hz, 1H), 2.56 (s, 3H), 2.32 (d, J=10.2 Hz, 1H), 2.16 (d, J=12.7 Hz, 1H), 121 (s, 3H), 1.08 (s, 3H). MS (ET) Calculated for C₁₉H₁₉F₂N₄O₃[M+H]⁺, 389; found, 389.

### Example 2Q. Preparation of ((2S,5S)-9-((5-Fluoropyridin-3-yl)ethynyl)-2,3-dihydro-2,5-methanopyrido[3,4-f][1,4]oxazepin-4(5H)-yl)(4-(trifluoromethyl)bicyclo[2.2.1]heptan-1-yl)methanone (2-96)

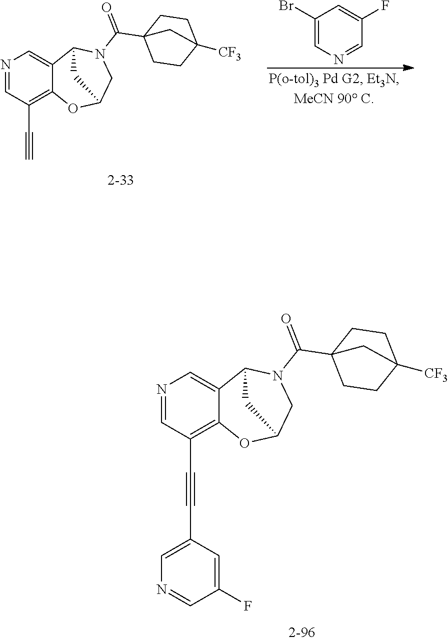 [SMILES: C#Cc1cncc2c1O[C@H]1C[C@@H]2N(C(=O)C23CCC(C(F)(F)F)(CC2)C3)C1 ; Fc1cncc(Br)c1 ; O=C(N1C[C@@H]2C[C@H]1c1cncc(C#Cc3cncc(F)c3)c1O2)C12CCC(C(F)(F)F)(CC1)C2]

A mixture of ((2S,5S)-9-ethynyl-2,3-dihydro-2,5-methanopyrido[3,4-f][1,4]oxazepin-4(5H)-yl)(4-(trifluoromethyl)bicyclo[2.2.1]heptan-1-yl)methanone (19 mg, 0.05 mmol) and chloro[tri(o-tolyl)phosphine][2-(2′-amino-1,1′-biphenyl)]palladium(II) (3.1 mg, 0.005 mmol) in a vial was degassed and backfilled with nitrogen. The solids were dissolved in MeCN (0.25 mL), then 3-bromo-5-fluoropyridine (13 mg, 0.075 mmol) and Et₃N (21 μL, 0.15 mmol) were added, and the reaction was heated to 90° C. with stirring overnight. The product was purified by reverse phase chromatography (MeCN/water with 0.1% TFA) to afford ((2S,5S)-9-((5-fluoropyridin-3-yl)ethynyl)-2,3-dihydro-2,5-methanopyrido[3,4-f][1,4]oxazepin-4(5H)-yl)(4-(trifluoromethyl)bicyclo[2.2.1]heptan-1-yl)methanone. ¹H NMR (500 MHz, Chloroform-d) δ 8.59 (s, 1H), 8.52 (s, 1H), 8.45 (d, J=2.5 Hz, 1H), 8.42 (s, 1H), 7.55 (d, J=8.8 Hz, 1H), 5.39 (s, 1H), 5.30 (s, 1H), 4.01 (d, J=11.7 Hz, 1H), 3.89 (d, J=9.4 Hz, 1H), 2.25 (s, 2H), 2.08-1.88 (m, 5H), 1.79 (m, 3H), 1.69-1.52 (m, 2H). MS (EI) Calculated for C₂₅H₂F₄N₃O₂ [M+H]⁺, 472; found, 472.

### Example 2R. Preparation of (4-Fluorobicyclo[2.2.1]heptan-1-yl)((2S,5S)-9-(phenylethynyl)-2,3-dihydro-2,5-methanopyrido[3,4-f][1,4]oxazepin-4(5H)-yl)methanone (2-97)

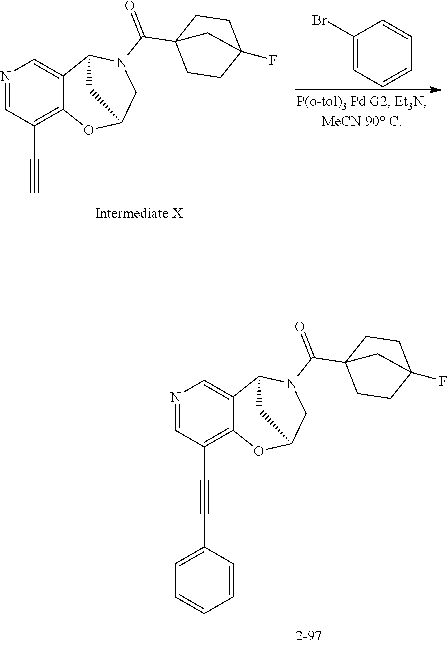 [SMILES: C#Cc1cncc2c1O[C@H]1C[C@@H]2N(C(=O)C23CCC(F)(CC2)C3)C1 ; Brc1ccccc1 ; O=C(N1C[C@@H]2C[C@H]1c1cncc(C#Cc3ccccc3)c1O2)C12CCC(F)(CC1)C2]

A mixture of Intermediate X (16 mg, 0.05 mmol) and chloro[tri(o-tolyl)phosphine][2-(2′-amino-1,1′-biphenyl)]palladium(II) (3.1 mg, 0.005 mmol) in a vial was degassed and backfilled with nitrogen. The solids were dissolved in MeCN (0.5 mL), then bromobenzene (11.8 mg, 0.075 mmol) and Et₃N (21 μL, 0.15 mmol) were added, and the reaction was heated to 90° C. with stirring overnight. The product was purified by reverse phase chromatography (MeCN/water with 0.1% TFA) to afford ((2S,5S)-9-((5-fluoropyridin-3-yl)ethynyl)-2,3-dihydro-2,5-methanopyrido[3,4-f][1,4]oxazepin-4(5H)-yl)(4-(trifluoromethyl)bicyclo[2.2.1]heptan-1-yl)methanone. ¹H NMR (500 MHz, DMSO-d₆) δ 8.42 (s, 1H), 8.19 (s, 1H), 7.54 (dd, J=6.6, 2.9 Hz, 2H), 7.48-7.40 (m, 3H), 5.35 (s, 1H), 5.18 (d, J=4.5 Hz, 1H), 3.92 (s, 2H), 2.29-2.22 (m, 1H), 2.10 (d, J=12.7 Hz, 1H), 1.98-1.62 (m, 10H). MS (EI) Calculated for C₂₅H₂₄FN₂O₂ [M+H]⁺, 403; found, 403.

### Example 2S. Preparation of 3,3-Difluoro-2,2-dimethyl-1-((2S,5S)-9-(3-methylbut-1-yn-1-yl)-2,3-dihydro-2,5-methanopyrido[3,4-f][1,4]oxazepin-4(5H)-yl)propan-1-one (2-106)

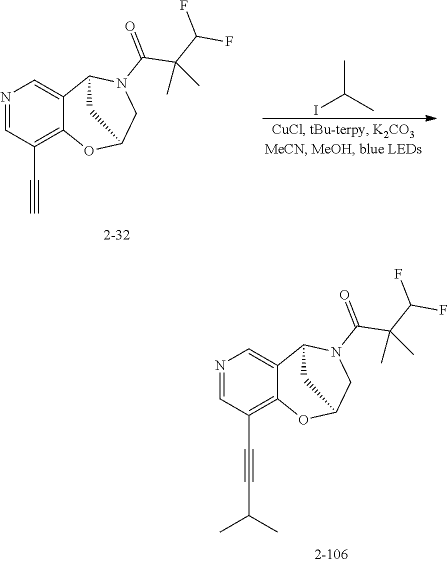

A vial was charged with 1-((2S,5S)-9-ethynyl-2,3-dihydro-2,5-methanopyrido[3,4-f][1,4]oxazepin-4(5H)-yl)-3,3-difluoro-2,2-dimethylpropan-1-one (15 mg, 0.05 mmol), copper(I) chloride (0.50 mg, 5.0 μmol), 4.4′,4″-tri-tert-butyl-2,2′; 6′,2″-terpyridine (4.0 mg, 0.01 mmol), and K₂CO₃ (21 mg, 0.15 mmol) then degassed and backfilled with nitrogen. MeCN (0.750 mL) and MeOH (0.25 mL) were added followed by 2-iodopropane (51.0 mg, 0.3 mmol), then the reaction mixture was irradiated with blue LEDs (450 nM) overnight. The crude mixture was filtered and concentrated. The product was purified by reverse phase chromatography (MeCN/water with 0.1% TFA) to afford 3,3-difluoro-2,2-dimethyl-1-((2S,5S)-9-(3-methylbut-1-yn-1-yl)-2,3-dihydro-2,5-methanopyrido[3,4-f][1,4]oxazepin-4(5H)-yl)propan-1-one. ¹H NMR (500 MHz, DMSO-d₆) δ 8.24 (s, 1H), 8.15 (s, 1H), 6.20 (t, J=56.5 Hz, 1H), 5.29 (s, 1H), 5.19 (s, 1H), 2.82 (p, J=6.9 Hz, 1H), 2.41-2.18 (m, 1H), 2.15-1.91 (m 1H), 1.21 (s, 6H), 1.19 (s, 6H). MS (EI) Calculated for C₁₉H₂₃F₂N₂O₂ [M+H]⁺, 349; found, 349.

### Example 2T. Preparation of ((2S,5S)-9-(Cyclopropylethynyl)-2,3-dihydro-2,5-methanopyrido[3,4-f][1,4]oxazepin-4(5H)-yl)(4-fluorobicyclo[2.2.1]heptan-1-yl)methanone (2-115)

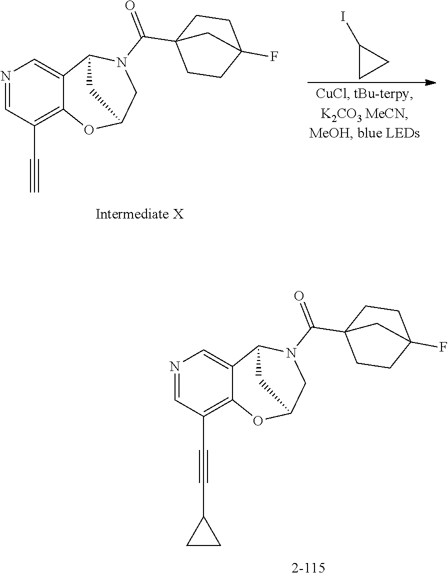

A vial was charged with ((2S,5S)-9-ethynyl-2,3-dihydro-2,5-methanopyrido[3,4-f][1,4]oxazepin-4(5H)-yl)(4-fluorobicyclo[2.2.1]heptan-1-yl)methanone (33 mg, 0.1 mmol), copper(I) chloride (0.99 mg, 10 μmol), 4,4′,4″-tri-tert-butyl-2,2′;6′,2″-terpyridine (8.0 mg, 0.02 mmol), and K₂CO₃ (42 mg, 0.3 mmol) then degassed and backfilled with nitrogen. MeCN (0.75 mL) and MeOH (0.25 mL) were added followed by iodocyclopropane (34 mg, 0.2 mmol), then the reaction mixture was irradiated with blue LEDs (450 nM) overnight. The crude mixture was filtered and concentrated. The product was purified by reverse phase chromatography (MeCN/water with 0.1% TFA) to afford ((2S,5S)-9-(cyclopropylethynyl)-2,3-dihydro-2,5-methanopyrido[3,4-f][1,4]oxazepin-4(5H)-yl)(4-fluorobicyclo[2.2.1]heptan-1-yl)methanone. ¹H NMR (500 MHz, DMSO-d₆) δ 8.22 (s, 1H), 8.09 (s, 1H), 5.28 (s, 1H), 5.13 (d. J=4.4 Hz, 1H), 3.87 (s, 2H), 2.21 (d, J=12.9 Hz, 1H), 2.12-2.00 (m, 1H), 1.99-1.47 (m, 11H), 0.98-0.84 (m, 2H), 0.72 (q, J=6.4, 5.2 Hz, 2H). MS (EI) Calculated for C₂₂H₂₄FN₂O₂[M+H]⁺, 367; found, 367.

### Example 2U. Preparation of ((2S,5S)-9-((4-Fluorophenyl)ethynyl)-2,3-dihydro-2,5-methanopyrido[3,4-f][1,4]oxazepin-4(5H)-yl)(1-methylcyclopentyl)methanone (2-121)

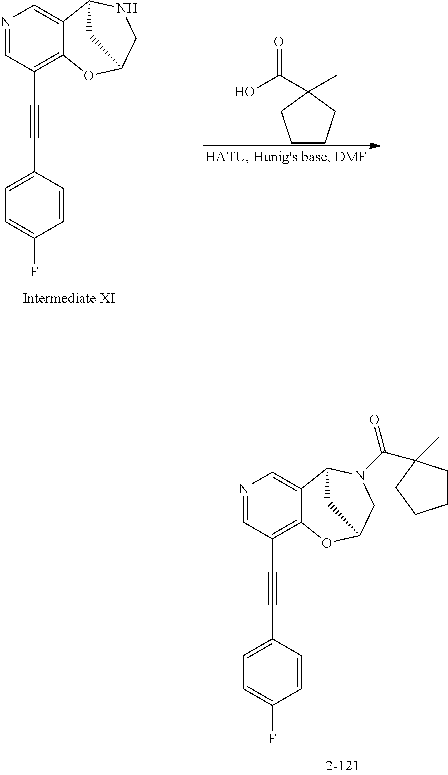

A solution of Intermediate XI (10 mg, 0.032 mmol), 1-methylcyclopentane-1-carboxylic acid (6.1 mg, 0.047 mmol) in DMF (630 μL) was treated with Hunig's Base (28 μL, 0.16 mmol) then HATU (24 mg, 0.063 mmol) and stirred at RT overnight. The crude mixture was filtered and purified by reverse phase chromatography (MeCN/water with 0.1% TFA) to afford ((2S,5S)-9-((4-fluorophenyl)ethynyl)-2,3-dihydro-2,5-methanopyrido[3,4-f][1,4]oxazepin-4(5H)-yl)(1-methylcyclopentyl)methanone. ¹H NMR (500 MHz, DMSO-d₆) δ 8.43 (s, 1H), 8.21 (s, 1H), 7.63-7.58 (m, 2H), 7.29 (t, J=8.8 Hz, 2H), 5.33 (s, 1H), 5.19 (s, 1H), 3.93 (s, 2H), 2.28 (d, J=10.0 Hz, 1H), 2.11 (d, J=12.6 Hz, 1H), 1.95 (s, 1H), 1.82 (s, 1H), 1.50 (m, 6H), 1.02 (s, 3H). MS (ET) Calculated for C₂₄H₂₄FN₂O₂ [M+H]⁺, 391; found, 391.

Compounds 2-4 to 2-6 were prepared in a fashion analogous to the preparation of 2-3, substituting 4-fluorobicyclo[2.2.1]heptane-1-carboxylic acid for the corresponding carboxylic acid.

Compounds 2-9, 2-10, 2-11 and 2-12 were prepared in a fashion analogous to the preparation of 2-3, substituting 4-fluorobicyclo[2.2.1]heptane-1-carboxylic acid for the corresponding acid.

Compounds 2-14, 2-15, and 2-16 were prepared in a fashion analogous to the preparation of 2-7, substituting 4-fluorobicyclo[2.2.1]heptane-1-carboxylic acid for the corresponding acid.

Compounds 2-18 and 2-19 were prepared together as a racemate in a fashion analogous to the preparation of 2-7, substituting 4-fluorobicyclo[2.2.1]heptane-1-carboxylic acid for (S and R)-2,2-difluorobicyclo[2.2.1]heptane-1-carboxylic acid. The racemate was resolved using chiral SFC chromatography. Conditions: racemate dissolved in 1:1 MeCN/MeOH; IC 21×250 mm, 5 um column: 215 nm detection; 70 mL/min of 35% MeOH/CO₂ with 0.1% NH₄OH. Peak 1 (2.70 min) isolated as 2-18, peak 2 (3.30 min) isolated as 2-19.

Compounds 2-21 to 2-24 as well as 2-30 were prepared in a fashion analogous to the synthesis of 2-7, substituting 4-fluorobicyclo[2.2.1]heptane-1-carboxylic acid for the corresponding carboxylic acid.

Compounds 2-25, 2-26, 2-29 were prepared in a fashion analogous to the synthesis of 2-3, substituting 4-fluorobicyclo[2.2.1]heptane-1-carboxylic acid for the corresponding carboxylic acid.

Compound 2-28 was prepared in a fashion analogous to the synthesis of 2-27, substituting 4-trifluoromethylbicyclo[2.2.1]heptane-1-carboxylic acid for the corresponding carboxylic acid.

Compound 2-33 was prepared in a fashion analogous to the synthesis of 2-32 starting from Intermediate IX. ¹H NMR (500 MHz, Chloroform-d) δ 8.50 (s, 1H), 8.44 (s, 1H), 5.38 (s, 2H), 4.03 (d, J=11.2 Hz, 1H), 3.90 (d, J=11.6 Hz, 1H), 3.49 (s, 1H), 2.32 (d, J=13.0 Hz, 1H), 2.25 (d, J=12.7 Hz, 1H), 2.01-1.87 (m, 5H), 1.77 (m, 3H), 1.63 (m, 2H). MS (EI) Calculated for C₂₀H₂₀F₃N₂O₂ [M+H]⁺, 377; found, 377.

Compounds 2-35 to 2-46 were prepared in a fashion analogous to the preparation of 2-34, substituting 6-ethynylquinoxaline for the corresponding alkyne.

Compounds 2-48 and 2-49 were prepared in a fashion analogous to the preparation of 2-47, substituting ethynylbenzene for the corresponding alkyne.

Compounds 2-51 to 2-60 were prepared in a fashion analogous to the preparation of 2-50, substituting 5-ethynylpyrimidine for the corresponding alkyne.

Compounds 2-62 to 2-70 were prepared in a fashion analogous to the preparation of 2-61, substituting 2-ethynylpyrazine for the corresponding alkyne.

Compounds 2-72 to 2-95 were prepared in a fashion analogous to the preparation of 2-71, substituting 2-bromo-5-methyl-1,3,4-oxadiazole for the corresponding aryl bromide.

Compounds 2-98 to 2-105 were prepared in a fashion analogous to the preparation of 2-97, substituting bromobenzene for the corresponding aryl bromide.

Compounds 2-107 to 2-114 were prepared in a fashion analogous to the preparation of 2-106, substituting 2-iodopropane for the corresponding alkyl iodide.

Compounds 2-116 to 2-120 were prepared in a fashion analogous to the preparation of 2-115, substituting iodocyclopropane for the corresponding alkyl iodide.

Compounds 2-122 and 2-123 were prepared in a fashion analogous to the preparation of 2-121, substituting 1-methylcyclopentane-1-carboxylic acid for the corresponding carboxylic acid.

TABLE 2

| Compound | | | Exact Mass |
|---|---|---|---|
| Number | Structure | Name | [M + H]⁺ |
| 2-1 | 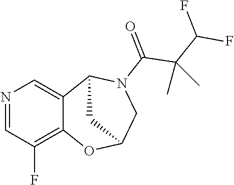 [SMILES: CC(C)(C(=O)N1C[C@@H]2C[C@H]1c1cncc(F)c1O2)C(F)F] | 3,3-difluoro-1-[(2S,5S)-9- fluoro-2,3-dihydro-2,5- methanopyrido[3,4- f][1,4]oxazepin-4(5H)-yl]- 2,2-dimethylpropan-1-one | Calculated 301, found 301 |
| 2-2 | 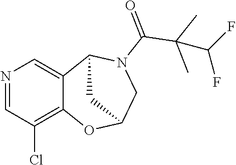 [SMILES: CC(C)(C(=O)N1C[C@@H]2C[C@H]1c1cncc(Cl)c1O2)C(F)F] | 1-[(2S,5S)-9-chloro-2,3- dihydro-2,5- methanopyrido[3,4- f][1,4]oxazepin-4(5H)-yl]- 3,3-difluoro-2,2- dimethylpropan-1-one | Calculated 317, found 317 |
| 2-3 | 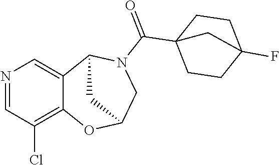 [SMILES: O=C(N1C[C@@H]2C[C@H]1c1cncc(Cl)c1O2)C12CCC(F)(CC1)C2] | [(2S,5S)-9-chloro-2,3- dihydro-2,5- methanopyrido[3,4- f][1,4]oxazepin-4(5H)- yl](4- fluorobicyclo[2.2.1]heptan- 1-yl)methanone | Calculated 337, found 337 |
| 2-4 | 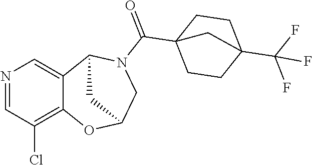 [SMILES: O=C(N1C[C@@H]2C[C@H]1c1cncc(Cl)c1O2)C12CCC(C(F)(F)F)(CC1)C2] | [(2S,5S)-9-chloro-2,3- dihydro-2,5- methanopyrido[3,4- f][1,4]oxazepin-4(5H)- yl][4- (trifluoromethyl)bicyclo [2.2.1]heptan-1-yl]methanone | Calculated 387, found 387 |
| 2-5 | 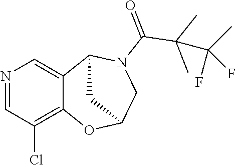 [SMILES: CC(F)(F)C(C)(C)C(=O)N1C[C@@H]2C[C@H]1c1cncc(Cl)c1O2] | 1-[(2S,5S)-9-chloro-2,3- dihydro-2,5- methanopyrido[3,4- f][1,4]oxazepin-4(5H)-yl]- 3,3-difluoro-2,2- dimethylbutan-1-one | Calculated 331, found 331 |
| 2-6 | 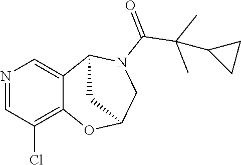 [SMILES: CC(C)(C(=O)N1C[C@@H]2C[C@H]1c1cncc(Cl)c1O2)C1CC1] | 1-[(2S,5S)-9-chloro-2,3- dihydro-2,5- methanopyrido[3,4- f][1,4]oxazepin-4(5H)-y]]- 2-cyclopropyl-2- methylpropan-1-one | Calculated 307, found 307 |
| 2-7 | 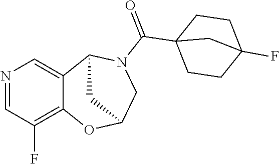 [SMILES: O=C(N1C[C@@H]2C[C@H]1c1cncc(F)c1O2)C12CCC(F)(CC1)C2] | (4- fluorobicyclo[2.2.1]heptan- 1-yl)[(2S,5S)-9-fluoro- 2,3-dihydro-2,5- methanopyrido[3,4- f][1,4]oxazepin-4(5H)- yl]methanone | Calculated 321, found 321 |
| 2-8 | 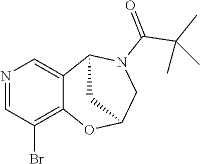 [SMILES: CC(C)(C)C(=O)N1C[C@@H]2C[C@H]1c1cncc(Br)c1O2] | 1-((2S,5S)-9-bromo-2,3- dihydro-2,5- methanopyrido[3,4- f][1,4]oxazepin-4(5H)-yl)- 2,2-dimethylpropan-1-one | Calculated 325 and 327, found 325 and 327 |
| 2-9 | 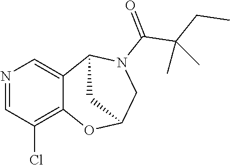 [SMILES: CCC(C)(C)C(=O)N1C[C@@H]2C[C@H]1c1cncc(Cl)c1O2] | 1-((2S,5S)-9-chloro-2,3- dihydro-2,5- methanopyrido[3,4- f][1,4]oxazepin-4(5H)-yl)- 2,2-dimethylbutan-1-one | Calculated 295, found 295 |
| 2-10 | 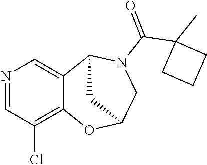 [SMILES: CC1(C(=O)N2C[C@@H]3C[C@H]2c2cncc(Cl)c2O3)CCC1] | ((2S,5S)-9-chloro-2,3- dihydro-2,5- methanopyrido[3,4- f][1,4]oxazepin-4(5H)- yl)(1- methylcyclobutyl)methanone | Calculated 293, found 293 |
| 2-11 | 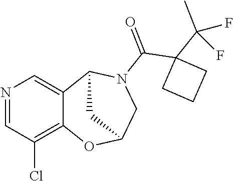 [SMILES: CC(F)(F)C1(C(=O)N2C[C@@H]3C[C@H]2c2cncc(Cl)c2O3)CCC1] | ((2S,5S)-9-chloro-2,3- dihydro-2,5- methanopyrido[3,4- f][1,4]oxazepin-4(5H)- yl)(1-(1,1- difluoroethyl)cyclobutyl) methanone | Calculated 343, found 343 |
| 2-12 | 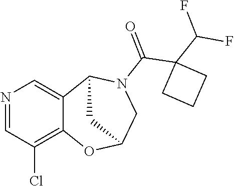 [SMILES: O=C(N1C[C@@H]2C[C@H]1c1cncc(Cl)c1O2)C1(C(F)F)CCC1] | ((2S,5S)-9-chloro-2,3- dihydro-2,5- methanopyrido[3,4- f][1,4]oxazepin-4(5H)- yl)(1- (difluoromethyl)cyclobutyl) methanone | Calculated 329, found 329 |
| 2-13 | 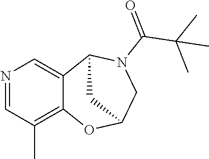 [SMILES: Cc1cncc2c1O[C@H]1C[C@@H]2N(C(=O)C(C)(C)C)C1] | 2,2-dimethyl-1-((2S,5S)-9- methyl-2,3-dihydro-2,5- methanopyrido[3,4- f][1,4]oxazepin-4(5H)- yl)propan-1-one | Calculated 261, found 261 |
| 2-14 | 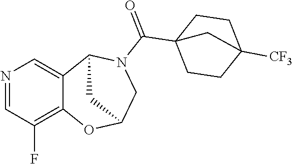 [SMILES: O=C(N1C[C@@H]2C[C@H]1c1cncc(F)c1O2)C12CCC(C(F)(F)F)(CC1)C2] | ((2S,5S)-9-fluoro-2,3- dihydro-2,5- methanopyrido[3,4- f][1,4]oxazepin-4(5H)- yl)(4- (trifluoromethyl)bicyclo [2.2.1]heptan-1-yl)methanone | Calculated 371, found 371 |
| 2-15 | 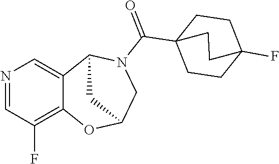 [SMILES: O=C(N1C[C@@H]2C[C@H]1c1cncc(F)c1O2)C12CCC(F)(CC1)CC2] | ((2S,5S)-9-fluoro-2,3- dihydro-2,5- methanopyrido[3,4- f][1,4]oxazepin-4(5H)- yl)(4- fluorobicyclo[2.2.2]octan- 1-yl)methanone | Calculated 335, found 335 |
| 2-16 | 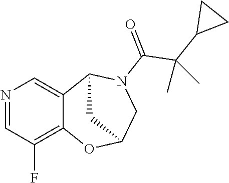 [SMILES: CC(C)(C(=O)N1C[C@@H]2C[C@H]1c1cncc(F)c1O2)C1CC1] | 2-cyclopropyl-1-((2S,5S)- 9-fluoro-2,3-dihydro-2,5- methanopyrido[3,4- f][1,4]oxazepin-4(5H)-yl)- 2-methylpropan-1-one | Calculated 335, found 335 |
| 2-17 | 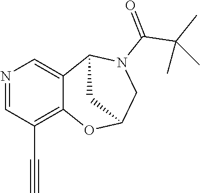 [SMILES: C#Cc1cncc2c1O[C@H]1C[C@@H]2N(C(=O)C(C)(C)C)C1] | 1-((2S,5S)-9-ethynyl-2,3- dihydro-2,5- methanopyrido[3,4- f][1,4]oxazepin-4(5H)-yl)- 2,2-dimethylpropan-1-one | Calculated 271, found 271 |
| 2-18 | 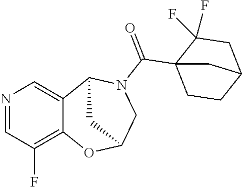 [SMILES: O=C(N1C[C@@H]2C[C@H]1c1cncc(F)c1O2)C12CCC(CC1(F)F)C2] | (2,2-(S or R)- difluorobicyclo[2.2.1]heptan- 1-yl)((28,5S)-9-fluoro- 2,3-dihydro-2,5- methanopyrido[3,4- f][1,4]oxazepin-4(5H)- yl)methanone | Calculated 339, found 339 |
| 2-19 | 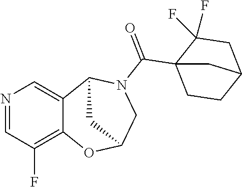 [SMILES: O=C(N1C[C@@H]2C[C@H]1c1cncc(F)c1O2)C12CCC(CC1(F)F)C2] | (2,2-(S or R)- difluorobicyclo[2.2.1]heptan- 1-yl)((2S,5S)-9-fluoro- 2,3-dihydro-2,5- methanopyrido[3,4- f][1,4]oxazepin-4(5H)- yl)methanone | Calculated 339, found 339 |
| 2-20 | 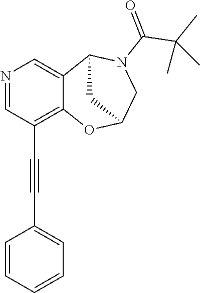 [SMILES: CC(C)(C)C(=O)N1C[C@@H]2C[C@H]1c1cncc(C#Cc3ccccc3)c1O2] | 2,2-dimethyl-1-((2S,5S)-9- (phenylethynyl)-2,3- dihydro-2,5- methanopyrido[3,4- f][1,4]oxazepin-4(5H)- yl)propan-1-one | Calculated 347, found 347 |
| 2-21 | 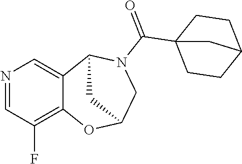 [SMILES: O=C(N1C[C@@H]2C[C@H]1c1cncc(F)c1O2)C12CCC(CC1)C2] | bicyclo[2.2.1]heptan-1- yl((2S,5S)-9-fluoro-2,3- dihydro-2,5- methanopyrido[3,4- f][1,4]oxazepin-4(5H)- yl)methanone | Calc'd 303, found 303 |
| 2-22 | 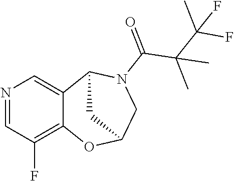 [SMILES: CC(F)(F)C(C)(C)C(=O)N1C[C@@H]2C[C@H]1c1cncc(F)c1O2] | 3,3-difluoro-1-((2S,5S)-9- fluoro-2,3-dihydro-2,5- methanopyrido[3,4- f][1,4]oxazepin-4(5H)-yl)- 2,2-dimethylbutan-1-one | Calc'd 315, found 315 |
| 2-23 | 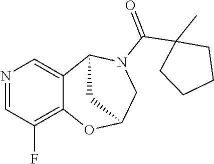 [SMILES: CC1(C(=O)N2C[C@@H]3C[C@H]2c2cncc(F)c2O3)CCCC1] | ((2S,5S)-9-fluoro-2,3- dihydro-2,5- methanopyrido[3,4- f][1,4]oxazepin-4(5H)- yl)(1- methylcyclopentyl) methanone | Calc'd 291, found 291 |
| 2-24 | 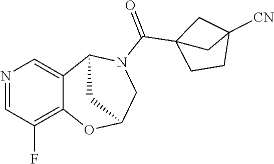 [SMILES: N#CC12CCC(C(=O)N3C[C@@H]4C[C@H]3c3cncc(F)c3O4)(C1)C2] | 4-((2S,5S)-9-fluoro- 2,3,4,5-tetrahydro-2,5- methanopyrido[3,4- f][1,4]oxazepine-4- carbonyl)bicyclo[2.1.1] hexane-1-carbonitrile | Calc'd 314, found 314 |
| 2-25 | 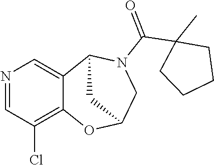 [SMILES: CC1(C(=O)N2C[C@@H]3C[C@H]2c2cncc(Cl)c2O3)CCCC1] | ((2S,5S)-9-chloro-2,3- dihydro-2,5- methanopyrido[3,4- f][1,4]oxazepin-4(5H)- yl)(1- methylcyclopentyl) methanone | Calc'd 307, found 307 |
| 2-26 | 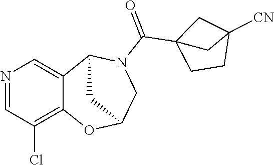 [SMILES: N#CC12CCC(C(=O)N3C[C@@H]4C[C@H]3c3cncc(Cl)c3O4)(C1)C2] | 4-((2S,5S)-9-chloro- 2,3,4,5-tetrahydro-2,5- methanopyrido[3,4- f][1,4]oxazepine-4- carbonyl)bicyclo[2.1.1] hexane-1-carbonitrile | Calc'd 330, found 330 |
| 2-27 | 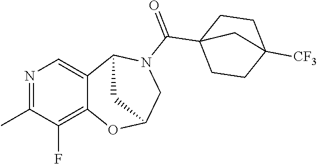 [SMILES: Cc1ncc2c(c1F)O[C@H]1C[C@@H]2N(C(=O)C23CCC(C(F)(F)F)(CC2)C3)C1] | ((2S,5S)-9-fluoro-8- methyl-2,3-dihydro-2,5- methanopyrido[3,4- f][1,4]oxazepin-4(5H)- yl)(4- (trifluoromethyl)bicyclo [2.2.1]heptan-1-yl)methanone | Calc'd 385, found 385 |
| 2-28 | 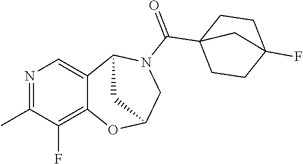 [SMILES: Cc1ncc2c(c1F)O[C@H]1C[C@@H]2N(C(=O)C23CCC(F)(CC2)C3)C1] | ((2S,5S)-9-fluoro-8- methyl-2,3-dihydro-2,5- methanopyrido[3,4- f][1,4]oxazepin-4(5H)- yl)(4- fluorobicyclo[2.2.1]heptan- 1-yl)methanone | Calc'd 335, found 335 |
| 2-29 | 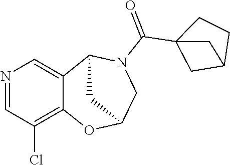 [SMILES: O=C(N1C[C@@H]2C[C@H]1c1cncc(Cl)c1O2)C12CCC(C1)C2] | bicyclo[2.1.1]hexan-1- yl((2S,5S)-9-chloro-2,3- dihydro-2,5- methanopyrido[3,4- f][1,4]oxazepin-4(5H)- yl)methanone | Calc'd 305, found 305 |
| 2-30 | 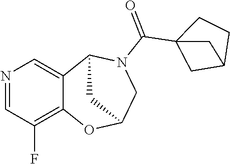 [SMILES: O=C(N1C[C@@H]2C[C@H]1c1cncc(F)c1O2)C12CCC(C1)C2] | bicyclo[2.1.1]hexan-1- yl((2S,5S)-9-fluoro-2,3- dihydro-2,5- methanopyrido[3,4- f][1,4]oxazepin-4(5H)- yl)methanone | Calc'd 289, found 289 |
| 2-31 | 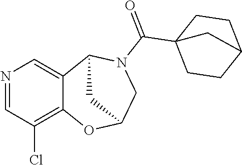 [SMILES: O=C(N1C[C@@H]2C[C@H]1c1cncc(Cl)c1O2)C12CCC(CC1)C2] | bicyclo[2.2.1]heptan-1- yl((2S,5S)-9-chloro-2,3- dihydro-2,5- methanopyrido[3,4- f][1,4]oxazepin-4(5H)- yl)methanone | Calc'd 320, found 320 |
| 2-32 | 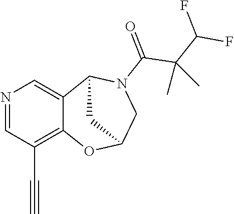 [SMILES: C#Cc1cncc2c1O[C@H]1C[C@@H]2N(C(=O)C(C)(C)C(F)F)C1] | 1-((2S,5S)-9-ethynyl-2,3- dihydro-2,5- methanopyrido[3,4- f][1,4]oxazepin-4(5H)-yl)- 3,3-difluoro-2,2- dimethylpropan-1-one | Calc'd 307, found 307 |
| 2-33 | 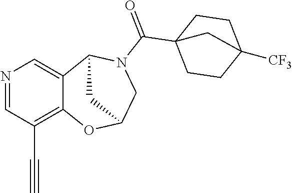 [SMILES: C#Cc1cncc2c1O[C@H]1C[C@@H]2N(C(=O)C23CCC(C(F)(F)F)(CC2)C3)C1] | ((2S,5S)-9-ethynyl-2,3- dihydro-2,5- methanopyrido[3,4- f][1,4]oxazepin-4(5H)- yl)(4- (trifluoromethyl)bicyclo [2.2.1]heptan-1-yl)methanone | Calc'd 377, found 377 |
| 2-34 | 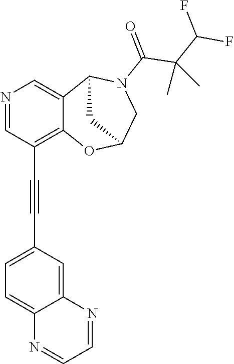 [SMILES: CC(C)(C(=O)N1C[C@@H]2C[C@H]1c1cncc(C#Cc3ccc4nccnc4c3)c1O2)C(F)F] | 3,3-difluoro-2,2-dimethyl- 1-((2S,5S)-9-(quinoxalin- 6-ylethynyl)-2,3-dihydro- 2,5-methanopyrido[3,4- f][1,4]oxazepin-4(5H)- yl)propan-1-one | Calc'd 435, found 435 |
| 2-35 | 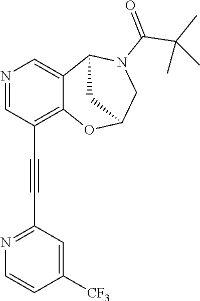 [SMILES: CC(C)(C)C(=O)N1C[C@@H]2C[C@H]1c1cncc(C#Cc3cc(C(F)(F)F)ccn3)c1O2] | 2,2-dimethyl-1-((2S,5S)-9- ((4- (trifluoromethyl)pyridin-2- yl)ethynyl)-2,3-dihydro- 2,5-methanopyrido[3,4- f][1,4]oxazepin-4(5H)- yl)propan-1-one | Calc'd 416, found 416 |
| 2-36 | 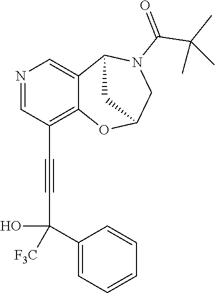 [SMILES: CC(C)(C)C(=O)N1C[C@@H]2C[C@H]1c1cncc(C#CC(O)(c3ccccc3)C(F)(F)F)c1O2] | 2,2-dimethyl-1-((2S,5S)-9- (4,4,4-trifluoro-3-hydroxy- 3-phenylbut-1-yn-1-yl)- 2,3-dihydro-2,5- methanopyrido[3,4- f][1,4]oxazepin-4(5H)- yl)propan-1-one | Calc'd 445, found 445 |
| 2-37 | 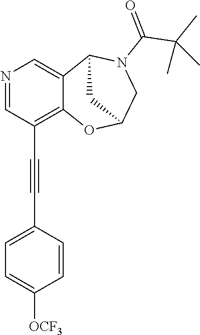 [SMILES: CC(C)(C)C(=O)N1C[C@@H]2C[C@H]1c1cncc(C#Cc3ccc(OC(F)(F)F)cc3)c1O2] | 2,2-dimethyl-1-((2S,5S)-9- ((4- (trifluoromethoxy)phenyl) ethynyl)-2,3-dihydro-2,5- methanopyrido[3,4- f][1,4]oxazepin-4(5H)- yl)propan-1-one | Calc'd 431, found 431 |
| 2-38 | 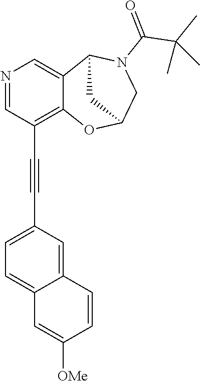 [SMILES: COc1ccc2cc(C#Cc3cncc4c3O[C@H]3C[C@@H]4N(C(=O)C(C)(C)C)C3)ccc2c1] | 1-((2S,5S)-9-((6- methoxynaphthalen-2- yl)ethynyl)-2,3-dihydro- 2,5-methanopyrido[3,4- f][1,4]oxazepin-4(5H)-yl)- 2,2-dimethylpropan-1-one | Calc'd 427, found 427 |
| 2-39 | 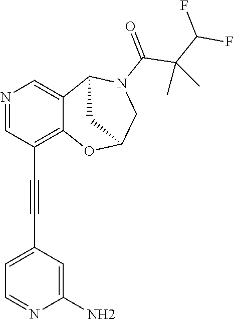 [SMILES: CC(C)(C(=O)N1C[C@@H]2C[C@H]1c1cncc(C#Cc3ccnc(N)c3)c1O2)C(F)F] | 1-((2S,5S)-9-((2- aminopyridin-4- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 399, found 399 |
| 2-40 | 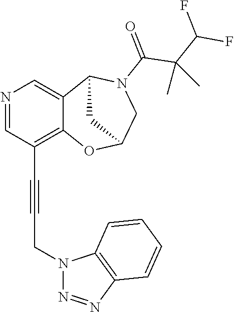 [SMILES: CC(C)(C(=O)N1C[C@@H]2C[C@H]1c1cncc(C#CCn3nnc4ccccc43)c1O2)C(F)F] | 1-((2S,5S)-9-(3-(1H- benzo[d][1,2,3]triazol-1- yl)prop-1-yn-1-yl)-2,3- dihydro-2,5- methanopyrido[3,4- f][1,4]oxazepin-4(5H)-yl)- 3,3-difluoro-2,2- dimethylpropan-1-one | Calc'd 438, found 438 |
| 2-41 | 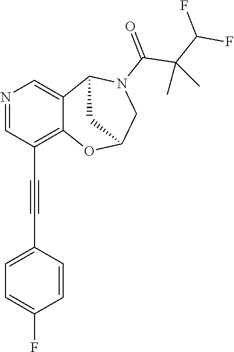 [SMILES: CC(C)(C(=O)N1C[C@@H]2C[C@H]1c1cncc(C#Cc3ccc(F)cc3)c1O2)C(F)F] | 3,3-difluoro-1-((2S,5S)-9- ((4-fluorophenyl)ethynyl)- 2,3-dihydro-2,5- methanopyrido[3,4- f][1,4]oxazepin-4(5H)-yl)- 2,2-dimethylpropan-1-one | Calc'd 401, found 401 |
| 2-42 | 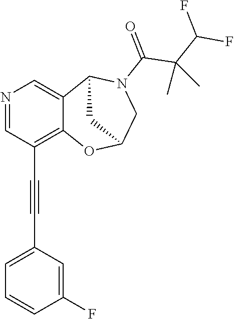 [SMILES: CC(C)(C(=O)N1C[C@@H]2C[C@H]1c1cncc(C#Cc3cccc(F)c3)c1O2)C(F)F] | 3,3-difluoro-1-((2S,5S)-9- ((3-fluorophenyl)ethynyl)- 2,3-dihydro-2,5- methanopyrido[3,4- f][1,4]oxazepin-4(5H)-yl)- 2,2-dimethylpropan-1-one | Calc'd 401, found 401 |
| 2-43 | 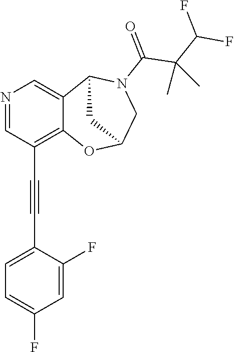 [SMILES: CC(C)(C(=O)N1C[C@@H]2C[C@H]1c1cncc(C#Cc3ccc(F)cc3F)c1O2)C(F)F] | 1-((2S,5S)-9-((2,4- difluorophenyl)ethynyl)- 2,3-dihydro-2,5- methanopyrido[3,4- f][1,4]oxazepin-4(5H)-yl)- 3,3-difluoro-2,2- dimethylpropan-1-one | Calc'd 419, found 419 |
| 2-44 | 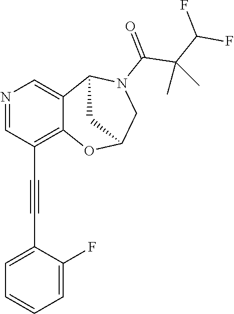 [SMILES: CC(C)(C(=O)N1C[C@@H]2C[C@H]1c1cncc(C#Cc3ccccc3F)c1O2)C(F)F] | 3,3-difluoro-1-((2S,5S)-9- ((2-fluorophenyl)ethynyl)- 2,3-dihydro-2,5- methanopyrido[3,4- f][1,4]oxazepin-4(5H)-yl)- 2,2-dimethylpropan-1-one | Calc'd 401, found 401 |
| 2-45 | 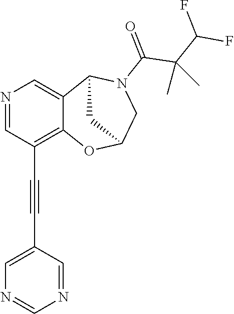 [SMILES: CC(C)(C(=O)N1C[C@@H]2C[C@H]1c1cncc(C#Cc3cncnc3)c1O2)C(F)F] | 3,3-difluoro-2,2-dimethyl- 1-((2S,5S)-9-(pyrimidin-5- ylethynyl)-2,3-dihydro- 2,5-methanopyrido[3,4- f][1,4]oxazepin-4(5H)- yl)propan-1-one | Calc'd 385, found 385 |
| 2-46 | 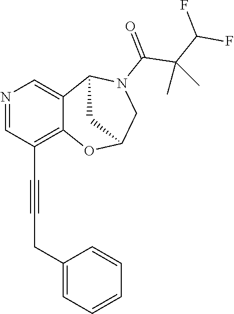 [SMILES: CC(C)(C(=O)N1C[C@@H]2C[C@H]1c1cncc(C#CCc3ccccc3)c1O2)C(F)F] | 3,3-difluoro-2,2-dimethyl- 1-((2S,5S)-9-(3- phenylprop-1-yn-1-yl)-2,3- dihydro-2,5- methanopyrido[3,4- f][1,4]oxazepin-4(5H)- yl)propan-1-one | Calc'd 397, found 397 |
| 2-47 | 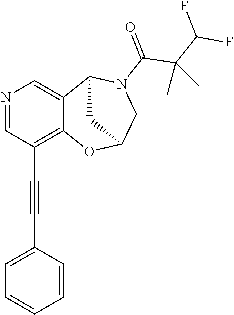 [SMILES: CC(C)(C(=O)N1C[C@@H]2C[C@H]1c1cncc(C#Cc3ccccc3)c1O2)C(F)F] | 3,3-difluoro-2,2-dimethyl- 1-((2S,5S)-9- (phenylethynyl)-2,3- dihydro-2,5- methanopyrido[3,4- f][1,4]oxazepin-4(5H)- yl)propan-1-one | Calc'd 383, found 383 |
| 2-48 | 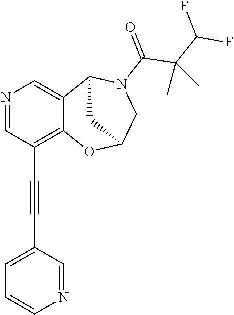 [SMILES: CC(C)(C(=O)N1C[C@@H]2C[C@H]1c1cncc(C#Cc3cccnc3)c1O2)C(F)F] | 3,3-difluoro-2,2-dimethyl- 1-((2S,5S)-9-(pyridin-3- ylethynyl)-2,3-dihydro- 2,5-methanopyrido[3,4- f][1,4]oxazepin-4(5H)- yl)propan-1-one | Calc'd 384, found 384 |
| 2-49 | 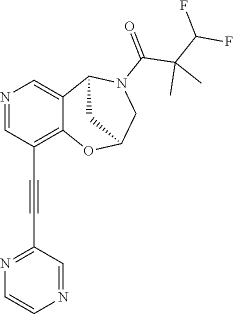 [SMILES: CC(C)(C(=O)N1C[C@@H]2C[C@H]1c1cncc(C#Cc3cnccn3)c1O2)C(F)F] | 3,3-difluoro-2,2-dimethyl- 1-((2S,5S)-9-(pyrazin-2- ylethynyl)-2,3-dihydro- 2,5-methanopyrido[3,4- f][1,4]oxazepin-4(5H)- yl)propan-1-one | Calc'd 385, found 385 |
| 2-50 | 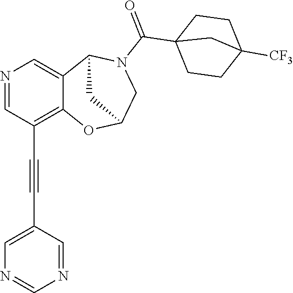 [SMILES: O=C(N1C[C@@H]2C[C@H]1c1cncc(C#Cc3cncnc3)c1O2)C12CCC(C(F)(F)F)(CC1)C2] | ((2S,5S)-9-(pyrimidin-5- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 455, found 455 |
| 2-51 | 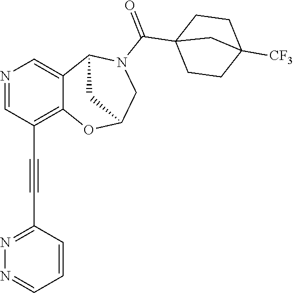 [SMILES: O=C(N1C[C@@H]2C[C@H]1c1cncc(C#Cc3cccnn3)c1O2)C12CCC(C(F)(F)F)(CC1)C2] | ((2S,5S)-9-(pyridazin-3- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 455, found 455 |
| 2-52 | 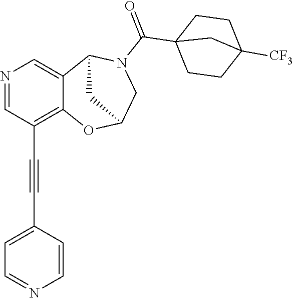 [SMILES: O=C(N1C[C@@H]2C[C@H]1c1cncc(C#Cc3ccncc3)c1O2)C12CCC(C(F)(F)F)(CC1)C2] | ((2S,5S)-9-(pyridin-4- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 454, found 454 |
| 2-53 | 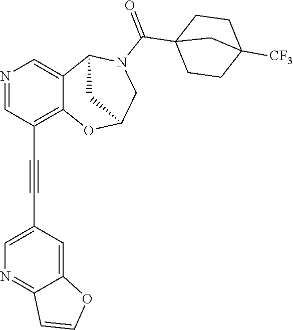 [SMILES: O=C(N1C[C@@H]2C[C@H]1c1cncc(C#Cc3cnc4ccoc4c3)c1O2)C12CCC(C(F)(F)F)(CC1)C2] | ((2S,5S)-9-(furo[3,2- b]pyridin-6-ylethynyl)-2,3- dihydro-2,5- methanopyrido[3,4- f][1,4]oxazepin-4(5H)- yl)(4- (trifluoromethyl)bicyclo [2.2.1]heptan-1-yl)methanone | Calc'd 494, found 494 |
| 2-54 | 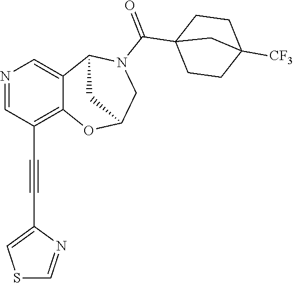 [SMILES: O=C(N1C[C@@H]2C[C@H]1c1cncc(C#Cc3cscn3)c1O2)C12CCC(C(F)(F)F)(CC1)C2] | ((2S,5S)-9-(thiazol-4- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 460, found 460 |
| 2-55 | 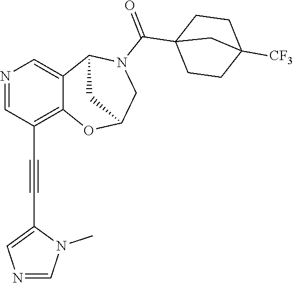 [SMILES: Cn1cncc1C#Cc1cncc2c1O[C@H]1C[C@@H]2N(C(=O)C23CCC(C(F)(F)F)(CC2)C3)C1] | ((2S,5S)-9-((1-methyl-1H- imidazol-5-yl)ethynyl)-2,3- dihydro-2,5- methanopyrido[3,4- f][1,4]oxazepin-4(5H)- yl)(4- (trifluoromethyl)bicyclo [2.2.1]heptan-1-yl)methanone | Calc'd 457, found 457 |
| 2-56 | 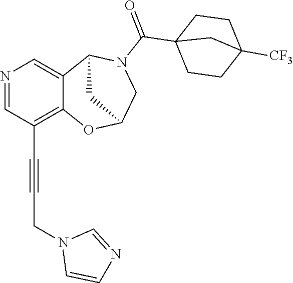 [SMILES: O=C(N1C[C@@H]2C[C@H]1c1cncc(C#CCn3ccnc3)c1O2)C12CCC(C(F)(F)F)(CC1)C2] | ((2S,5S)-9-(3-(1H- imidazol-1-yl)prop-1-yn-1- yl)-2,3-dihydro-2,5- methanopyrido[3,4- f][1,4]oxazepin-4(5H)- yl)(4- (trifluoromethyl)bicyclo [2.2.1]heptan-1-yl)methanone | Calc'd 457, found 457 |
| 2-57 | 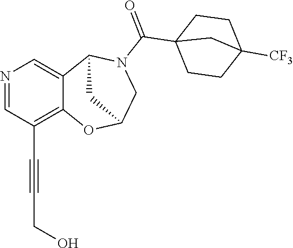 [SMILES: O=C(N1C[C@@H]2C[C@H]1c1cncc(C#CCO)c1O2)C12CCC(C(F)(F)F)(CC1)C2] | ((2S,5S)-9-(3- hydroxyprop-1-yn-1-yl)- 2,3-dihydro-2,5- methanopyrido[3,4- f][1,4]oxazepin-4(5H)- yl)(4- (trifluoromethyl)bicyclo[2.2.1] heptan-1-yl)methanone | Calc'd 407, found 407 |
| 2-58 | 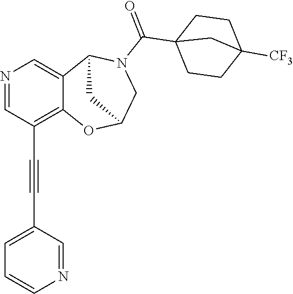 [SMILES: O=C(N1C[C@@H]2C[C@H]1c1cncc(C#Cc3cccnc3)c1O2)C12CCC(C(F)(F)F)(CC1)C2] | ((2S,5S)-9-(pyridin-3- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 454, found 454 |
| 2-59 | 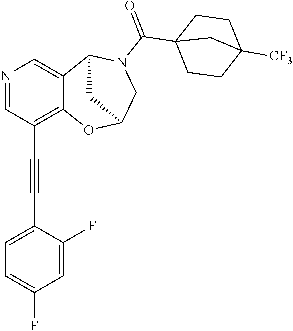 [SMILES: O=C(N1C[C@@H]2C[C@H]1c1cncc(C#Cc3ccc(F)cc3F)c1O2)C12CCC(C(F)(F)F)(CC1)C2] | ((2S,5S)-9-((2,4- difluorophenyl)ethynyl)- 2,3-dihydro-2,5- methanopyrido[3,4- f][1,4]oxazepin-4(5H)- yl)(4- (trifluoromethyl)bicyclo[2.2.1] heptan-1-yl)methanone | Calc'd 489, found 489 |
| 2-60 | 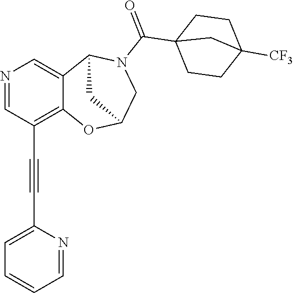 [SMILES: O=C(N1C[C@@H]2C[C@H]1c1cncc(C#Cc3ccccn3)c1O2)C12CCC(C(F)(F)F)(CC1)C2] | ((2S,5S)-9-(pyridin-2- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 454, found 454 |
| 2-61 | 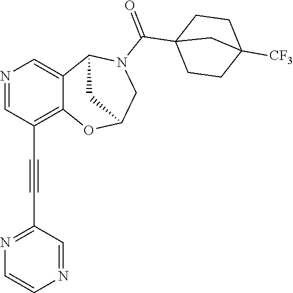 [SMILES: O=C(N1C[C@@H]2C[C@H]1c1cncc(C#Cc3cnccn3)c1O2)C12CCC(C(F)(F)F)(CC1)C2] | ((2S,5S)-9-(pyrazin-2- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 455, found 455 |
| 2-62 | 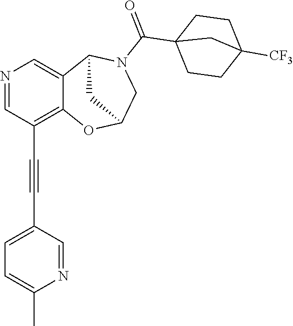 [SMILES: Cc1ccc(C#Cc2cncc3c2O[C@H]2C[C@@H]3N(C(=O)C34CCC(C(F)(F)F)(CC3)C4)C2)cn1] | ((2S,5S)-9-((6- methylpyridin-3- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 468, found 468 |
| 2-63 | 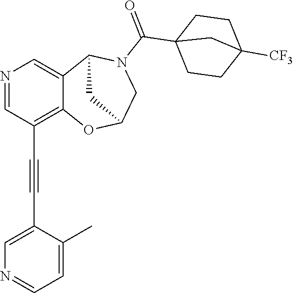 [SMILES: Cc1ccncc1C#Cc1cncc2c1O[C@H]1C[C@@H]2N(C(=O)C23CCC(C(F)(F)F)(CC2)C3)C1] | ((2S,5S)-9-((4- methylpyridin-3- yl)ethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 468, found 468 |
| 2-64 | 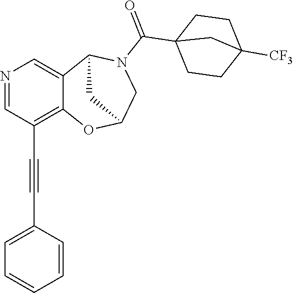 [SMILES: O=C(N1C[C@@H]2C[C@H]1c1cncc(C#Cc3ccccc3)c1O2)C12CCC(C(F)(F)F)(CC1)C2] | ((2S,5S)-9- (phenylethynyl)-2,3- dihydro-2,5- methanopyrido[3,4- f][1,4]oxazepin-4(5H)- yl)(4- (trifluoromethyl)bicyclo [2.2.1]heptan-1-yl)methanone | Calc'd 453, found 453 |
| 2-65 | 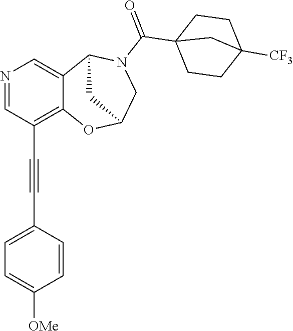 [SMILES: COc1ccc(C#Cc2cncc3c2O[C@H]2C[C@@H]3N(C(=O)C34CCC(C(F)(F)F)(CC3)C4)C2)cc1] | ((2S,5S)-9-((4- methoxyphenyl)ethynyl)- 2,3-dihydro-2,5- methanopyrido[3,4- f][1,4]oxazepin-4(5H)- yl)(4- (trifluoromethyl)bicyclo [2.2.1]heptan-1-yl)methanone | Calc'd 483, found 483 |
| 2-66 | 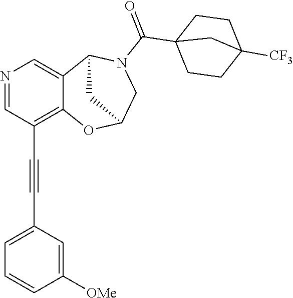 [SMILES: COc1cccc(C#Cc2cncc3c2O[C@H]2C[C@@H]3N(C(=O)C34CCC(C(F)(F)F)(CC3)C4)C2)c1] | ((2S,5S)-9-((3- methoxyphenyl)ethynyl)- 2,3-dihydro-2,5- methanopyrido[3,4- f][1,4]oxazepin-4(5H)- yl)(4- (trifluoromethyl)bicyclo [2.2.1]heptan-1-yl)methanone | Calc'd 483, found 483 |
| 2-67 | 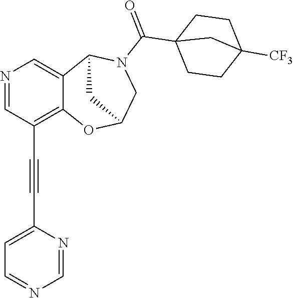 [SMILES: O=C(N1C[C@@H]2C[C@H]1c1cncc(C#Cc3ccncn3)c1O2)C12CCC(C(F)(F)F)(CC1)C2] | ((2S,5S)-9-(pyrimidin-4- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 455, found 455 |
| 2-68 | 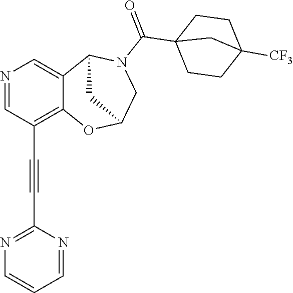 [SMILES: O=C(N1C[C@@H]2C[C@H]1c1cncc(C#Cc3ncccn3)c1O2)C12CCC(C(F)(F)F)(CC1)C2] | ((2S,5S)-9-(pyrimidin-2- ylethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 455, found 455 |
| 2-69 | 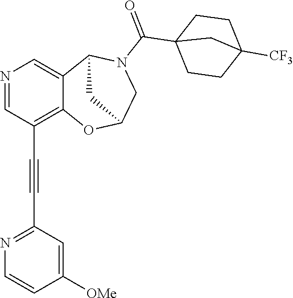 [SMILES: COc1ccnc(C#Cc2cncc3c2O[C@H]2C[C@@H]3N(C(=O)C34CCC(C(F)(F)F)(CC3)C4)C2)c1] | ((2S,5S)-9-((4- methoxypyridin-2- yl)ethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 484, found 484 |
| 2-70 | 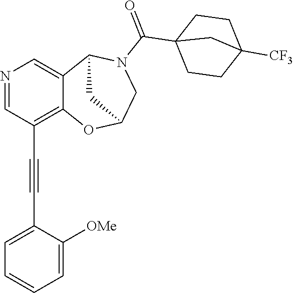 [SMILES: COc1ccccc1C#Cc1cncc2c1O[C@H]1C[C@@H]2N(C(=O)C23CCC(C(F)(F)F)(CC2)C3)C1] | ((2S,5S)-9-((2- methoxyphenyl)ethynyl)- 2,3-dihydro-2,5- methanopyrido[3,4- f][1,4]oxazepin-4(5H)- yl)(4- (trifluoromethyl)bicyclo [2.2.1]heptan-1-yl)methanone | Calc'd 483, found 483 |
| 2-71 | 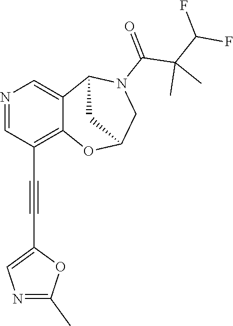 [SMILES: Cc1ncc(C#Cc2cncc3c2O[C@H]2C[C@@H]3N(C(=O)C(C)(C)C(F)F)C2)o1] | 3,3-difluoro-2,2-dimethyl- 1-((2S,5S)-9-((5-methyl- 1,3,4-oxadiazol-2- yl)ethynyl)-2,3-dihydro- 2,5-methanopyrido[3,4- f][1,4]oxazepin-4(5H)- yl)propan-1-one | Calc'd 389, found 389 |
| 2-72 | 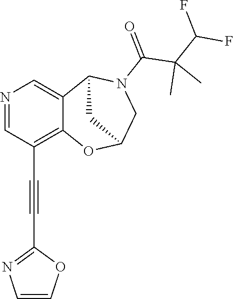 [SMILES: CC(C)(C(=O)N1C[C@@H]2C[C@H]1c1cncc(C#Cc3ncco3)c1O2)C(F)F] | 3,3-difluoro-2,2-dimethyl- 1-((2S,5S)-9-(oxazol-2- ylethynyl)-2,3-dihydro- 2,5-methanopyrido[3,4- f\|[1,4]oxazepin-4(5H)- yl)propan-1-one | Calc'd 374, found 374 |
| 2-73 | 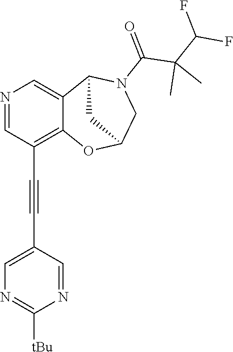 [SMILES: CC(C)(C)c1ncc(C#Cc2cncc3c2O[C@H]2C[C@@H]3N(C(=O)C(C)(C)C(F)F)C2)cn1] | 1-((2S,5S)-9-((2-(tert- butyl)pyrimidin-5- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 441, found 441 |
| 2-74 | 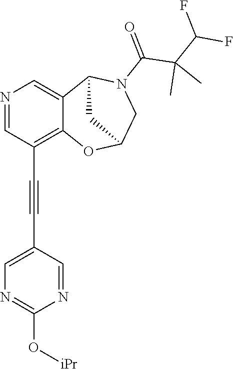 [SMILES: CC(C)Oc1ncc(C#Cc2cncc3c2O[C@H]2C[C@@H]3N(C(=O)C(C)(C)C(F)F)C2)cn1] | 3,3-difluoro-1-((2S,5S)-9- ((2-isopropoxypyrimidin- 5-yl)ethynyl)-2,3-dihydro- 2,5-methanopyrido[3,4- f][1,4]oxazepin-4(5H)-yl)- 2,2-dimethylpropan-1-one | Calc'd 443, found 443 |
| 2-75 | 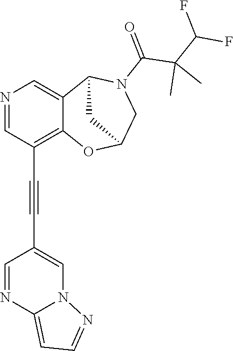 [SMILES: CC(C)(C(=O)N1C[C@@H]2C[C@H]1c1cncc(C#Cc3cnc4ccnn4c3)c1O2)C(F)F] | 3,3-difluoro-2,2-dimethyl- 1-((2S,5S)-9- (pyrazolo[1,5-a]pyrimidin- 6-ylethynyl)-2,3-dihydro- 2,5-methanopyrido[3,4- f][1,4]oxazepin-4(5H)- yl)propan-1-one | Calc'd 424, found 424 |
| 2-76 | 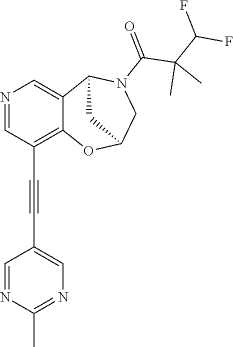 [SMILES: Cc1ncc(C#Cc2cncc3c2O[C@H]2C[C@@H]3N(C(=O)C(C)(C)C(F)F)C2)cn1] | 3,3-difluoro-2,2-dimethyl- 1-((2S,5S)-9-((2- methylpyrimidin-5- yl)ethynyl)-2,3-dihydro- 2,5-methanopyrido[3,4- f][1,4]oxazepin-4(5H)- yl)propan-1-one | Calc'd 399, found 399 |
| 2-77 | 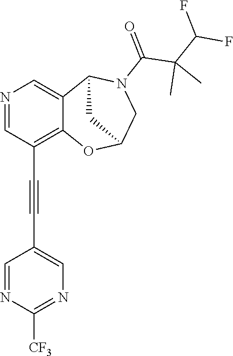 [SMILES: CC(C)(C(=O)N1C[C@@H]2C[C@H]1c1cncc(C#Cc3cnc(C(F)(F)F)nc3)c1O2)C(F)F] | 3,3-difluoro-2,2-dimethyl- 1-((2S,5S)-9-((2- (trifluoromethyl)pyrimidin- 5-yl)ethynyl)-2,3-dihydro- 2,5-methanopyrido[3,4- f][1,4]oxazepin-4(5H)- yl)propan-1-one | Calc'd 453, found 453 |
| 2-78 | 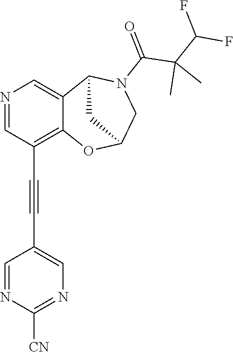 [SMILES: CC(C)(C(=O)N1C[C@@H]2C[C@H]1c1cncc(C#Cc3cnc(C#N)nc3)c1O2)C(F)F] | 5-(((2S,5S)-4-(3,3- difluoro-2,2- dimethylpropanoyl)- 2,3,4,5-tetrahydro-2,5- methanopyrido[3,4- f][1,4]oxazepin-9- yl)ethynyl)pyrimidine-2- carbonitrile | Calc'd 410, found 410 |
| 2-79 | 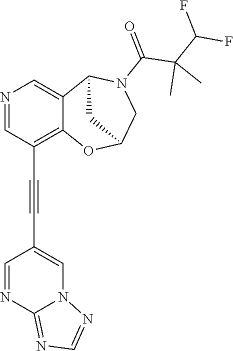 [SMILES: CC(C)(C(=O)N1C[C@@H]2C[C@H]1c1cncc(C#Cc3cnc4ncnn4c3)c1O2)C(F)F] | 1-((2S,5S)-9- ([1,2,4]triazolo[1,5- a]pyrimidin-6-ylethynyl)- 2,3-dihydro-2,5- methanopyrido[3,4- f][1,4]oxazepin-4(5H)-yl)- 3,3-difluoro-2,2- dimethylpropan-1-one | Calc'd 425, found 425 |
| 2-80 | 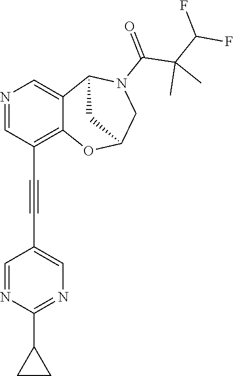 [SMILES: CC(C)(C(=O)N1C[C@@H]2C[C@H]1c1cncc(C#Cc3cnc(C4CC4)nc3)c1O2)C(F)F] | 1-((2S,5S)-9-((2- cyclopropylpyrimidin-5- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 425, found 425 |
| 2-81 | 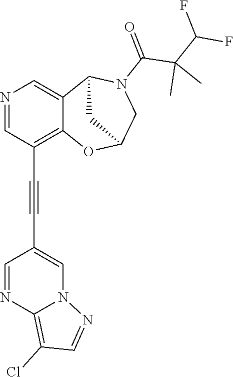 [SMILES: CC(C)(C(=O)N1C[C@@H]2C[C@H]1c1cncc(C#Cc3cnc4c(Cl)cnn4c3)c1O2)C(F)F] | 1-((2S,5S)-9-((3- chloropyrazolo[1,5- a]pyrimidin-6-yl)ethynyl)- 2,3-dihydro-2,5- methanopyrido[3,4- f][1,4]oxazepin-4(5H)-yl)- 3,3-difluoro-2,2- dimethylpropan-1-one | Calc'd 458, found 458 |
| 2-82 | 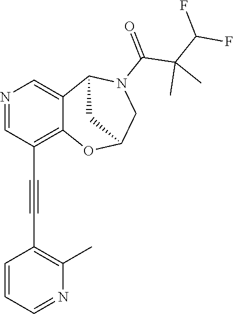 [SMILES: Cc1ncccc1C#Cc1cncc2c1O[C@H]1C[C@@H]2N(C(=O)C(C)(C)C(F)F)C1] | 3,3-difluoro-2,2-dimethyl- 1-((2S,5S)-9-((2- methylpyridin-3- yl)ethynyl)-2,3-dihydro- 2,5-methanopyrido[3,4- f][1,4]oxazepin-4(5H)- yl)propan-1-one | Calc'd 398, found 398 |
| 2-83 | 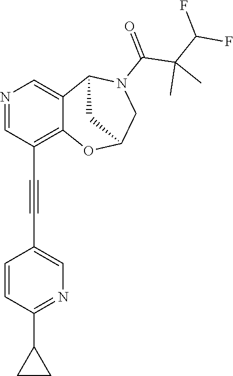 [SMILES: CC(C)(C(=O)N1C[C@@H]2C[C@H]1c1cncc(C#Cc3ccc(C4CC4)nc3)c1O2)C(F)F] | 1-((2S,5S)-9-((6- cyclopropylpyridin-3- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 424, found 424 |
| 2-84 | 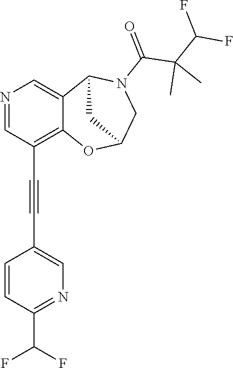 [SMILES: CC(C)(C(=O)N1C[C@@H]2C[C@H]1c1cncc(C#Cc3ccc(C(F)F)nc3)c1O2)C(F)F] | 1-((2S,5S)-9-((6- (difluoromethyl)pyridin-3- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 434, found 434 |
| 2-85 | 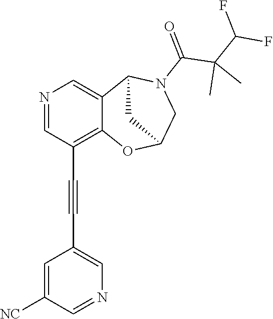 [SMILES: CC(C)(C(=O)N1C[C@@H]2C[C@H]1c1cncc(C#Cc3cncc(C#N)c3)c1O2)C(F)F] | 5-(((2S,5S)-4-(3,3- difluoro-2,2- dimethylpropanoyl)- 2,3,4,5-tetrahydro-2,5- methanopyrido[3,4- f][1,4]oxazepin-9- yl)ethynyl)nicotinonitrile | Calc'd 409, found 409 |
| 2-86 | 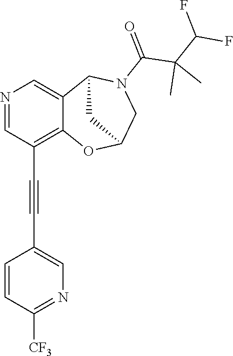 [SMILES: CC(C)(C(=O)N1C[C@@H]2C[C@H]1c1cncc(C#Cc3ccc(C(F)(F)F)nc3)c1O2)C(F)F] | 3,3-difluoro-2,2-dimethyl- 1-((2S,5S)-9-((6- (trifluoromethyl)pyridin-3- yl)ethynyl)-2,3-dihydro- 2,5-methanopyrido[3,4- f][1,4]oxazepin-4(5H)- yl)propan-1-one | Calc'd 452, found 452 |
| 2-87 | 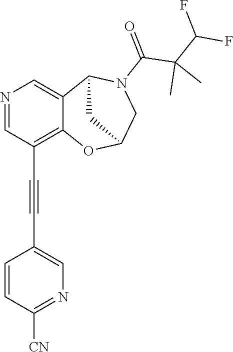 [SMILES: CC(C)(C(=O)N1C[C@@H]2C[C@H]1c1cncc(C#Cc3ccc(C#N)nc3)c1O2)C(F)F] | 5-(((2S,5S)-4-(3,3- difluoro-2,2- dimethylpropanoyl)- 2,3,4,5-tetrahydro-2,5- methanopyrido[3,4- f][1,4]oxazepin-9- yl)ethynyl)picolinonitrile | Calc'd 409, found 409 |
| 2-88 | 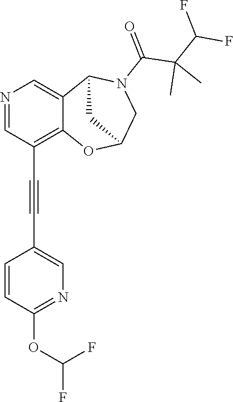 [SMILES: CC(C)(C(=O)N1C[C@@H]2C[C@H]1c1cncc(C#Cc3ccc(OC(F)F)nc3)c1O2)C(F)F] | 1-((2S,5S)-9-((6- (difluoromethoxy)pyridin- 3-yl)ethynyl)-2,3-dihydro- 2,5-methanopyrido[3,4- f][1,4]oxazepin-4(5H)-yl)- 3,3-difluoro-2,2- dimethylpropan-1-one | Calc'd 450, found 450 |
| 2-89 | 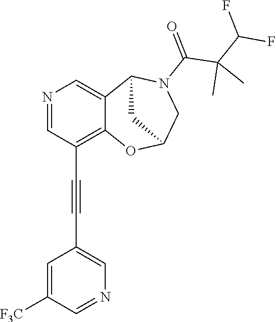 [SMILES: CC(C)(C(=O)N1C[C@@H]2C[C@H]1c1cncc(C#Cc3cncc(C(F)(F)F)c3)c1O2)C(F)F] | 3,3-difluoro-2,2-dimethyl- 1-((2S,5S)-9-((5- (trifluoromethyl)pyridin-3- yl)ethynyl)-2,3-dihydro- 2,5-methanopyrido[3,4- f][1,4]oxazepin-4(5H)- yl)propan-1-one | Calc'd 452, found 452 |
| 2-90 | 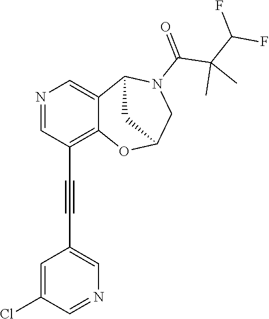 [SMILES: CC(C)(C(=O)N1C[C@@H]2C[C@H]1c1cncc(C#Cc3cncc(Cl)c3)c1O2)C(F)F] | 1-((2S,5S)-9-((5- chloropyridin-3- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 418, found 418 |
| 2-91 | 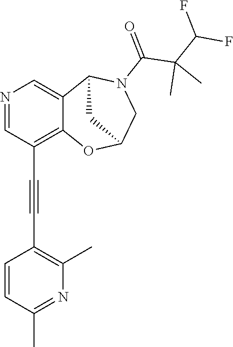 [SMILES: Cc1ccc(C#Cc2cncc3c2O[C@H]2C[C@@H]3N(C(=O)C(C)(C)C(F)F)C2)c(C)n1] | 1-((2S,5S)-9-((2,6- dimethylpyridin-3- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 412, found 412 |
| 2-92 | 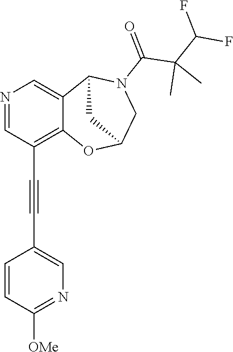 [SMILES: COc1ccc(C#Cc2cncc3c2O[C@H]2C[C@@H]3N(C(=O)C(C)(C)C(F)F)C2)cn1] | 3,3-difluoro-1-((2S,5S)-9- ((6-methoxypyridin-3- yl)ethynyl)-2,3-dihydro- 2,5-methanopyrido[3,4- f][1,4]oxazepin-4(5H)-yl)- 2,2-dimethylpropan-1-one | Calc'd 414, found 414 |
| 2-93 | 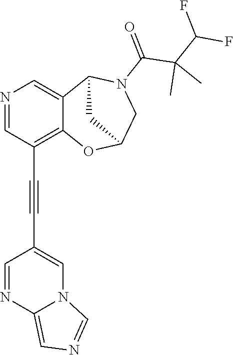 [SMILES: CC(C)(C(=O)N1C[C@@H]2C[C@H]1c1cncc(C#Cc3cnc4cncn4c3)c1O2)C(F)F] | 3,3-difluoro-1-((2S,5S)-9- (imidazo[1,5-a]pyrimidin- 3-ylethynyl)-2,3-dihydro- 2,5-methanopyrido[3,4- f][1,4]oxazepin-4(5H)-yl)- 2,2-dimethylpropan-1-one | Calc'd 424, found 424 |
| 2-94 | 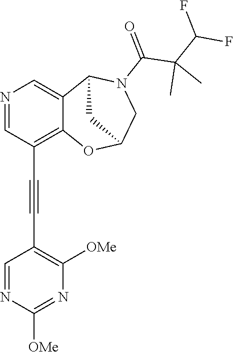 [SMILES: COc1ncc(C#Cc2cncc3c2O[C@H]2C[C@@H]3N(C(=O)C(C)(C)C(F)F)C2)c(OC)n1] | 1-((2S,5S)-9-((2,4- dimethoxypyrimidin-5- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 445, found 445 |
| 2-95 | 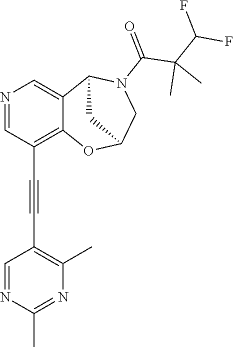 [SMILES: Cc1ncc(C#Cc2cncc3c2O[C@H]2C[C@@H]3N(C(=O)C(C)(C)C(F)F)C2)c(C)n1] | 1-((2S,5S)-9-((2,4- dimethylpyrimidin-5- yl)ethynyl)-2,3-dihydro- 2,5-methanopyrido[3,4- f][1,4]oxazepin-4(5H)-yl)- 3,3-difluoro-2,2- dimethylpropan-1-one | Calc'd 413, found 413 |
| 2-96 | 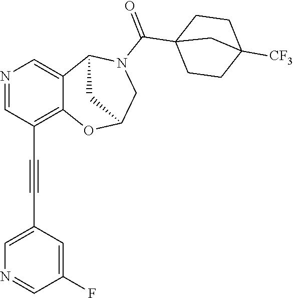 [SMILES: O=C(N1C[C@@H]2C[C@H]1c1cncc(C#Cc3cncc(F)c3)c1O2)C12CCC(C(F)(F)F)(CC1)C2] | ((2S,5S)-9-((5- fluoropyridin-3- yl)ethynyl)-2,3-dihydro- 2,5-methanopyrido[3,4- f][1,4]oxazepin-4(5H)- yl)(4- (trifluoromethyl)bicyclo [2.2.1]heptan-1-yl)methanone | Calc'd 472, found 472 |
| 2-97 | 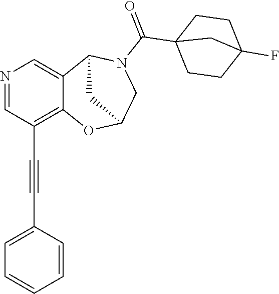 [SMILES: O=C(N1C[C@@H]2C[C@H]1c1cncc(C#Cc3ccccc3)c1O2)C12CCC(F)(CC1)C2] | (4- fluorobicyclo[2.2.1]heptan- 1-yl)((2S,5S)-9- (phenylethynyl)-2,3- dihydro-2,5- methanopyrido[3,4- f][1,4]oxazepin-4(5H)- yl)methanone | Calc'd 403, found 403 |
| 2-98 | 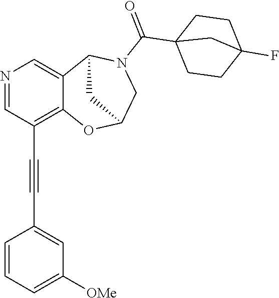 [SMILES: COc1cccc(C#Cc2cncc3c2O[C@H]2C[C@@H]3N(C(=O)C34CCC(F)(CC3)C4)C2)c1] | (4- fluorobicyclo[2.2.1]heptan- 1-yl)((2S,5S)-9-((3- methoxyphenyl)ethynyl)- 2,3-dihydro-2,5- methanopyrido[3,4- f][1,4]oxazepin-4(5H)- yl)methanone | Calc'd 433, found 433 |
| 2-99 | 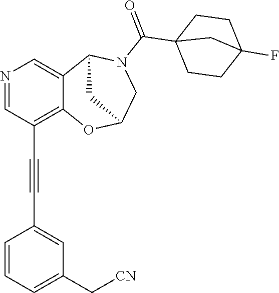 [SMILES: N#CCc1cccc(C#Cc2cncc3c2O[C@H]2C[C@@H]3N(C(=O)C34CCC(F)(CC3)C4)C2)c1] | 2-(3-(((2S,5S)-4-(4- fluorobicyclo[2.2.1]heptane- 1-carbonyl)-2,3,4,5- tetrahydro-2,5- methanopyrido[3,4- f][1,4]oxazepin-9- yl)ethynyl)phenyl) acetonitrile | Calc'd 442, found 442 |
| 2-100 | 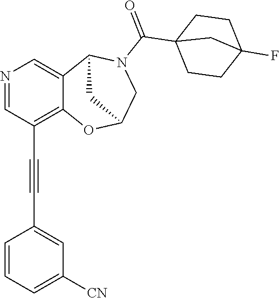 [SMILES: N#Cc1cccc(C#Cc2cncc3c2O[C@H]2C[C@@H]3N(C(=O)C34CCC(F)(CC3)C4)C2)c1] | 3-(((2S,5S)-4-(4- fluorobicyclo[2.2.1]heptane- 1-carbonyl)-2,3,4,5- tetrahydro-2,5- methanopyrido[3,4- f][1,4]oxazepin-9- yl)ethynyl)benzonitrile | Calc'd 428, found 428 |
| 2-101 | 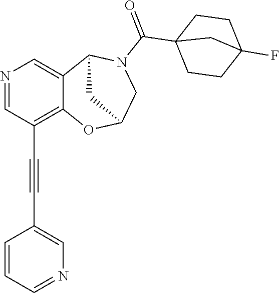 [SMILES: O=C(N1C[C@@H]2C[C@H]1c1cncc(C#Cc3cccnc3)c1O2)C12CCC(F)(CC1)C2] | (4- fluorobicyclo[2.2.1]heptan- 1-yl)((2S,5S)-9-(pyridin- 3-ylethynyl)-2,3-dihydro- 2,5-methanopyrido[3,4- f][1,4]oxazepin-4(5H)- yl)methanone | Calc'd 404, found 404 |
| 2-102 | 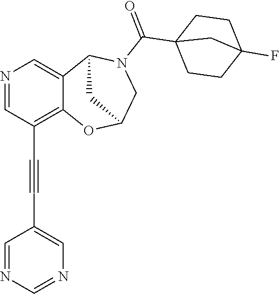 [SMILES: O=C(N1C[C@@H]2C[C@H]1c1cncc(C#Cc3cncnc3)c1O2)C12CCC(F)(CC1)C2] | (4- fluorobicyclo[2.2.1]heptan- 1-yl)((2S,5S)-9- (pyrimidin-5-ylethynyl)- 2,3-dihydro-2,5- methanopyrido[3,4- f][1,4]oxazepin-4(5H)- yl)methanone | Calc'd 405, found 405 |
| 2-103 | 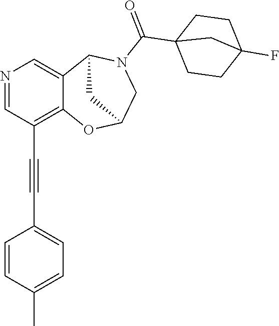 [SMILES: Cc1ccc(C#Cc2cncc3c2O[C@H]2C[C@@H]3N(C(=O)C34CCC(F)(CC3)C4)C2)cc1] | (4- fluorobicyclo[2.2.1]heptan- 1-yl)((2S,5S)-9-(p- tolylethynyl)-2,3-dihydro- 2,5-methanopyrido[3,4- f][1,4]oxazepin-4(5H)- yl)methanone | Calc'd 417, found 417 |
| 2-104 | 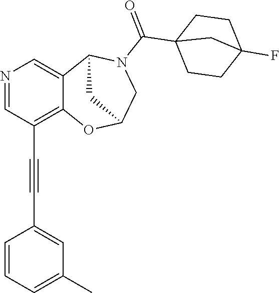 [SMILES: Cc1cccc(C#Cc2cncc3c2O[C@H]2C[C@@H]3N(C(=O)C34CCC(F)(CC3)C4)C2)c1] | (4- fluorobicyclo[2.2.1]heptan- 1-yl)((2S,5S)-9-(m- tolylethynyl)-2,3-dihydro- 2,5-methanopyrido[3,4- f][1,4]oxazepin-4(5H)- yl)methanone | Calc'd 417, found 417 |
| 2-105 | 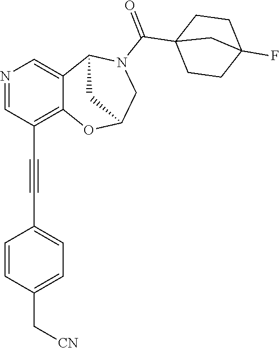 [SMILES: N#CCc1ccc(C#Cc2cncc3c2O[C@H]2C[C@@H]3N(C(=O)C34CCC(F)(CC3)C4)C2)cc1] | 2-(4-(((2S,5S)-4-(4- fluorobicyclo[2.2.1]heptane- 1-carbonyl)-2,3,4,5- tetrahydro-2,5- methanopyrido[3,4- f\|[1,4]oxazepin-9- yl)ethynyl)phenyl) acetonitrile | Calc'd 442, found 442 |
| 2-106 | 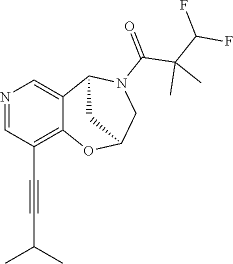 [SMILES: CC(C)C#Cc1cncc2c1O[C@H]1C[C@@H]2N(C(=O)C(C)(C)C(F)F)C1] | 3,3-difluoro-2,2-dimethyl- 1-((2S,5S)-9-(3-methylbut- 1-yn-1-yl)-2,3-dihydro- 2,5-methanopyrido[3,4- f][1,4]oxazepin-4(SH)- yl)propan-1-one | Calc'd 349, found 349 |
| 2-107 | 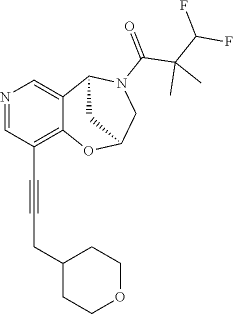 [SMILES: CC(C)(C(=O)N1C[C@@H]2C[C@H]1c1cncc(C#CCC3CCOCC3)c1O2)C(F)F] | 3,3-difluoro-2,2-dimethyl- 1-((2S,5S)-9-(3- (tetrahydro-2H-pyran-4- yl)prop-1-yn-1-yl)-2,3- dihydro-2,5- methanopyrido[3,4- f][1,4]oxazepin-4(5H)- yl)propan-1-one | Calc'd 405, found 405 |
| 2-108 | 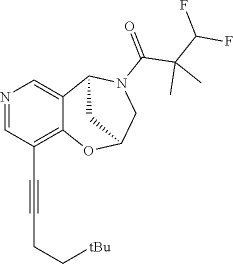 [SMILES: CC(C)(C)CCC#Cc1cncc2c1O[C@H]1C[C@@H]2N(C(=O)C(C)(C)C(F)F)C1] | 1-((2S,5S)-9-(5,5- dimethylhex-1-yn-1-yl)- 2,3-dihydro-2,5- methanopyrido[3,4- f][1,4]oxazepin-4(5H)-yl)- 3,3-difluoro-2,2- dimethylpropan-1-one | Calc'd 391, found 391 |
| 2-109 | 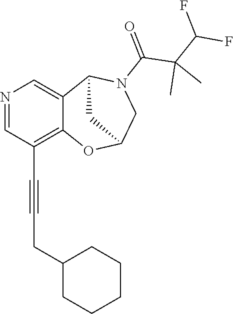 [SMILES: CC(C)(C(=O)N1C[C@@H]2C[C@H]1c1cncc(C#CCC3CCCCC3)c1O2)C(F)F] | 1-((2S,5S)-9-(3- cyclohexylprop-1-yn-1-yl)- 2,3-dihydro-2,5- methanopyrido[3,4- f][1,4]oxazepin-4(5H)-yl)- 3,3-difluoro-2,2- dimethylpropan-1-one | Calc'd 403, found 403 |
| 2-110 | 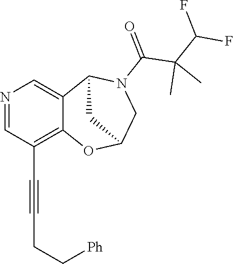 [SMILES: CC(C)(C(=O)N1C[C@@H]2C[C@H]1c1cncc(C#CCCc3ccccc3)c1O2)C(F)F] | 3,3-difluoro-2,2-dimethyl- 1-((2S,5S)-9-(4-phenylbut- 1-yn-1-yl)-2,3-dihydro- 2,5-methanopyrido[3,4- f][1,4]oxazepin-4(5H)- yl)propan-1-one | Calc'd 411, found 411 |
| 2-111 | 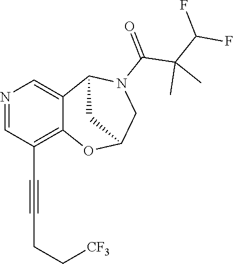 [SMILES: CC(C)(C(=O)N1C[C@@H]2C[C@H]1c1cncc(C#CCCC(F)(F)F)c1O2)C(F)F] | 3,3-difluoro-2,2-dimethyl- 1-((2S,5S)-9-(5,5,5- trifluoropent-1-yn-1-yl)- 2,3-dihydro-2,5- methanopyrido[3,4- f][1,4]oxazepin-4(5H)- yl)propan-1-one | Calc'd 403, found 403 |
| 2-112 | 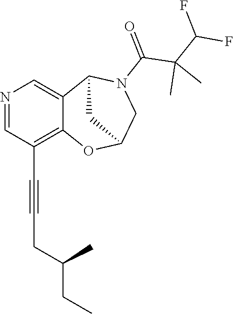 [SMILES: CC[C@H](C)CC#Cc1cncc2c1O[C@H]1C[C@@H]2N(C(=O)C(C)(C)C(F)F)C1] | 3,3-difluoro-2,2-dimethyl- 1-((2S,5S)-9-((S)-4- methylhex-1-yn-1-yl)-2,3- dihydro-2,5- methanopyrido[3,4- f][1,4]oxazepin-4(5H)- yl)propan-1-one | Calc'd 377, found 377 |
| 2-113 | 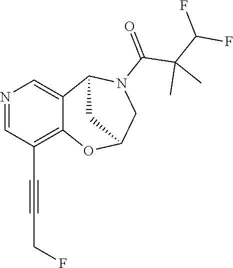 [SMILES: CC(C)(C(=O)N1C[C@@H]2C[C@H]1c1cncc(C#CCF)c1O2)C(F)F] | 3,3-difluoro-1-((2S,5S)-9- (3-fluoroprop-1-yn-1-yl)- 2,3-dihydro-2,5- methanopyrido[3,4- f][1,4]oxazepin-4(5H)-yl)- 2,2-dimethylpropan-1-one | Calc'd 339, found 339 |
| 2-114 | 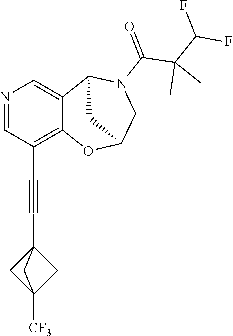 [SMILES: CC(C)(C(=O)N1C[C@@H]2C[C@H]1c1cncc(C#CC34CC(C(F)(F)F)(C3)C4)c1O2)C(F)F] | 3,3-difluoro-2,2-dimethyl- 1-((2S,5S)-9-((3- (trifluoromethyl)bicyclo [1.1.1]pentan-1-yl)ethynyl)- 2,3-dihydro-2,5- methanopyrido[3,4- f][1,4]oxazepin-4(5H)- yl)propan-1-one | Calc'd 441, found 441 |
| 2-115 | 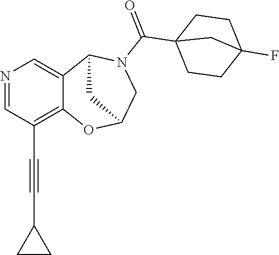 [SMILES: O=C(N1C[C@@H]2C[C@H]1c1cncc(C#CC3CC3)c1O2)C12CCC(F)(CC1)C2] | ((2S,5S)-9- (cyclopropylethynyl)-2,3- dihydro-2,5- methanopyrido[3,4- f][1,4]oxazepin-4(5H)- yl)(4- fluorobicyclo[2.2.1]heptan- 1-yl)methanone | Calc'd 367, found 367 |
| 2-116 | 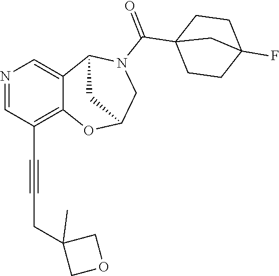 [SMILES: CC1(CC#Cc2cncc3c2O[C@H]2C[C@@H]3N(C(=O)C34CCC(F)(CC3)C4)C2)COC1] | (4- fluorobicyclo[2.2.1]heptan- 1-yl)((2S,5S)-9-(3-(3- methyloxetan-3-yl)prop-1- yn-1-yl)-2,3-dihydro-2,5- methanopyrido[3,4- f][1,4]oxazepin-4(5H)- yl)methanone | Calc'd 411, found 411 |
| 2-117 | 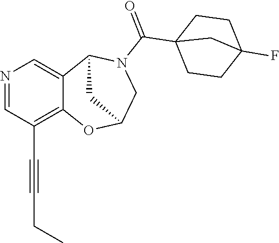 [SMILES: CCC#Cc1cncc2c1O[C@H]1C[C@@H]2N(C(=O)C23CCC(F)(CC2)C3)C1] | ((2S,5S)-9-(but-1-yn-1-y])- 2,3-dihydro-2,5- methanopyrido[3,4- f][1,4]oxazepin-4(5H)- yl)(4- fluorobicyclo[2.2.1]heptan- 1-yl)methanone | Calc'd 355, found 355 |
| 2-118 | 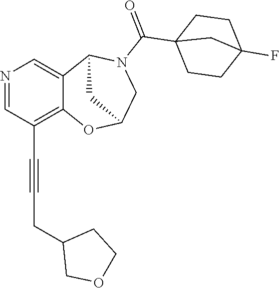 [SMILES: O=C(N1C[C@@H]2C[C@H]1c1cncc(C#CCC3CCOC3)c1O2)C12CCC(F)(CC1)C2] | (4- fluorobicyclo[2.2.1]heptan- 1-yl)((2S,5S)-9-(3- (tetrahydrofuran-3-yl)prop- 1-yn-1-yl)-2,3-dihydro- 2,5-methanopyrido[3,4- f][1,4]oxazepin-4(5H)- yl)methanone | Calc'd 411, found 411 |
| 2-119 | 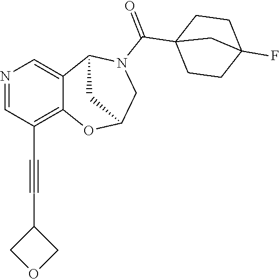 [SMILES: O=C(N1C[C@@H]2C[C@H]1c1cncc(C#CC3COC3)c1O2)C12CCC(F)(CC1)C2] | (4- fluorobicyclo[2.2.1]heptan- 1-yl)((2S,5S)-9-(oxetan-3- ylethynyl)-2,3-dihydro- 2,5-methanopyrido[3,4- f][1,4]oxazepin-4(5H)- yl)methanone | Calc'd 383, found 383 |
| 2-120 | 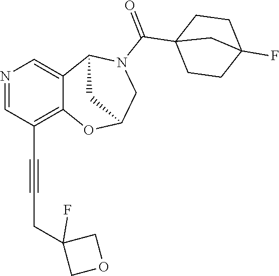 [SMILES: O=C(N1C[C@@H]2C[C@H]1c1cncc(C#CCC3(F)COC3)c1O2)C12CCC(F)(CC1)C2] | (4- fluorobicyclo[2.2.1]heptan- 1-yl)((2S,5S)-9-(3-(3- fluorooxetan-3-yl)prop-1- yn-1-yl)-2,3-dihydro-2,5- methanopyrido[3,4- f][1,4]oxazepin-4(5H)- yl)methanone | Calc'd 415, found 415 |
| 2-121 | 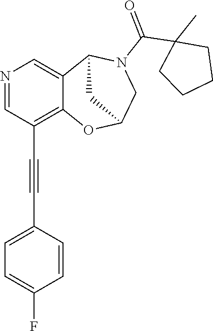 [SMILES: CC1(C(=O)N2C[C@@H]3C[C@H]2c2cncc(C#Cc4ccc(F)cc4)c2O3)CCCC1] | ((2S,5S)-9-((4- fluorophenyl)ethynyl)-2,3- dihydro-2,5- methanopyrido[3,4- f][1,4]oxazepin-4(5H)- yl)(1- methylcyclopentyl) methanone | Calc'd 391, found 391 |
| 2-122 | 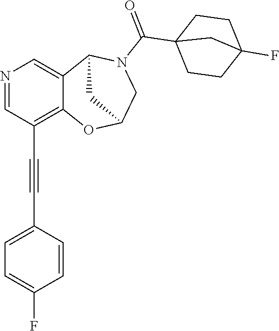 [SMILES: O=C(N1C[C@@H]2C[C@H]1c1cncc(C#Cc3ccc(F)cc3)c1O2)C12CCC(F)(CC1)C2] | (4- fluorobicyclo[2.2.1]heptan- 1-yl)((2S,5S)-9-((4- fluorophenyl)ethynyl)-2,3- dihydro-2,5- methanopyrido[3,4- f][1,4]oxazepin-4(5H)- yl)methanone | Calc'd 421, found 421 |
| 2-123 | 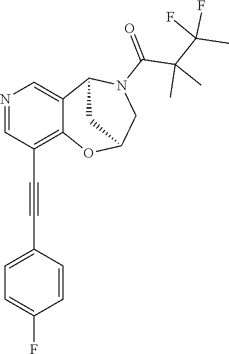 [SMILES: CC(F)(F)C(C)(C)C(=O)N1C[C@@H]2C[C@H]1c1cncc(C#Cc3ccc(F)cc3)c1O2] | 3,3-difluoro-1-((25,5S)-9- ((4-fluorophenyl)ethynyl)- 2,3-dihydro-2,5- methanopyrido[3,4- f][1,4]oxazepin-4(5H)-yl)- 2,2-dimethylbutan-1-one | Calc'd 415, found 415 |

### Table 3 Examples

### Example 3A. Preparation of 3,3-Difluoro-1-((2S,5S)-7-fluoro-2,3-dihydro-2,5-methanopyrido[3,2-f][1,4]oxazepin-4(5H)-yl)-2,2-dimethylpropan-1-one (3-1)

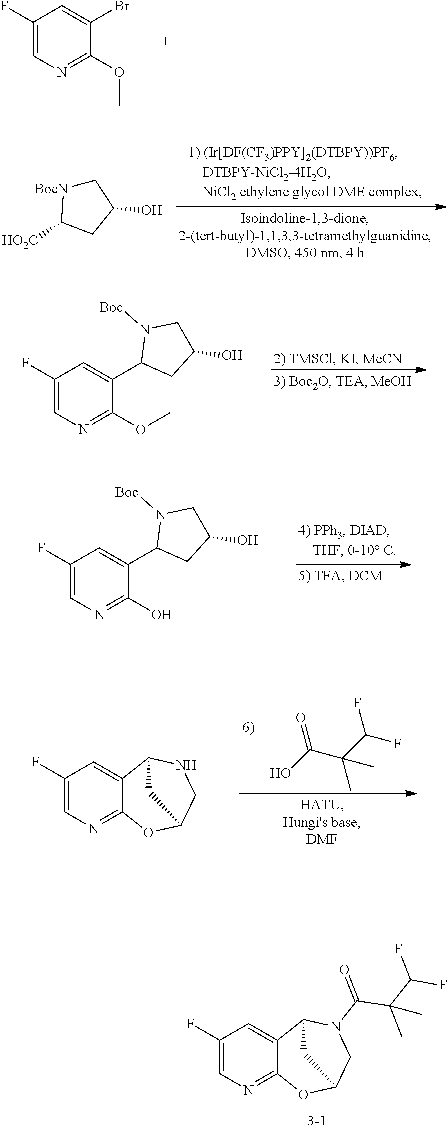

Step 1. A solution of 3-bromo-5-fluoro-2-methoxypyridine (1.2 g, 5.82 mmol), (2R,4R)-1-(tert-butoxycarbonyl)-4-hydroxypyrrolidine-2-carboxylic acid (1.62 g, 6.99 mmol), isoindoline-1,3-dione (0.857 g, 5.82 mmol), DTBPY-NiCl₂-4H₂O (0.348 g, 0.874 mmol), NiCl₂ ethylene glycol DME complex (0.128 g, 0.582 mmol), 2-(tert-butyl)-1,1,3,3-tetramethylguanidine (1.50 g, 8.74 mmol) in DMSO (5 mL) was treated with (Ir[DF(CF₃)PPY]₂(DTBPY))PF₆ (0.131 g, 0.116 mmol). The reaction mixture was stirred in a photoreactor for 4 h with 450 nm irradiation. The mixture was diluted with water (15 mL) and extracted with EtOAc (3×10 mL). The combined organic layers were washed with brine, dried over anhydrous Na₂SO₄, filtered and concentrated. The residue was purified by chromatography on silica gel (0-30% EtOAc/petroleum ether) to give tert-butyl (4R)-2-(5-fluoro-2-methoxypyridin-3-yl)-4-hydroxypyrrolidine-1-carboxylate. MS (EI) calculated for C₁₅H₂₂FN₂O₄[M+H]⁺, 313; found, 313.

Step 2. A mixture of tert-butyl (4R)-2-(5-fluoro-2-methoxypyridin-3-yl)-4-hydroxypyrrolidine-1-carboxylate (300 mg, 0.960 mmol) in MeCN (1 mL) was treated with KI (638 mg, 3.84 mmol) and TMSCl (0.25 mL, 1.9 mmol). The mixture was stirred at 20° C. for 12 h and concentrated, giving 5-fluoro-3-((4R)-4-hydroxypyrrolidin-2-yl)pyridin-2-ol. MS (EI) calculated for C₉H₁₂FN₂O₂[M+H]⁺, 199; found, 199.

Step 3. A mixture containing 5-fluoro-3-((4R)-4-hydroxypyrrolidin-2-yl)pyridin-2-ol (190 mg, 0.959 mmol) in MeOH (5 mL) was treated with di-tert-butyl dicarbonate (0.334 mL, 1.44 mmol) and TEA (0.40 mL, 2.9 mmol). The mixture was stirred at 20° C. for 1 h, then purified by reverse phase chromatography (gradient of 18 to 48% MeCN/water with 0.05% NH₃H₂O with 10 mM NH₄HCO₃), to give tert-butyl (4R)-2-(5-fluoro-2-hydroxypyridin-3-yl)-4-hydroxypyrrolidine-1-carboxylate. MS (EI) calculated for C₁₄H₂₀FN₂O₄[M+H]⁺, 299; found, 299.

Step 4. A solution of tert-butyl (4R)-2-(5-fluoro-2-hydroxypyridin-3-yl)-4-hydroxypyrrolidine-1-carboxylate (80 mg, 0.27 mmol) in THF (5 mL) was treated with Ph₃P (106 mg, 0.402 mmol), DIAD (0.078 mL, 0.40 mmol) at 0° C. The mixture was stirred at 20° C. for 15 h, concentrated and purified by chromatography on silica gel (1:1 EtOAc/petroleum ether) to give tert-butyl (2S,5S)-7-fluoro-2,3-dihydro-2,5-methanopyrido[3,2-f][1,4]oxazepine-4(5H)-carboxylate. MS (EI) calculated for C₁₄H₁₈FN₂O₃[M+H]⁺, 281; found, 281.

Step 5. A solution of tert-butyl (2S,5S)-7-fluoro-2,3-dihydro-2,5-methanopyrido[3,2-f][1,4]oxazepine-4(5H)-carboxylate (25 mg, 0.089 mmol) in DCM (2 mL) and TFA (0.5 mL) was stirred at 25° C. for 1 h. The mixture was concentrated to give (2S,5S)-7-fluoro-2,3,4,5-tetrahydro-2,5-methanopyrido[3,2-f][1,4]oxazepine.

Step 6. A mixture containing (2S,5S)-7-fluoro-2,3,4,5-tetrahydro-2,5-methanopyrido[3,2-f][1,4]oxazepine (15 mg, 0.083 mmol), 2-(3H-[1,2,3]triazolo[4,5-b]pyridin-3-yl)-1,1,3,3-tetramethylisouronium hexafluorophosphate(V) (63 mg, 0.17 mmol) and N-ethyl-N-isopropylpropan-2-amine (0.073 mL, 0.42 mmol) in DMF (1 mL) was treated with 3,3-difluoro-2,2-dimethylpropanoic acid (17 mg, 0.13 mmol) and the mixture stirred for 12 h. The mixture was purified by reverse phase chromatography (gradient of 23-53% MeCN/water with 0.1% TFA) to give 3,3-difluoro-1-((2S,5S)-7-fluoro-2,3-dihydro-2,5-methanopyrido[3,2-f][1,4]oxazepin-4(5H)-yl)-2,2-dimethylpropan-1-one. ¹H NMR (400 MHz, CDCl₃) δ 8.00 (s, 1H), 7.50 (m, 1H), 6.05 (t, J=57 Hz, 1H), 5.30 (m, 1H), 5.22 (m, 1H), 4.09 (d, J=12 Hz, 1H), 3.94 (d, J=12 Hz, 1H), 2.23 (m, 2H), 1.26 (s, 3H), 1.19 (s, 3H); MS (EI) calculated for C₁₄H₁₆F₃N₂O₂[M+H]⁺, 301; found, 301.

### Example 3B. Preparation of ((5S,8S)-7,8-Dihydro-5,8-methanopyrimido[5,4-f][1,4]oxazepin-6(5H)-yl)(4-fluorobicyclo[2.2.1]heptan-1-yl)methanone (3-3)

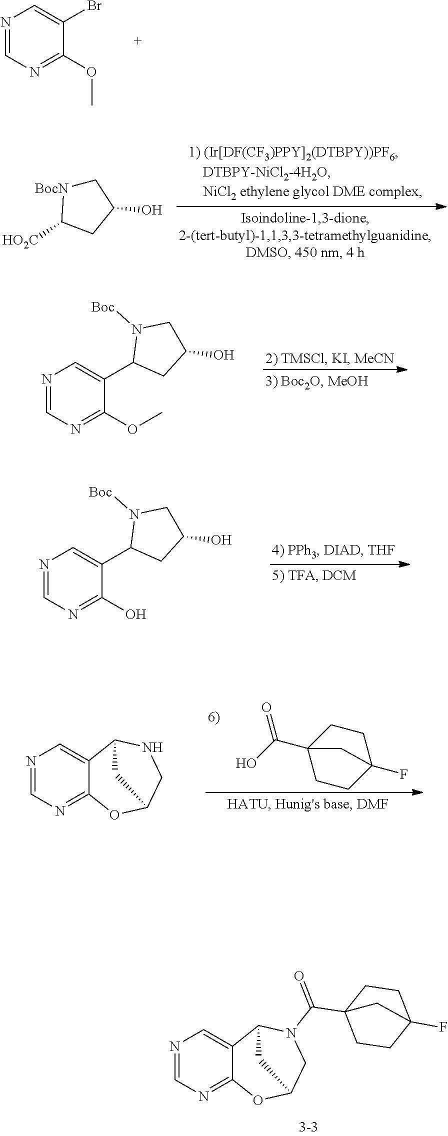

Step 1. A solution of 5-bromo-4-methoxypyrimidine (100 mg, 0.529 mmol), (2R,4R)-1-(tert-butoxycarbonyl)-4-hydroxypyrrolidine-2-carboxylic acid (147 mg, 0.635 mmol), isoindoline-1,3-dione (78 mg, 0.53 mmol), DTBPY-NiCl₂-4H₂O (32 mg, 0.079 mmol), NiCl₂ ethylene glycol DME complex (12 mg, 0.053 mmol), 2-(tert-butyl)-1,1,3,3-tetramethylguanidine (136 mg, 0.794 mmol) in DMSO (2.5 mL) was treated with (Ir[DF(CF₃)PPY]₂(DTBPY))PF₆ (12 mg, 11 μmol). The reaction mixture was stirred in a photoreactor for 4 h with 450 nm irradiation. The mixture was diluted with water (15 mL) and extracted with EtOAc (3×10 mL). The combined organic layers were washed with brine, dried over anhydrous Na₂SO₄, filtered and concentrated. The residue was purified by chromatography on silica gel (25% EtOAc/petroleum ether) followed by reverse phase chromatography (gradient of 20-40% MeCN/water with 0.1% TFA) to give tert-butyl (4R)-4-hydroxy-2-(4-methoxypyrimidin-5-yl)pyrrolidine-1-carboxylate. ¹H NMR (400 MHz, DMSO-do) S 8.69 (s, 1H), 8.33-8.40 (m, 1H), 4.78-4.92 (m, 1H), 4.26 (br s, 1H), 3.91-4.00 (m, 3H), 3.32-3.60 (m, 2H), 2.12-2.24 (m, 1H), 1.70-1.97 (m, 1H), 1.31-1.41 (m, 3H), 1.02-1.12 (m, 6H); MS (EI) calculated for C₁₄H₂₂N₃O₄ [M+H]⁺, 296; found, 296.

Step 2. A mixture of tert-butyl (4R)-4-hydroxy-2-(4-methoxypyrimidin-5-yl)pyrrolidine-1-carboxylate (30 mg, 0.10 mmol), chlorotrimethylsilane (22 mg, 0.20 mmol) and KI (67 mg, 0.41 mmol) in MeCN (2 mL) was stirred for 1 h. The reaction was filtered and the filter cake was dried to give 5-((4R)-4-hydroxypyrrolidin-2-yl)pyrimidin-4-ol. ¹H NMR (400 MHz, CD₃OD) δ 8.57 (s, 1H), 8.14 (s, 1H), 4.82 (br s, 1H), 4.67 (br s, 1H), 3.60 (dd, J=12, 3.4 Hz, 1H), 3.34 (m, 1H), 2.39-2.50 (m, 1H), 2.28 (br dd, J=14, 6.4 Hz, 1H).

Step 3. A mixture of 5-((4R)-4-hydroxypyrrolidin-2-yl)pyrimidin-4-ol (15 mg, 0.083 mmol) in MeOH (0.5 mL) was treated with di-tert-butyl dicarbonate (0.023 mL, 0.099 mmol). The mixture was stirred for 16 h, then purified by reverse phase chromatography (gradient of 0-20% MeCN/water with 0.225% formic acid) to provide tert-butyl (4R)-4-hydroxy-2-(4-hydroxypyrimidin-5-yl)pyrrolidine-1-carboxylate. ¹H NMR (500 MHz, CD₃OD) δ 8.08-8.23 (m, 1H), 7.70-7.89 (m, 1H), 4.84 (br s, 1H), 4.26-4.46 (m, 1H), 3.53-3.71 (m, 2H), 1.98-2.42 (m, 2H), 1.23-1.49 (m, 9H); MS (EI) calculated for C₁₃H₂₀N₃O₄ [M+H]⁺, 282; found, 282.

Step 4. A solution of tert-butyl (4R)-4-hydroxy-2-(4-hydroxypyrimidin-5-yl)pyrrolidine-1-carboxylate (112 mg, 0.398 mmol) in THF (1 mL) was treated with PPh₃ (627 mg, 2.39 mmol) and DIAD (0.474 mL, 2.39 mmol). The mixture was stirred for 22 h, then purified by reverse phase chromatography (gradient of 1545% MeCN/water with 0.1% TFA) to give tert-butyl (5S,8S)-7,8-dihydro-5,8-methanopyrimido[5,4-f][1,4]oxazepine-6(5H)-carboxylate. ¹H NMR (500 MHz. CD₃OD) δ 8.57-8.72 (m, 1H), 8.32-8.48 (m, 1H), 5.28 (br s, 1H), 3.61-3.87 (m, 2H), 2.36-2.54 (m, 1H), 2.16-2.31 (m, 2H), 1.25 (br s, 9H); MS (ET) calculated for CoHlaN303 [M+H]⁺, 264; found, 264.

Step 5. A mixture of tert-butyl (5S,8S)-7,8-dihydro-5,8-methanopyrimido[5,4-f][1,4]oxazepine-6(5H)-carboxylate (67 mg, 0.25 mmol) and TFA (0.8 mL) in DCM (4 mL) was stirred for 2 h. The solvent was evaporated to give (5S,8S)-5,6,7,8-tetrahydro-5,8-methanopyrimido[5,4-f][1,4]oxazepane.

Step 6. A mixture of (5S,8S)-5,6,7,8-tetrahydro-5,8-methanopyrimido[5,4-f][1,4]oxazepine (20 mg, 0.12 mmol), 4-fluorobicyclo[2.2.1]heptane-1-carboxylic acid (27 mg, 0.17 mmol), TEA (0.085 mL, 0.613 mmol) in DMF (1.5 mL) was treated with HATU (93 mg, 0.25 mmol), and then stirred for 16 h. The mixture was purified by reverse phase chromatography (gradient of 15 to 45% MeCN/water with 0.1% TFA to give ((5S,8S)-7,8-dihydro-5,8-methanopyrimido[5,4-f][1,4]oxazepin-6(5H)-yl)(4-fluorobicyclo[2.2.1]heptan-1-yl)methanone. ¹H NMR (400 MHz, CD₃OD) δ 8.70 (s, 1H), 8.49 (s, 1H), 5.42 (br s, 1H), 5.29 (m, 1H), 4.07-4.17 (m, 1H), 3.96-4.05 (m, 1H), 2.34-2.46 (m, 1H), 2.20-2.31 (m, 1H), 1.69-2.14 (m, 10H); MS (EI) calculated for C₁₆H₁₉FN₃O₂ [M+H]⁺, 304; found, 304.

### Example 3C. Preparation of (4-fluorobicyclo[2.2.1]heptan-1-yl)((5S,8S)-2-methyl-7,8-dihydro-5,8-methanopyrimido[5,4-f][1,4]oxazepin-6(5H)-yl)methanone (3-4)

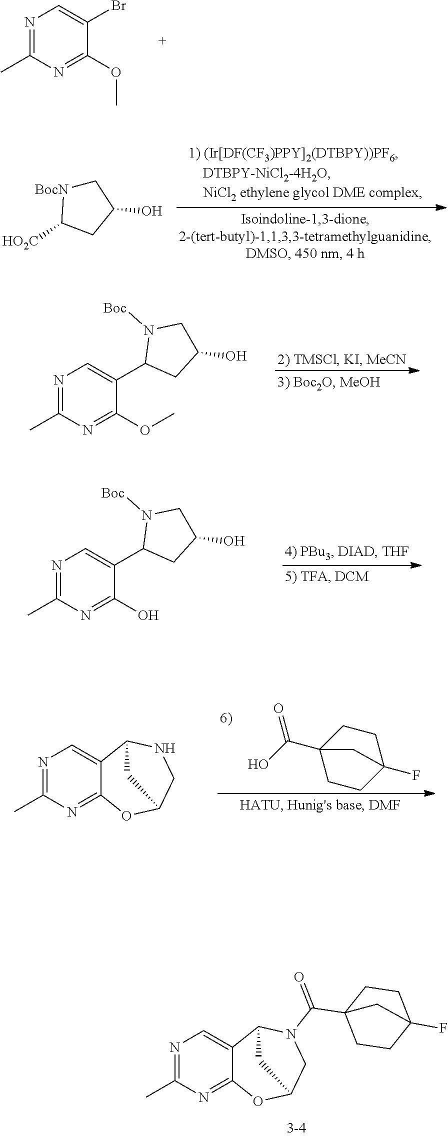

Step 1. A solution of 5-bromo-4-methoxy-2-methylpyrimidine (500 mg, 2.46 mmol), (2R,4R)-1-(tert-butoxycarbonyl)-4-hydroxypyrrolidine-2-carboxylic acid (683 mg, 2.96 mmol), isoindoline-1,3-dione (362 mg, 2.46 mmol), DTBPY-NiCl₂-4H₂O (147 mg, 0.369 mmol), NiCl₂ ethylene glycol DME complex (54 mg, 0.25 mmol), 2-(tert-butyl)-1,1,3,3-tetramethylguanidine (633 mg, 3.69 mmol) in DMSO (5 mL) was treated with (Ir[DF(CF₃)PPY]₂(DTBPY))PF₆ (50 mg, 50 μmol). The reaction mixture was stirred in a photoreactor for 4 h with 450 nm irradiation. The mixture was diluted with water (15 mL) and extracted with EtOAc (3×10 mL). The combined organic layers were washed with brine, dried over anhydrous Na₂SO₄, filtered and concentrated. The residue was purified by chromatography on silica gel (25% EtOAc/petroleum ether) to give tert-butyl (4R)-4-hydroxy-2-(4-methoxy-2-methylpyrimidin-5-yl)pyrrolidine-1-carboxylate. MS (EI) calculated for C₃H₂₄N₃O₄ [M+H], 310; found, 310.

Step 2. A mixture of tert-butyl (4R)-4-hydroxy-2-(4-methoxy-2-methylpyrimidin-5-yl)pyrrolidine-1-carboxylate (360 mg, 1.16 mmol), chlorotrimethylsilane (253 mg, 2.33 mmol) and KI (773 mg, 4.65 mmol) in MeCN (10 mL) was stirred for 16 h. The reaction was filtered and the filter cake was dried to give 5-((4R)-4-hydroxypyrrolidin-2-yl)-2-methylpyrimidin-4-ol.

Step 3. A mixture of 5-((4R)-4-hydroxypyrrolidin-2-yl)-2-methylpyrimidin-4-ol (360 mg, 1.84 mmol) in MeOH (10 mL) was treated with di-tert-butyl dicarbonate (0.514 mL, 2.21 mmol).

The mixture was stirred for 16 h, then purified by reverse phase chromatography (gradient of 5-15% MeCN/water with 0.225% formic acid) to give tert-butyl (4R)-4-hydroxy-2-(4-hydroxy-2-methylpyrimidin-5-yl)pyrrolidine-1-carboxylate. ¹H NMR (400 MHz, DMSO-d₅) δ 12.41 (br s, 1H), 7.48-7.66 (m, 1H), 4.93 (br s, 1H), 4.66 (s, 1H), 4.22 (br s, 1H), 3.48 (br d. J=13 Hz, 1H), 2.25 (br s, 3H), 2.07 (m, 2H), 1.21 and 1.37 (2 s, 9H); MS (EI) calculated for C₁₄H₂₂N₃O₄ [M+H]⁺, 296; found, 2%.

Step 4. A solution of tert-butyl (4R)-4-hydroxy-2-(4-hydroxy-2-methylpyrimidin-5-yl)pyrrolidine-1-carboxylate (120 mg, 0.406 mmol) in THF (1 mL) was treated with PBu₃ (493 mg, 2.44 mmol) and DIAD (0.483 mL, 2.44 mmol). The mixture was stirred for 16 h, then purified by reverse phase chromatography (gradient of 18-38% MeCN/water with 0.1% TFA) to give tert-butyl (5S,8S)-2-methyl-7,8-dihydro-5,8-methanopyrimido[5,4-f][1,4]oxazepine-6(5H)-carboxylate. ¹H NMR (400 MHz, CD₃OD) δ 8.25-8.58 (m, 1H), 5.41 (m, 1H), 5.01 (dd, J=13, 4.7 Hz, 1H), 3.63-3.88 (m, 2H), 2.62 (2 s, 3H), 2.50 (m, 1H), 2.28 (m, 1H), 1.44 (m, 9H); MS (EI) calculated for C₁₄H₂₀N₃O₃ [M+H]⁺, 278; found, 278.

Step 5. A mixture of tert-butyl (5S,8S)-2-methyl-7,8-dihydro-5,8-methanopyrimido[5,4-f][1,4]oxazepine-6(5H)-carboxylate (90 mg, 0.33 mmol) and TFA (0.6 mL) in DCM (3 mL) was stirred for 2 h. The solvent was evaporated to give (5S,8S)-2-methyl-5,6,7,8-tetrahydro-5,8-methanopyrimido[5,4-f][1,4]oxazepine.

Step 6. A mixture of (5S,8S)-2-methyl-5,6,7,8-tetrahydro-5,8-methanopyrimido[5,4-f][1,4]oxazepine (20 mg, 0.11 mmol), 4-fluorobicyclo[2.2.1]heptane-1-carboxylic acid (25 mg, 0.16 mmol), TEA (0.080 mL, 0.56 mmol) in DMF (1.5 mL) was treated with HATU (86 mg, 0.23 mmol), then stirred for 16 h. The mixture was purified by reverse phase chromatography (gradient of 12-42% MeCN/water with 0.1% TFA) to give (4-fluorobicyclo[2.2.1]heptan-1-yl)((5S,8S)-2-methyl-7,8-dihydro-5,8-methanopyrimido[5,4-f][1,4]oxazepin-6(5H)-yl)methanone. ¹H NMR (400 MHz, CD₃OD) δ 8.46 (s, 1H), 5.48 (br s, 1H), 5.30 (m, 1H), 4.08-4.18 (m, 1H), 3.95-4.07 (m, 1H), 2.61 (s, 3H), 2.37-2.48 (m, 1H), 2.26 (m, 1H), 1.84-2.06 (m, 8H), 1.70-1.83 (m, 2H); MS (EI) calculated for C₁₇H₂₁FN₃O₂ [M+H]⁺, 318. found, 318.

Compound 3-2 was prepared in a fashion similar to 3-3, substituting 4-fluorobicyclo[2.2.1]heptane-1-carboxylic acid for 3,3-difluoro-2,2-dimethylbutanoic acid.

TABLE 3

| Compound | | | Exact Mass |
|---|---|---|---|
| Number | Structure | Name | [M + H]⁺ |
| 3-1 | 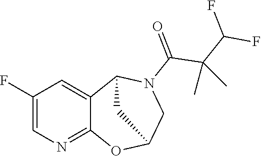 [SMILES: CC(C)(C(=O)N1C[C@@H]2C[C@H]1c1cc(F)cnc1O2)C(F)F] | 3,3-difluoro-1-((2S,5S)-7- fluoro-2,3-dihydro-2,5- methanopyrido[3,2- f][1,4]oxazepin-4(5H)-yl)- 2,2-dimethylpropan-1-one | Calculated 301, found 301 |
| 3-2 | 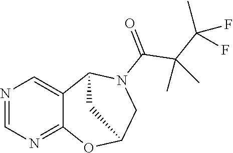 [SMILES: CC(F)(F)C(C)(C)C(=O)N1C[C@@H]2C[C@H]1c1cncnc1O2] | 1-((5S,8S)-7,8-dihydro-5,8- methanopyrimido[5,4- f][1,4]oxazepin-6(5H)-yl)- 3,3-difluoro-2,2- dimethylbutan-1-one | Calculated 298, found 298 |
| 3-3 | 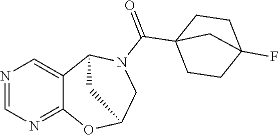 [SMILES: O=C(N1C[C@@H]2C[C@H]1c1cncnc1O2)C12CCC(F)(CC1)C2] | ((5S,8S)-7,8-dihydro-5,8- methanopyrimido[5,4- f][1,4]oxazepin-6(5H)-yl)(4- fluorobicyclo[2.2.1]heptan- 1-yl)methanone | Calculated 304, found 304 |
| 3-4 | 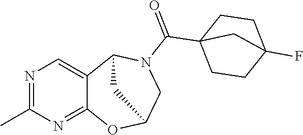 [SMILES: Cc1ncc2c(n1)O[C@H]1C[C@@H]2N(C(=O)C23CCC(F)(CC2)C3)C1] | (4- fluorobicyclo[2.2.1]heptan- 1-yl)((5S,8S)-2-methyl-7,8- dihydro-5,8- methanopyrimido[5,4- f][1,4]oxazepin-6(5H)- yl)methanone | Calculated 318, found 318 |

### Table 4 Examples

### Example 4A. Preparation of (2S,5S)-4-(4-Fluorobicyclo[2.2.1]heptane-1-carbonyl)-2,3,4,5-tetrahydro-2,5-methanopyrido[3,4-f][1,4]oxazepine-9-carbonitrile (4-3)

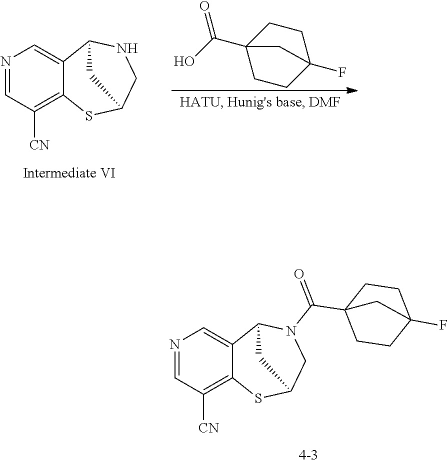 [SMILES: N#Cc1cncc2c1S[C@@H]1CN[C@H]2C1 ; O=C(O)C12CCC(F)(CC1)C2 ; N#Cc1cncc2c1S[C@H]1C[C@@H]2N(C(=O)C23CCC(F)(CC2)C3)C1]

A mixture containing 4-fluorobicyclo[2.2.1]heptane-1-carboxylic acid (14 mg, 0.089 mmol) in DMF (1 mL) was treated with Hunig's base (0.064 mL, 0.37 mmol) and HATU (56 mg, 0.15 mmol). The mixture was stirred for 15 min, then treated with Intermediate VI (15 mg, 0.074 mmol) and stirred overnight. The mixture was then purified by reverse phase chromatography (gradient of 30-60% MeCN/water with 0.1% TFA) to give (2S,5S)-4-(4-fluorobicyclo[2.2.1]heptane-1-carbonyl)-2,3,4,5-tetrahydro-2,5-methanopyrido[3,4-f][1,4]thiazepine-9-carbonitrile as a solid. ¹H NMR (500 MHz, CDCl₃) δ 8.65 (s, 1H), 8.61 (s, 1H), 5.34 (d, J=6 Hz, 1H), 4.23 (d, J=11 Hz, 1H), 4.07 (m, 1H), 3.97 (m, 1H), 2.66 (m, 1H), 2.20 (m, 1H), 1.95-2.03 (m, 6H), 1.82-1.87 (m, 4H); MS (EI) cal'd for C₁₈H₁₉FN₃OS [M+H]⁺, 344; found, 344.

Compounds 4-1, 4-2, 4-4 to 4-8 were prepared in a fashion analogous to the synthesis of 4-3, substituting 4-fluoromethylbicyclo[2.2.1]heptane-1-carboxylic acid for the corresponding carboxylic acid.

Compounds 4-9 and 4-10 were prepared as a mixture in a fashion analogous to the synthesis of 4-3, substituting 4-fluoromethylbicyclo[2.2.1]heptane-1-carboxylic acid for 2-cyano-2-methylbutanoic acid. The mixture was resolved using chiral SFC chromatography (OJ-H 250×21 mm, 5 um column; 70 mL/min of 15% MeOH/CO₂ with 0.1% NH₄OH; 215 nm detection) giving 4-9 (peak 1, 3.10 min) and 4-10 (peak 2, 3.90 min).

TABLE 4

| Compound | | | Exact Mass |
|---|---|---|---|
| Number | Structure | Name | [M + H]⁺ |
| 4-1 | 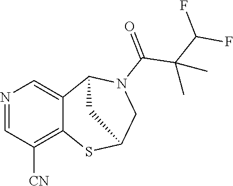 [SMILES: CC(C)(C(=O)N1C[C@@H]2C[C@H]1c1cncc(C#N)c1S2)C(F)F] | (2S,5S)-4-(3,3-difluoro-2,2- dimethylpropanoyl)-2,3,4,5- tetrahydro-2,5- methanopyrido[3,4- f][1,4]thiazepine-9-carbonitrile | Calc'd 324, found 324 |
| 4-2 | 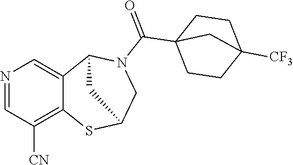 [SMILES: N#Cc1cncc2c1S[C@H]1C[C@@H]2N(C(=O)C23CCC(C(F)(F)F)(CC2)C3)C1] | (2S,5S)-4-(4- (trifluoromethyl)bicyclo[2.2.1] heptane-1-carbonyl)-2,3,4,5- tetrahydro-2,5- methanopyrido[3,4- f][1,4]thiazepine-9-carbonitrile | Calc'd 394, found 394 |
| 4-3 | 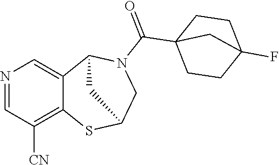 [SMILES: N#Cc1cncc2c1S[C@H]1C[C@@H]2N(C(=O)C23CCC(F)(CC2)C3)C1] | (2S,5S)-4-(4- fluorobicyclo[2.2.1]heptane-1- carbonyl)-2,3,4,5-tetrahydro- 2,5-methanopyrido[3,4- f][1,4]thiazepine-9-carbonitrile | Calc'd 344, found 344 |
| 4-4 | 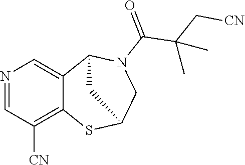 [SMILES: CC(C)(CC#N)C(=O)N1C[C@@H]2C[C@H]1c1cncc(C#N)c1S2] | (2S,5S)-4-(3-cyano-2,2- dimethylpropanoyl)-2,3,4,5- tetrahydro-2,5- methanopyrido[3,4- f][1,4]thiazepine-9-carbonitrile | Calc'd 313, found 313 |
| 4-5 | 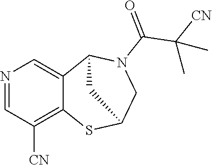 [SMILES: CC(C)(C#N)C(=O)N1C[C@@H]2C[C@H]1c1cncc(C#N)c1S2] | (2S,5S)-4-(2-cyano-2- methylpropanoyl)-2,3,4,5- tetrahydro-2,5- methanopyrido[3,4- f][1,4]thiazepine-9-carbonitrile | Calc'd 299, found 299 |
| 4-6 | 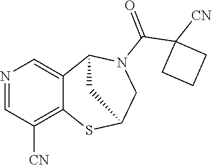 [SMILES: N#Cc1cncc2c1S[C@H]1C[C@@H]2N(C(=O)C2(C#N)CCC2)C1] | (2S,5S)-4-(1- cyanocyclobutane-1-carbonyl)- 2,3,4,5-tetrahydro-2,5- methanopyrido[3,4- f][1,4]thiazepine-9-carbonitrile | Calc'd 311, found 311 |
| 4-7 | 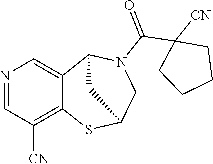 [SMILES: N#Cc1cncc2c1S[C@H]1C[C@@H]2N(C(=O)C2(C#N)CCCC2)C1] | (2S,5S)-4-(1- cyanocyclopentane-1- carbonyl)-2,3,4,5-tetrahydro- 2,5-methanopyrido[3,4- f][1,4]thiazepine-9-carbonitrile | Calc'd 325, found 325 |
| 4-8 | 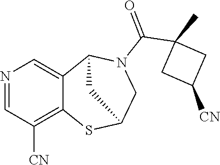 [SMILES: C[C@]1(C(=O)N2C[C@@H]3C[C@H]2c2cncc(C#N)c2S3)C[C@H](C#N)C1] | (2S,5S)-4-((1r,3S)-3-cyano-1- methylcyclobutane-1- carbonyl)-2,3,4,5-tetrahydro- 2,5-methanopyrido[3,4- f][1,4]thiazepine-9-carbonitrile | Calc'd 325, found 325 |
| 4-9 | 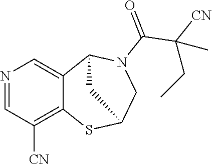 [SMILES: CCC(C)(C#N)C(=O)N1C[C@@H]2C[C@H]1c1cncc(C#N)c1S2] | (2S,5S)-4-(2-(R or S)-cyano-2- methylbutanoyl)-2,3,4,5- tetrahydro-2,5- methanopyrido[3,4- f][1,4]thiazepine-9-carbonitrile | Calc'd 313, found 313 |
| 4-10 | 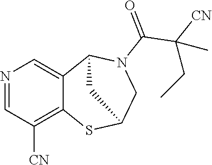 [SMILES: CCC(C)(C#N)C(=O)N1C[C@@H]2C[C@H]1c1cncc(C#N)c1S2] | (2S,5S)-4-(2-(R or S)-cyano-2- methylbutanoyl)-2,3,4,5- tetrahydro-2,5- methanopyrido[3,4- f][1,4]thiazepine-9-carbonitrile | Calc'd 313, found 313 |

### Table 5 Examples

### Example 5A. Preparation of (3R,6S)-11-(4-Fluorobicyclo[2.2.1]heptane-1-carbonyl)-3,4,5,6-tetrahydro-2H-3,6-epiminooxocino[3,2-c]pyridine-10-carbonitrile (5-1)

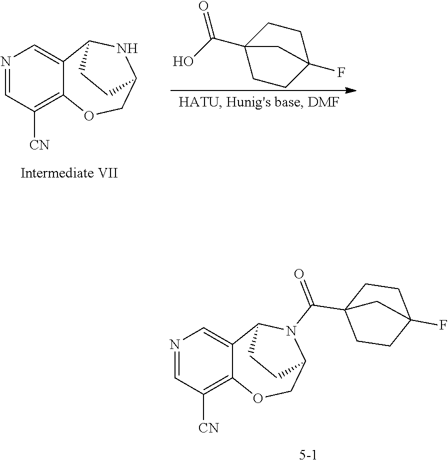 [SMILES: N#Cc1cncc2c1OC[C@H]1CC[C@@H]2N1 ; O=C(O)C12CCC(F)(CC1)C2 ; N#Cc1cncc2c1OC[C@H]1CC[C@@H]2N1C(=O)C12CCC(F)(CC1)C2]

A solution of 4-fluorobicyclo[2.2.1]heptane-1-carboxylic acid (20 mg, 0.13 mmol), HATU (57 mg, 0.15 mmol) and DIEA (0.052 mL, 0.30 mmol) in DMF (1 mL) was treated with Intermediate VII, (3R,6S)-3,4,5,6-tetrahydro-2H-3,6-epiminooxocino[3,2-c]pyridine-10-carbonitrile. (20 mg, 0.099 mmol). The mixture was stirred at 20° C. for 16 h, filtered and purified by reverse phase chromatography (27-57% MeCN/water with 10 mM NH₄HCO₃) to give (3R,6S)-11-(4-fluorobicyclo[2.2.1]heptane-1-carbonyl)-3,4,5,6-tetrahydro-2H-3,6-epiminooxocino[3,2-c]pyridine-10-carbonitrile as an oil. ¹H NMR (400 MHz, CDCl₃) δ 8.67 (br s, 2H), 5.41 (br s, 1H), 4.52-4.81 (m, 2H), 3.98 (br s, 1H), 2.36-2.47 (m, 1H), 2.05 (br d, 0.1=6 Hz, 7H), 1.85 (br s, 5H); MS (EI) cal'd for C₁₉H₂₁FN₃O₂ [M+H]⁺, 342; found, 342.

### Example 5B. Preparation of (2R,5R,10S)-4-(3,3-Difluoro-2,2-dimethylpropanoyl)-10-methyl-2,3,4,5-tetrahydro-2,5-methanopyrido[3,4-f][1,4]oxazepine-9-carbonitrile (5-3)

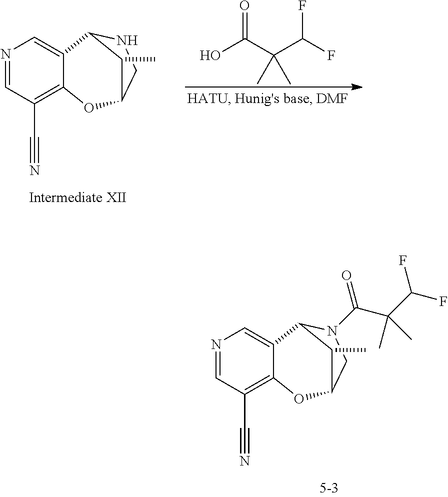

To a solution of 3,3-difluoro-2,2-dimethylpropanoic acid (24.7 mg, 0.179 mmol), HATU (85 mg, 0.22 mmol) and DIEA (0.078 mL, 0.48 mmol) in DMF (1 mL) was added Intermediate XII (30 mg, 0.15 mmol) at 25° C. Then it was stirred at 25° C. for 16 h. The mixture was filtered and purified by preparative HPLC (Instrument EJ; Method Column Boston Green ODS 150 mm*30 mm*5 um; Condition water (TFA)-ACN Begin B 33 End B 63 Gradient Time (min) 10; 100% B Hold Time (min) 2 FlowRate (mL/min) 25; Injections 1) to give (2R,5R,10S)-4-(3,3-difluoro-2,2-dimethylpropanoyl)-10-methyl-2,3,4,5-tetrahydro-2,5-methanopyrido[3,4-f][1,4]oxazepine-9-carbonitrile as a racemate. The racemate was then separated by SFC (Instrument SFC-17; Method Column DAICEL CHIRALPAK IG (250 mm*30 mm, 10 um); Condition 0.1% NH₃H₂O MEOH Begin B 50% End B 50%, FlowRate (mL/min) 80; Injections 30) to give (2R,5R,10S)-4-(3,3-difluoro-2,2-dimethylpropanoyl)-10-methyl-2,3,4,5-tetrahydro-2,5-methanopyrido[3,4-f][1,4]oxazepine-9-carbonitrile. Peak 2 was isolated as the desired isomer. ¹H NMR (400 MHz, Methanol-d₄) δ 8.57 (s, 1H), 8.46 (br s, 1H), 5.86-6.26 (m, 1H), 4.91-5.20 (m, 2H), 4.08-4.30 (m, 2H), 2.56 (q, J=6.4 Hz, 1H), 1.18-1.38 (m, 6H), 1.16 (br d, J=7.2 Hz, 2H), 1.13-1.18 (m, 1H). MS (ESI) Calculated for C₁₆H₁₈F₂N₃O₂ [M+H]⁺, 322; found, 322.

### Example 5C. Preparation of (6S,9S)-8-(3,3-Difluoro-2,2-dimethylpropanoyl)-6,7,8,9-tetrahydro-5H-6,9-methanopyrido[3,4-c]azepine-4-carbonitrile (5-5)

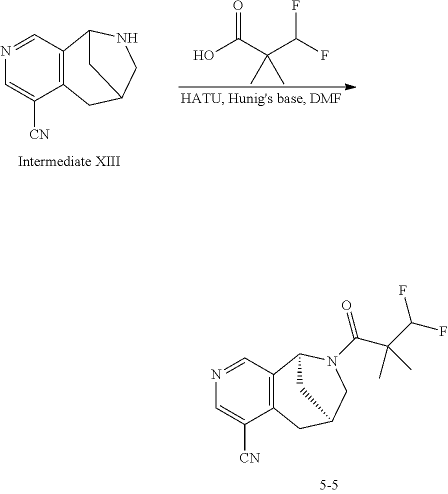

To a solution of Intermediate XIII (23 mg, 0.10 mmol) and 3,3-difluoro-2,2-dimethylpropanoic acid (25.7 mg, 0.186 mmol) in DMF (1 mL) was added DIEA (0.065 mL, 0.373 mmol) and HATU (70.8 mg, 0.186 mmol). The mixture was stirred at 20° C. for 16 h, then purified by prep-HPLC (Column Waters XSELECT C18 150 mm*30 mm*5 um; Condition water (0.1% TFA)-ACN Begin B 22 End B 52 Gradient Time (min) 10 100% B Hold Time (min) 2 FlowRate (mL/min) 25; Injections 3) to give 8-(3,3-difluoro-2,2-dimethylpropanoyl)-6,7,8,9-tetrahydro-5H-6,9-methanopyrido[3,4-c]azepine-4-carbonitrile as a racemate. The racemate was then separated by SFC (Instrument SFC-22; Method Column DAICEL CHIRALPAK AD (250 mm*30 mm, 10 um); Condition 0.1% NH₃H₂O ETOH Begin B 15% End B 15%; FlowRate (mL/min) 60) to give 8-(3,3-difluoro-2,2-dimethylpropanoyl)-6,7,8,9-tetrahydro-5H-6,9-methanopyrido[3,4-c]azepine-4-carbonitrile. Peak 2 was isolated as the desired isomer. ¹H NMR (400 MHz, Chloroform-d) δ 8.76 (s, 1H), 8.69 (s, 1H), 5.85-6.21 (m, 1H), 5.27 (d, J=5.2 Hz, 1H), 3.88-4.01 (m, 1H), 3.57 (d, J=10.0 Hz, 1H), 3.36 (br dd, J=5.2, 19.2 Hz, 1H), 2.98-3.11 (m, 2H), 2.12-2.26 (m, 1H), 1.83 (d, J=12.0 Hz, 1H), 1.31 (s, 3H), 1.14 (s, 3H). MS (ESI) Calculated for C₁₆H₁₈F₂N₃O[M+H]⁺, 306; found, 306.

Compound 5-2 was prepared in a fashion analogous to the synthesis of 5-1, substituting 4-fluoromethylbicyclo[2.2.1]heptane-1-carboxylic acid for the corresponding carboxylic acid.

Compound 5-4 was prepared in a fashion analogous to the preparation of 5-3, substituting 3,3-difluoro-2,2-dimethylpropanoic acid for the corresponding carboxylic acid. The racemate was resolved using chiral SFC chromatography (Instrument SFC-21; Method Column DAICEL CHIRALPAK AD (250 mm*30 mm, 10 um) Condition 0.1% NH₃H₂O IPA Begin B 25% End B 25%; FlowRate (mL/min) 70; Injections 60). Peak 1 was isolated as the desired isomer.

Compound 5-6 was prepared in a fashion analogous to the preparation of 5-5, substituting 3,3-difluoro-2,2-dimethylpropanoic acid for the corresponding carboxylic acid. The racemate was resolved using chiral SFC chromatography (Instrument SFC-17; Method Column DAICEL CHIRALPAK IG (250 mm*30 mm, 10 urn); Condition 0.1% NH₃H₂O ETOH Begin B 50% End B 50%; FlowRate (mL/min) 80). Peak 2 was isolated as the desired isomer.

TABLE 5

| Compound | | | Exact Mass |
|---|---|---|---|
| Number | Structure | Name | [M + H]⁺ |
| 5-1 | 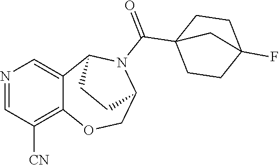 [SMILES: N#Cc1cncc2c1OC[C@H]1CC[C@@H]2N1C(=O)C12CCC(F)(CC1)C2] | (3R,6S)-11-(4- fluorobicyclo[2.2.1]heptane-1- carbonyl)-3,4,5,6-tetrahydro- 2H-3,6-epiminooxocino[3,2- c]pyridine-10-carbonitrile | Calc'd 342, found 342 |
| 5-2 | 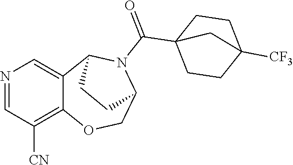 [SMILES: N#Cc1cncc2c1OC[C@H]1CC[C@@H]2N1C(=O)C12CCC(C(F)(F)F)(CC1)C2] | (3R,6S)-11-(4- (trifluoromethyl)bicyclo[2.2.1] heptane-1-carbonyl)-3,4,5,6- tetrahydro-2H-3,6- epiminooxocino[3,2- c]pyridine-10-carbonitrile | Calc'd 392, found 392 |
| 5-3 | 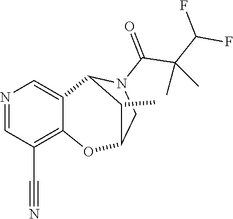 | (2R,5R,10S)-4-(3,3-difluoro- 2,2-dimethylpropanoyl)-10- methyl-2,3,4,5-tetrahydro-2,5- methanopyrido[3,4- f][1,4]oxazepine-9- carbonitrile | Calc'd 322, found 322 |
| 5-4 | 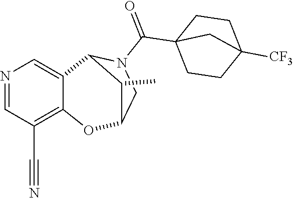 | (2R,5R,10S)-10-methyl-4-(4- (trifluoromethyl)bicyclo[2.2.1] heptane-1-carbonyl)-2,3,4,5- tetrahydro-2,5- methanopyrido[3,4- f][1,4]oxazepine-9- carbonitrile | Calc'd 392, found 392 |
| 5-5 | 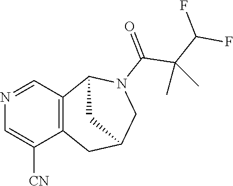 [SMILES: CC(C)(C(=O)N1C[C@H]2Cc3c(C#N)cncc3[C@@H]1C2)C(F)F] | (6S,9S)-8-(3,3-difluoro-2,2- dimethylpropanoyl)-6,7,8,9- tetrahydro-5H-6,9- methanopyrido[3,4-c]azepine- 4-carbonitrile | Calc'd 306, found 306 |
| 5-6 | 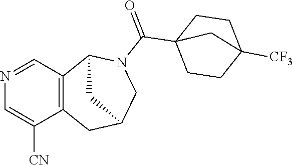 [SMILES: N#Cc1cncc2c1C[C@H]1C[C@@H]2N(C(=O)C23CCC(C(F)(F)F)(CC2)C3)C1] | (6S,9S)-8-(4- (trifluoromethyl)bicyclo[2.2.1] heptane-1-carbonyl)-6,7,8,9- tetrahydro-5H-6,9- methanopyrido[3,4-c]azepine- 4-carbonitrile | Calc'd 376, found 376 |

### RIPK1-ADP-GLO Enzymatic Assay

The enzymatic activity of RIPK1 is measured using an assay derived from ADP-Glo kit (Promega™), which provides a luminescent-based ADP detection system. Specifically, the ADP generated by RIPK1 kinase is proportionally detected as luminescent signals in a homogenous fashion. In this context, the assessment of the inhibitory effect of small molecules (EC₅₀) is measured by the effectiveness of the compounds to inhibit the ATP to ADP conversion by RIPK1.

In this assay, the potency (EC₅₀) of each compound was determined from a ten-point (1:3 serial dilution; top compound concentration of 100000 nM) titration curve using the following outlined procedure. To each well of a white ProxiPlus 384 well-plate, 30 μfIL of compound (1% DMSO in final assay volume of 3 μL) was dispensed, followed by the addition of 2 μL of 1× assay buffer (25 mM Hepes 7.3, 20 mM MgCl₂, 50 mM NaCl, 1 mM DTT, 0.005% Tween20, and 0.02% BSA) containing 37.5 nM of GST-RIPK1 (recombinant GST-RIPK1 kinase domain (residues 1-327) enzyme produced from baculovirus-transfected Sf21 cells: MW=62 kDa). Plates were placed in an ambient temperature humidified chamber for a 30 minutes pre-incubation with compound. Subsequently, each reaction was initiated by the addition of 1 μL 1×assay buffer containing 900 μM ATP and 3 μM dephosphorylated-MBP substrate. The final reaction in each well of 3 μL consists of 25 nM of GST-RIPK1, 300 μM ATP, and 3 μM dephosphorylated-MBP. Kinase reactions were allowed to proceed for 150 minutes prior to adding ADP-Glo reagents per Promega's outlined kit protocol. Dose-response curves were generated by plotting percent effect (% product conversion; Y-axis) vs. Log₁₀ compound concentrations (X-axis). EC₅₀ values were calculated using a non-linear regression, four-parameters sigmoidal dose-response model.

### RIPK1-ADP-Glo Enzymatic Assay Data

| Compound | RIPK1-ADP-Glo |
|---|---|
| Number | IC₅₀ (nM) |
| 1-1 | 32 |
| 1-2 | 7 |
| 1-3 | 50 |
| 1-4 | 13 |
| 1-5 | 22 |
| 1-6 | 11 |
| 1-7 | 18 |
| 1-8 | 29 |
| 1-9 | 20 |
| 1-10 | 21 |
| 1-11 | 62 |
| 1-12 | 47 |
| 1-13 | 130 |
| 1-14 | 51 |
| 1-15 | 99 |
| 1-16 | 10 |
| 1-17 | 24 |
| 1-18 | 84 |
| 1-19 | 24 |
| 1-20 | 89 |
| 1-21 | 366 |
| 1-22 | 55 |
| 1-23 | 88 |
| 1-24 | 288 |
| 1-25 | 119 |
| 1-26 | 313 |
| 1-27 | 221 |
| 1-28 | 201 |
| 1-29 | 376 |
| 1-30 | 66 |
| 1-31 | 54 |
| 1-32 | 290 |
| 1-33 | 94 |
| 1-34 | 24 |
| 1-35 | 483 |
| 1-36 | 973 |
| 1-37 | 136 |
| 1-38 | 21 |
| 1-39 | 31 |
| 1-40 | 62 |
| 1-41 | 27 |
| 1-42 | 114 |
| 1-43 | 491 |
| 1-44 | 254 |
| 1-45 | 330 |
| 1-46 | 78 |
| 1-47 | 22 |
| 1-48 | 5 |
| 1-49 | 70 |
| 1-50 | 300 |
| 1-51 | 367 |
| 1-52 | 116 |
| 1-53 | 226 |
| 1-54 | 116 |
| 1-55 | 18 |
| 1-56 | 41 |
| 1-57 | 35 |
| 1-58 | 74 |
| 1-59 | 587 |
| 1-60 | 230 |
| 1-61 | 34 |
| 1-62 | 444 |
| 1-63 | 58 |
| 1-64 | 729 |
| 1-65 | 76 |
| 1-66 | 514 |
| 1-67 | 640 |
| 1-68 | 259 |
| 1-69 | 77 |
| 1-70 | 35 |
| 1-71 | 232 |
| 1-72 | 35 |
| 1-73 | 501 |
| 1-74 | 347 |
| 1-75 | 545 |
| 1-76 | 780 |
| 1-77 | 139 |
| 1-78 | 77 |
| 1-79 | 446 |
| 1-80 | 332 |
| 1-81 | 93 |
| 1-82 | 13 |
| 1-83 | 30 |
| 1-84 | 47 |
| 1-85 | 19 |
| 1-86 | 33 |
| 1-87 | 68 |
| 1-88 | 96 |
| 1-89 | 43 |
| 1-90 | 85 |
| 1-91 | 64 |
| 1-92 | 84 |
| 1-93 | 88 |
| 1-94 | 15 |
| 1-95 | 30 |
| 1-96 | 148 |
| 1-97 | 92 |
| 1-98 | 57 |
| 1-99 | 48 |
| 1-100 | 28 |
| 1-101 | 132 |
| 1-102 | 76 |
| 1-103 | 163 |
| 1-104 | 203 |
| 1-105 | 92 |
| 1-106 | 60 |
| 1-107 | 23 |
| 1-108 | 269 |
| 1-109 | 241 |
| 1-110 | 129 |
| 1-111 | 184 |
| 1-112 | 252 |
| 1-113 | 156 |
| 2-1 | 184 |
| 2-2 | 139 |
| 2-3 | 8 |
| 2-4 | 10 |
| 2-5 | 18 |
| 2-6 | 24 |
| 2-7 | 26 |
| 2-8 | 394 |
| 2-9 | 41 |
| 2-10 | 227 |
| 2-11 | 57 |
| 2-12 | 124 |
| 2-13 | 1100 |
| 2-14 | 20 |
| 2-15 | 81 |
| 2-16 | 74 |
| 2-17 | 50 |
| 2-18 | 90 |
| 2-19 | 453 |
| 2-20 | 72 |
| 2-21 | 37 |
| 2-22 | 67 |
| 2-23 | 74 |
| 2-24 | 111 |
| 2-25 | 79 |
| 2-26 | 87 |
| 2-27 | 47 |
| 2-28 | 59 |
| 2-29 | 124 |
| 2-30 | 156 |
| 2-31 | 20 |
| 2-32 | 167 |
| 2-33 | 38 |
| 2-34 | 15 |
| 2-35 | 176 |
| 2-36 | 525 |
| 2-37 | 47 |
| 2-38 | 25 |
| 2-39 | 24 |
| 2-40 | 140 |
| 2-41 | 56 |
| 2-42 | 42 |
| 2-43 | 48 |
| 2-44 | 34 |
| 2-45 | 32 |
| 2-46 | 98 |
| 2-47 | 66 |
| 2-48 | 36 |
| 2-49 | 83 |
| 2-50 | 15 |
| 2-51 | 17 |
| 2-52 | 30 |
| 2-53 | 17 |
| 2-54 | 18 |
| 2-55 | 26 |
| 2-56 | 23 |
| 2-57 | 78 |
| 2-58 | 12 |
| 2-59 | 22 |
| 2-60 | 73 |
| 2-61 | 22 |
| 2-62 | 17 |
| 2-63 | 14 |
| 2-64 | 16 |
| 2-65 | 18 |
| 2-66 | 18 |
| 2-67 | 32 |
| 2-68 | 134 |
| 2-69 | 41 |
| 2-70 | 72 |
| 2-71 | 89 |
| 2-72 | 68 |
| 2-73 | 19 |
| 2-74 | 18 |
| 2-75 | 27 |
| 2-76 | 33 |
| 2-77 | 49 |
| 2-78 | 19 |
| 2-79 | 72 |
| 2-80 | 24 |
| 2-81 | 22 |
| 2-82 | 66 |
| 2-83 | 24 |
| 2-84 | 26 |
| 2-85 | 38 |
| 2-86 | 31 |
| 2-87 | 32 |
| 2-88 | 35 |
| 2-89 | 34 |
| 2-90 | 28 |
| 2-91 | 65 |
| 2-92 | 19 |
| 2-93 | 58 |
| 2-94 | 49 |
| 2-95 | 36 |
| 2-96 | 48 |
| 2-97 | 18 |
| 2-98 | 13 |
| 2-99 | 10 |
| 2-100 | 12 |
| 2-101 | 12 |
| 2-102 | 13 |
| 2-103 | 21 |
| 2-104 | 23 |
| 2-105 | 18 |
| 2-106 | 197 |
| 2-107 | 150 |
| 2-108 | 163 |
| 2-109 | 184 |
| 2-110 | 197 |
| 2-111 | 266 |
| 2-112 | 83 |
| 2-113 | 116 |
| 2-114 | 1529 |
| 2-115 | 32 |
| 2-116 | 376 |
| 2-117 | 43 |
| 2-118 | 64 |
| 2-119 | 57 |
| 2-120 | 95 |
| 2-121 | 34 |
| 2-122 | 24 |
| 2-123 | 34 |
| 3-1 | 317 |
| 3-2 | 1210 |
| 3-3 | 377 |
| 3-4 | 1230 |
| 4-1 | 24 |
| 4-2 | 15 |
| 4-3 | 12 |
| 4-4 | 42 |
| 4-5 | 99 |
| 4-6 | 49 |
| 4-7 | 27 |
| 4-8 | 117 |
| 4-9 | 61 |
| 4-10 | 43 |
| 5-1 | 95 |
| 5-2 | 114 |
| 5-3 | 432 |
| 5-4 | 165 |
| 5-5 | 1360 |
| 5-6 | 123 |

1. A compound of Formula I: 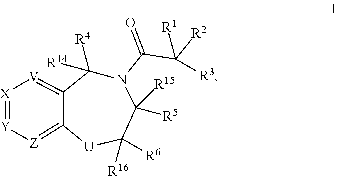
or a pharmaceutically acceptable salt thereof, wherein:
U is O, S, NR¹¹ or CR¹²R¹³;
V is N or CR⁷;
X is N or CR⁸;
Y is N or CR⁹;
Z is N or CR¹⁰; wherein at least one of V, X, Y or Z is N and wherein V, X, Y and Z are not simultaneously N;
R¹ is hydrogen, OH, C₁-C₆alkylOH, CN, C₁-C₆alkylCN, C₁-C₆alkyl, C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl) or alkoxy optionally substituted with 1-4 halogens, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁴ is hydrogen, C₁-C₆alkyl, or wherein when taken with R⁵ or R⁶ form a —CH₂—, —CH₂(CH₂)— or —CH₂CH₂— bridge;
R⁵ is hydrogen, C₁-C₆alkyl, or wherein when taken with R⁴ form a —CH₂— or —CH₂CH₂— bridge;
R⁶ is hydrogen, C₁-C₆alkyl, or wherein when taken with R⁴ form a —CH₂—, —CH₂(CH₂)— or —CH₂CH₂— bridge;
R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, C₃-C₁₀cycloalkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R¹¹ is hydrogen, C₁-C₆alkyl, or C₃-C₆cycloalkyl;
R¹² is hydrogen, OH, C₁-C₆alkylOH, CN, C₁-C₆alkylCN, C₁-C₆alkyl, haloC₁-C₆alkyl, halogen, or alkoxy;
R¹³ is hydrogen, OH, C₁-C₆alkylOH, CN, C₁-C₆alkylCN, C₁-C₆alkyl, haloC₁-C₆alkyl, halogen, or alkoxy;
R¹⁴ is hydrogen, OH, C₁-C₆alkylOH, CN, C₁-C₆alkylCN, C₁-C₆alkyl, haloC₁-C₆alkyl, halogen, or alkoxy;
R¹⁵ is hydrogen, OH, C₁-C₆alkylOH, CN, C₁-C₆alkylCN, C₁-C₆alkyl, haloC₁-C₆alkyl, halogen, or alkoxy; and
R¹⁶ is hydrogen, OH, C₁-C₆alkylOH, CN, C₁-C₆alkylCN, C₁-C₆alkyl, haloC₁-C₆alkyl, halogen, or alkoxy.

2. The compound of claim 1, wherein when U is O or CR¹²R¹³ and R⁴ forms a —CH₂— with R⁶.

3. The compound of claim 1, wherein U is O.

4. The compound of claim 1, wherein V is N, X is CH, Y is CH and Z is CR¹⁰.

5. The compound of claim 1, wherein V is CH, X is CR⁸, Y is CH and Z is N.

6. The compound of claim 1, wherein V is CR⁷, X is N, Y is CR⁹ and Z is N.

7. The compound of claim 1, wherein V is CR⁷, X is CR⁸, Y is N and Z is CH.

8. The compound of claim 1, wherein V is N, X is CH, Y is CR⁹ and Z is CH.

9. The compound of claim 1, wherein V is N, X is CR⁸, Y is N and Z is CH.

10. The compound of claim 1, wherein R¹, R² and R³ are independently selected from the group consisting of methyl, ethyl, propyl, fluoromethyl, difluoromethyl, ethenyl, propenyl, ethynyl, propynyl, cyclopropyl, cyclobutyl, fluorine, difluoroethyl, trifluoromethyl, trifluoroethyl, difluororpropyl, methoxy, CH₂phenyl, CN and CH₂CN.

11. The compound of claim 1, wherein R² is taken with R³ or R¹ and R³ and forms a C₃-C₁₀cycloalkyl or heterocycloalkyl, wherein the C₃-C₁₀cycloalkyl or heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, alkoxy, CN, C₁-C₆alkyl, aryl, C₃-C₆cycloalkyl, and haloC₁-C₆alkyl.

12. The compound of claim 1, wherein when R³ is taken with R² or R¹ and R² and forms a C₃-C₁₀cycloalkyl or heterocycloalkyl, wherein the C₃-C₁₀cycloalkyl or heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, alkoxy, CN, C₁-C₆alkyl, aryl, C₃-C₆cycloalkyl, and haloC₁-C₆alkyl.

13. The compound of claim 11, wherein the C₃-C₁₀cycloalkyl is 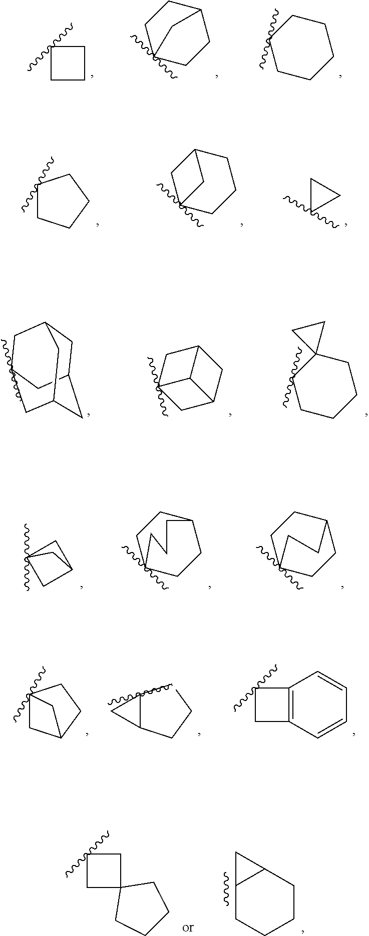
and wherein the C₃-C₁₀cycloalkyl is unsubstituted or substituted with one to four substituents selected from the group halogen, C₁-C₆alkyl, haloC₁-C₆alkyl, alkoxy, CN, COOC₁-C₆alkyl.

14. The compound of claim 1, wherein when R³ is taken with R² or R¹ and R² and forms a heterocycloalkyl, wherein the heterocycloalkyl is unsubstituted or substituted with one to four substituents selected from the group halogen, C₁-C₆alkyl, haloC₁-C₆alkyl, alkoxy, CN, COOC₁-C₆alkyl, aryl, COaryl, aryl and heteroaryl, wherein the heteroaryl is unsubstituted or substituted with one to four substituents selected from the group halogen, CN, C₁-C₆alkyl, alkoxy, C3-C6 cycloalkyl and haloC₁-C₆alkyl.

15. The compound of claim 1, wherein when R² is taken with R³ or R¹ and R³ and forms a heterocycloalkyl, wherein the heterocycloalkyl is unsubstituted or substituted with one to four substituents selected from the group halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group halogen, CN, C₁-C₆alkyl and haloC₁-C₆alkyl.

16. The compound of claim 12, wherein the heterocycloalkyl is 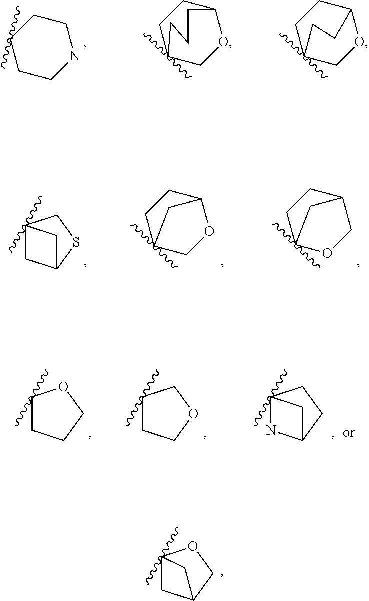
and wherein the heterocycloalkyl is unsubstituted or substituted with one to four substituents selected from the group halogen, C₁-C₆alkyl, haloC₁-C₆alkyl, alkoxy, CN, COOC₁-C₆alkyl, and heteroaryl, wherein the heteroaryl is unsubstituted or substituted with one to four substituents selected from the group halogen, CN, C₁-C₆alkyl and haloC₁-C₆alkyl.

17. The compound of claim 14, wherein the heterocycloalkyl is 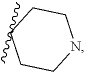
and wherein the 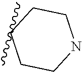
is substituted with heteroaryl, and wherein the heteroaryl is 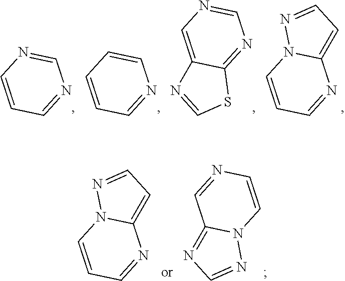
wherein the heteroaryl is unsubstituted or substituted with CN.

18. The compound of claim 1, wherein R¹, R² and R³ are independently selected from the group consisting or methyl, difluoromethyl, CH₂OCF₃, cyclobutyl, difluoroethyl, ethyl, cyclopropyl, CN, CH₂CN, CH₂phenyl and CONHCH₂phenyl, wherein the CH₂phenyl or CONHCH₂phenyl is substituted with one substituent selected from the group consisting of chlorine and methoxy.

19. A compound of Formula II: 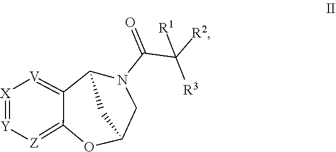
or a pharmaceutically acceptable salt thereof, wherein:
V is N or CR⁷;
X is N or CR⁸;
Y is N or CR⁹;
Z is N or CR¹⁰; wherein at least one of V, X, Y or Z is N and wherein V, X, Y and Z are not simultaneously N;
R¹ is hydrogen, OH, C₁-C₆alkylOH, CN, C₁-C₆alkylCN, C₁-C₆alkyl, C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl) or alkoxy optionally substituted with 1-4 halogens, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R⁷ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy;
R⁸ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy;
R⁹ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy; and
R¹⁰ is hydrogen, OH, C₁-C₆alkylOH, CN, C₁-C₆alkylCN, C₁-C₆alkyl, C₁-C₆alkylaryl, C₂-C₆alkynyl, haloC₁-C₆alkyl, halogen, or alkoxy.

20. A compound of Formula III: 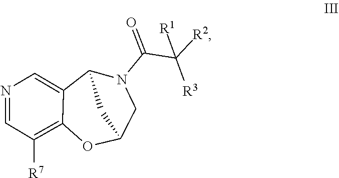
or a pharmaceutically acceptable salt thereof, wherein:
R¹ is hydrogen, OH, C₁-C₆alkylOH, CN, C₁-C₆alkylCN, C₁-C₆alkyl, C₂-C₆alkenyl, C₂-C₆alkynyl, C₃-C₆cycloalkyl, haloC₁-C₆alkyl, halogen, NH₂, N(C₁-C₆alkyl)₂, NH(C₁-C₆alkyl) or alkoxy optionally substituted with 1-4 halogens, or wherein R¹ is taken with R² and forms an oxo group, or wherein when R¹ is taken with R² or R² and R³ and forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, Oaryl, aryl and heteroaryl, wherein the aryl and heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R² is hydrogen, OH, C₁-C₆alkylOH, CN, C₁-C₆alkylCN, C₁-C₆alkyl, C₃-C₆cycloalkyl, haloC₁-C₆alkyl, halogen, NH₂, N(C₁-C₆alkyl)₂, NH(C₁-C₆alkyl) or alkoxy, or wherein R² is taken with R¹ and forms an oxo group, or wherein when R² is taken with R¹ or R¹ and R³, forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl, aryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl;
R³ is OH, C₁-C₆alkylOH, CN, C₁-C₆alkylCN, C₁-C₆alkyl, C₃-C₆cycloalkyl, haloC₁-C₆alkyl, halogen, C₁-C₆alkylOhaloC₁-C₆alkyl, alkoxy, NHC₁-C₆alkylaryl, CONHC₁-C₆alkylaryl, aryl, or C₁-C₆alkylaryl, wherein the CONHC₁-C₆alkylaryl, aryl, or C₁-C₆alkylaryl is unsubstituted or substituted with one to four substituents selected from the group consisting of halogen, C₁-C₆alkyl and haloC₁-C₆alkyl, or wherein when R³ is taken with R² or R¹ and R², forms a C₃-C₁₀cycloalkyl, aryl, heteroaryl or heterocycloalkyl, wherein the C₃-C₁₀cycloalkyl, aryl, heteroaryl or heterocycloalkyl is unsubstituted or substituted with one to four substituents selected from the group consisting of halogen, C₁-C₆alkyl, haloC₁-C₆alkyl, alkoxy, CN, COOC₁-C₆alkyl, COaryl and heteroaryl, wherein the heteroaryl is unsubstituted or substituted with one to four substituents selected from the group consisting of halogen, CN, C₁-C₆alkyl, alkoxy, aryl, C₃-C₆cycloalkyl and haloC₁-C₆alkyl; and
R⁷ is hydrogen, OH, C₁-C₆alkylOH, CN, C₁-C₆alkylCN, C₁-C₆alkyl, C₁-C₆alkylaryl, C₁-C₆alkylaryl, C₂-C₆alkynyl, haloC₁-C₆alkyl, halogen, or alkoxy, wherein the C₂-C₆alkynyl is unsubstituted or substituted with one to three substituents selected from the group consisting of OH, C₁-C₆alkylOH, CN, C₁-C₆alkylCN, NH₂, NHC₁-C₆alkyl, N(C₁-C₆alkyl)₂, haloC₁-C₆alkyl, halogen, alkoxy, haloalkoxy, aryl, C₃-C₁₀cycloalkyl, heteroaryl and heterocycloalkyl, wherein the aryl, C₃-C₁₀cycloalkyl, heteroaryl and heterocycloalkyl are unsubstituted or substituted with one to three substituents selected from the group consisting of OH, C₁-C₆alkylOH, CN, C₁-C₆alkylCN, C₁-C₆alkyl, NH₂, NHC₁-C₆alkyl, N(C₁-C₆alkyl)₂, haloC₁-C₆alkyl, halogen, alkoxy, haloalkoxy.

21. The compound of claim 20, or a pharmaceutically acceptable salt thereof, wherein R⁷ is hydrogen, CN, chlorine, bromine, fluorine, methyl, ethynyl or ethynylphenyl.

22. The compound of claim 20, or a pharmaceutically acceptable salt thereof, wherein R⁸ is hydrogen, CN, chlorine, bromine, fluorine, methyl, ethynyl or ethynylphenyl.

23. The compound of claim 20, or a pharmaceutically acceptable salt thereof, wherein R⁹ is hydrogen, CN, chlorine, bromine, fluorine, methyl, ethynyl or ethynylphenyl.

24. The compound of claim 20, or a pharmaceutically acceptable salt thereof, wherein R¹⁰ is hydrogen, CN, chlorine, bromine, fluorine, methyl, ethynyl or ethynylphenyl.

25. A compound, having the following structure: 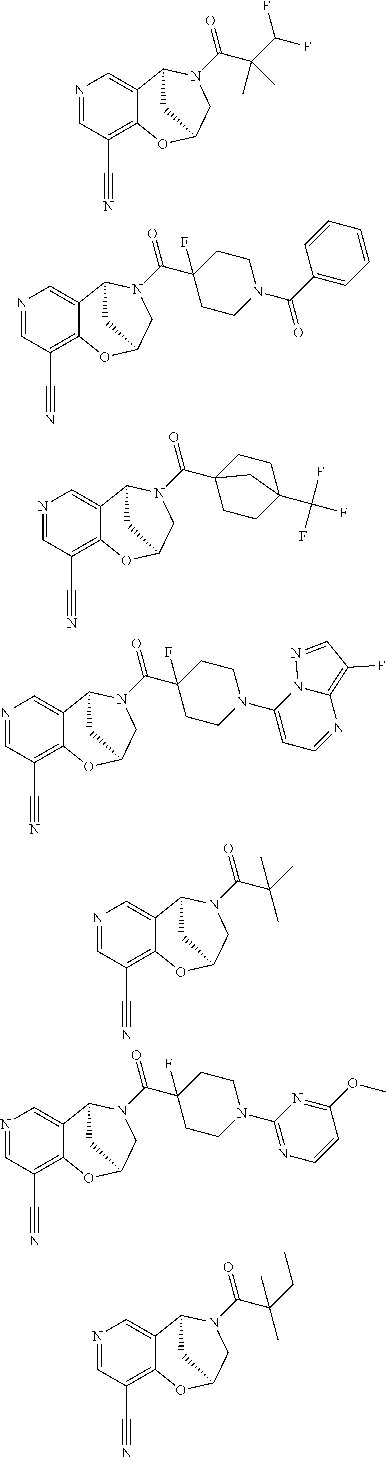 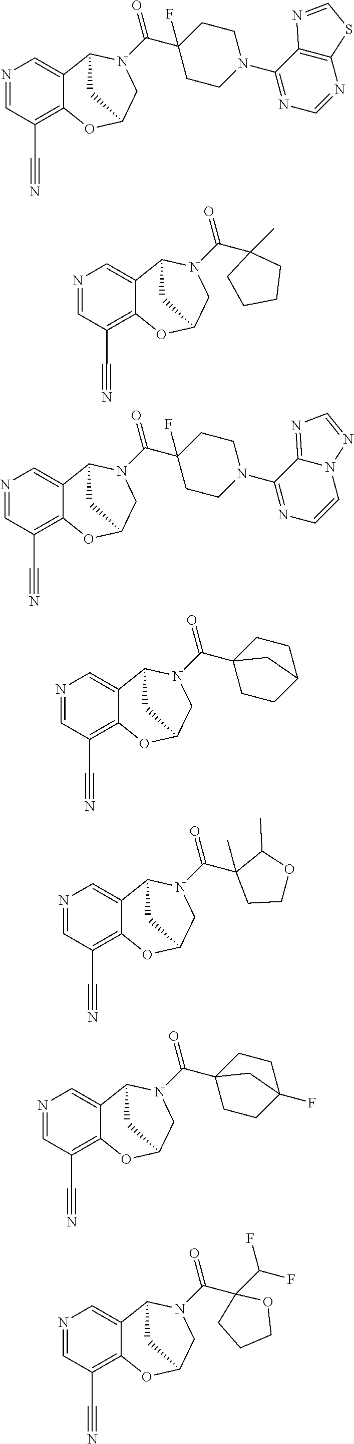 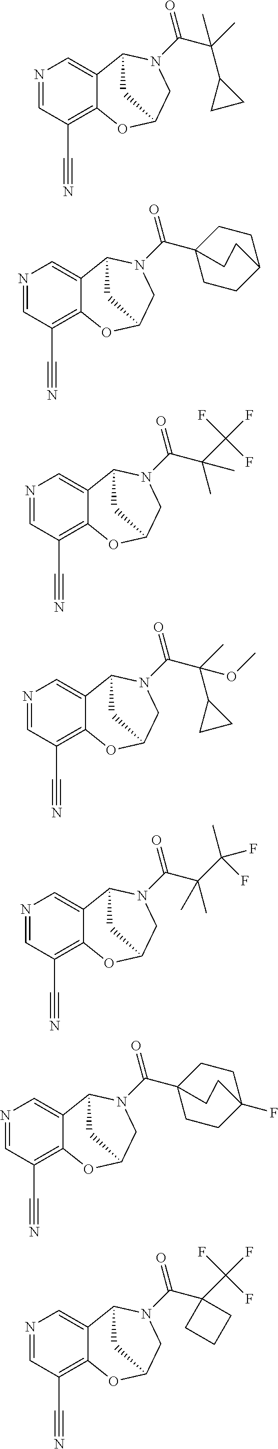 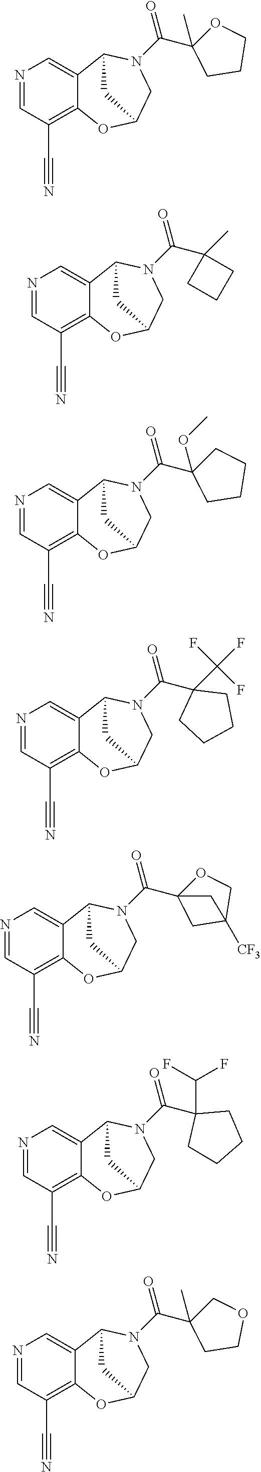 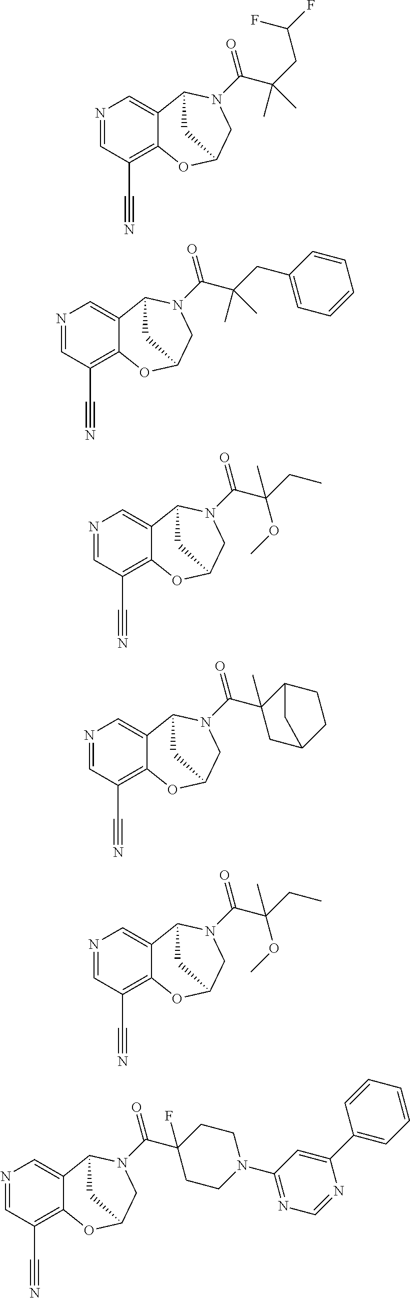 [SMILES: CC(C)(CC(F)F)C(=O)N1C[C@@H]2C[C@H]1c1cncc(C#N)c1O2 ; CC(C)(Cc1ccccc1)C(=O)N1C[C@@H]2C[C@H]1c1cncc(C#N)c1O2 ; CCC(C)(OC)C(=O)N1C[C@@H]2C[C@H]1c1cncc(C#N)c1O2 ; CC1(C(=O)N2C[C@@H]3C[C@H]2c2cncc(C#N)c2O3)CC2CCC1C2 ; CCC(C)(OC)C(=O)N1C[C@@H]2C[C@H]1c1cncc(C#N)c1O2 ; N#Cc1cncc2c1O[C@H]1C[C@@H]2N(C(=O)C2(F)CCN(c3cc(-c4ccccc4)ncn3)CC2)C1] 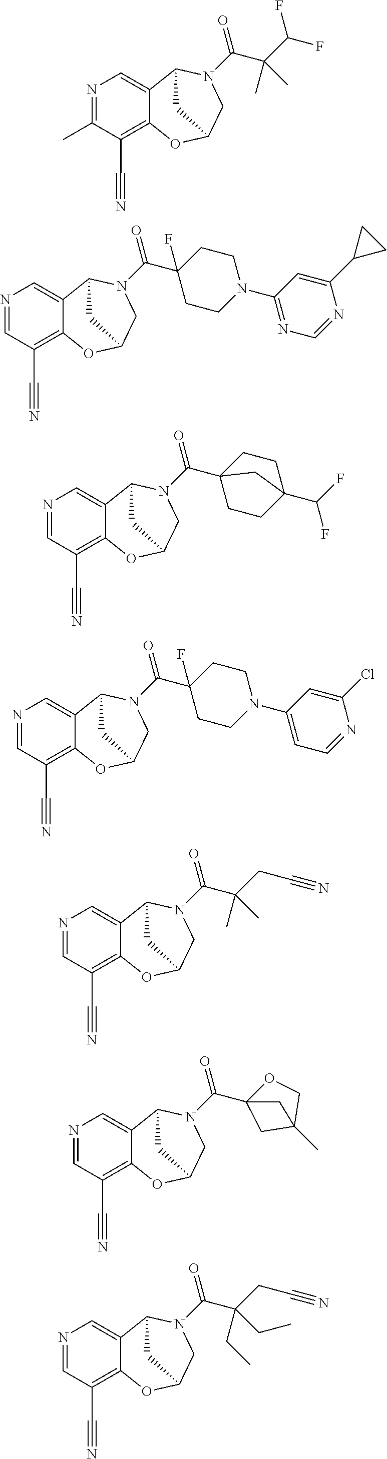 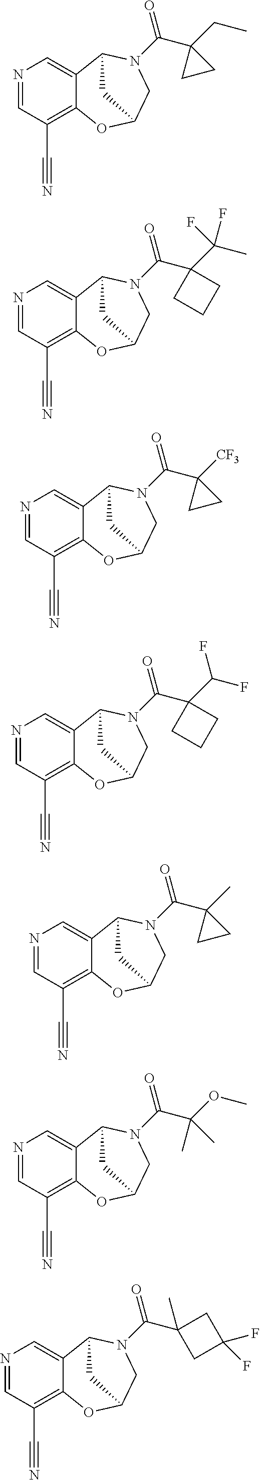 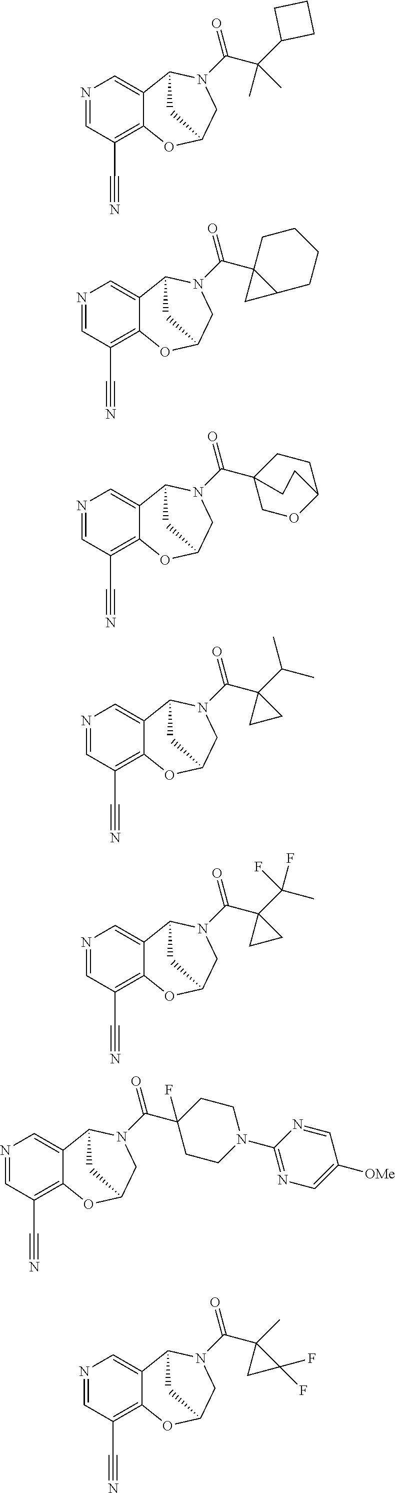 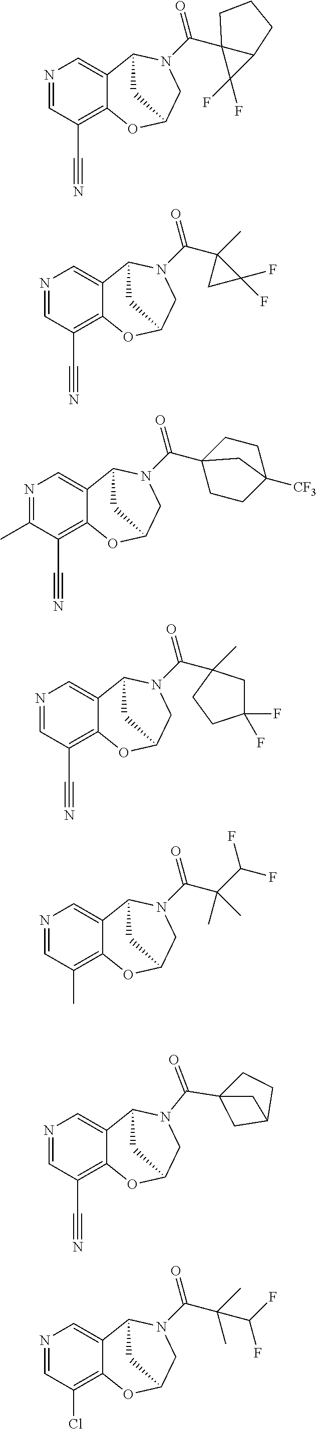 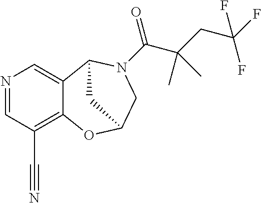 [SMILES: CC(C)(CC(F)(F)F)C(=O)N1C[C@@H]2C[C@H]1c1cncc(C#N)c1O2] 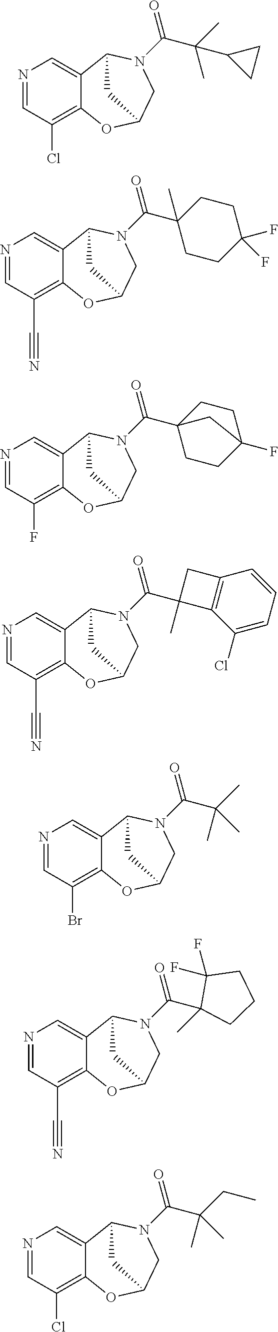 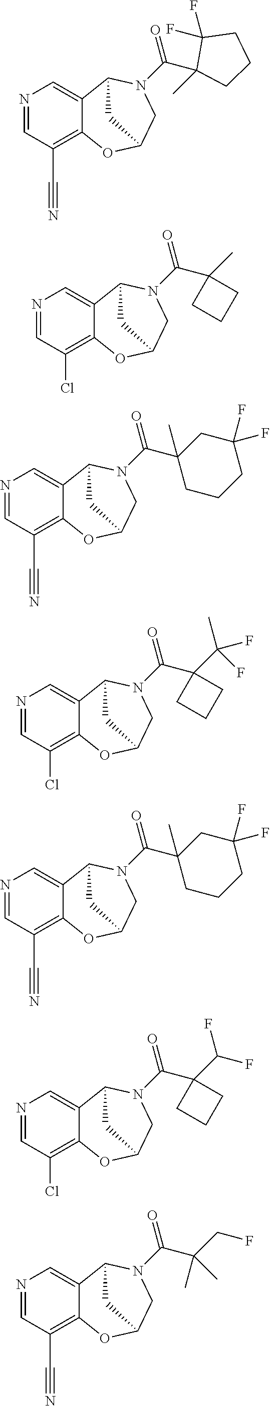 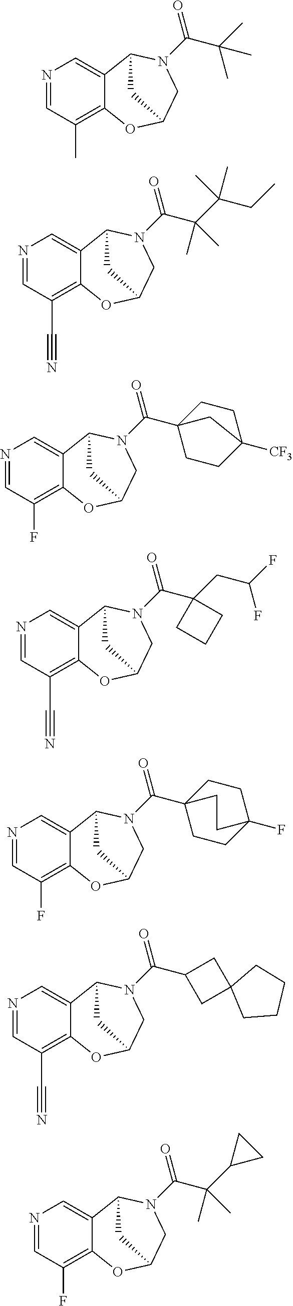 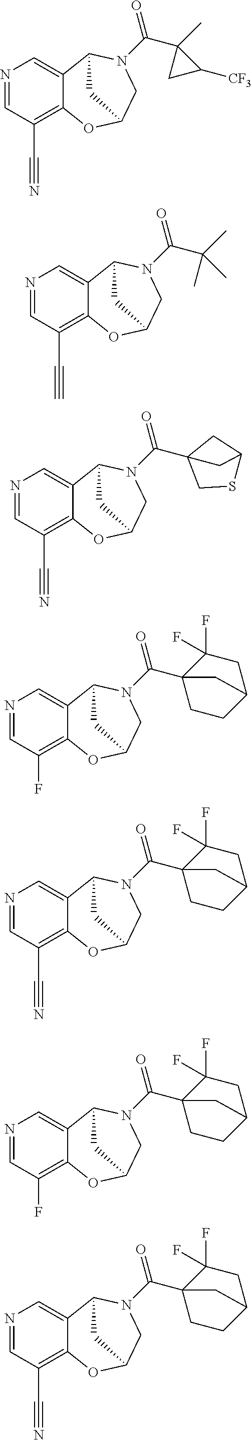 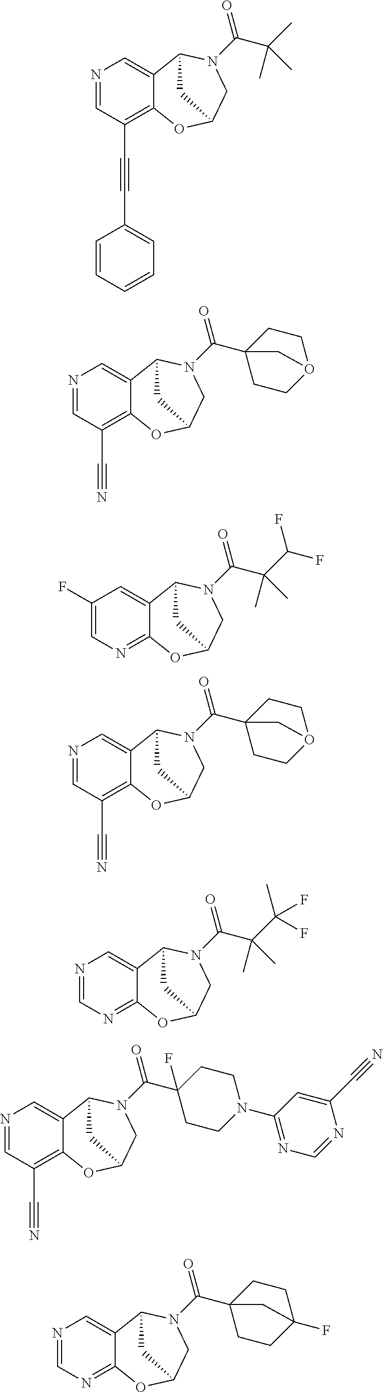 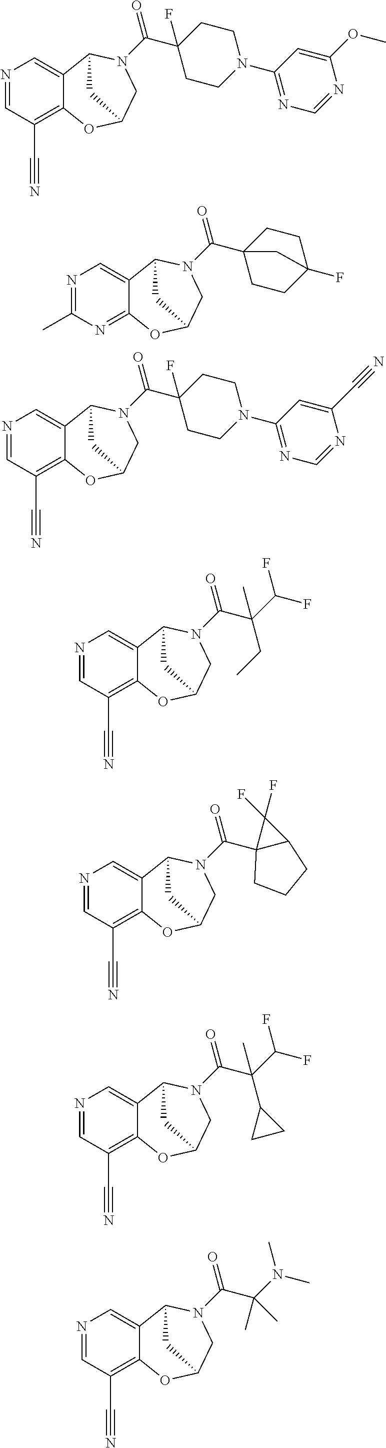 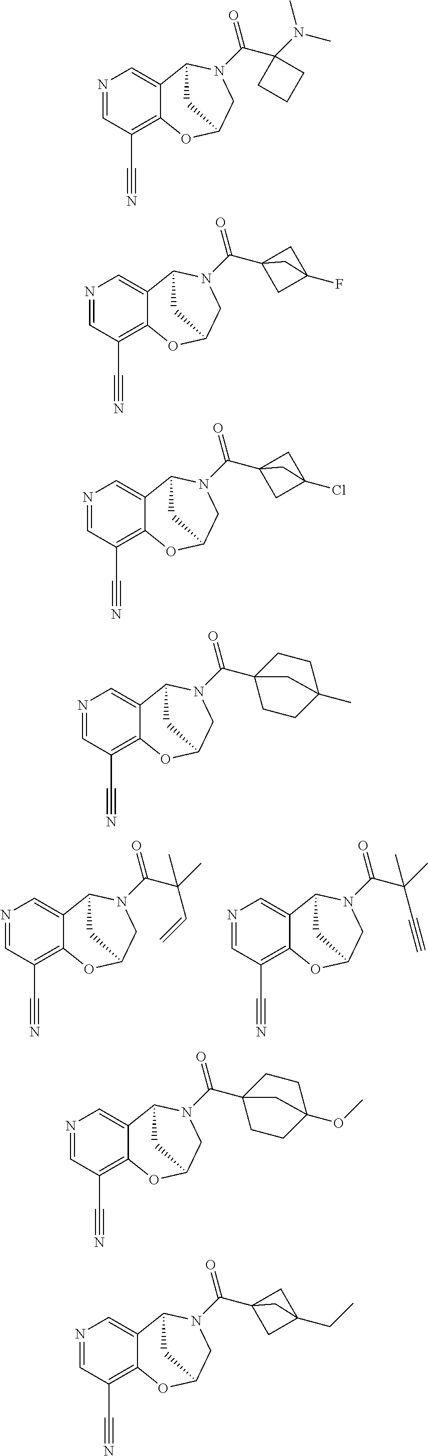 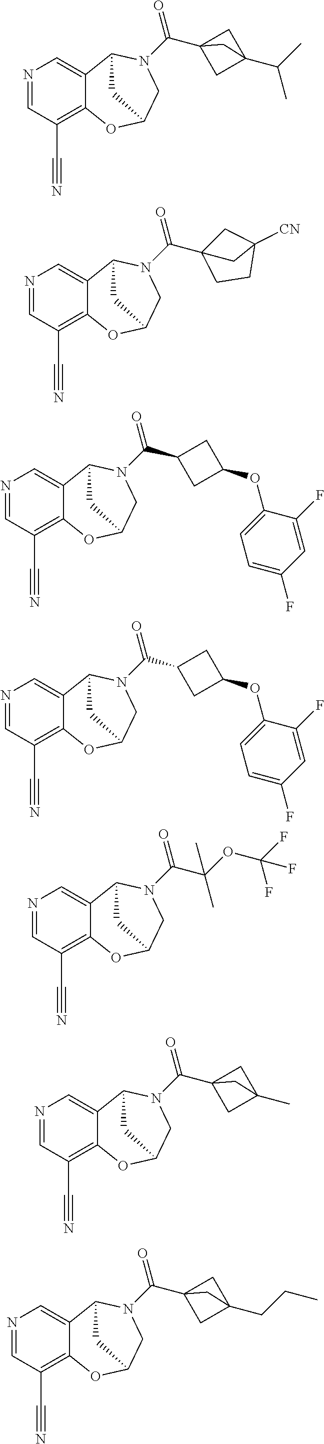 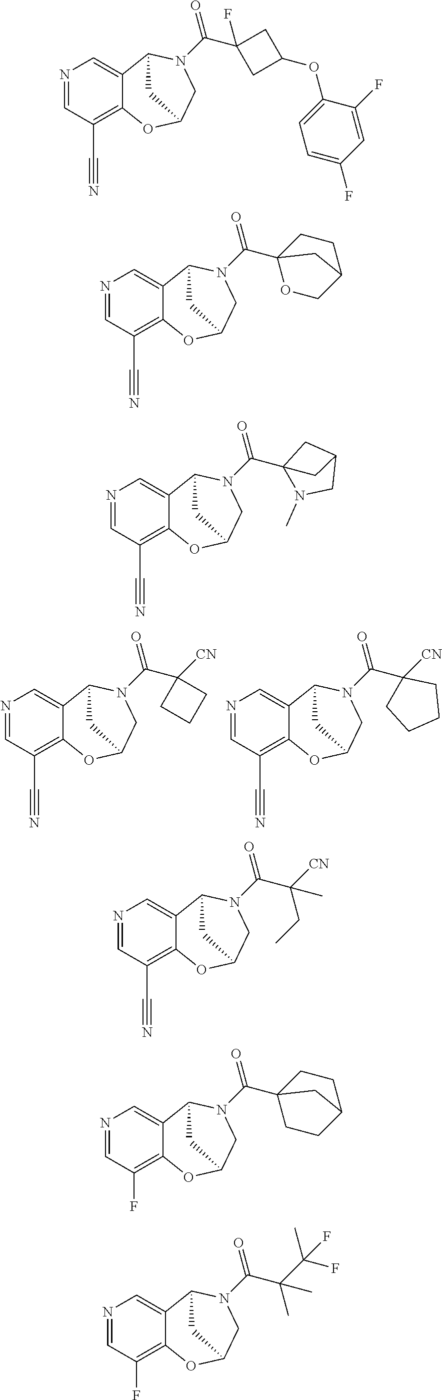 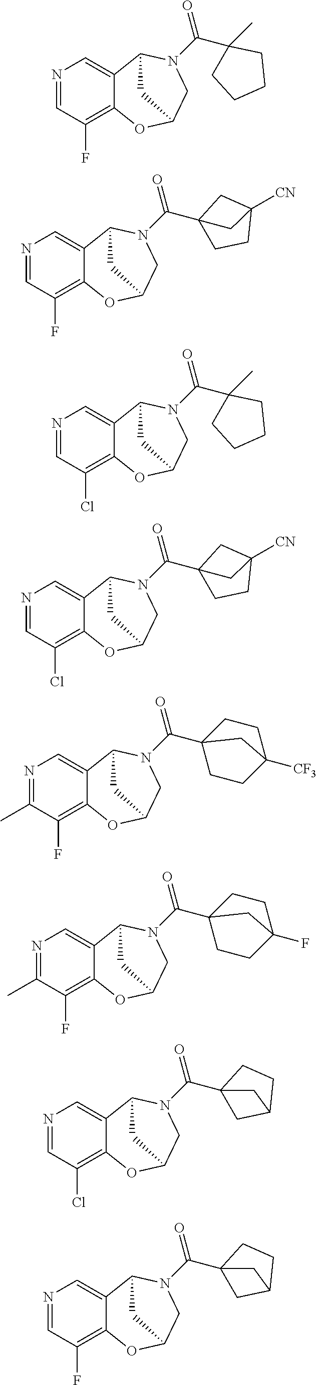 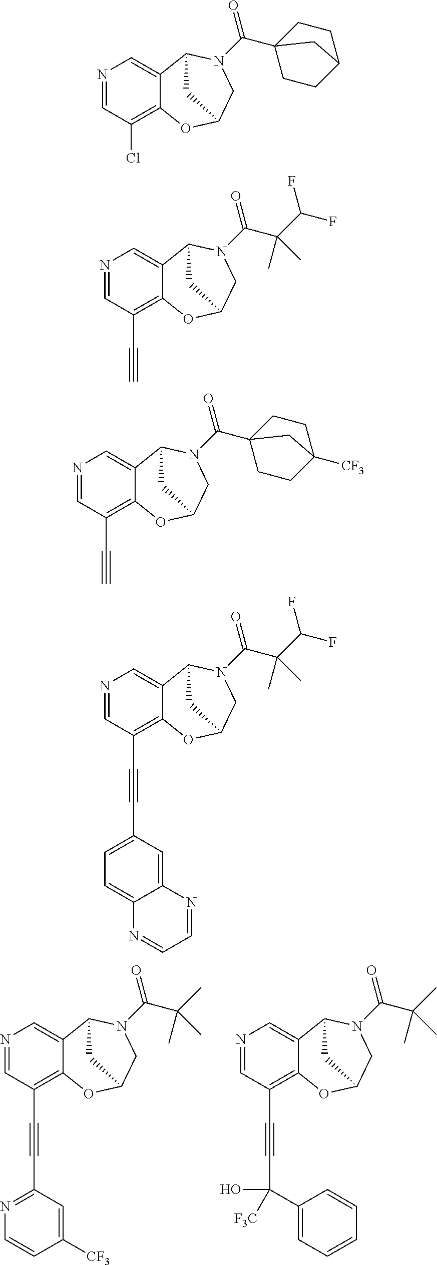 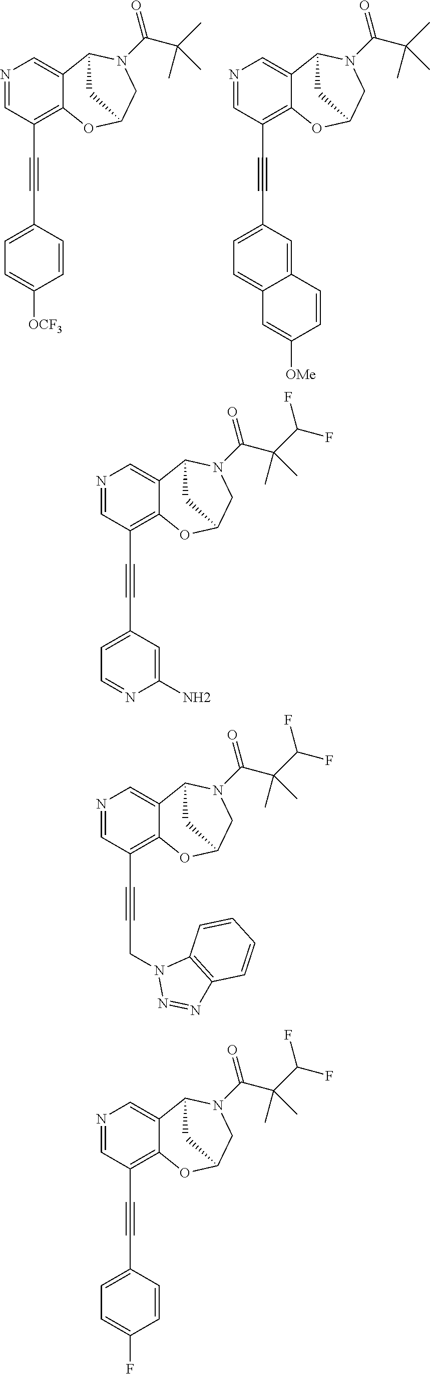 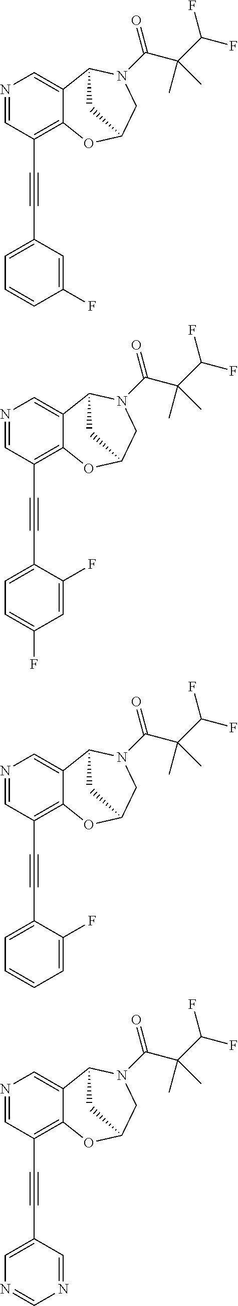 [SMILES: CC(C)(C(=O)N1C[C@@H]2C[C@H]1c1cncc(C#Cc3cccc(F)c3)c1O2)C(F)F ; CC(C)(C(=O)N1C[C@@H]2C[C@H]1c1cncc(C#Cc3ccc(F)cc3F)c1O2)C(F)F ; CC(C)(C(=O)N1C[C@@H]2C[C@H]1c1cncc(C#Cc3ccccc3F)c1O2)C(F)F ; CC(C)(C(=O)N1C[C@@H]2C[C@H]1c1cncc(C#Cc3cncnc3)c1O2)C(F)F] 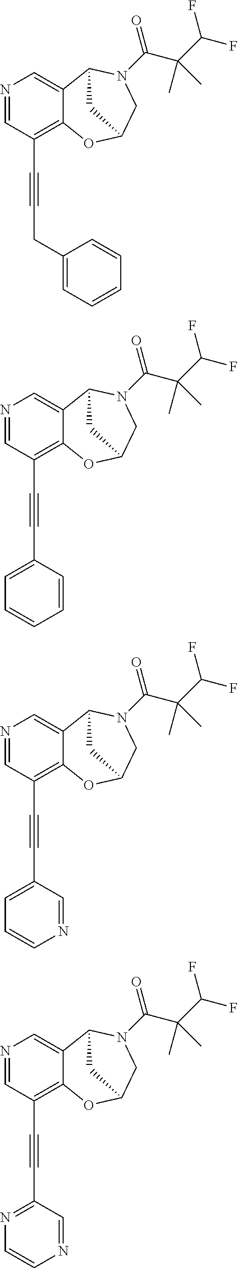 [SMILES: CC(C)(C(=O)N1C[C@@H]2C[C@H]1c1cncc(C#CCc3ccccc3)c1O2)C(F)F ; CC(C)(C(=O)N1C[C@@H]2C[C@H]1c1cncc(C#Cc3ccccc3)c1O2)C(F)F ; CC(C)(C(=O)N1C[C@@H]2C[C@H]1c1cncc(C#Cc3cccnc3)c1O2)C(F)F ; CC(C)(C(=O)N1C[C@@H]2C[C@H]1c1cncc(C#Cc3cnccn3)c1O2)C(F)F] 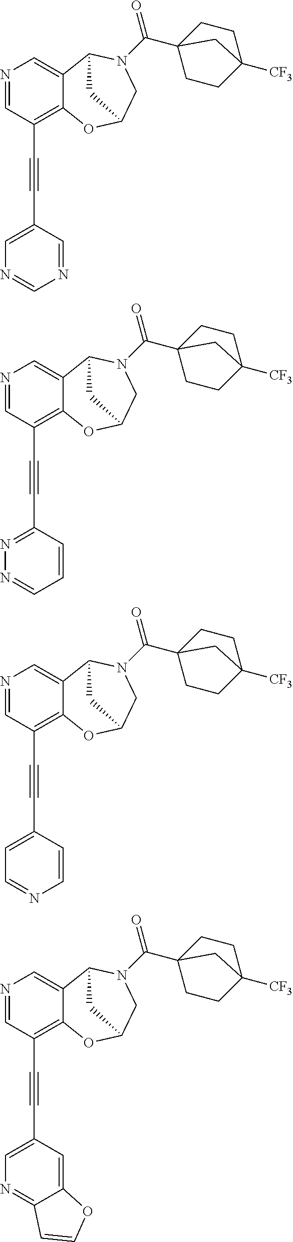 [SMILES: O=C(N1C[C@@H]2C[C@H]1c1cncc(C#Cc3cncnc3)c1O2)C12CCC(C(F)(F)F)(CC1)C2 ; O=C(N1C[C@@H]2C[C@H]1c1cncc(C#Cc3cccnn3)c1O2)C12CCC(C(F)(F)F)(CC1)C2 ; O=C(N1C[C@@H]2C[C@H]1c1cncc(C#Cc3ccncc3)c1O2)C12CCC(C(F)(F)F)(CC1)C2 ; O=C(N1C[C@@H]2C[C@H]1c1cncc(C#Cc3cnc4ccoc4c3)c1O2)C12CCC(C(F)(F)F)(CC1)C2] 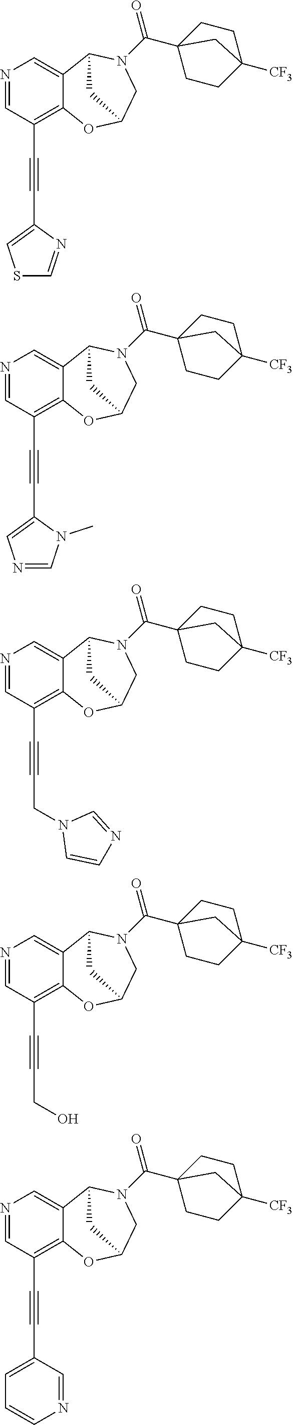 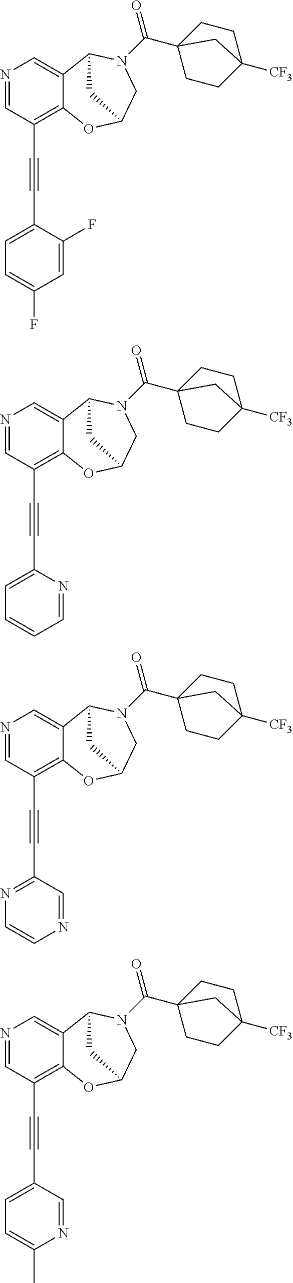 [SMILES: O=C(N1C[C@@H]2C[C@H]1c1cncc(C#Cc3ccc(F)cc3F)c1O2)C12CCC(C(F)(F)F)(CC1)C2 ; O=C(N1C[C@@H]2C[C@H]1c1cncc(C#Cc3ccccn3)c1O2)C12CCC(C(F)(F)F)(CC1)C2 ; O=C(N1C[C@@H]2C[C@H]1c1cncc(C#Cc3cnccn3)c1O2)C12CCC(C(F)(F)F)(CC1)C2 ; Cc1ccc(C#Cc2cncc3c2O[C@H]2C[C@@H]3N(C(=O)C34CCC(C(F)(F)F)(CC3)C4)C2)cn1] 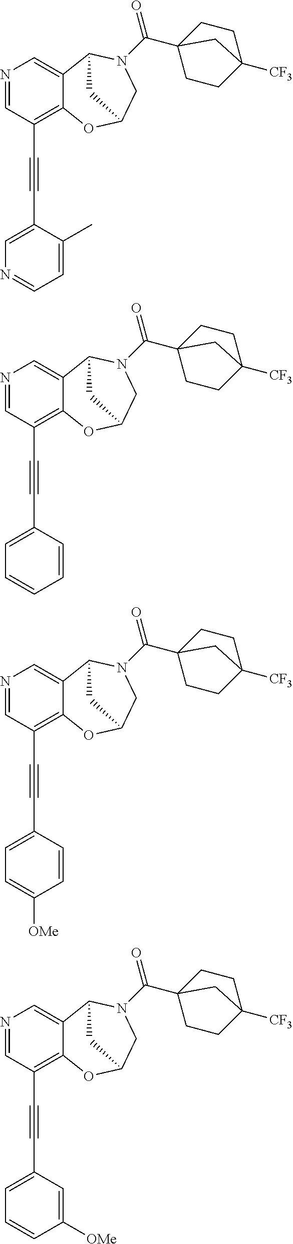 [SMILES: Cc1ccncc1C#Cc1cncc2c1O[C@H]1C[C@@H]2N(C(=O)C23CCC(C(F)(F)F)(CC2)C3)C1 ; O=C(N1C[C@@H]2C[C@H]1c1cncc(C#Cc3ccccc3)c1O2)C12CCC(C(F)(F)F)(CC1)C2 ; COc1ccc(C#Cc2cncc3c2O[C@H]2C[C@@H]3N(C(=O)C34CCC(C(F)(F)F)(CC3)C4)C2)cc1 ; COc1cccc(C#Cc2cncc3c2O[C@H]2C[C@@H]3N(C(=O)C34CCC(C(F)(F)F)(CC3)C4)C2)c1] 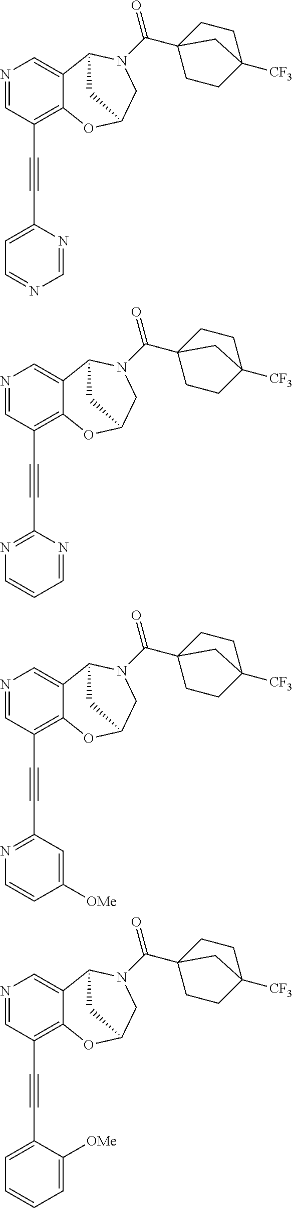 [SMILES: O=C(N1C[C@@H]2C[C@H]1c1cncc(C#Cc3ccncn3)c1O2)C12CCC(C(F)(F)F)(CC1)C2 ; O=C(N1C[C@@H]2C[C@H]1c1cncc(C#Cc3ncccn3)c1O2)C12CCC(C(F)(F)F)(CC1)C2 ; COc1ccnc(C#Cc2cncc3c2O[C@H]2C[C@@H]3N(C(=O)C34CCC(C(F)(F)F)(CC3)C4)C2)c1 ; COc1ccccc1C#Cc1cncc2c1O[C@H]1C[C@@H]2N(C(=O)C23CCC(C(F)(F)F)(CC2)C3)C1] 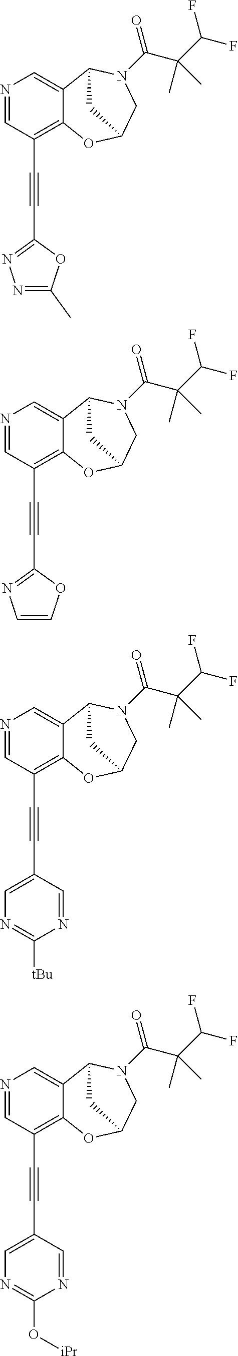 [SMILES: Cc1nnc(C#Cc2cncc3c2O[C@H]2C[C@@H]3N(C(=O)C(C)(C)C(F)F)C2)o1 ; CC(C)(C(=O)N1C[C@@H]2C[C@H]1c1cncc(C#Cc3ncco3)c1O2)C(F)F ; CC(C)(C)c1ncc(C#Cc2cncc3c2O[C@H]2C[C@@H]3N(C(=O)C(C)(C)C(F)F)C2)cn1 ; CC(C)Oc1ncc(C#Cc2cncc3c2O[C@H]2C[C@@H]3N(C(=O)C(C)(C)C(F)F)C2)cn1] 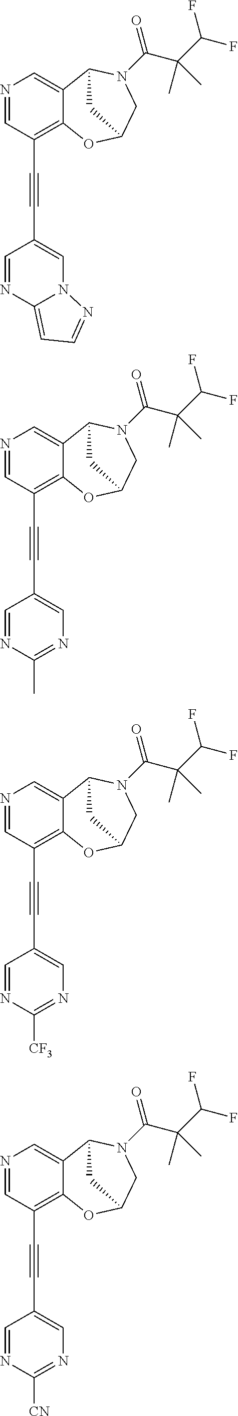 [SMILES: CC(C)(C(=O)N1C[C@@H]2C[C@H]1c1cncc(C#Cc3cnc4ccnn4c3)c1O2)C(F)F ; Cc1ncc(C#Cc2cncc3c2O[C@H]2C[C@@H]3N(C(=O)C(C)(C)C(F)F)C2)cn1 ; CC(C)(C(=O)N1C[C@@H]2C[C@H]1c1cncc(C#Cc3cnc(C(F)(F)F)nc3)c1O2)C(F)F ; CC(C)(C(=O)N1C[C@@H]2C[C@H]1c1cncc(C#Cc3cnc(C#N)nc3)c1O2)C(F)F] 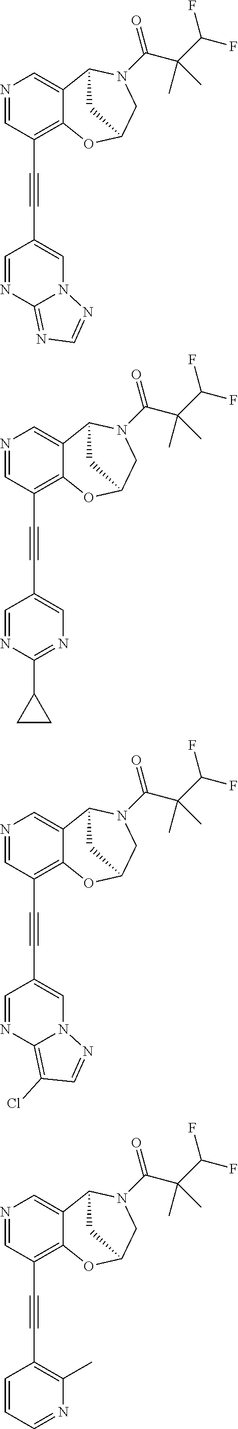 [SMILES: CC(C)(C(=O)N1C[C@@H]2C[C@H]1c1cncc(C#Cc3cnc4ncnn4c3)c1O2)C(F)F ; CC(C)(C(=O)N1C[C@@H]2C[C@H]1c1cncc(C#Cc3cnc(C4CC4)nc3)c1O2)C(F)F ; CC(C)(C(=O)N1C[C@@H]2C[C@H]1c1cncc(C#Cc3cnc4c(Cl)cnn4c3)c1O2)C(F)F ; Cc1ncccc1C#Cc1cncc2c1O[C@H]1C[C@@H]2N(C(=O)C(C)(C)C(F)F)C1] 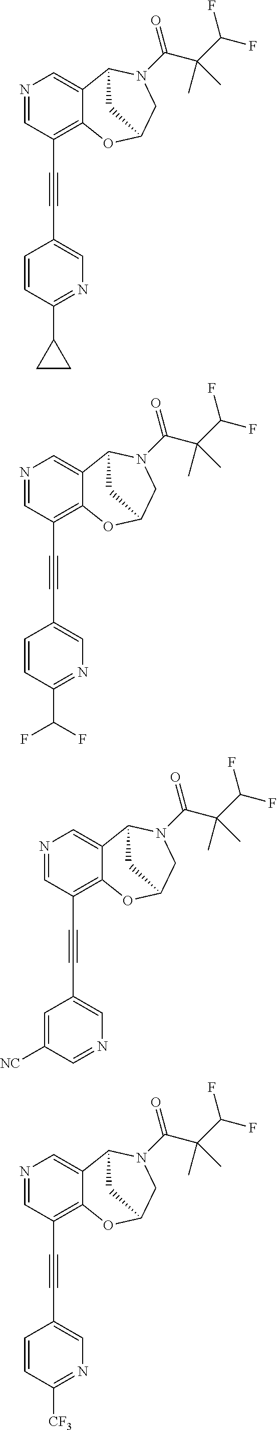 [SMILES: CC(C)(C(=O)N1C[C@@H]2C[C@H]1c1cncc(C#Cc3ccc(C4CC4)nc3)c1O2)C(F)F ; CC(C)(C(=O)N1C[C@@H]2C[C@H]1c1cncc(C#Cc3ccc(C(F)F)nc3)c1O2)C(F)F ; CC(C)(C(=O)N1C[C@@H]2C[C@H]1c1cncc(C#Cc3cncc(C#N)c3)c1O2)C(F)F ; CC(C)(C(=O)N1C[C@@H]2C[C@H]1c1cncc(C#Cc3ccc(C(F)(F)F)nc3)c1O2)C(F)F] 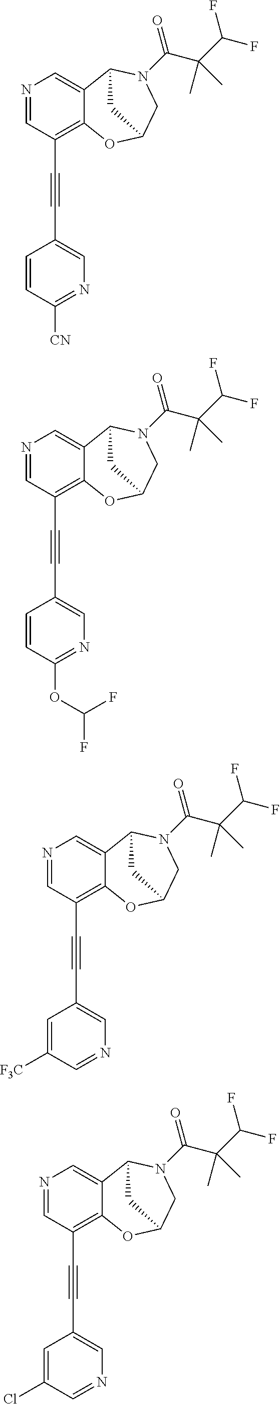 [SMILES: CC(C)(C(=O)N1C[C@@H]2C[C@H]1c1cncc(C#Cc3ccc(C#N)nc3)c1O2)C(F)F ; CC(C)(C(=O)N1C[C@@H]2C[C@H]1c1cncc(C#Cc3ccc(OC(F)F)nc3)c1O2)C(F)F ; CC(C)(C(=O)N1C[C@@H]2C[C@H]1c1cncc(C#Cc3cncc(C(F)(F)F)c3)c1O2)C(F)F ; CC(C)(C(=O)N1C[C@@H]2C[C@H]1c1cncc(C#Cc3cncc(Cl)c3)c1O2)C(F)F] 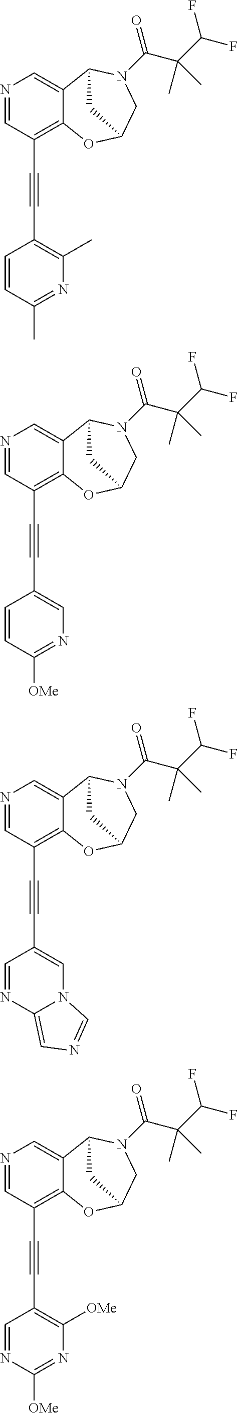 [SMILES: Cc1ccc(C#Cc2cncc3c2O[C@H]2C[C@@H]3N(C(=O)C(C)(C)C(F)F)C2)c(C)n1 ; COc1ccc(C#Cc2cncc3c2O[C@H]2C[C@@H]3N(C(=O)C(C)(C)C(F)F)C2)cn1 ; CC(C)(C(=O)N1C[C@@H]2C[C@H]1c1cncc(C#Cc3cnc4cncn4c3)c1O2)C(F)F ; COc1ncc(C#Cc2cncc3c2O[C@H]2C[C@@H]3N(C(=O)C(C)(C)C(F)F)C2)c(OC)n1] 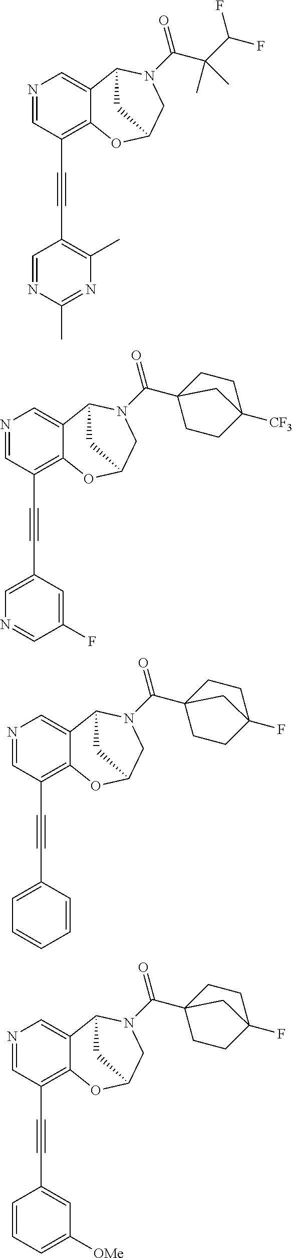 [SMILES: Cc1ncc(C#Cc2cncc3c2O[C@H]2C[C@@H]3N(C(=O)C(C)(C)C(F)F)C2)c(C)n1 ; O=C(N1C[C@@H]2C[C@H]1c1cncc(C#Cc3cncc(F)c3)c1O2)C12CCC(C(F)(F)F)(CC1)C2 ; O=C(N1C[C@@H]2C[C@H]1c1cncc(C#Cc3ccccc3)c1O2)C12CCC(F)(CC1)C2 ; COc1cccc(C#Cc2cncc3c2O[C@H]2C[C@@H]3N(C(=O)C34CCC(F)(CC3)C4)C2)c1] 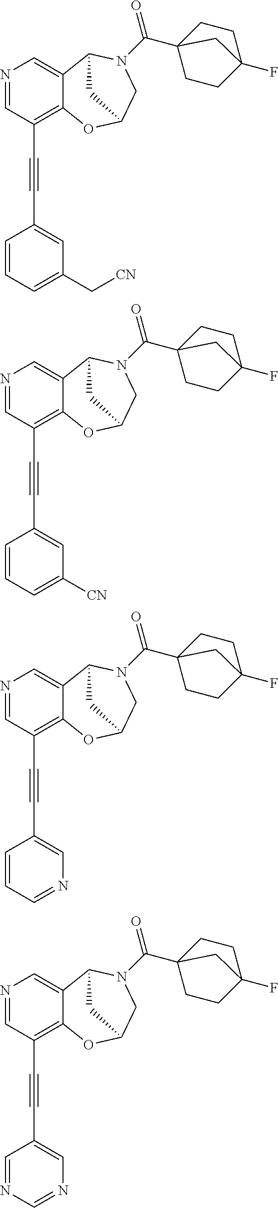 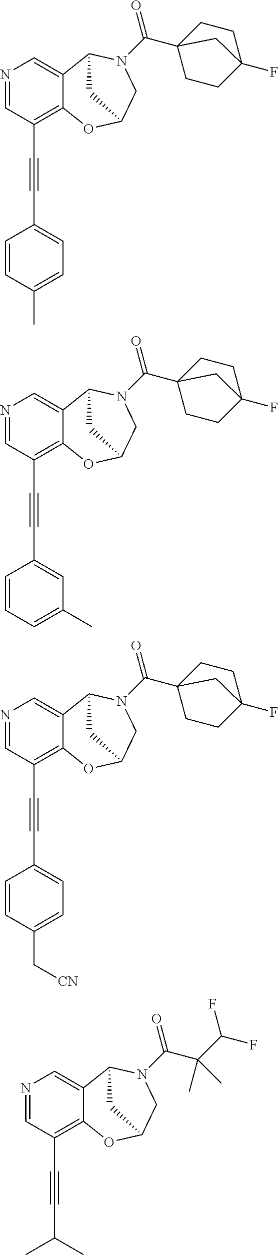 [SMILES: Cc1ccc(C#Cc2cncc3c2O[C@H]2C[C@@H]3N(C(=O)C34CCC(F)(CC3)C4)C2)cc1 ; Cc1cccc(C#Cc2cncc3c2O[C@H]2C[C@@H]3N(C(=O)C34CCC(F)(CC3)C4)C2)c1 ; N#CCc1ccc(C#Cc2cncc3c2O[C@H]2C[C@@H]3N(C(=O)C34CCC(F)(CC3)C4)C2)cc1 ; CC(C)C#Cc1cncc2c1O[C@H]1C[C@@H]2N(C(=O)C(C)(C)C(F)F)C1] 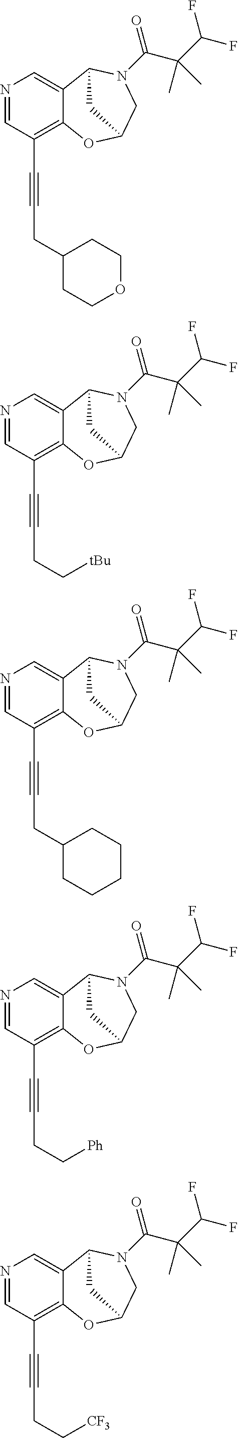 [SMILES: CC(C)(C(=O)N1C[C@@H]2C[C@H]1c1cncc(C#CCC3CCOCC3)c1O2)C(F)F ; CC(C)(C)CCC#Cc1cncc2c1O[C@H]1C[C@@H]2N(C(=O)C(C)(C)C(F)F)C1 ; CC(C)(C(=O)N1C[C@@H]2C[C@H]1c1cncc(C#CCC3CCCCC3)c1O2)C(F)F ; CC(C)(C(=O)N1C[C@@H]2C[C@H]1c1cncc(C#CCCc3ccccc3)c1O2)C(F)F ; CC(C)(C(=O)N1C[C@@H]2C[C@H]1c1cncc(C#CCCC(F)(F)F)c1O2)C(F)F] 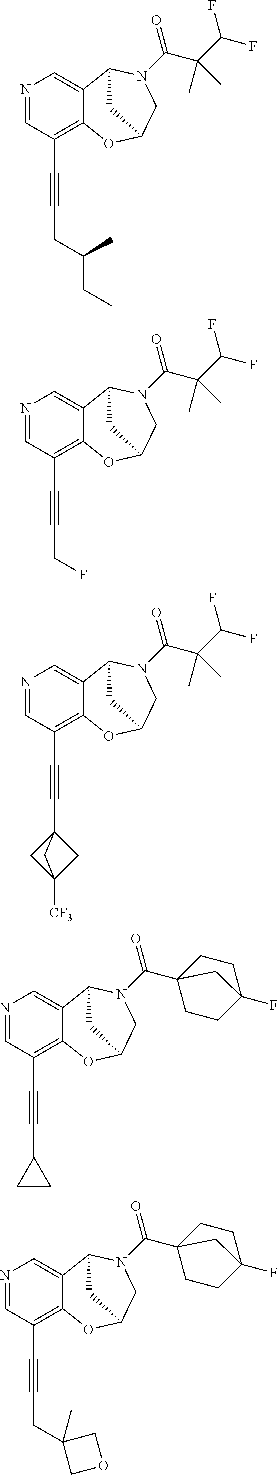 [SMILES: CC[C@H](C)CC#Cc1cncc2c1O[C@H]1C[C@@H]2N(C(=O)C(C)(C)C(F)F)C1 ; CC(C)(C(=O)N1C[C@@H]2C[C@H]1c1cncc(C#CCF)c1O2)C(F)F ; CC(C)(C(=O)N1C[C@@H]2C[C@H]1c1cncc(C#CC34CC(C(F)(F)F)(C3)C4)c1O2)C(F)F ; O=C(N1C[C@@H]2C[C@H]1c1cncc(C#CC3CC3)c1O2)C12CCC(F)(CC1)C2 ; CC1(CC#Cc2cncc3c2O[C@H]2C[C@@H]3N(C(=O)C34CCC(F)(CC3)C4)C2)COC1] 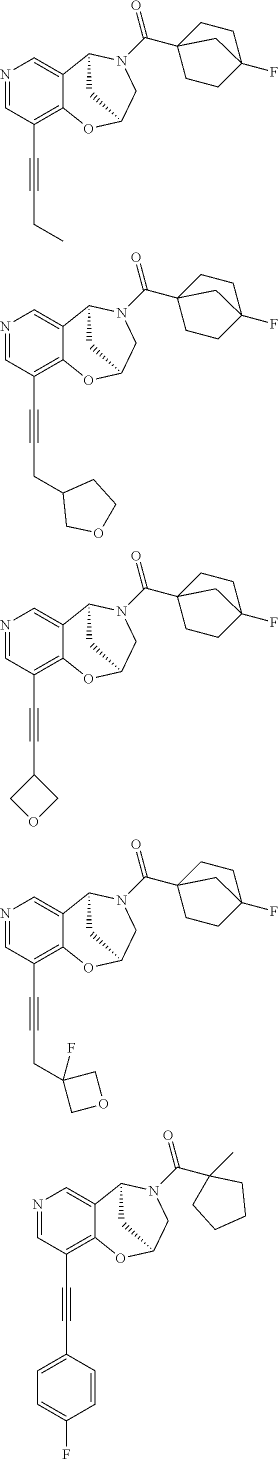 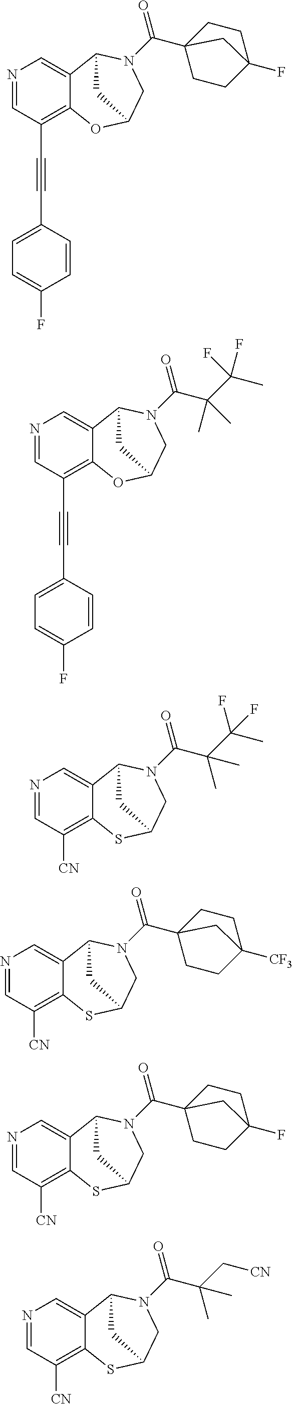 [SMILES: O=C(N1C[C@@H]2C[C@H]1c1cncc(C#Cc3ccc(F)cc3)c1O2)C12CCC(F)(CC1)C2 ; CC(F)(F)C(C)(C)C(=O)N1C[C@@H]2C[C@H]1c1cncc(C#Cc3ccc(F)cc3)c1O2 ; CC(F)(F)C(C)(C)C(=O)N1C[C@@H]2C[C@H]1c1cncc(C#N)c1S2 ; N#Cc1cncc2c1S[C@H]1C[C@@H]2N(C(=O)C23CCC(C(F)(F)F)(CC2)C3)C1 ; N#Cc1cncc2c1S[C@H]1C[C@@H]2N(C(=O)C23CCC(F)(CC2)C3)C1 ; CC(C)(CC#N)C(=O)N1C[C@@H]2C[C@H]1c1cncc(C#N)c1S2] 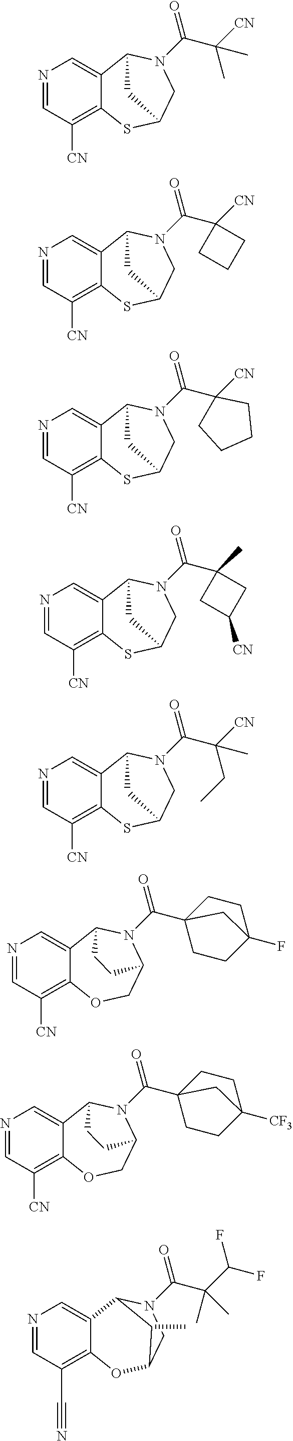 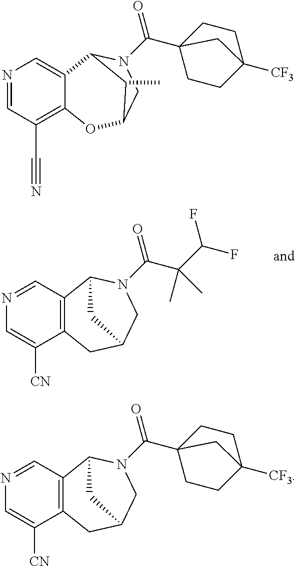
or a pharmaceutically acceptable salt thereof.

26. A method for treating RIPK1 dependent inflammation and cell death that occurs in inherited and sporadic diseases including Alzheimer's disease, amyotrophic lateral sclerosis, multiple sclerosis, Parkinson's disease, chronic traumatic encephalopathy, rheumatoid arthritis, ulcerative colitis, inflammatory bowel disease, psoriasis as well as acute tissue injury caused by stroke, traumatic brain injury, encephalitis comprising administering to a patient in need thereof a compound, or pharmaceutically acceptable salt thereof, of claim 1.

27. A method of treating amyotrophic lateral sclerosis comprising administering to a patient in need thereof a compound, or pharmaceutically acceptable salt thereof, of claim 1.

28. (canceled)

29. A pharmaceutical composition comprising a compound of claim 1, or a pharmaceutically acceptable salt thereof and a pharmaceutically acceptable carrier.

30. (canceled)

31. The compound of claim 12, wherein the C₃-C₁₀cycloalkyl is 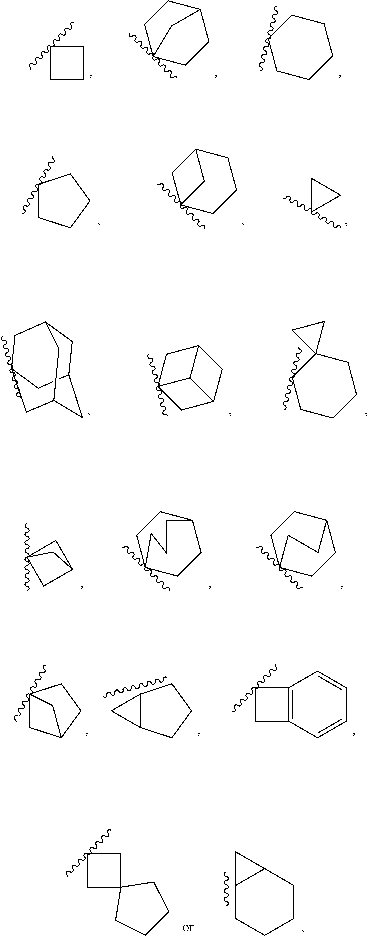
and wherein the C₃-C₁₀cycloalkyl is unsubstituted or substituted with one to four substituents selected from the group halogen, C₁-C₆alkyl, haloC₁-C₆alkyl, alkoxy, CN, COOC₁-C₆alkyl.
